(19) United States
(12) Patent Application Publication
(10) Pub. No.: US 2025/0032476 A1
(43) Pub. Date: Jan. 30, 2025

(54) ALPHA-2A ADRENERGIC RECEPTOR MODULATORS AND USES THEREOF

(71) Applicants: The Regents of the University of California, Oakland, CA (US); Friedrich-Alexander-Universität Erlangen-Nürnberg, Erlangen (DE); Chinese University of Hong Kong, Shenzhen, Shenzhen (CN)

(72) Inventors: Brian K. Shoichet, Kentfield, CA (US); Elissa A. Fink, San Francisco, CA (US); Allan I. Basbaum, San Rafael, CA (US); Joao M. Braz, San Francisco, CA (US); Peter Gmeiner, Buckenhof (DE); Harald Hübner, Heroldsbach (DE); Philipp Seemann, Erlangen (DE); Tara Pfeiffer, Erlangen (DE); Yang Du, Shenzhen (CN); Jun XU, Palo Alto, CA (US)

(21) Appl. No.: 18/707,537

(22) PCT Filed: Nov. 4, 2022
(86) PCT No.: PCT/US2022/079276

§ 371 Date: May 3, 2024

Related U.S. Application Data
(60) Provisional application No. 63/276,399, filed on Nov. 5, 2021.
(60) Provisional application No. 63/410,577, filed on Sep. 27, 2022.
(60) Provisional application No. 63/410,578, filed on Sep. 27, 2022.
(60) Provisional application No. 63/410,580, filed on Sep. 27, 2022.

(51) Int. Cl. A61K 31/4725; A61K 31/195; A61K 31/4409; A61K 31/4436; A61K 31/445; A61K 31/4709; A61P 25/04; C07D 213/68; C07D 213/74; C07D 401/04; C07D 401/12; C07D 409/12; C07D 471/04

(52) U.S. Cl. CPC A61K 31/4725; A61K 31/195; A61K 31/4409; A61K 31/4436; A61K 31/445; A61K 31/4709; A61P 25/04; C07D 213/68; C07D 213/74; C07D 401/04; C07D 401/12; C07D 409/12; C07D 471/04

(57) ABSTRACT

Described herein, inter alia, are 0.2 A adrenergic receptor agonists and uses thereof.

### CROSS-REFERENCES TO RELATED APPLICATIONS

This application claims the benefit of U.S. Provisional Application No. 63/410,580, filed Sep. 27, 2022, U.S. Provisional Application No. 63/410,578, filed Sep. 27, 2022, U.S. Provisional Application No. 63/410,577, filed Sep. 27, 2022, and U.S. Provisional Application No. 63/276,399, filed Nov. 5, 2021, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under grant no. HR0011-19-2-0020 awarded by The Defense Advanced Research Projects Agency, grant no. R35 GM122481 awarded by The National Institutes of Health, and grant no. R35 NS097306 awarded by The National Institutes of Health. The government has certain rights in the invention.

### REFERENCE TO AN ELECTRONIC SEQUENCE LISTING

The contents of the electronic sequence listing (048536-719001WO_Sequence_Listing_ST26.xml; Size: 2,606 bytes; and Date of Creation: Oct. 27, 2022) is hereby incorporated by reference in its entirety.

### BACKGROUND

New therapeutics acting through non-opioid receptors are much sought after as novel analgesics. Among these is the α_{2A}-adrenergic receptor (α_{2A}AR), the primary target of dexmedetomidine, widely used in hospital settings but otherwise restricted owing to its sedative properties and its intravenous dosing. Disclosed herein, inter alia, are solutions to these and other problems in the art.

### BRIEF SUMMARY

In an aspect is provided a compound, or a pharmaceutically acceptable salt thereof, having the formula:

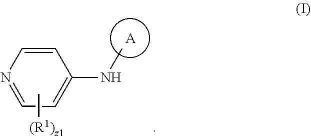

Ring A is substituted or unsubstituted cycloalkyl, substituted or unsubstituted heterocycloalkyl, substituted or unsubstituted aryl, or substituted or unsubstituted heteroaryl.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl.

X¹ is independently —F, —Cl, —Br, or —I.

The symbol n1 is an integer from 0 to 4. The symbols m1 and v1 are independently 1 or 2. The symbol z1 is an integer from 0 to 4.

In an aspect is provided a compound, or a pharmaceutically acceptable salt thereof, having the formula:

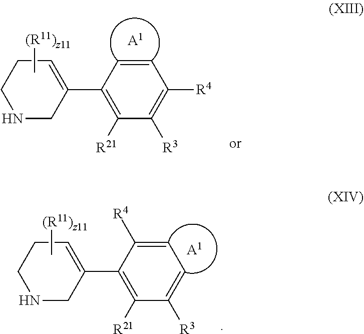

Ring A¹ is a substituted or unsubstituted cycloalkyl, substituted or unsubstituted heterocycloalkyl, substituted or unsubstituted aryl, or substituted or unsubstituted heteroaryl.

R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹¹ substituents may optionally be joined to form a substituted or unsubstituted cycloalkyl, substituted or unsubstituted heterocycloalkyl, substituted or unsubstituted aryl, or substituted or unsubstituted heteroaryl.

R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R²¹ and R³ substituents may optionally be joined to form a substituted or unsubstituted cycloalkyl, substituted or unsubstituted heterocycloalkyl, substituted or unsubstituted aryl, or substituted or unsubstituted heteroaryl; or R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z11 is an integer from 0 to 8.

In an aspect is provided a compound, or a pharmaceutically acceptable salt thereof, having the formula:

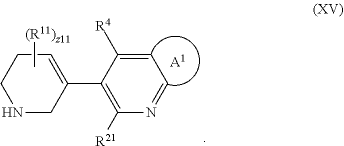

Ring A¹, R¹¹, z11, R²¹, and R⁴ are as described herein, including in embodiments.

In an aspect is provided a compound, or a pharmaceutically acceptable salt thereof, having the formula:

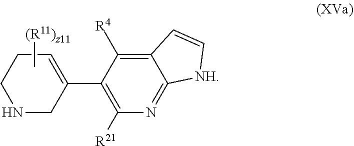

R¹¹, z11, R²¹, and R⁴ are as described herein, including in embodiments.

In an aspect is provided a compound, or a pharmaceutically acceptable salt thereof, having the formula:

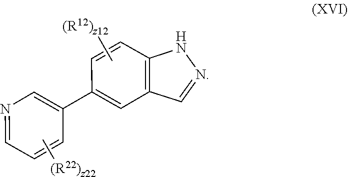

R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹² substituents may optionally be joined to form a substituted or unsubstituted cycloalkyl, substituted or unsubstituted heterocycloalkyl, substituted or unsubstituted aryl, or substituted or unsubstituted heteroaryl.

R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R²² substituents may optionally be joined to form a substituted or unsubstituted cycloalkyl, substituted or unsubstituted heterocycloalkyl, substituted or unsubstituted aryl, or substituted or unsubstituted heteroaryl.

The symbol z12 is an integer from 0 to 5. The symbol z22 is an integer from 0 to 4.

In an aspect is provided a compound, or a pharmaceutically acceptable salt thereof, having the formula:

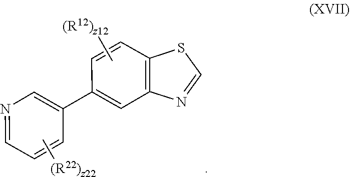

R¹², z12, R²², and z22 are as described herein, including in embodiments.

In an aspect is provided a compound, or a pharmaceutically acceptable salt thereof, having the formula:

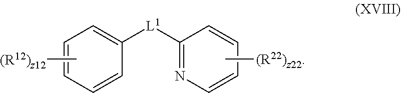

L¹ is —O—, —NR¹⁰—, or substituted or unsubstituted alkylene.

R¹⁰ is hydrogen or unsubstituted C₁-C₄ alkyl.

R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹² substituents may optionally be joined to form a substituted or unsubstituted cycloalkyl, substituted or unsubstituted heterocycloalkyl, substituted or unsubstituted aryl, or substituted or unsubstituted heteroaryl.

R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R²² substituents may optionally be joined to form a substituted or unsubstituted cycloalkyl, substituted or unsubstituted heterocycloalkyl, substituted or unsubstituted aryl, or substituted or unsubstituted heteroaryl.

The symbol z12 is an integer from 0 to 5. The symbol z22 is an integer from 0 to 4.

In an aspect is provided a pharmaceutical composition including a compound described herein, or a pharmaceutically acceptable salt thereof, and a pharmaceutically acceptable excipient.

In an aspect is provided a method of treating pain in a subject in need thereof, the method including administering to the subject in need thereof a therapeutically effective amount of a compound described herein, or a pharmaceutically acceptable salt thereof.

In an aspect is provided a method of increasing the level of activity of α_{2A} adrenergic receptor in a cell, said method comprising contacting the cell with an effective of a compound described herein, or a pharmaceutically acceptable salt thereof.

### BRIEF DESCRIPTION OF THE DRAWINGS

FIGS. 1A-1D. New α_{2A}AR agonists from ultra-large library docking. FIG. 1A: 301 million molecules were docked against the active state of α_{2B}AR. Lead-like molecules often spilled out of the orthosteric site, while fragment molecules are well-complemented by that site. FIG. 1B: The αAR pharmacophore model (9) overlaid on known α_{2A}AR agonists dexmedetomidine, clonidine, and norepinephrine and new agonists from docking. FIG. 1C: Gᵢ activation and β-arrestin-2 recruitment for norepinephrine (NorEpi), dexmedetomidine (dex), and clonidine (clon), and potent new docking agonists. FIG. 1D: Docked poses of potent docking agonists with hydrogen bonds to key recognition residues shown as black dashed lines. For FIG. 1C: Data are mean±s.e.m. of normalized results (n=4 to 17 measurements for Gᵢ and n=3 to 8 measurements for β-arrestin-2).

FIGS. 2A-2D. Docking-predicted poses of ‘9087 and ‘4622 superpose well on the cryo-EM structure of ‘9087-α_{2A}AR Goa and ‘4622-α_{2A}AR-Goa. FIG. 2A: Cryo-EM structure of the ‘9087-α_{2A}AR-Goa complex. FIG. 2B: Experimental ‘9087 structure superposed on the docked pose (RMSD 1.14 Å). Hydrogen bonds and ion pairs are shown with dashed lines to F427^{7.39} and D128^{3.32}, respectively. FIG. 2C: Cryo-EM structure of the ‘4622-α_{2A}AR-Goa complex. FIG. 2D: Experimental ‘4622 structure superposed on the docked pose (RMSD 1.14 Å). Hydrogen bond shown with dashed black lines to D128^{3.32} For FIG. 2B and FIG. 2D: Side chains of residues within 4 Å of ligands are shown as sticks.

FIGS. 3A-3D. Structure-based optimization of ‘9087. FIG. 3A: Strategies for analoging ‘9087 (left). Analogs of the pyridine and lipophilic nature of the bicyclic ring revealed their importance for ‘9087 activity (middle). Trying alternate lipophilic bicyclic rings and modifying their substituents identified eight more potent agonists (right). EC₅₀ values shown for Gᵢ activation. FIG. 3B: Gᵢ and β-arrestin-2 recruitment for ‘9087 and two potent analogs, ‘7075 and PS75. FIG. 3C: Modeled poses of ‘7075 and PS75 based on ‘9087-α_{2A}AR structure with substituents oriented towards open space in the orthosteric site. Hydrogen bonds and ionic interactions are shown with dashed lines to F427^{7.39} and D128^{3.32}, respectively. FIG. 3D: Strategies for analoging ‘9087 (left). Analogs of the pyridine, exocyclic nitrogen, and lipophilic nature of the bicyclic ring revealed their importance for ‘9087 activity (middle). Sampling alternate lipophilic bicyclic rings and modifying their substituents identified eight more potent agonists (right). EC₅₀ values shown for Gᵢ activation. For FIG. 3A—indicates minimal activity at high concentrations or inactive. For FIG. 3B: Data are mean±s.e.m. of normalized results (n=7 to 17 measurements for Gᵢ and n=4 to 8 measurements for β-arrestin-2). For FIG. 3D, Gᵢ and β-arrestin-2 recruitment data for analogs shown in FIGS. 13A-13D, FIGS. 14A-14H, and Table 3.

FIGS. 4A-4J. Novel docking agonists are antinociceptive in neuropathic, inflammatory, and acute thermal pain, but are not sedating. FIGS. 4A-4C: Effect of novel agonists in neuropathic pain model in mice following spared nerve injury (SNI) with mechanical allodynia. FIG. 4A: Novel agonist ‘9087 and PS75 administered in naïve mice (Baseline vs ‘9087 5 mg/kg, baseline and PS75 5 mg/kg, baseline; one-way ANOVA; ns=not significant, **** p<0.0001), dose response of ‘9087 in SNI-mice and analogs ‘7075 and PS75 compared to their vehicles (20% kolliphor vs. all ‘9087 doses; 20% cyclodextran vs. ‘7075 and PS75; one-way ANOVA; ns=not significant, ** p<0.01, *** p<0.001, **** p<0.0001) with positive control dexmedetomidine (DEX), and ‘9087 administered orally compared to its vehicle (40% captisol; two-tailed t-test; ** p<0.01). FIG. 4B: Effect of additional agonists ‘4622, ‘0172, ‘2998 compared to their vehicles (20% kolliphor vs. ‘4622 5 mg/kg, ‘4622 10 mg/kg, and ‘0172 5 mg/kg; one-way ANOVA; 20% cyclodextran vs. ‘2998; two-tailed t-test; ns=not significant, * p<0.05, ** p<0.01, **** p<0.0001) and positive control DEX. FIG. 4C: Administration of α_{2A}AR antagonist atipamezole (ATPZ) to block agonist efficacy in neuropathic pain model (‘9087 without ATPZ vs. ‘9087 with ATPZ; ‘7074 without ATPZ vs. ‘7075 with ATPZ; PS75 without ATPZ vs. PS75 with ATPZ; ‘0172 without ATPZ vs. ‘0172 with ATPZ; ‘4622 without ATPZ vs. ‘4622 with ATPZ; ‘2998 without ATPZ vs. ‘2998 with ATPZ; DEX without ATPZ vs. DEX with ATPZ; two-tailed t-test; ns=not significant, * p<0.05, ** p<0.01). FIG. 4D: Diminished analgesia in α_{2A}AR D79N mice in the 50° C. tail flick assay for acute thermal (heat) pain. The mutation does not affect morphine analgesia but substantially decreases the analgesia by DEX, ‘9087, and PS75 (Baseline WT vs. D79N, Morphine WT vs. D79N, DEX WT vs. D79N, ‘9087 WT vs. D79N, PS75 WT vs. D79N; two-tailed t-test; ns=not significant, * p<0.05, ** p<0.01). FIG. 4E: Analgesia of ‘9087 and PS75 in 50° C. tail flick assay for acute thermal (heat) pain compared to its vehicle (20% Kolliphor vs ‘9087 and PS75; one-way ANOVA; ns=not significant, **** p<0.0001). FIG. 4F: Analgesia of ‘9087 in 55° C. hot plate assay for acute thermal (heat) pain compared to its vehicle (20% kolliphor vs. ‘9087; two-tailed t-test; *** p<0.001). FIG. 4G: Efficacy of novel agonists in CFA-induced hyperalgesia compared to the vehicle (vehicle vs ‘9087, ‘2998, and ‘0172; one-way ANOVA; ns=not significant, * p<0.05, *** p<0.001). FIG. 4H: Evaluating motor impairment and sedation of novel agonists in the rotarod motor test. Only ‘4622 causes slight motor impairment while other agonists do not. DEX causes significant impairment and complete sedation at higher doses. All compounds compared to their vehicles (20% kolliphor vs. ‘9087, ‘0172, ‘4622; 20% cyclodextran vs ‘2298, ‘7075 and PS75; saline vs. DEX; one-way ANOVA; ns=not significant, * p<0.05, ** p<0.01, **** p<0.0001). FIG. 4I: The new agonists ‘9087 and PS75 administered in naïve mice (baseline vs ‘9087 5 mg/kg, baseline vs PS75 5 mg/kg; one-way ANOVA; ns=not significant, **** p<0.0001), dose response of ‘9087 in SNI-mice and analogs ‘7075 and PS75 compared to their vehicles (20% kolliphor vs all ‘9087 doses; 20% cyclodextran vs ‘7075 and PS75; one-way ANOVA; ns=not significant, ** p<0.01, *** p<0.001, **** p<0.0001) with positive control dexmedetomidine (DEX), and ‘9087 administered orally (p.o.) compared to its vehicle (40% captisol vs ‘9087 doses; one-way ANOVA; ns=not significant, **** p<0.0001). FIG. 4J: Evaluating motor impairment and sedation of novel agonists in the rotarod motor test. Only ‘4622 causes slight motor impairment while other agonists do not. DEX causes significant impairment and complete sedation at higher doses. All compounds compared to their vehicles (20% Kolliphor vs ‘9087, ‘0172, ‘4622; 20% cyclodextran vs ‘2298, ‘7075 and PS75; saline vs DEX; one-way ANOVA; ns=not significant, * p<0.05, ** p<0.01, **** p<0.0001). For FIG. 4A: All compounds were administered s.c. unless otherwise indicated. Data are shown as individual data points and mean+/−s.e.m. (n=5 to 15 measurements). For FIGS. 4B-4G and FIG. 4I, all compounds were administered s.c. unless otherwise indicated. Data are shown as individual data points and mean±s.e.m. (n=5 to 25 measurements).

FIGS. 5A-5C. Analogs of ‘9087 reveal key SAR. FIGS. 5A-5B: Analogs with changes to the pyridine of ‘9087 reduced or eliminated Gᵢ recruitment. FIG. 5C: Additional analogs with improved potency for Gᵢ recruitment. For FIGS. 5B-5C: Data are mean±s.e.m. of normalized results.

FIGS. 6A-6G. Functional data for docking hits against α_{2A}AR. FIGS. 6A-6B: Gᵢ signaling for docking hits against human α_{2A}AR in the IP-One assay. FIGS. 6C-6E: Gᵢ activation for docking hits against murine α_{2A}AR in the IP-One assay. FIGS. 6F-6G: β-arrestin-2 recruitment for docking hits against human α_{2A}AR in the PathHunter assay. For FIGS. 6A-6G, data are mean±s.e.m. of normalized results (n=3-11 measurements).

FIGS. 7A-7L. Functional data for docking hits against α_{2B}AR. FIGS. 7A-7C and FIGS. 7G-7I: Gᵢ signaling for docking hits against human α_{2B}AR in the IP-One assay. FIGS. 7D-7F and FIGS. 7J-7L: β-arrestin-2 recruitment for docking hits against human α_{2B}AR in the PathHunter assay. For FIGS. 7A-7L, data are mean±s.e.m. of normalized results (n=3-9 measurements).

FIG. 8. Gᵢ-activation induced cAMP inhibition assay against α_{2A}AR. Gᵢ signaling for docking hits against human α_{2A}AR in the DiscoverX HitHunter cAMP assay. Data are mean±s.e.m. of normalized results (n=2 measurements).

FIGS. 9A-9F. Functional properties of norepinephrine, selected docking agonists and the bespoken synthesized analog PS 75 are dependent on receptor density. Comparison of EC₅₀ and Eₘₐₓ values at the standard receptor density of 200 ng transfected cDNA and results derived at receptor expression at 50 ng and 10 ng of DNA for norepinephrine (NorEpi) (FIG. 9A), ‘9087 (FIG. 9B), ‘7075 (FIG. 9C), and PS75 (FIG. 9D) for α_{2A}AR. FIG. 9E: Relative surface expression of α_{2A}AR coexpressed with Gα_{i1-RlucII}, Gβ₁ and Gγ_{2-GFP10} determined by ELISA directed against the N-terminal FLAG-tag. Individual data points are shown relative to cells transfected with 200 ng α_{2A}AR-plasmid, only. FIG. 9F: summary of all activation data for compounds at different α_{2A}AR receptor densities. For FIGS. 9A-9D and FIG. 9F, all data shown are for Gᵢ activation monitored in a BRET-biosensor based assay with 5 to 18 experiments in duplicates; mean EC₅₀ values are displayed as in [nM±s.e.m.]. Normalization was done relative to the maximum effect of norepinephrine (NE) and is displayed in [%±s.e.m.]. For FIG. 9E, data are mean±s.e.m. of normalized results (n=4 of quadruplicates).

FIGS. 10A-10C. EMTA coupling panel for select docking compounds against α_{2A}AR. G-protein and β-Arrestin signaling profiles for docking compounds in BRET biosensor-based assays in HEK293 cells expressing the human α_{2A}AR. Data are mean±s.e.m. of normalized results (n=3-5 measurements). Endogenous G_{i/o} indicates activation of the protein family in the absence of heterologously expressed G proteins.

FIG. 11. Relative activities for select docking compounds against α_{2A}AR EMTA coupling panel. G-protein and β-Arrestin signaling profiles for docking compounds in BRET biosensor-based assays in HEK293 cells expressing the human α_{2A}AR.

FIGS. 12A-12E. Internalization behavior of α_{2A}AR following compound treatment. FIG. 12A: Kinetics of α_{2A}AR disappearance from the plasma membrane following 100 μM compound treatment using an human α_{2A}AR-RlucII/rGFP-CAAX biosensor. FIG. 12B: Kinetics of α_{2A}AR relocalization in endosomes following 100 μM compound treatment using a human α_{2A}AR-RlucII/rGFP-FYVE biosensor. FIG. 12C: Concentration-response curves of α_{2A}AR disappearance from the plasma membrane using an human α_{2A}AR-RlucII/rGFP-CAAX biosensor. FIG. 12D: Concentration-response curves of α_{2A}AR relocalization in endosomes using an human α_{2A}AR-RlucII/rGFP-CAAX biosensor. FIG. 12E: Summary of biosensor data for compounds. For FIGS. 12C-12D, normalization was done relative to the maximum effect of norepinephrine. Data are mean±s.e.m. of normalized results (n=3 measurements).

FIGS. 13A-13D. Analogs of ‘9087 reveal key SAR. Gᵢ activation for human α_{2A}AR of analogs with changes to the pyridine ring, exocyclic nitrogen, and isoquinoline of ‘9087 monitored in a BRET-biosensor based assay. Data are mean±s.e.m. of normalized results (n=3-9 measurements).

FIGS. 14A-14H. Functional data for selected docking hits and references against α_{2A}AR. Gᵢ activation (FIGS. 14A-14B and FIGS. 14E-14H) and 0-arrestin-2 recruitment (FIGS. 14C-14D) determined in BRET-biosensor based assays in HEK293T cells expressing the human α_{2A}AR wild-type. Data are mean±s.e.m. (n=3 to 17 measurements) displayed as delta BRET values corresponding to functional data of selected docking hits and references referred to in FIG. 1C, FIG. 3B (FIGS. 14A-14D), and FIGS. 13A-13D (FIGS. 14E-14H) in the order of their appearance.

FIGS. 15A-15E. Off-target activity for α_{2A}AR agonists. FIG. 15A: GPCRome of ‘9087. Labeled targets indicate an increase of 3-fold or higher signaling compared to basal activity. Positive control is shown with D2R_{long} and quinpirole. FIG. 15B: D2R_{long} Gᵢ and β-arrestin-2 (arr) recruitment for ‘9087 with EC₅₀=4.5 μM and Eₘₐₓ=57% for Gᵢ signaling and EC₅₀=16 μM and Eₘₐₓ=21% for arrestin recruitment, respectively. Positive control quinpirole (Quin) also shown. FIG. 15C: hERG inhibition of ‘9087 and positive control dofetilide. FIG. 15D: μOR binding of α_{2A}AR docking agonists and analogs. FIG. 15E: I2R binding of α_{2A}AR agonists. For FIGS. 15A-15E, data are shown as mean±s.e.m. (n=3 to 4 measurements).

FIG. 16. Phase I metabolism of ‘9087, ‘7075, and PS75 in male rat liver microsomes. Rotigotine and imipramine serve as positive controls for extensive phase I metabolism. Data are percent of non-metabolized compound remaining shown as mean±s.e.m. (n=4 individual experiments for imipramine and rotigotine, n=5 for ‘9087, ‘7075, PS75).

FIGS. 17A-17B. In vivo side effects of constipation and body weight. FIG. 17A: Constipation monitored up to 6 hours following vehicle or compound i.p. injection (two-way ANOVA; time×treatment interaction: F(15,80)=1.501, P=0.1250; time: F(1.071, 17.14)=111.7, P<0.0001; treatment: F(3,16))=3.784, P=0.0316; all treatment groups (n=6)); each compound time point compared to vehicle, asterisks define difference between morphine and vehicle at 1 h (P=0.0209), 2 h (P=0.0372) and 3 h (P=0.0417) for simplicity, all other points compared to vehicle are not significant; * p<0.05). Data are mean±s.e.m. FIG. 17B: Body weight measured over 48 hours following vehicle or compound i.p. injection (two-way ANOVA; time×treatment interaction: F(6,32)=0.5174, P=0.7907; time: F(1.161, 18.57)=3.177, P=0.0863; treatment: F(3,16))=0.2854, P=0.8358; all treatment groups (n=3)); time points compared within same treatment groups, all comparisons are not significant). Data are mean±s.e.m.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom.

Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

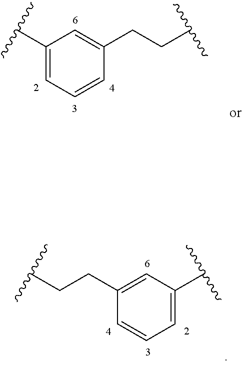 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2 m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{1A.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.2}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

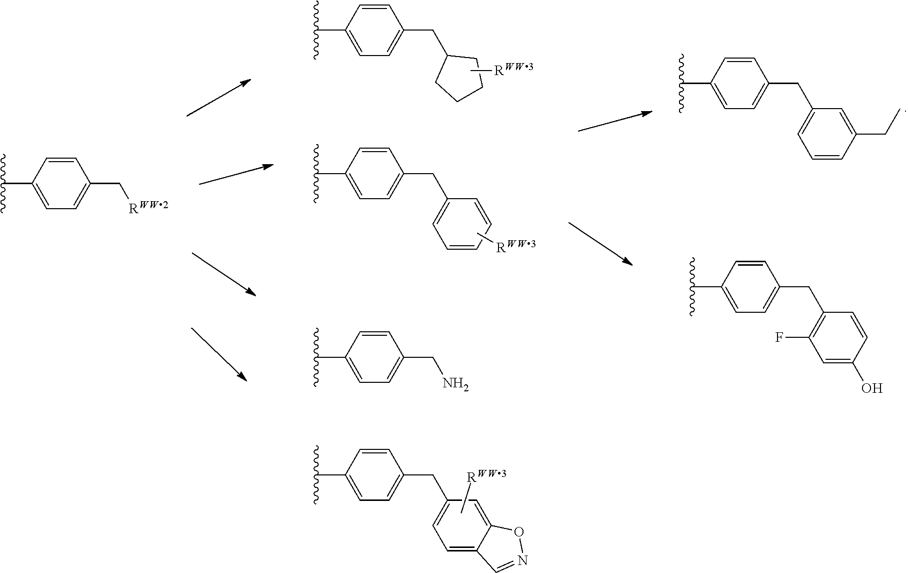

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, OCX^{LWW.1}₃, —OCH₂X^{LWW.1}, OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms.

The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is pain.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

The term “metabolic disease” or “metabolic disorder” refers to a disorder characterized by one or more abnormal metabolic processes in a subject. In embodiments, a metabolic disorder may be associated with, related to, or may be diabetes (e.g., type 1 diabetes or type 2 diabetes), insulin resistance, metabolic syndrome, obesity, hyperlipidemia, hyperglycemia, high serum triglycerides, and/or high blood pressure. In embodiments, a metabolic disorder may be associated with, related to, or may be a diabetes associated disease selected from nephropathy, retinopathy, neuropathy, cardiovascular disease, or inflammation. In embodiments, a metabolic disorder may be associated with, related to, or may be nephropathy, retinopathy, neuropathy, cardiovascular disease, or inflammation.

As used herein, the term “infectious disease” or “infection” refers to a disease or condition related to the presence of an organism (the agent or infectious agent) within or contacting the subject or patient. Examples include a bacterium, fungus, virus, or other microorganism. A “bacterial infectious disease” or “bacterial disease” is an infectious disease wherein the organism is a bacterium. A “viral infectious disease” or “viral disease” is an infectious disease wherein the organism is a virus. Examples of infectious diseases that may be treated with a compound or method described herein include nosocomial infections, bacteremia, Cutaneous anthrax, Pulmonary anthrax, Gastrointestinal anthrax, Whooping cough, bacterial pneumonia, bacteremia, Lyme disease, Brucellosis, Acute enteritis, Community-acquired respiratory infection, Nongonococcal urethritis (NGU), Lymphogranuloma venereum (LGV), Trachoma, Inclusion conjunctivitis of the newborn (ICN), Psittacosis, Botulism, Pseudomembranous colitis, Gas gangrene, Acute food poisoning, Anaerobic cellulitis, Tetanus, Diphtheria, Nosocomial infections, Urinary tract infections (UTI), Diarrhea, Meningitis in infants, Traveller's diarrhea, Diarrhea in infants, Hemorrhagic colitis, Hemolytic-uremic syndrome, Tularemia, Bacterial meningitis, Upper respiratory tract infections, Pneumonia, bronchitis, Peptic ulcer, gastric carcinoma, gastric B-cell lymphoma, Legionnaire's Disease, Pontiac fever, Leptospirosis, Listeriosis, Leprosy (Hansen's disease), Tuberculosis, Mycoplasma pneumonia, Gonorrhea, Ophthalmia neonatorum, Septic arthritis, Meningococcal disease, Waterhouse-Friderichsen syndrome, Pseudomonas infection, Bacteremia, endocarditis, Rocky mountain spotted fever, Typhoid fever type salmonellosis (dysentery, colitis), Salmonellosis, gastroenteritis, enterocolitis, Bacillary dysentery/Shigellosis, Coagulase-positive staphylococcal infections, Impetigo, Acute infective endocarditis, Septicemia, Necrotizing pneumonia, Toxinoses, Toxic shock syndrome, Staphylococcal food poisoning, Cystitis, Meningitis, septicemia, Endometritis, Opportunistic infections, Acute bacterial pneumonia, Otitis media, sinusitis, Streptococcal pharyngitis, Scarlet fever, Rheumatic fever, erysipelas, Puerperal fever, Necrotizing fasciitis, Syphilis, Congenital syphilis, Cholera, Plague, Bubonic plague, Pneumonic plague, Iraq war infection caused by Acinetobacter baumannii (i.e., Iraq war-related Acinetobacter baumannii infection), necrotizing fasciitis, tuberculosis, hospital-acquired pneumonia, gastroenteritis, or sepsis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Z, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴, ¹⁹⁸Au, ¹⁹⁹Au, ²¹¹At, ²¹¹Pb ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., pain) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., pain) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to D128 of α_{2A} adrenergic receptor when the selected residue occupies the same essential spatial or other structural relationship as D128 of α_{2A} adrenergic receptor. In some embodiments, where a selected protein is aligned for maximum homology with the α_{2A} adrenergic receptor, the position in the aligned selected protein aligning with D128 is said to correspond to D128. Instead of a primary sequence alignment, a three dimensional structural alignment can also be used, e.g., where the structure of the selected protein is aligned for maximum correspondence with the α_{2A} adrenergic receptor and the overall structures compared. In this case, an amino acid that occupies the same essential position as D128 in the structural model is said to correspond to the D128 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “α_{2A} adrenergic receptor” or “alpha-2A adrenergic receptor” or “α_{2A}AR” refers to a receptor (including homologs, isoforms, and functional fragments thereof) that plays a role in regulating neurotransmitter release from sympathetic nerves and from adrenergic neurons in the central nervous system. The term includes any recombinant or naturally-occurring form of α_{2A}AR variants thereof that maintain α_{2A}AR activity (e.g., within at least 30%, 40%, 50%, 60%, 70%, 80%, 90%, 95%, or 100% activity compared to wildtype α_{2A}AR). In embodiments, the α_{2A}AR protein encoded by the ADRA2A gene has the amino acid sequence set forth in or corresponding to Entrez 150, UniProt P08913, or RefSeq (protein) NP_000672.3. In embodiments, the ADRA2A gene has the nucleic acid sequence set forth in RefSeq (mRNA) NM_000681.3. In embodiments, the amino acid sequence or nucleic acid sequence is the sequence known at the time of filing of the present application. In embodiments, the α_{2A}AR has the following amino acid sequence:

(SEQ ID NO: 1)
MFRQEQPLAEGSFAPMGSLQPDAGNASWNGTEAPGGGARATPYSLQV
TLTLVCLAGLLMLLTVFGNVLVIIAVFTSRALKAPQNLFLVSLASAD
ILVATLVIPFSLANEVMGYWYFGKAWCEIYLALDVLFCTSSIVHLCA
ISLDRYWSITQAIEYNLKRTPRRIKAIIITVWVISAVISFPPLISIE
KKGGGGGPQPAEPRCEINDQKWYVISSCIGSFFAPCLIMILVYVRIY
QIAKRRTRVPPSRRGPDAVAAPPGGTERRPNGLGPERSAGPGGAEAE
PLPTQLNGAPGEPAPAGPRDTDALDLEESSSSDHAERPPGPRRPERG
PRGKGKARASQVKPGDSLPRRGPGATGIGTPAAGPGEERVGAAKASR
WRGRQNREKRFTFVLAVVIGVFVVCWFPFFFTYTLTAVGCSVPRTLF
KFFFWFGYCNSSLNPVIYTIFNHDFRRAFKKILCRGDRKRIV.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward α_{2A}AR would preferentially inhibit α_{2A}AR over other adrenergic receptors). In embodiments, an “α_{2A}AR-selective compound” refers to a compound (e.g., compound described herein) having selectivity towards α_{2A}AR. In embodiments, the compound (e.g., compound described herein) is about 5-fold, 10-fold, 20-fold, 30-fold, 40-fold, 50-fold, or about 100-fold more selective for α_{2A}AR over α_{2B}AR. In embodiments, the compound (e.g., compound described herein) is at least 5-fold, 10-fold, 20-fold, 30-fold, 40-fold, 50-fold, or at least 100-fold more selective for α_{2A}AR over α_{2B}AR. In embodiments, the compound (e.g., compound described herein) is about 5-fold, 10-fold, 20-fold, 30-fold, 40-fold, 50-fold, or about 100-fold more selective for α_{2A}AR over off-target proteins (e.g., hERG ion channel, beta-2 adrenergic receptor). In embodiments, the compound (e.g., compound described herein) is at least 5-fold, 10-fold, 20-fold, 30-fold, 40-fold, 50-fold, or at least 100-fold more selective for α_{2A}AR over off-target proteins (e.g., hERG ion channel, beta-2 adrenergic receptor).

### II. Compounds

In an aspect is provided a compound, or a pharmaceutically acceptable salt thereof, having the formula:

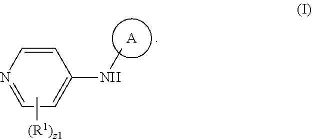

Ring A is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹ is independently —F, —Cl, —Br, or —I.

The symbol n1 is an integer from 0 to 4.

The symbols m1 and v1 are independently 1 or 2.

The symbol z1 is an integer from 0 to 4.

In embodiments, the compound has the formula:

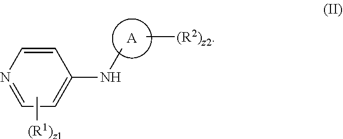

R¹ and z1 are as described herein, including in embodiments.

In embodiments, Ring A is cycloalkyl, heterocycloalkyl, aryl, or heteroaryl.

R² is independently oxo, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X² is independently —F, —Cl, —Br, or —I.

The symbol n2 is an integer from 0 to 4.

The symbols m2 and v2 are independently 1 or 2.

The symbol z2 is an integer from 0 to 15.

In embodiments, a substituted Ring A (e.g., substituted cycloalkyl, substituted heterocycloalkyl, substituted aryl, and/or substituted heteroaryl) is substituted with at least one substituent group, size-limited substituent group, or lower substituent group; wherein if the substituted Ring A is substituted with a plurality of groups selected from substituent groups, size-limited substituent groups, and lower substituent groups; each substituent group, size-limited substituent group, and/or lower substituent group may optionally be different. In embodiments, when Ring A is substituted, it is substituted with at least one substituent group. In embodiments, when Ring A is substituted, it is substituted with at least one size-limited substituent group. In embodiments, when Ring A is substituted, it is substituted with at least one lower substituent group.

In embodiments, Ring A is substituted or unsubstituted cycloalkyl. In embodiments, Ring A is substituted or unsubstituted heterocycloalkyl. In embodiments, Ring A is substituted or unsubstituted aryl. In embodiments, Ring A is substituted or unsubstituted heteroaryl. In embodiments, Ring A is substituted or unsubstituted naphthyl. In embodiments, Ring A is substituted or unsubstituted 1-naphthyl. In embodiments, Ring A is substituted or unsubstituted isoquinolinyl. In embodiments, Ring A is substituted or unsubstituted 1-isoquinolinyl. In embodiments, Ring A is substituted or unsubstituted 3-isoquinolinyl. In embodiments, Ring A is substituted or unsubstituted 4-isoquinolinyl. In embodiments, Ring A is substituted or unsubstituted 5-isoquinolinyl. In embodiments, Ring A is substituted or unsubstituted quinolinyl. In embodiments, Ring A is substituted or unsubstituted 2-quinolinyl. In embodiments, Ring A is substituted or unsubstituted 3-quinolinyl. In embodiments, Ring A is substituted or unsubstituted 4-quinolinyl. In embodiments, Ring A is substituted or unsubstituted benzothiophenyl. In embodiments, Ring A is substituted or unsubstituted 7-benzothiophenyl. In embodiments, Ring A is substituted or unsubstituted dihydroisoquinolinonyl. In embodiments, Ring A is substituted or unsubstituted dihydroquinolinonyl. In embodiments, Ring A is substituted or unsubstituted dihydronaphthyridinonyl.

In embodiments, Ring A is cycloalkyl. In embodiments, Ring A is heterocycloalkyl. In embodiments, Ring A is aryl. In embodiments, Ring A is heteroaryl. In embodiments, Ring A is naphthyl. In embodiments, Ring A is 1-naphthyl. In embodiments, Ring A is isoquinolinyl. In embodiments, Ring A is 1-isoquinolinyl. In embodiments, Ring A is 3-isoquinolinyl. In embodiments, Ring A is 4-isoquinolinyl. In embodiments, Ring A is 5-isoquinolinyl. In embodiments, Ring A is quinolinyl. In embodiments, Ring A is 2-quinolinyl. In embodiments, Ring A is 3-quinolinyl. In embodiments, Ring A is 4-quinolinyl. In embodiments, Ring A is benzothiophenyl. In embodiments, Ring A is 7-benzothiophenyl. In embodiments, Ring A is dihydroisoquinolinonyl. In embodiments, Ring A is dihydroquinolinonyl. In embodiments, Ring A is dihydronaphthyridinonyl.

In embodiments, is

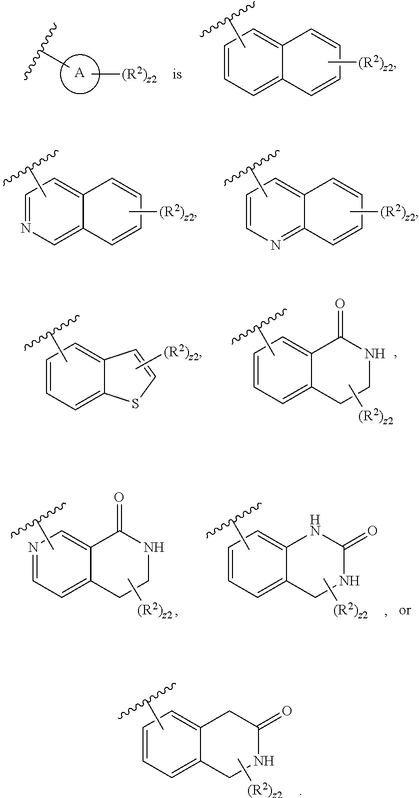

R² and z2 are as described herein, including in embodiments.

In embodiments,

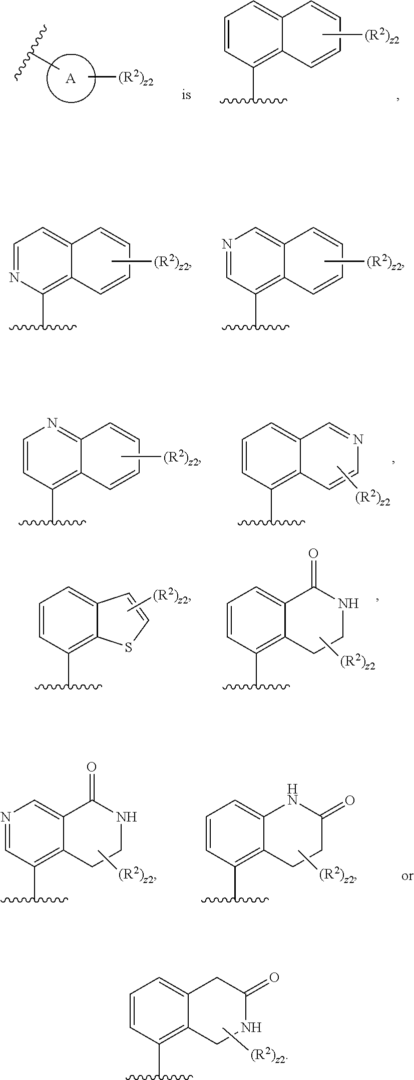

R² and z2 are as described herein, including in embodiments.

In embodiments, the compound has the formula:

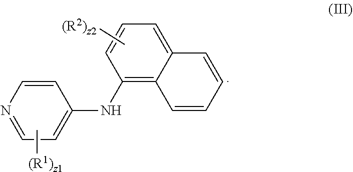

R¹, z1, R², and z2 are as described herein, including in embodiments.

In embodiments, the compound has the formula:

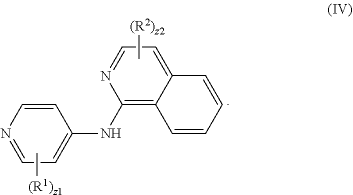

R¹, z1, R², and z2 are as described herein, including in embodiments.

In embodiments, the compound has the formula:

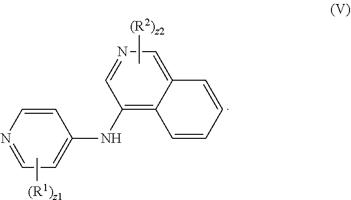

R¹, z1, R², and z2 are as described herein, including in embodiments.

In embodiments, the compound has the formula:

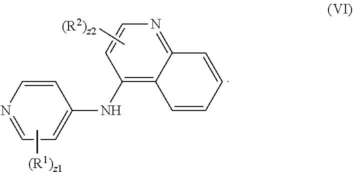

R¹, z1, R², and z2 are as described herein, including in embodiments.

In embodiments, the compound has the formula:

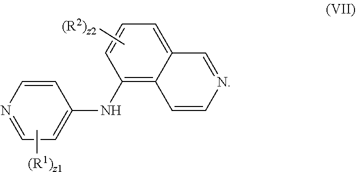

R¹, z1, R², and z2 are as described herein, including in embodiments.

In embodiments, the compound has the formula:

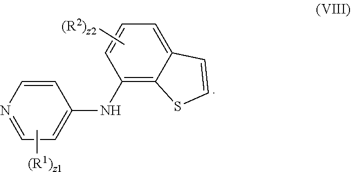

R¹, z1, R², and z2 are as described herein, including in embodiments.

In embodiments, the compound has the formula:

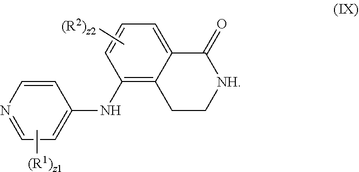

R¹, z1, R², and z2 are as described herein, including in embodiments.

In embodiments, the compound has the formula:

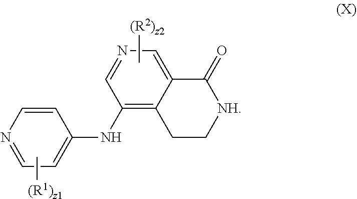

R¹, z1, R², and z2 are as described herein, including in embodiments.

In embodiments, the compound has the formula:

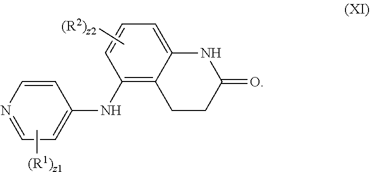

R¹, z1, R², and z2 are as described herein, including in embodiments.

In embodiments, the compound has the formula:

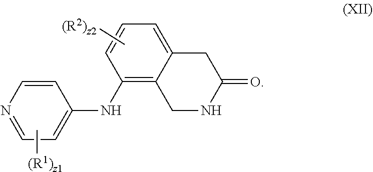

R¹, z1, R², and z2 are as described herein, including in embodiments.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —NR^{1A}R^{1B} In embodiments, R¹ is independently —NH₂. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 6 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy.

In embodiments, z1 is 0. In embodiments, z1 is 1. In embodiments, z1 is 2. In embodiments, z1 is 3. In embodiments, z1 is 4.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently oxo, halogen, —CF₃, —OR^{2D}, or unsubstituted C₁-C₄ alkyl. In embodiments, R² is independently oxo, —F, —Cl, —CF₃, —OH, —OCH₃, or unsubstituted methyl. In embodiments, R² is independently oxo. In embodiments, R² is independently halogen. In embodiments, R² is independently —F. In embodiments, R² is independently —Cl. In embodiments, R² is independently —Br. In embodiments, R² is independently —I. In embodiments, R² is independently —CF₃. In embodiments, R² is independently —OR^{2D}. In embodiments, R² is independently —OH. In embodiments, R² is independently —OCH₃. In embodiments, R² is independently unsubstituted C₁-C₄ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted isobutyl. In embodiments, R² is independently unsubstituted tert-butyl. In embodiments, R² is independently unsubstituted 2 to 6 membered heteroalkyl. In embodiments, R² is independently unsubstituted methoxy. In embodiments, R² is independently unsubstituted ethoxy. In embodiments, R² is independently unsubstituted propoxy. In embodiments, R² is independently unsubstituted n-propoxy. In embodiments, R² is independently unsubstituted isopropoxy. In embodiments, R² is independently unsubstituted butoxy.

In embodiments, R^{2D} is independently hydrogen. In embodiments, R^{2D} is independently unsubstituted C₁-C₄ alkyl. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted n-propyl. In embodiments, R^{2D} is independently unsubstituted isopropyl. In embodiments, R^{2D} is independently unsubstituted butyl. In embodiments, R^{2D} is independently unsubstituted n-butyl. In embodiments, R^{2D} is independently unsubstituted isobutyl. In embodiments, R^{2D} is independently unsubstituted tert-butyl.

In embodiments, z2 is 0. In embodiments, z2 is 1. In embodiments, z2 is 2. In embodiments, z2 is 3. In embodiments, z2 is 4. In embodiments, z2 is 5. In embodiments, z2 is 6. In embodiments, z2 is 7. In embodiments, z2 is 8. In embodiments, z2 is 9. In embodiments, z2 is 10. In embodiments, z2 is 11. In embodiments, z2 is 12. In embodiments, z2 is 13. In embodiments, z2 is 14. In embodiments, z2 is 15.

In embodiments, the compound has the formula:

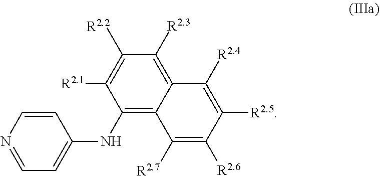

R^{2.1}, R^{2.2}, R^{2.3}, R^{2.4}, R^{2.5}, R^{2.6}, and R^{2.7} am independently hydrogen or any value of R² as described herein, including in embodiments.

In embodiments, at least one of R^{2.1}, R^{2.2}, R^{2.3}, R^{2.4}, R^{2.5}, R^{2.6}, and R^{2.7} is not hydrogen.

In embodiments, R^{2.1}, R^{2.2}, R^{2.3}, R^{2.4}, R^{2.5}, R^{2.6}, and R^{2.7} are independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{2.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.1} is substituted with a plurality of groups selected from substituent groups, size-limited substituent groups, and lower substituent groups; each substituent group, size-limited substituent group, and/or lower substituent group may optionally be different. In embodiments, when R^{2.1} is substituted, it is substituted with at least one substituent group. In embodiments, when R^{2.1} is substituted, it is substituted with at least one size-limited substituent group. In embodiments, when R^{2.1} is substituted, it is substituted with at least one lower substituent group.

In embodiments, a substituted R^{2.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.2} is substituted with a plurality of groups selected from substituent groups, size-limited substituent groups, and lower substituent groups; each substituent group, size-limited substituent group, and/or lower substituent group may optionally be different. In embodiments, when R^{2.2} is substituted, it is substituted with at least one substituent group. In embodiments, when R^{2.2} is substituted, it is substituted with at least one size-limited substituent group. In embodiments, when R^{2.2} is substituted, it is substituted with at least one lower substituent group.

In embodiments, a substituted R^{2.3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.3} is substituted with a plurality of groups selected from substituent groups, size-limited substituent groups, and lower substituent groups; each substituent group, size-limited substituent group, and/or lower substituent group may optionally be different. In embodiments, when R^{2.3} is substituted, it is substituted with at least one substituent group. In embodiments, when R^{2.3} is substituted, it is substituted with at least one size-limited substituent group. In embodiments, when R^{2.3} is substituted, it is substituted with at least one lower substituent group.

In embodiments, a substituted R^{2.4} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.4} is substituted with a plurality of groups selected from substituent groups, size-limited substituent groups, and lower substituent groups; each substituent group, size-limited substituent group, and/or lower substituent group may optionally be different. In embodiments, when R^{2.4} is substituted, it is substituted with at least one substituent group. In embodiments, when R^{2.4} is substituted, it is substituted with at least one size-limited substituent group. In embodiments, when R^{2.4} is substituted, it is substituted with at least one lower substituent group.

In embodiments, a substituted R^{2.5} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.5} is substituted with a plurality of groups selected from substituent groups, size-limited substituent groups, and lower substituent groups; each substituent group, size-limited substituent group, and/or lower substituent group may optionally be different. In embodiments, when R^{2.5} is substituted, it is substituted with at least one substituent group. In embodiments, when R^{2.5} is substituted, it is substituted with at least one size-limited substituent group. In embodiments, when R^{2.5} is substituted, it is substituted with at least one lower substituent group.

In embodiments, a substituted R^{2.6} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.6} is substituted with a plurality of groups selected from substituent groups, size-limited substituent groups, and lower substituent groups; each substituent group, size-limited substituent group, and/or lower substituent group may optionally be different. In embodiments, when R^{2.6} is substituted, it is substituted with at least one substituent group. In embodiments, when R^{2.6} is substituted, it is substituted with at least one size-limited substituent group. In embodiments, when R^{2.6} is substituted, it is substituted with at least one lower substituent group.

In embodiments, a substituted R^{2.7} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2.7} is substituted with a plurality of groups selected from substituent groups, size-limited substituent groups, and lower substituent groups; each substituent group, size-limited substituent group, and/or lower substituent group may optionally be different. In embodiments, when R^{2.7} is substituted, it is substituted with at least one substituent group. In embodiments, when R^{2.7} is substituted, it is substituted with at least one size-limited substituent group. In embodiments, when R^{2.7} is substituted, it is substituted with at least one lower substituent group.

In embodiments, the compound has the formula:

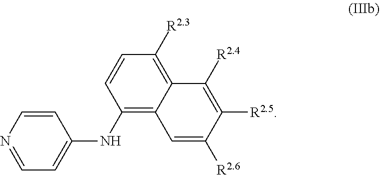

R^{2.3}, R^{2.4}, R^{2.5}, and R^{2.6} are independently hydrogen or any value of R² as described herein, including in embodiments. In embodiments, at least one of R^{2.3}, R^{2.4}, R^{2.5}, and R^{2.6} is not hydrogen.

In embodiments, R^{2.3} is hydrogen, halogen, —OR^{2D}, or unsubstituted alkyl. In embodiments, R^{2.3} is —F or —OH. In embodiments, R^{2.3} is hydrogen. In embodiments, R^{2.3} is halogen. In embodiments, R^{2.3} is —F. In embodiments, R^{2.3} is —C₁. In embodiments, R^{2.3} is —Br. In embodiments, R^{2.3} is —I. In embodiments, R^{2.3} is —OR^{2D}. In embodiments, R^{2.3} is —OH. In embodiments, R^{2.3} is unsubstituted C₁-C₄ alkyl. In embodiments, R^{2.3} is unsubstituted methyl. In embodiments, R^{2.3} is unsubstituted ethyl. In embodiments, R^{2.3} is unsubstituted propyl. In embodiments, R^{2.3} is unsubstituted n-propyl. In embodiments, R^{2.3} is unsubstituted isopropyl. In embodiments, R^{2.3} is unsubstituted butyl. In embodiments, R^{2.3} is unsubstituted n-butyl. In embodiments, R^{2.3} is unsubstituted isobutyl. In embodiments, R^{2.3} is unsubstituted tert-butyl. In embodiments, R^{2.3} is unsubstituted 2 to 6 membered heteroalkyl. In embodiments, R^{2.3} is unsubstituted methoxy. In embodiments, R^{2.3} is unsubstituted ethoxy. In embodiments, R^{2.3} is unsubstituted propoxy. In embodiments, R^{2.3} is unsubstituted n-propoxy. In embodiments, R^{2.3} is unsubstituted isopropoxy. In embodiments, R^{2.3} is unsubstituted butoxy.

In embodiments, R^{2.4} is hydrogen, —OR^{2D}, or unsubstituted alkyl. In embodiments, R^{2.4} is hydrogen. In embodiments, R^{2.4} is halogen. In embodiments, R^{2.4} is —F. In embodiments, R^{2.4} is —C₁. In embodiments, R^{2.4} is —Br. In embodiments, R^{2.4} is —I. In embodiments, R^{2.4} is —OR^{2D}. In embodiments, R^{2.4} is —OH. In embodiments, R^{2.4} is unsubstituted C₁-C₄ alkyl. In embodiments, R^{2.4} is unsubstituted methyl. In embodiments, R^{2.4} is unsubstituted ethyl. In embodiments, R^{2.4} is unsubstituted propyl. In embodiments, R^{2.4} is unsubstituted n-propyl. In embodiments, R^{2.4} is unsubstituted isopropyl. In embodiments, R^{2.4} is unsubstituted butyl. In embodiments, R^{2.4} is unsubstituted n-butyl. In embodiments, R^{2.4} is unsubstituted isobutyl. In embodiments, R^{2.4} is unsubstituted tert-butyl.

In embodiments, R^{2.4} is unsubstituted 2 to 6 membered heteroalkyl. In embodiments, R^{2.4} is unsubstituted methoxy. In embodiments, R^{2.4} is unsubstituted ethoxy. In embodiments, R^{2.4} is unsubstituted propoxy. In embodiments, R^{2.4} is unsubstituted n-propoxy. In embodiments, R^{2.4} is unsubstituted isopropoxy. In embodiments, R^{2.4} is unsubstituted butoxy.

In embodiments, R^{2.5} is hydrogen, halogen, —OR^{2D}, or unsubstituted alkyl. In embodiments, R^{2.5} is —OH or —OCH₃. In embodiments, R^{2.5} is hydrogen. In embodiments, R^{2.5} is halogen. In embodiments, R^{2.5} is —F. In embodiments, R^{2.5} is —C₁. In embodiments, R^{2.5} is —Br. In embodiments, R^{2.5} is —I. In embodiments, R^{2.5} is —OR^{2D}. In embodiments, R^{2.5} is —OH. In embodiments, R^{2.5} is unsubstituted C₁-C₄ alkyl. In embodiments, R^{2.5} is unsubstituted methyl. In embodiments, R^{2.5} is unsubstituted ethyl. In embodiments, R^{2.5} is unsubstituted propyl. In embodiments, R^{2.5} is unsubstituted n-propyl. In embodiments, R^{2.5} is unsubstituted isopropyl. In embodiments, R^{2.5} is unsubstituted butyl. In embodiments, R^{2.5} is unsubstituted n-butyl. In embodiments, R^{2.5} is unsubstituted isobutyl. In embodiments, R^{2.5} is unsubstituted tert-butyl. In embodiments, R^{2.5} is unsubstituted 2 to 6 membered heteroalkyl. In embodiments, R^{2.5} is unsubstituted methoxy. In embodiments, R^{2.5} is unsubstituted ethoxy. In embodiments, R^{2.5} is unsubstituted propoxy. In embodiments, R^{2.5} is unsubstituted n-propoxy. In embodiments, R^{2.5} is unsubstituted isopropoxy. In embodiments, R^{2.5} is unsubstituted butoxy.

In embodiments, R^{2.6} is hydrogen, halogen, —OR^{2D}, or unsubstituted alkyl. In embodiments, R^{2.6} is —F, —Cl, —OCH₃, or unsubstituted C₁-C₄ alkyl. In embodiments, R^{2.6} is hydrogen. In embodiments, R^{2.6} is halogen. In embodiments, R^{2.6} is —F. In embodiments, R^{2.6} is —C₁. In embodiments, R^{2.6} is —Br. In embodiments, R^{2.6} is —I. In embodiments, R²0.6 is —OR^{2D}. In embodiments, R^{2.6} is —OH. In embodiments, R^{2.6} is unsubstituted C₁-C₄ alkyl. In embodiments, R^{2.6} is unsubstituted methyl. In embodiments, R^{2.6} is unsubstituted ethyl. In embodiments, R^{2.6} is unsubstituted propyl. In embodiments, R^{2.6} is unsubstituted n-propyl. In embodiments, R^{2.6} is unsubstituted isopropyl. In embodiments, R^{2.6} is unsubstituted butyl. In embodiments, R^{2.6} is unsubstituted n-butyl. In embodiments, R^{2.6} is unsubstituted isobutyl. In embodiments, R^{2.6} is unsubstituted tert-butyl. In embodiments, R^{2.6} is unsubstituted 2 to 6 membered heteroalkyl. In embodiments, R^{2.6} is unsubstituted methoxy. In embodiments, R^{2.6} is unsubstituted ethoxy. In embodiments, R^{2.6} is unsubstituted propoxy. In embodiments, R^{2.6} is unsubstituted n-propoxy. In embodiments, R^{2.6} is unsubstituted isopropoxy. In embodiments, R^{2.6} is unsubstituted butoxy.

In embodiments, the compound has the formula:

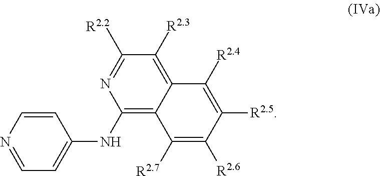

R^{2.2}, R^{2.3}, R^{2.4}, R^{2.5}, R^{2.6}, and R^{2.7} are independently hydrogen or any value of R² as described herein, including in embodiments. In embodiments, R^{2.2}, R^{2.3}, R^{2.4}, R^{2.5}, R^{2.6}, and R^{2.7} are as described herein, including in embodiments. In embodiments, at least one of R^{2.2}, R^{2.3}, R^{2.4}, R^{2.5}, R^{2.6}, and R^{2.7} is not hydrogen.

In embodiments, the compound has the formula:

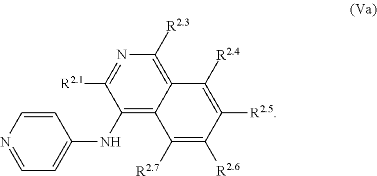

R^{2.1}, R^{2.3}, R^{2.4}, R^{2.5}, R^{2.6}, and R^{2.7} are independently hydrogen or any value of R² as described herein, including in embodiments. In embodiments, R^{2.1}, R^{2.3}, R^{2.4}, R^{2.5}, R^{2.6}, and R^{2.7} are as described herein, including in embodiments. In embodiments, at least one of R^{2.1}, R^{2.3}, R^{2.4}, R^{2.5}, R^{2.6}, and R^{2.7} is not hydrogen. In embodiments, R^{2.1} is not —C₁. In embodiments, R^{2.4} is not —C(O)OR^{2C}.

In embodiments, R^{2.4} is —F or —OCH₃.

In embodiments, R^{2.6} is —F or —CF₃.

In embodiments, the compound has the formula:

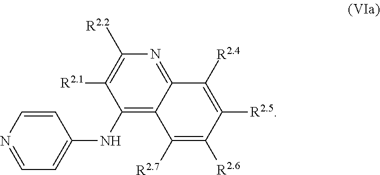

R^{2.1}, R^{2.2}, R^{2.4}, R^{2.5}, R^{2.6}, and R^{2.7} are independently hydrogen or any value of R² as described herein, including in embodiments. In embodiments, R^{2.1}, R^{2.2}, R^{2.4}, R^{2.5}, R^{2.6}, and R^{2.7} are as described herein, including in embodiments. In embodiments, at least one of R^{2.1}, R^{2.2}, R^{2.4}, R^{2.5}, R^{2.6}, and R^{2.7} is not hydrogen.

In embodiments, the compound has the formula:

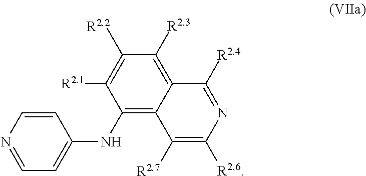

R^{2.1}, R^{2.2}, R^{2.3}, R^{2.4}, R^{2.6}, and R^{2.7} are independently hydrogen or any value of R² as described herein, including in embodiments. In embodiments, R^{2.1}, R^{2.3}, R^{2.4}, R^{2.6}, and R^{2.7} are as described herein, including in embodiments. In embodiments, at least one of R^{2.1}, R^{2.2}, R^{2.3}, R^{2.4}, R^{2.6}, and R^{2.7} is not hydrogen.

In embodiments, the compound has the formula:

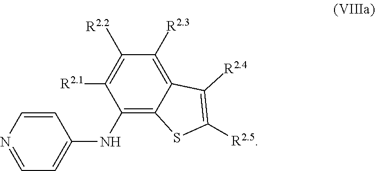

R^{2.1}, R^{2.2}, R^{2.3}, R^{2.4}, and R^{2.5} are independently hydrogen or any value of R² as described herein, including in embodiments. In embodiments, R^{2.1}, R^{2.2}, R^{2.3}, R^{2.4}, and R^{2.5} are as described herein, including in embodiments.

In embodiments, the compound has the formula:

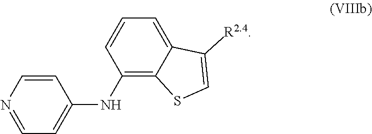

R^{2.4} is hydrogen or any value of R² as described herein, including in embodiments. In embodiments, R^{2.4} is as described herein, including in embodiments.

In embodiments, the compound has the formula:

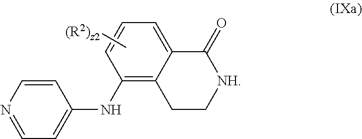

R² and z2 are as described herein, including in embodiments.

In embodiments, the compound has the formula:

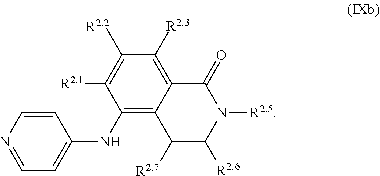

R^{2.1}, R^{2.2}, R^{2.3}, R^{2.5}, R^{2.6}, and R^{2.7} are independently hydrogen or any value of R² as described herein, including in embodiments. In embodiments, R^{2.1}, R^{2.2}, R^{2.3}, R^{2.5}, R^{2.6}, and R^{2.7} are as described herein, including in embodiments.

In embodiments, the compound has the formula:

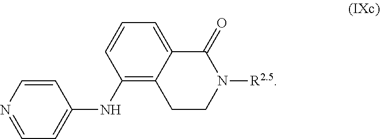

R^{2.5} is hydrogen or any value of R² as described herein, including in embodiments. In embodiments, R^{2.5} is as described herein, including in embodiments.

In embodiments, R^{2.5} is hydrogen or unsubstituted C₁-C₄ alkyl.

In embodiments, the compound has the formula:

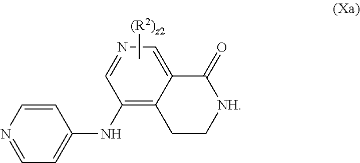

R² and z2 are as described herein, including in embodiments.

In embodiments, the compound has the formula:

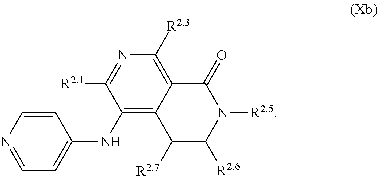

R^{2.1}, R^{2.3}, R^{2.5}, R^{2.6}, and R^{2.7} are independently hydrogen or any value of R² as described herein, including in embodiments. In embodiments, R^{2.1}, R^{2.3}, R^{2.5}, R^{2.6}, and R^{2.7} are as described herein, including in embodiments.

In embodiments, the compound has the formula:

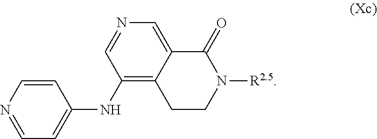

R^{2.5} is hydrogen or any value of R² as described herein, including in embodiments. In embodiments, R^{2.5} is as described herein, including in embodiments.

In embodiments, the compound has the formula:

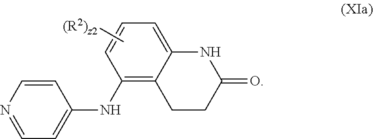

R² and z2 are as described herein, including in embodiments.

In embodiments, the compound has the formula:

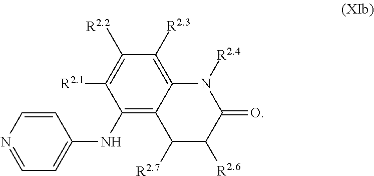

R^{2.1}, R^{2.2}, R^{2.3}, R^{2.4}, R^{2.6}, and R^{2.7} are independently hydrogen or any value of R² as described herein, including in embodiments. In embodiments, R^{2.1}, R^{2.2}, R^{2.3}, R^{2.4}, R^{2.6}, and R^{2.7} are as described herein, including in embodiments.

In embodiments, the compound has the formula:

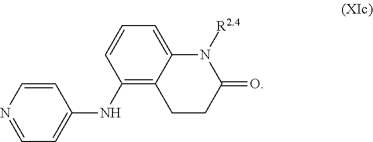

R^{2.4} is hydrogen or any value of R² as described herein, including in embodiments. In embodiments, R^{2.4} is as described herein, including in embodiments.

In embodiments, R^{2.4} is hydrogen or unsubstituted C₁-C₄ alkyl.

In embodiments, the compound has the formula:

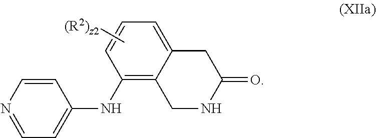

R² and z2 are as described herein, including in embodiments.

In embodiments, the compound has the formula:

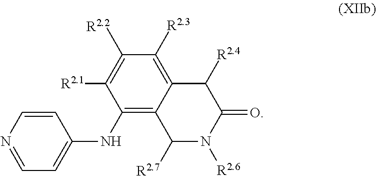

R^{2.1}, R^{2.2}, R^{2.3}, R^{2.4}, R^{2.6}, and R^{2.7} are independently hydrogen or any value of R² as described herein, including in embodiments. In embodiments, R^{2.1}, R^{2.2}, R^{2.3}, R^{2.4}, R^{2.6}, and R^{2.7} are as described herein, including in embodiments.

In embodiments, the compound has the formula:

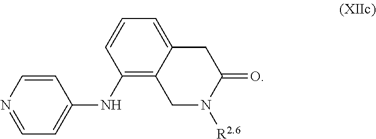

R^{2.6} is hydrogen or any value of R² as described herein, including in embodiments. In embodiments, R^{2.6} is as described herein, including in embodiments.

In embodiments, R^{2.6} is hydrogen or unsubstituted C₁-C₄ alkyl.

In an aspect is provided a compound, or a pharmaceutically acceptable salt thereof, having the formula:

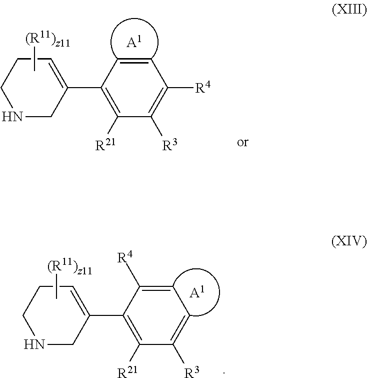

Ring A¹ is a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R²¹ and R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or R³ and R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z11 is an integer from 0 to 8.

In embodiments, the compound has the formula:

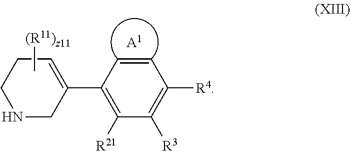

Ring A¹, R¹¹, z11, R²¹, R³, and R⁴ are as described herein, including in embodiments.

In embodiments, the compound has the formula:

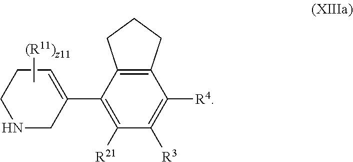

R¹¹, z11, R²¹, R³, and R⁴ are as described herein, including in embodiments.

In embodiments, the compound has the formula:

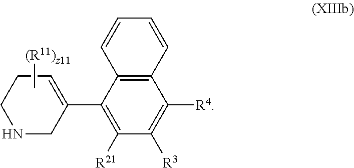

R¹¹, z11, R²¹, R³, and R⁴ are as described herein, including in embodiments.

In embodiments, the compound has the formula:

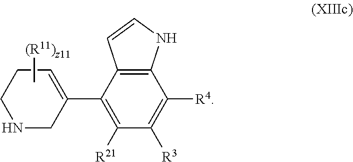

R¹¹, z11, R²¹, R³, and R⁴ are as described herein, including in embodiments.

In embodiments, the compound has the formula:

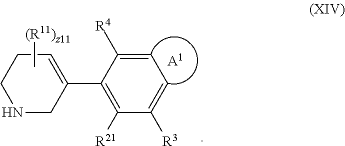

Ring A¹, R¹¹, z11, R²¹, R³, and R⁴ are as described herein, including in embodiments.

In embodiments, the compound has the formula:

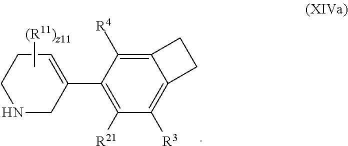

R¹¹, z11, R²¹, R³, and R⁴ are as described herein, including in embodiments.

In embodiments, the compound has the formula:

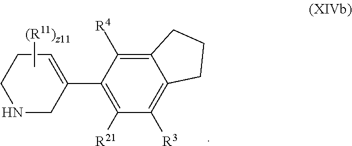

R¹¹, z11, R²¹, R³, and R⁴ are as described herein, including in embodiments.

In embodiments, the compound has the formula:

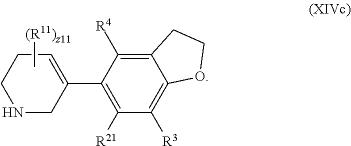

R¹¹, z11, R²¹, R³, and R⁴ are as described herein, including in embodiments.

In embodiments, the compound has the formula:

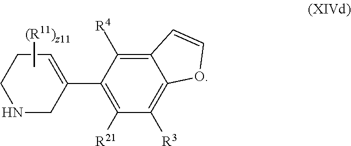

R¹¹, z11, R²¹, R³, and R⁴ are as described herein, including in embodiments.

In an aspect is provided a compound, or a pharmaceutically acceptable salt thereof, having the formula:

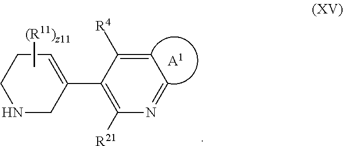

Ring A¹, R¹¹, z11, R²¹, and R⁴ are as described herein, including in embodiments.

In an aspect is provided a compound, or a pharmaceutically acceptable salt thereof, having the formula:

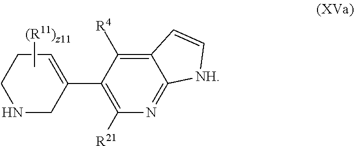

R¹¹, z11, R²¹, and R⁴ are as described herein, including in embodiments.

In embodiments, a substituted Ring A¹(e.g., substituted cycloalkyl, substituted heterocycloalkyl, substituted aryl, and/or substituted heteroaryl) is substituted with at least one substituent group, size-limited substituent group, or lower substituent group; wherein if the substituted Ring A¹ is substituted with a plurality of groups selected from substituent groups, size-limited substituent groups, and lower substituent groups; each substituent group, size-limited substituent group, and/or lower substituent group may optionally be different. In embodiments, when Ring A¹ is substituted, it is substituted with at least one substituent group. In embodiments, when Ring A¹ is substituted, it is substituted with at least one size-limited substituent group. In embodiments, when Ring A¹ is substituted, it is substituted with at least one lower substituent group.

In embodiments, Ring A¹ is a substituted or unsubstituted cycloalkyl (e.g., C₃-C₈, C₃-C₆, C₄-C₆, or C₅-C₆). In embodiments, Ring A¹ is a substituted cycloalkyl. In embodiments, Ring A¹ is oxo-substituted cycloalkyl. In embodiments, Ring A¹ is substituted or unsubstituted heterocycloalkyl. In embodiments, Ring A¹ is substituted or unsubstituted aryl. In embodiments, Ring A¹ is substituted or unsubstituted heteroaryl.

In embodiments, Ring A¹ is substituted or unsubstituted C₃-C₈ cycloalkyl. In embodiments, Ring A¹ is substituted or unsubstituted cyclopropyl. In embodiments, Ring A¹ is substituted or unsubstituted cyclobutyl. In embodiments, Ring A¹ is substituted or unsubstituted cyclopentyl. In embodiments, Ring A¹ is substituted or unsubstituted cyclohexyl. In embodiments, Ring A¹ is substituted or unsubstituted cycloheptyl. In embodiments, Ring A¹ is substituted or unsubstituted cyclooctyl.

In embodiments, Ring A¹ is a substituted cycloalkyl, wherein the substituent is oxo, halogen, —NH₂, —OH, substituted or unsubstituted C₁-C₄ alkyl, or substituted or unsubstituted 2 to 4 membered heteroalkyl.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, a substituted ring formed when two R¹¹ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R¹¹ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R¹¹ substituents are joined is substituted, it is substituted with at least one substituent group. In embodiments, when the substituted ring formed when two R¹¹ substituents are joined is substituted, it is substituted with at least one size-limited substituent group. In embodiments, when the substituted ring formed when two R¹¹ substituents are joined is substituted, it is substituted with at least one lower substituent group.

In embodiments, R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted C₁-C₆ alkyl, substituted or unsubstituted 2 to 6 membered heteroalkyl, substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, R¹¹ is independently oxo. In embodiments, R¹¹ is independently halogen. In embodiments, R¹¹ is independently —F. In embodiments, R¹¹ is independently —C₁. In embodiments, R¹¹ is independently —Br. In embodiments, R¹¹ is independently —I.

In embodiments, R¹¹ is independently —OH. In embodiments, R¹¹ is independently —NH₂. In embodiments, R¹¹ is independently unsubstituted C₁-C₄ alkyl. In embodiments, R¹¹ is independently unsubstituted methyl. In embodiments, R¹¹ is independently unsubstituted ethyl. In embodiments, R¹¹ is independently unsubstituted propyl. In embodiments, R¹¹ is independently unsubstituted n-propyl. In embodiments, R¹¹ is independently unsubstituted isopropyl. In embodiments, R¹¹ is independently unsubstituted butyl. In embodiments, R¹¹ is independently unsubstituted n-butyl. In embodiments, R¹¹ is independently unsubstituted isobutyl. In embodiments, R¹¹ is independently unsubstituted tert-butyl. In embodiments, R¹¹ is independently unsubstituted 2 to 6 membered heteroalkyl. In embodiments, R¹¹ is independently unsubstituted methoxy. In embodiments, R¹¹ is independently unsubstituted ethoxy. In embodiments, R¹¹ is independently unsubstituted propoxy. In embodiments, R″ is independently unsubstituted n-propoxy. In embodiments, R¹¹ is independently unsubstituted isopropoxy. In embodiments, R¹¹ is independently unsubstituted butoxy.

In embodiments, z11 is 0. In embodiments, z11 is 1. In embodiments, z11 is 2. In embodiments, z11 is 3. In embodiments, z11 is 4. In embodiments, z11 is 5. In embodiments, z11 is 6. In embodiments, z11 is 7. In embodiments, z11 is 8.

In embodiments, a substituted R²¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²¹ is substituted with a plurality of groups selected from substituent groups, size-limited substituent groups, and lower substituent groups; each substituent group, size-limited substituent group, and/or lower substituent group may optionally be different. In embodiments, when R²¹ is substituted, it is substituted with at least one substituent group. In embodiments, when R²¹ is substituted, it is substituted with at least one size-limited substituent group. In embodiments, when R²¹ is substituted, it is substituted with at least one lower substituent group.

In embodiments, a substituted ring formed when R²¹ and R³ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R²¹ and R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R²¹ and R³ substituents are joined is substituted, it is substituted with at least one substituent group. In embodiments, when the substituted ring formed when R²¹ and R³ substituents are joined is substituted, it is substituted with at least one size-limited substituent group. In embodiments, when the substituted ring formed when R²¹ and R³ substituents are joined is substituted, it is substituted with at least one lower substituent group.

In embodiments, R²¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or R^{2.1} and R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R²¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²¹ is halogen, —OH, —NH₂, substituted or unsubstituted C₁-C₄ alkyl, or substituted or unsubstituted 2 to 4 membered heteroalkyl. In embodiments, R²¹ is —F, —Cl, —OH, —NH₂, or unsubstituted methyl. In embodiments, R²¹ is halogen. In embodiments, R²¹ is —F. In embodiments, R²¹ is —C₁. In embodiments, R²¹ is —Br. In embodiments, R²¹ is —I. In embodiments, R²¹ is —OH. In embodiments, R²¹ is —NH₂. In embodiments, R²¹ is unsubstituted C₁-C₄ alkyl. In embodiments, R²¹ is unsubstituted methyl. In embodiments, R²¹ is unsubstituted ethyl. In embodiments, R²¹ is unsubstituted propyl. In embodiments, R²¹ is unsubstituted n-propyl. In embodiments, R²¹ is unsubstituted isopropyl. In embodiments, R²¹ is unsubstituted butyl. In embodiments, R²¹ is unsubstituted n-butyl. In embodiments, R²¹ is unsubstituted isobutyl. In embodiments, R²¹ is unsubstituted tert-butyl. In embodiments, R²¹ is unsubstituted 2 to 4 membered heteroalkyl. In embodiments, R²¹ is unsubstituted methoxy. In embodiments, R²¹ is unsubstituted ethoxy. In embodiments, R²¹ is unsubstituted propoxy. In embodiments, R²¹ is unsubstituted n-propoxy. In embodiments, R²¹ is unsubstituted isopropoxy. In embodiments, R²¹ is unsubstituted butoxy.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted ring formed when R³ and R⁴ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R³ and R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R³ and R⁴ substituents are joined is substituted, it is substituted with at least one substituent group. In embodiments, when the substituted ring formed when R³ and R⁴ substituents are joined is substituted, it is substituted with at least one size-limited substituent group. In embodiments, when the substituted ring formed when R³ and R⁴ substituents are joined is substituted, it is substituted with at least one lower substituent group.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or R³ and R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen. In embodiments, R³ is halogen, —OH, —NH₂, substituted or unsubstituted C₁-C₄ alkyl, or substituted or unsubstituted 2 to 4 membered heteroalkyl. In embodiments, R³ is —F, —Cl, —OH, —NH₂, or unsubstituted methyl. In embodiments, R³ is halogen. In embodiments, R³ is —F. In embodiments, R³ is —C₁. In embodiments, R³ is —Br. In embodiments, R³ is —I. In embodiments, R³ is —OH. In embodiments, R³ is —NH₂. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is unsubstituted 2 to 4 membered heteroalkyl. In embodiments, R³ is unsubstituted methoxy. In embodiments, R³ is unsubstituted ethoxy. In embodiments, R³ is unsubstituted propoxy. In embodiments, R³ is unsubstituted n-propoxy. In embodiments, R³ is unsubstituted isopropoxy. In embodiments, R³ is unsubstituted butoxy.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered), or R³ and R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is hydrogen, halogen, —CCl₃, —CBr₃, —CF₃, —C₁₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen. In embodiments, R⁴ is halogen, —OH, —NH₂, substituted or unsubstituted C₁-C₄ alkyl, or substituted or unsubstituted 2 to 4 membered heteroalkyl. In embodiments, R⁴ is —F, —Cl, —OH, —NH₂, or unsubstituted methyl. In embodiments, R⁴ is halogen. In embodiments, R⁴ is —F. In embodiments, R⁴ is —C₁. In embodiments, R⁴ is —Br. In embodiments, R⁴ is —I. In embodiments, R⁴ is —OH. In embodiments, R⁴ is —NH₂. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted 2 to 4 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy.

In an aspect is provided a compound, or a pharmaceutically acceptable salt thereof, having the formula:

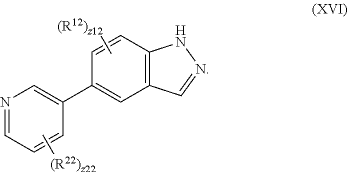

R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹² substituents may optionally be joined to form a substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R²² substituents may optionally be joined to form a substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z12 is an integer from 0 to 5.

The symbol z22 is an integer from 0 to 4.

In embodiments, the compound has the formula:

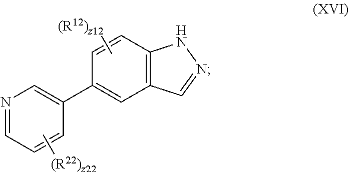

wherein R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2.2} is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); or two R^{2.2} substituents may optionally be joined to form a substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); z1 is an integer from 0 to 5; and z2 is an integer from 0 to 4; with the proviso that R¹ is not methyl.

In embodiments, the compound has the formula:

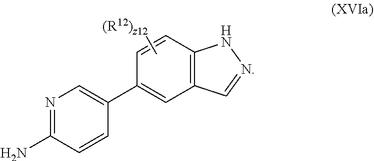

R¹² and z12 are as described herein, including in embodiments.

In an aspect is provided a compound, or a pharmaceutically acceptable salt thereof, having the formula:

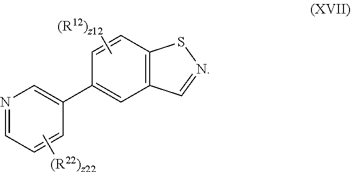

R¹², z12, R²², and z22 are as described herein, including in embodiments.

In embodiments, the compound has the formula:

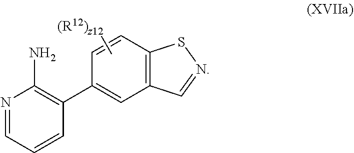

R¹² and z12 are as described herein, including in embodiments.

In embodiments, R¹² is not C₁-C₄-alkyl. In embodiments, R¹² is not unsubstituted C₁-C₄ alkyl.

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, a substituted ring formed when two R¹² substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R¹² substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R¹² substituents are joined is substituted, it is substituted with at least one substituent group. In embodiments, when the substituted ring formed when two R¹² substituents are joined is substituted, it is substituted with at least one size-limited substituent group. In embodiments, when the substituted ring formed when two R¹² substituents are joined is substituted, it is substituted with at least one lower substituent group.

In embodiments, R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is independently halogen. In embodiments, R¹² is independently —F. In embodiments, R¹² is independently —Cl. In embodiments, R¹² is independently —Br. In embodiments, R¹² is independently —I. In embodiments, R¹² is independently —CF₃. In embodiments, R¹² is independently —CN. In embodiments, R¹² is independently —NO₂. In embodiments, R¹² is independently substituted C₁-C₄ alkyl. In embodiments, R¹² is independently substituted methyl. In embodiments, R¹² is independently substituted ethyl. In embodiments, R¹² is independently substituted propyl. In embodiments, R¹² is independently substituted n-propyl. In embodiments, R¹² is independently substituted isopropyl. In embodiments, R¹² is independently substituted butyl. In embodiments, R¹² is independently substituted n-butyl. In embodiments, R¹² is independently substituted isobutyl. In embodiments, R¹² is independently substituted tert-butyl. In embodiments, R¹² is independently unsubstituted C₁₂-C₄ alkyl. In embodiments, R¹² is independently unsubstituted methyl. In embodiments, R¹² is independently unsubstituted ethyl. In embodiments, R¹² is independently unsubstituted propyl. In embodiments, R¹² is independently unsubstituted n-propyl. In embodiments, R¹² is independently unsubstituted isopropyl. In embodiments, R¹² is independently unsubstituted butyl. In embodiments, R¹² is independently unsubstituted n-butyl. In embodiments, R¹² is independently unsubstituted isobutyl. In embodiments, R¹² is independently unsubstituted tert-butyl. In embodiments, R¹² is independently substituted or unsubstituted 2 to 4 membered heteroalkyl. In embodiments, R¹² is independently unsubstituted methoxy. In embodiments, R¹² is independently unsubstituted ethoxy. In embodiments, R¹² is independently unsubstituted propoxy. In embodiments, R¹² is independently unsubstituted n-propoxy. In embodiments, R¹² is independently unsubstituted isopropoxy. In embodiments, R¹² is independently unsubstituted butoxy.

In embodiments, z12 is 0. In embodiments, z12 is 1. In embodiments, z12 is 2. In embodiments, z12 is 3. In embodiments, z12 is 4. In embodiments, z12 is 5.

In embodiments, a substituted R²² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²² is substituted with a plurality of groups selected from substituent groups, size-limited substituent groups, and lower substituent groups; each substituent group, size-limited substituent group, and/or lower substituent group may optionally be different. In embodiments, when R²² is substituted, it is substituted with at least one substituent group. In embodiments, when R²² is substituted, it is substituted with at least one size-limited substituent group. In embodiments, when R²² is substituted, it is substituted with at least one lower substituent group.

In embodiments, a substituted ring formed when two R²² substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R²² substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R²² substituents are joined is substituted, it is substituted with at least one substituent group. In embodiments, when the substituted ring formed when two R²² substituents are joined is substituted, it is substituted with at least one size-limited substituent group. In embodiments, when the substituted ring formed when two R²² substituents are joined is substituted, it is substituted with at least one lower substituent group.

In embodiments, R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R²² is independently halogen, —OH, —NH₂, substituted or unsubstituted C₁-C₄ alkyl, or substituted or unsubstituted 2 to 4 membered heteroalkyl. In embodiments, R²² is independently —F, —Cl, —OH, —NH₂, or unsubstituted methyl. In embodiments, R²² is independently halogen. In embodiments, R²² is independently —F. In embodiments, R²² is independently —Cl. In embodiments, R²² is independently —Br. In embodiments, R²² is independently —I. In embodiments, R²² is independently —OH. In embodiments, R²² is independently —NH₂. In embodiments, R²² is independently unsubstituted C₁-C₄ alkyl. In embodiments, R²² is independently unsubstituted methyl. In embodiments, R²² is independently unsubstituted ethyl. In embodiments, R²² is independently unsubstituted propyl. In embodiments, R²² is independently unsubstituted n-propyl. In embodiments, R²² is independently unsubstituted isopropyl. In embodiments, R²² is independently unsubstituted butyl. In embodiments, R²² is independently unsubstituted n-butyl. In embodiments, R²² is independently unsubstituted isobutyl. In embodiments, R²² is independently unsubstituted tert-butyl. In embodiments, R²² is independently unsubstituted 2 to 4 membered heteroalkyl. In embodiments, R²² is independently unsubstituted methoxy. In embodiments, R²² is independently unsubstituted ethoxy. In embodiments, R²² is independently unsubstituted propoxy. In embodiments, R²² is independently unsubstituted n-propoxy. In embodiments, R²² is independently unsubstituted isopropoxy. In embodiments, R²² is independently unsubstituted butoxy.

In embodiments, z22 is 0. In embodiments, z22 is 1. In embodiments, z22 is 2. In embodiments, z22 is 3. In embodiments, z22 is 4.

In an aspect is provided a compound, or a pharmaceutically acceptable salt thereof, having the formula:

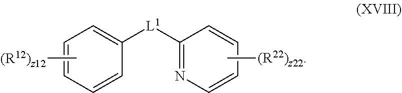

L¹ is —O—, —NR¹⁰—, or substituted or unsubstituted alkylene (e.g., C₁-C₈, C₁-C₆, C₁-C₄, or C₁-C₂).

R¹⁰ is hydrogen or unsubstituted C₁-C₄ alkyl.

R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹² substituents may optionally be joined to form a substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R²² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z12 is an integer from 0 to 5.

The symbol z22 is an integer from 0 to 4.

In embodiments, the compound has the formula:

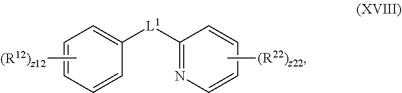

wherein L¹ is a substituted or unsubstituted alkylene (e.g., C₁-C₈, C₁-C₆, C₁-C₄, or C₁-C₂); R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R¹² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, —SF₅, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R^{2.2} substituents may optionally be joined to form a substituted or unsubstituted cycloalkyl (e.g., C₃-C₅, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); z12 is an integer from 0 to 5; and z22 is an integer from 0 to 4.

In embodiments, a substituted L¹(e.g., substituted alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is —O—. In embodiments, L¹ is —NR¹⁰—. In embodiments, L¹ is —NH—. In embodiments, L¹ is —N(CH₃)—. In embodiments, L¹ is a substituted alkylene. In embodiments, L¹ is an unsubstituted alkylene. In embodiments, L¹ is a substituted or unsubstituted C₁-C₄ alkylene. In embodiments, L¹ is a substituted or unsubstituted C₁-C₃ alkylene. In embodiments, L¹ is unsubstituted C₁-C₂ alkylene. In embodiments, L¹ is a substituted or unsubstituted methylene. In embodiments, L¹ is a substituted or unsubstituted ethylene. In embodiments, L¹ is a substituted or unsubstituted propylene. In embodiments, L¹ is a substituted or unsubstituted butylene. In embodiments, L¹ is a substituted or unsubstituted pentylene. In embodiments, L¹ is a substituted or unsubstituted hexylene. In embodiments, L¹ is a substituted or unsubstituted heptylene. In embodiments, L¹ is a substituted or unsubstituted octylene. In embodiments, L¹ is unsubstituted methylene. In embodiments, L¹ is unsubstituted ethylene. In embodiments, L¹ is unsubstituted propylene.

In embodiments, L¹ is unsubstituted butylene. In embodiments, L¹ is unsubstituted pentylene. In embodiments, L¹ is unsubstituted hexylene. In embodiments, L¹ is unsubstituted heptylene. In embodiments, L¹ is unsubstituted octylene. In embodiments, L¹ is unsubstituted ethenylene. In embodiments, L¹ is

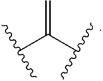 [SMILES: *C(*)=C]

In embodiments, L¹ is a substituted alkylene, wherein the substituent is a substituted or unsubstituted C₁-C₄ alkenyl.

In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl.

In embodiments, R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted C₁-C₆ alkyl, substituted or unsubstituted 2 to 6 membered heteroalkyl, substituted or unsubstituted C₃-C₅ cycloalkyl, substituted or unsubstituted 3 to 8 membered heterocycloalkyl, substituted or unsubstituted phenyl, or substituted or unsubstituted 5 to 6 membered heteroaryl; two R¹² substituents may optionally be joined to form a substituted or unsubstituted C₃-C₅ cycloalkyl, substituted or unsubstituted 3 to 8 membered heterocycloalkyl, substituted or unsubstituted phenyl, or substituted or unsubstituted 5 to 6 membered heteroaryl.

In embodiments, R¹² is independently halogen. In embodiments, R¹² is independently —F. In embodiments, R¹² is independently —Cl. In embodiments, R¹² is independently —Br. In embodiments, R¹² is independently —I. In embodiments, R¹² is independently —OH. In embodiments, R¹² is independently —NH₂. In embodiments, R¹² is independently —OCF₃. In embodiments, R¹² is independently substituted C₁₂-C₄ alkyl. In embodiments, R¹² is independently unsubstituted C₁-C₄ alkyl. In embodiments, R¹² is independently unsubstituted methyl. In embodiments, R¹² is independently unsubstituted ethyl. In embodiments, R¹² is independently unsubstituted propyl. In embodiments, R¹² is independently unsubstituted n-propyl. In embodiments, R¹² is independently unsubstituted isopropyl. In embodiments, R¹² is independently unsubstituted butyl. In embodiments, R¹² is independently unsubstituted n-butyl. In embodiments, R¹² is independently unsubstituted isobutyl. In embodiments, R¹² is independently unsubstituted tert-butyl. In embodiments, R¹² is independently unsubstituted 2 to 6 membered heteroalkyl. In embodiments, R¹² is independently unsubstituted methoxy. In embodiments, R¹² is independently unsubstituted ethoxy. In embodiments, R¹² is independently unsubstituted propoxy. In embodiments, R¹² is independently unsubstituted n-propoxy. In embodiments, R¹² is independently unsubstituted isopropoxy. In embodiments, R¹² is independently unsubstituted butoxy.

In embodiments, R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted C₁-C₆ alkyl, substituted or unsubstituted 2 to 6 membered heteroalkyl, substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl; two R²² substituents may optionally be joined to form a substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, when Ring A¹ is substituted, Ring A¹ is substituted with one or more first substituent groups denoted by R^{A.1} as explained in the definitions section above in the description of “first substituent group(s)”. In embodiments, when an R^{A.1} substituent group is substituted, the R^{A.1} substituent group is substituted with one or more second substituent groups denoted by R^{A.2} as explained in the definitions section above in the description of “first substituent group(s)”. In embodiments, when an R^{A.2} substituent group is substituted, the R^{A.2} substituent group is substituted with one or more third substituent groups denoted by R^{A.3} as explained in the definitions section above in the description of “first substituent group(s)”. In the above embodiments, Ring A¹, R^{A.1}, R^{A.2}, and R^{A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to Ring A, R^{A.1}, R^{A.2}, and R^{A.3}, respectively.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2.1} is substituted, R^{2.1} is substituted with one or more first substituent groups denoted by R^{2.1.1} as explained in the definitions section above in the description of “first substituent group(s)”. In embodiments, when an R^{2.1.1} substituent group is substituted, the R^{2.1.1} substituent group is substituted with one or more second substituent groups denoted by R^{2.1.2} as explained in the definitions section above in the description of “first substituent group(s)”. In embodiments, when an R^{2.1.2} substituent group is substituted, the R^{2.1.2} substituent group is substituted with one or more third substituent groups denoted by R^{2.1.3} as explained in the definitions section above in the description of “first substituent group(s)”. In the above embodiments, R^{2.1}, R^{2.1.1}, R^{2.1.2}, and R^{2.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.1}, R^{2.1.1}, R^{2.1.2}, and R^{2.1.3}, respectively.

In embodiments, when R^{2.2} is substituted, R^{2.2} is substituted with one or more first substituent groups denoted by R^{2.2.1} as explained in the definitions section above in the description of “first substituent group(s)”. In embodiments, when an R^{2.2.1} substituent group is substituted, the R^{2.2.1} substituent group is substituted with one or more second substituent groups denoted by R^{2.2.2} as explained in the definitions section above in the description of “first substituent group(s)”. In embodiments, when an R^{2.2.2} substituent group is substituted, the R^{2.2.2} substituent group is substituted with one or more third substituent groups denoted by R^{2.2.3} as explained in the definitions section above in the description of “first substituent group(s)”. In the above embodiments, R^{2.2}, R^{2.2.1}, R^{2.2.2}, and R^{2.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.2}, R^{2.2.1}, R^{2.2.2}, and R^{2.2.3}, respectively.

In embodiments, when R^{2.3} is substituted, R^{2.3} is substituted with one or more first substituent groups denoted by R^{2.3.1} as explained in the definitions section above in the description of “first substituent group(s)”. In embodiments, when an R^{2.3.1} substituent group is substituted, the R^{2.3.1} substituent group is substituted with one or more second substituent groups denoted by R^{2.3.2} as explained in the definitions section above in the description of “first substituent group(s)”. In embodiments, when an R^{2.3.2} substituent group is substituted, the R^{2.3.2} substituent group is substituted with one or more third substituent groups denoted by R^{2.3.3} as explained in the definitions section above in the description of “first substituent group(s)”. In the above embodiments, R^{2.3}, R^{2.3.1}, R^{2.3.2}, and R^{2.3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.3}, R^{2.3.1}, R^{2.3.2}, and R^{2.3.3}, respectively.

In embodiments, when R^{2.4} is substituted, R^{2.4} is substituted with one or more first substituent groups denoted by R^{2.4.1} as explained in the definitions section above in the description of “first substituent group(s)”. In embodiments, when an R^{2.4.1} substituent group is substituted, the R^{2.4.1} substituent group is substituted with one or more second substituent groups denoted by R^{2.4.2} as explained in the definitions section above in the description of “first substituent group(s)”. In embodiments, when an R^{2.4.2} substituent group is substituted, the R^{2.4.2} substituent group is substituted with one or more third substituent groups denoted by R^{2.4.3} as explained in the definitions section above in the description of “first substituent group(s)”. In the above embodiments, R^{2.4}, R^{2.4.1}, R^{2.4.2}, and R^{2.4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.4}, R^{2.4.1}, R^{2.4.2}, and R^{2.4.3}, respectively.

In embodiments, when R^{2.5} is substituted, R^{2.5} is substituted with one or more first substituent groups denoted by R^{2.5.1} as explained in the definitions section above in the description of “first substituent group(s)”. In embodiments, when an R^{2.5.1} substituent group is substituted, the R^{2.5.1} substituent group is substituted with one or more second substituent groups denoted by R^{2.5.2} as explained in the definitions section above in the description of “first substituent group(s)”. In embodiments, when an R^{2.5.2} substituent group is substituted, the R^{2.5.2} substituent group is substituted with one or more third substituent groups denoted by R^{2.5.3} as explained in the definitions section above in the description of “first substituent group(s)”. In the above embodiments, R^{2.5}, R^{2.5.1}, R^{2.5.2}, and R^{2.5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.5}, R^{2.5.1}, R^{2.5.2}, and R^{2.5.3}, respectively.

In embodiments, when R^{2.6} is substituted, R^{2.6} is substituted with one or more first substituent groups denoted by R^{2.6.1} as explained in the definitions section above in the description of “first substituent group(s)”. In embodiments, when an R^{2.6.1} substituent group is substituted, the R^{2.6.1} substituent group is substituted with one or more second substituent groups denoted by R^{2.6.2} as explained in the definitions section above in the description of “first substituent group(s)”. In embodiments, when an R^{2.6.2} substituent group is substituted, the R^{2.6.2} substituent group is substituted with one or more third substituent groups denoted by R^{2.6.3} as explained in the definitions section above in the description of “first substituent group(s)”. In the above embodiments, R^{2.6}, R^{2.6.1}, R^{2.6.2}, and R^{2.6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.6}, R^{2.6.1}, R^{2.6.2}, and R^{2.6.3}, respectively.

In embodiments, when R^{2.7} is substituted, R^{2.7} is substituted with one or more first substituent groups denoted by R^{2.7.1} as explained in the definitions section above in the description of “first substituent group(s)”. In embodiments, when an R^{2.7.1} substituent group is substituted, the R^{2.7.1} substituent group is substituted with one or more second substituent groups denoted by R^{2.7.2} as explained in the definitions section above in the description of “first substituent group(s)”. In embodiments, when an R^{2.7.2} substituent group is substituted, the R^{2.7.2} substituent group is substituted with one or more third substituent groups denoted by R^{2.7.3} as explained in the definitions section above in the description of “first substituent group(s)”. In the above embodiments, R^{2.7}, R^{2.7.1}, R^{2.7.2}, and R^{2.7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2.7}, R^{2.7.1}, R^{2.7.2}, and R^{2.73}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW}1, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R^{11.1}, R^{11.2}, and R^{11.3}, respectively.

In embodiments, when two R¹¹ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹¹, R¹¹, R^{11.2}, and R^{11.3}, respectively.

In embodiments, when R¹² is substituted, R¹² is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹², R^{12.1}, R^{12.2}, and R^{12.3}, respectively.

In embodiments, when two R¹² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹², R^{12.1}, R^{12.2}, and R¹²³, respectively.

In embodiments, when R²¹ is substituted, R²¹ is substituted with one or more first substituent groups denoted by R^{21.1} as explained in the definitions section above in the description of “first substituent group(s)”. In embodiments, when an R^{21.1} substituent group is substituted, the R^{21.1} substituent group is substituted with one or more second substituent groups denoted by R^{21.2} as explained in the definitions section above in the description of “first substituent group(s)”. In embodiments, when an R^{21.2} substituent group is substituted, the R^{21.2} substituent group is substituted with one or more third substituent groups denoted by R^{21.3} as explained in the definitions section above in the description of “first substituent group(s)”. In the above embodiments, R²¹, R^{21.1}, R^{21.2}, and R^{21.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²¹, R^{21.1}, R^{21.2}, and R^{21.3}, respectively.

In embodiments, when R²² is substituted, R²² is substituted with one or more first substituent groups denoted by R^{22.1} as explained in the definitions section above in the description of “first substituent group(s)”. In embodiments, when an R^{22.1} substituent group is substituted, the R^{22.1} substituent group is substituted with one or more second substituent groups denoted by R^{22.2} as explained in the definitions section above in the description of “first substituent group(s)”. In embodiments, when an R^{22.2} substituent group is substituted, the R^{22.2} substituent group is substituted with one or more third substituent groups denoted by R^{22.3} as explained in the definitions section above in the description of “first substituent group(s)”. In the above embodiments, R²², R^{22.1}, R^{22.2}, and R^{22.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²², R^{22.1}, R^{22.2}, and R^{22.3}, respectively.

In embodiments, when two R²² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{22.1} as explained in the definitions section above in the description of “first substituent group(s)”. In embodiments, when an R^{22.1} substituent group is substituted, the R^{22.1} substituent group is substituted with one or more second substituent groups denoted by R^{22.2} as explained in the definitions section above in the description of “first substituent group(s)”. In embodiments, when an R^{22.2} substituent group is substituted, the R^{22.2} substituent group is substituted with one or more third substituent groups denoted by R^{22.3} as explained in the definitions section above in the description of “first substituent group(s)”. In the above embodiments, R²², R^{22.1}, R^{22.2}, and R^{22.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²², R^{22.1}, R^{22.2}, and R^{22.3}, respectively.

In embodiments, when R³ is substituted, R³ is substituted with one or more first substituent groups denoted by R^{3.1} as explained in the definitions section above in the description of “first substituent group(s)”. In embodiments, when an R^{3A} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R³ as explained in the definitions section above in the description of “first substituent group(s)”. In the above embodiments, R³, R^{3.1}, R^{3.2}, and R³³ have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R²¹ and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{21.1} as explained in the definitions section above in the description of “first substituent group(s)”. In embodiments, when an R^{21.1} substituent group is substituted, the R^{21.1} substituent group is substituted with one or more second substituent groups denoted by R^{21.2} as explained in the definitions section above in the description of “first substituent group(s)”. In embodiments, when an R^{21.2} substituent group is substituted, the R^{21.2} substituent group is substituted with one or more third substituent groups denoted by R^{21.3} as explained in the definitions section above in the description of “first substituent group(s)”. In the above embodiments, R^{21.1}, R^{21.2}, and R^{21.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{21.1}, R^{21.2}, and R²¹³, respectively.

In embodiments, when R²¹ and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3A} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R^{4.1}, R^{4.2}, and R^{4.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4.1}, R^{4.2}, and R^{4.3}, respectively.

In embodiments, when L¹ is substituted, L¹ is substituted with one or more first substituent groups denoted by R^{L.1.1} as explained in the definitions section above in the description of “first substituent group(s)”. In embodiments, when an R^{L.1.1} substituent group is substituted, the R^{L1.1} substituent group is substituted with one or more second substituent groups denoted by R^{L1.2} as explained in the definitions section above in the description of “first substituent group(s)”. In embodiments, when an R^{L.1.2} substituent group is substituted, the R^{L1.2} substituent group is substituted with one or more third substituent groups denoted by R^{L.1.3} as explained in the definitions section above in the description of “first substituent group(s)”. In the above embodiments, L¹, R^{L.1.1}, R^{L.1.2}, and R^{L.1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L.1.1}, R^{L.1.2} and R^{L1..3}, respectively.

In embodiments, the compound has the formula:

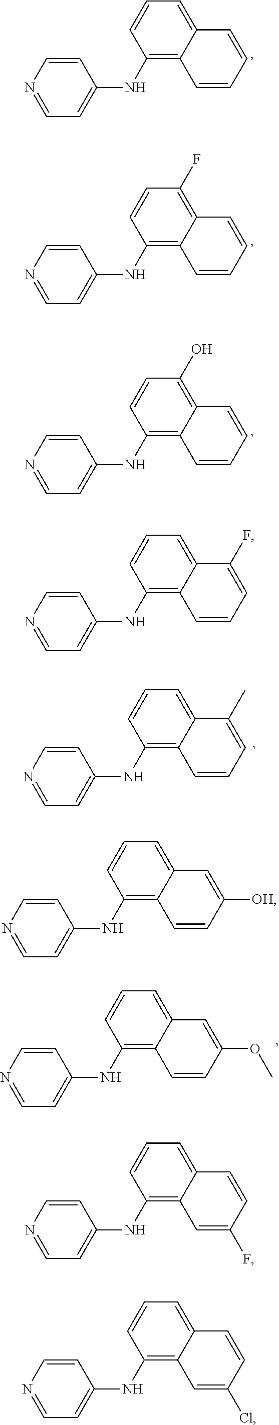

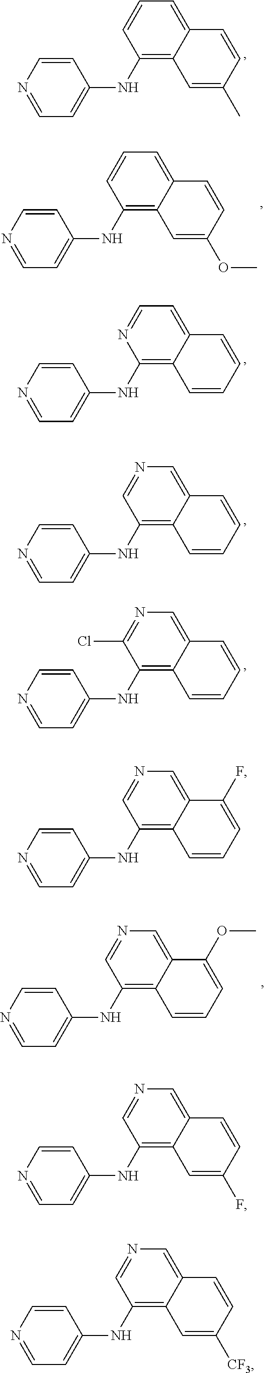

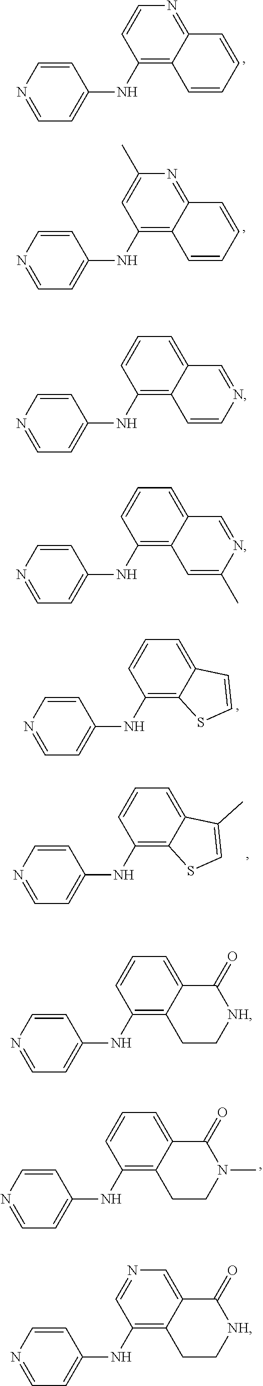

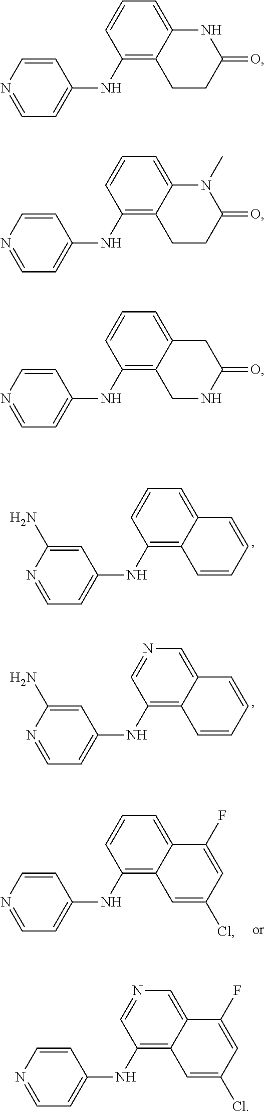

In embodiments, the compound has the formula:

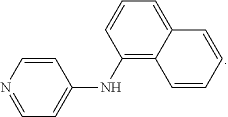 [SMILES: c1ccc2c(Nc3ccncc3)cccc2c1]

In embodiments, the compound has the formula:

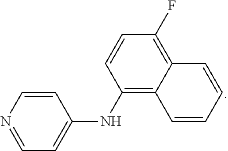 [SMILES: Fc1ccc(Nc2ccncc2)c2ccccc12]

In embodiments, the compound has the formula:

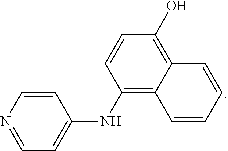 [SMILES: Oc1ccc(Nc2ccncc2)c2ccccc12]

In embodiments, the compound has the formula:

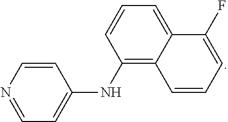 [SMILES: Fc1cccc2c(Nc3ccncc3)cccc12]

In embodiments, the compound has the formula:

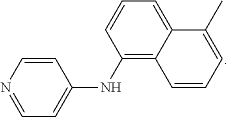 [SMILES: Cc1cccc2c(Nc3ccncc3)cccc12]

In embodiments, the compound has the formula:

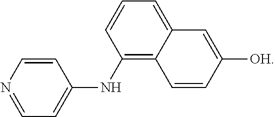 [SMILES: Oc1ccc2c(Nc3ccncc3)cccc2c1]

In embodiments, the compound has the formula:

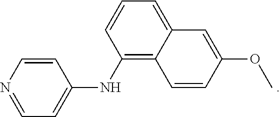 [SMILES: COc1ccc2c(Nc3ccncc3)cccc2c1]

In embodiments, the compound has the formula:

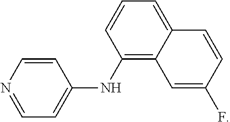 [SMILES: Fc1ccc2cccc(Nc3ccncc3)c2c1]

In embodiments, the compound has the formula:

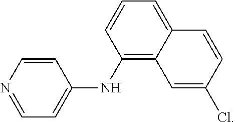 [SMILES: Clc1ccc2cccc(Nc3ccncc3)c2c1]

In embodiments, the compound has the formula:

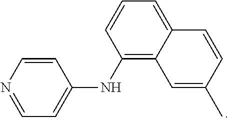 [SMILES: Cc1ccc2cccc(Nc3ccncc3)c2c1]

In embodiments, the compound has the formula:

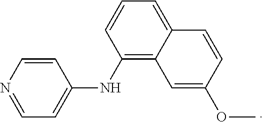 [SMILES: COc1ccc2cccc(Nc3ccncc3)c2c1]

In embodiments, the compound has the formula:

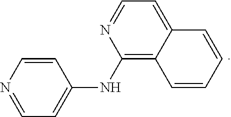 [SMILES: c1ccc2c(Nc3ccncc3)nccc2c1]

In embodiments, the compound has the formula:

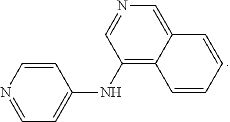 [SMILES: c1ccc2c(Nc3ccncc3)cncc2c1]

In embodiments, the compound has the formula:

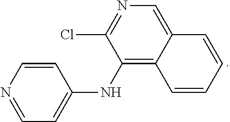 [SMILES: Clc1ncc2ccccc2c1Nc1ccncc1]

In embodiments, the compound has the formula:

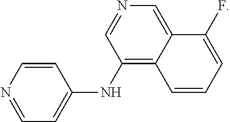 [SMILES: Fc1cccc2c(Nc3ccncc3)cncc12]

In embodiments, the compound has the formula:

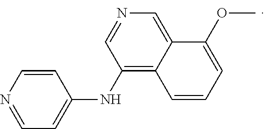 [SMILES: COc1cccc2c(Nc3ccncc3)cncc12]

In embodiments, the compound has the formula:

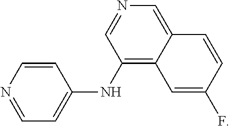 [SMILES: Fc1ccc2cncc(Nc3ccncc3)c2c1]

In embodiments, the compound has the formula:

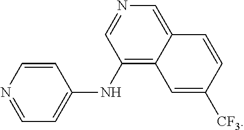 [SMILES: FC(F)(F)c1ccc2cncc(Nc3ccncc3)c2c1]

In embodiments, the compound has the formula:

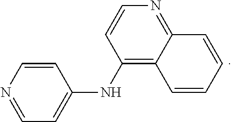 [SMILES: c1ccc2c(Nc3ccncc3)ccnc2c1]

In embodiments, the compound has the formula:

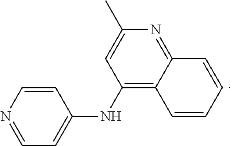 [SMILES: Cc1cc(Nc2ccncc2)c2ccccc2n1]

In embodiments, the compound has the formula:

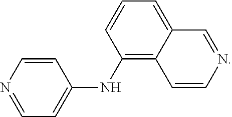 [SMILES: c1cc(Nc2ccncc2)c2ccncc2c1]

In embodiments, the compound has the formula:

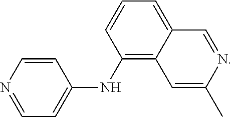 [SMILES: Cc1cc2c(Nc3ccncc3)cccc2cn1]

In embodiments, the compound has the formula:

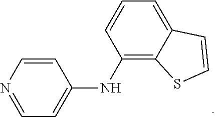 [SMILES: c1cc(Nc2ccncc2)c2sccc2c1]

In embodiments, the compound has the formula:

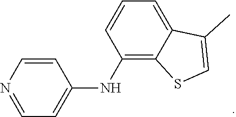 [SMILES: Cc1csc2c(Nc3ccncc3)cccc12]

In embodiments, the compound has the formula:

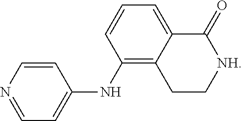 [SMILES: O=C1NCCc2c(Nc3ccncc3)cccc21]

In embodiments, the compound has the formula:

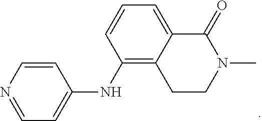 [SMILES: CN1CCc2c(Nc3ccncc3)cccc2C1=O]

In embodiments, the compound has the formula:

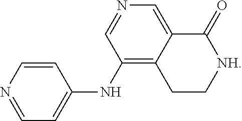 [SMILES: O=C1NCCc2c(Nc3ccncc3)cncc21]

In embodiments, the compound has the formula:

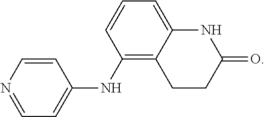 [SMILES: O=C1CCc2c(cccc2Nc2ccncc2)N1]

In embodiments, the compound has the formula:

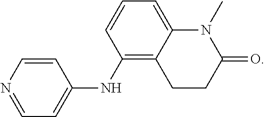 [SMILES: CN1C(=O)CCc2c(Nc3ccncc3)cccc21]

In embodiments, the compound has the formula:

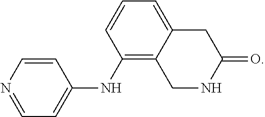 [SMILES: O=C1Cc2cccc(Nc3ccncc3)c2CN1]

In embodiments, the compound has the formula:

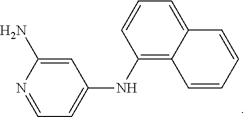 [SMILES: Nc1cc(Nc2cccc3ccccc23)ccn1]

In embodiments, the compound has the formula:

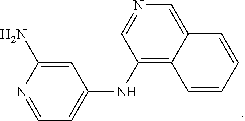 [SMILES: Nc1cc(Nc2cncc3ccccc23)ccn1]

In embodiments, the compound has the formula:

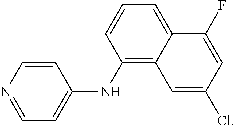 [SMILES: Fc1cc(Cl)cc2c(Nc3ccncc3)cccc12]

In embodiments, the compound has the formula:

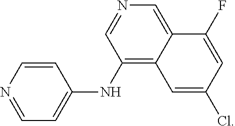 [SMILES: Fc1cc(Cl)cc2c(Nc3ccncc3)cncc12]

In embodiments, the compound has the formula:

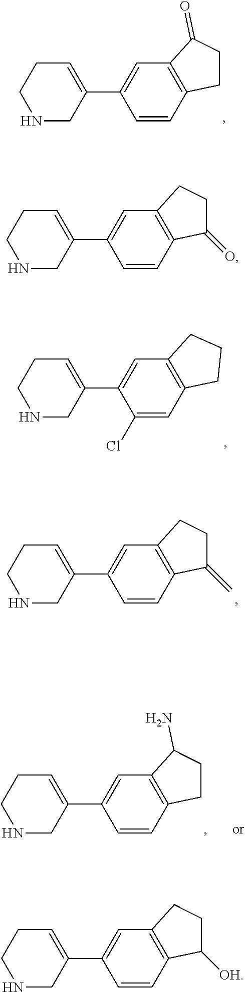 [SMILES: O=C1CCc2ccc(C3=CCCNC3)cc21 ; O=C1CCc2cc(C3=CCCNC3)ccc21 ; Clc1cc2c(cc1C1=CCCNC1)CCC2 ; C=C1CCc2cc(C3=CCCNC3)ccc21 ; NC1CCc2ccc(C3=CCCNC3)cc21 ; OC1CCc2cc(C3=CCCNC3)ccc21]

In embodiments, the compound has the formula:

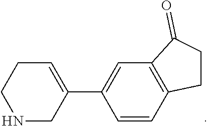 [SMILES: O=C1CCc2ccc(C3=CCCNC3)cc21]

In embodiments, the compound has the formula:

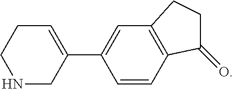 [SMILES: O=C1CCc2cc(C3=CCCNC3)ccc21]

In embodiments, the compound has the formula:

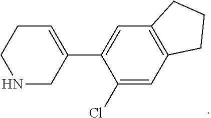 [SMILES: Clc1cc2c(cc1C1=CCCNC1)CCC2]

In embodiments, the compound has the formula:

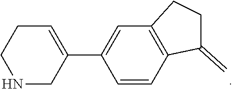 [SMILES: C=C1CCc2cc(C3=CCCNC3)ccc21]

In embodiments, the compound has the formula:

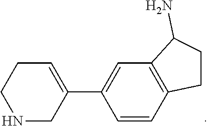 [SMILES: NC1CCc2ccc(C3=CCCNC3)cc21]

In embodiments, the compound has the formula:

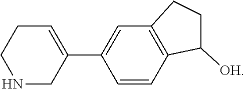 [SMILES: OC1CCc2cc(C3=CCCNC3)ccc21]

In embodiments, the compound has the formula:

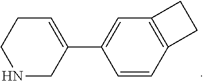 [SMILES: C1=C(c2ccc3c(c2)CC3)CNCC1]

In embodiments, the compound has the formula: H

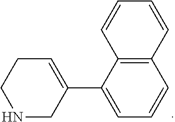 [SMILES: C1=C(c2cccc3ccccc23)CNCC1]

In embodiments, the compound has the formula:

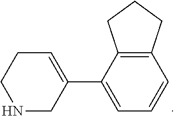 [SMILES: C1=C(c2cccc3c2CCC3)CNCC1]

In embodiments, the compound has the formula:

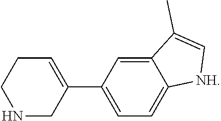 [SMILES: Cc1c[nH]c2ccc(C3=CCCNC3)cc12]

In embodiments, the compound has the formula:

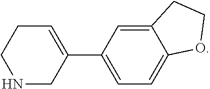 [SMILES: C1=C(c2ccc3c(c2)CCO3)CNCC1]

In embodiments, the compound has the formula:

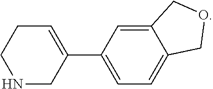 [SMILES: C1=C(c2ccc3c(c2)COC3)CNCC1]

In embodiments, the compound has the formula:

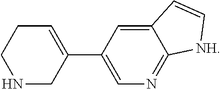 [SMILES: C1=C(c2cnc3[nH]ccc3c2)CNCC1]

In embodiments, the compound has the formula:

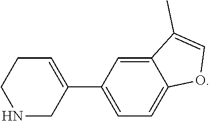 [SMILES: Cc1coc2ccc(C3=CCCNC3)cc12]

In embodiments, the compound has the formula:

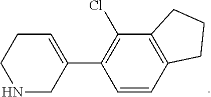 [SMILES: Clc1c(C2=CCCNC2)ccc2c1CCC2]

In embodiments, the compound has the formula:

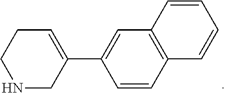 [SMILES: C1=C(c2ccc3ccccc3c2)CNCC1]

In embodiments, the compound has the formula:

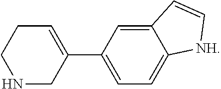 [SMILES: C1=C(c2ccc3[nH]ccc3c2)CNCC1]

In embodiments, the compound has the formula:

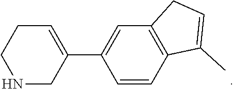 [SMILES: CC1=CCc2cc(C3=CCCNC3)ccc21]

In embodiments, the compound has the formula:

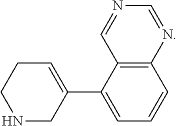 [SMILES: C1=C(c2cccc3ncncc23)CNCC1]

In embodiments, the compound has the formula:

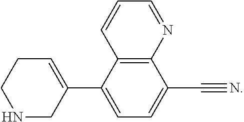 [SMILES: N#Cc1ccc(C2=CCCNC2)c2cccnc12]

In embodiments, the compound has the formula:

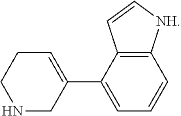 [SMILES: C1=C(c2cccc3[nH]ccc23)CNCC1]

In embodiments, the compound has the formula:

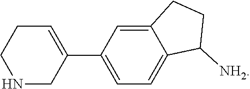 [SMILES: NC1CCc2cc(C3=CCCNC3)ccc21]

In embodiments, the compound has the formula:

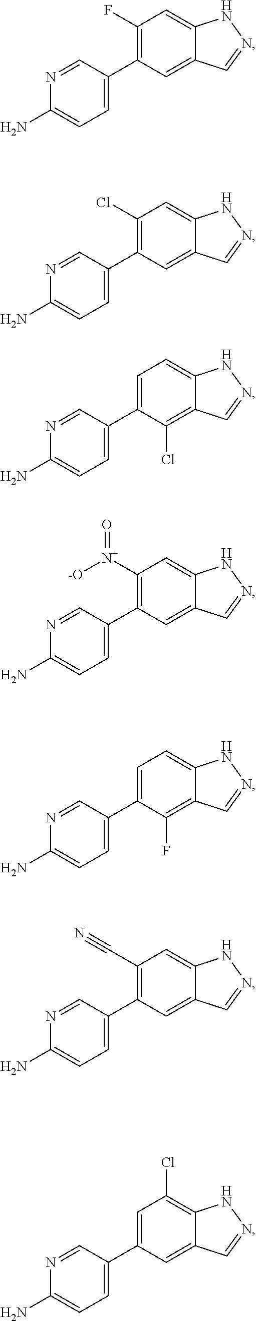

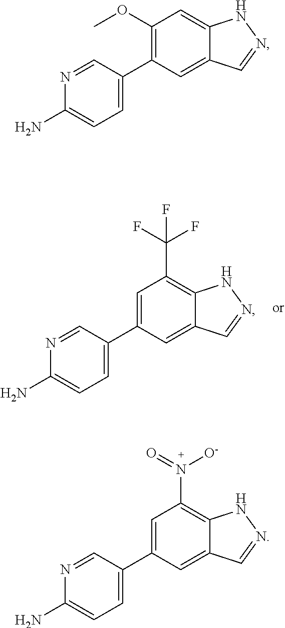

In embodiments, the compound has the formula:

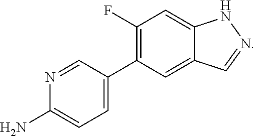 [SMILES: Nc1ccc(-c2cc3cn[nH]c3cc2F)cn1]

In embodiments, the compound has the formula:

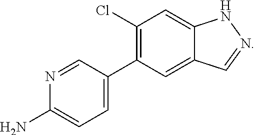 [SMILES: Nc1ccc(-c2cc3cn[nH]c3cc2Cl)cn1]

In embodiments, the compound has the formula:

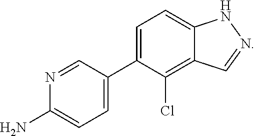 [SMILES: Nc1ccc(-c2ccc3[nH]ncc3c2Cl)cn1]

In embodiments, the compound has the formula:

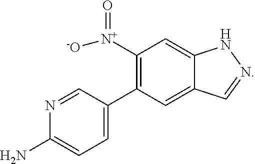

In embodiments, the compound has the formula:

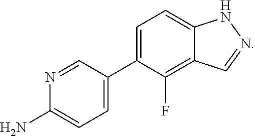 [SMILES: Nc1ccc(-c2ccc3[nH]ncc3c2F)cn1]

In embodiments, the compound has the formula:

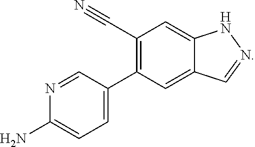 [SMILES: N#Cc1cc2[nH]ncc2cc1-c1ccc(N)nc1]

In embodiments, the compound has the formula:

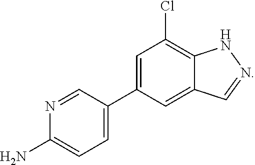 [SMILES: Nc1ccc(-c2cc(Cl)c3[nH]ncc3c2)cn1]

In embodiments, the compound has the formula:

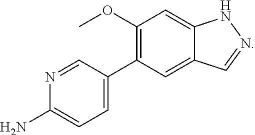 [SMILES: COc1cc2[nH]ncc2cc1-c1ccc(N)nc1]

In embodiments, the compound has the formula:

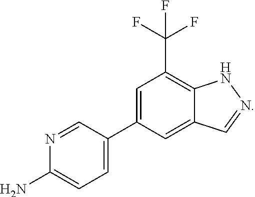 [SMILES: Nc1ccc(-c2cc(C(F)(F)F)c3[nH]ncc3c2)cn1]

In embodiments, the compound has the formula:

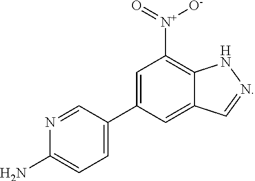 [SMILES: Nc1ccc(-c2cc([N+](=O)[O-])c3[nH]ncc3c2)cn1]

In embodiments, the compound has the formula:

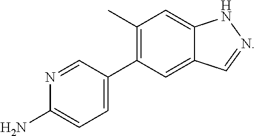 [SMILES: Cc1cc2[nH]ncc2cc1-c1ccc(N)nc1]

In embodiments, the compound has the formula:

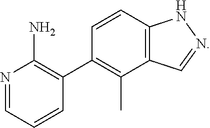 [SMILES: Cc1c(-c2cccnc2N)ccc2[nH]ncc12]

In embodiments, the compound has the formula:

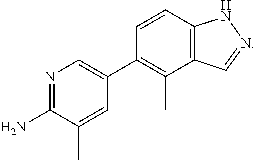 [SMILES: Cc1cc(-c2ccc3[nH]ncc3c2C)cnc1N]

In embodiments, the compound has the formula:

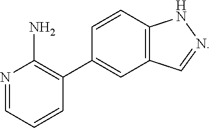 [SMILES: Nc1ncccc1-c1ccc2[nH]ncc2c1]

In embodiments, the compound has the formula:

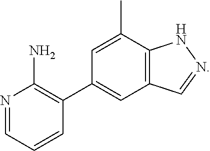 [SMILES: Cc1cc(-c2cccnc2N)cc2cn[nH]c12]

In embodiments, the compound has the formula:

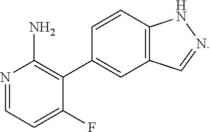 [SMILES: Nc1nccc(F)c1-c1ccc2[nH]ncc2c1]

In embodiments, the compound has the formula:

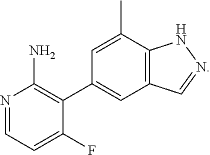 [SMILES: Cc1cc(-c2c(F)ccnc2N)cc2cn[nH]c12]

In embodiments, the compound has the formula:

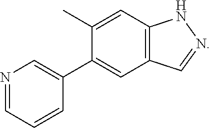 [SMILES: Cc1cc2[nH]ncc2cc1-c1cccnc1]

In embodiments, the compound has the formula:

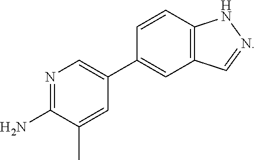 [SMILES: Cc1cc(-c2ccc3[nH]ncc3c2)cnc1N]

In embodiments, the compound has the formula:

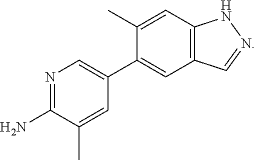 [SMILES: Cc1cc2[nH]ncc2cc1-c1cnc(N)c(C)c1]

In embodiments, the compound has the formula:

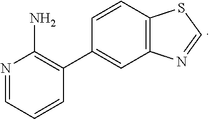 [SMILES: Nc1ncccc1-c1ccc2scnc2c1]

In embodiments, the compound has the formula:

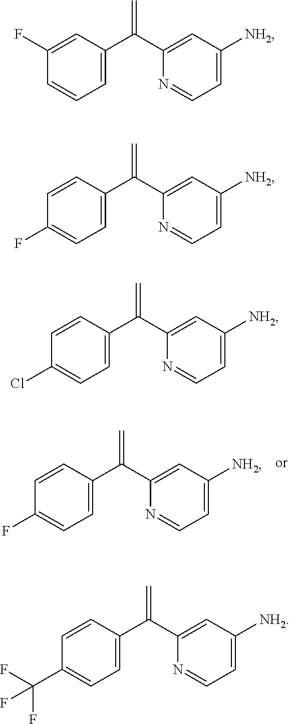 [SMILES: C=C(c1cccc(F)c1)c1cc(N)ccn1 ; C=C(c1ccc(F)cc1)c1cc(N)ccn1 ; C=C(c1ccc(Cl)cc1)c1cc(N)ccn1 ; C=C(c1ccc(F)cc1)c1cc(N)ccn1 ; "or" ; C=C(c1ccc(C(F)(F)F)cc1)c1cc(N)ccn1]

In embodiments, the compound has the formula:

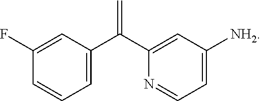 [SMILES: C=C(c1cccc(F)c1)c1cc(N)ccn1]

In embodiments, the compound has the formula:

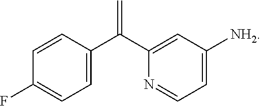 [SMILES: C=C(c1ccc(F)cc1)c1cc(N)ccn1]

In embodiments, the compound has the formula:

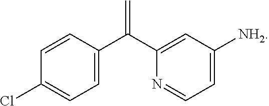 [SMILES: C=C(c1ccc(Cl)cc1)c1cc(N)ccn1]

In embodiments, the compound has the formula:

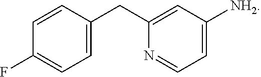 [SMILES: Nc1ccnc(Cc2ccc(F)cc2)c1]

In embodiments, the compound has the formula:

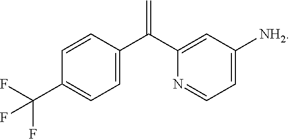 [SMILES: C=C(c1ccc(C(F)(F)F)cc1)c1cc(N)ccn1]

In embodiments, the compound has the formula:

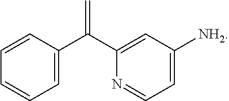

In embodiments, the compound has the formula:

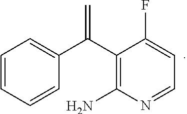 [SMILES: C=C(c1ccccc1)c1c(F)ccnc1N]

In embodiments, the compound has the formula:

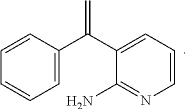

In embodiments, the compound has the formula:

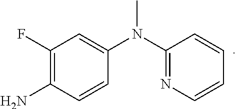 [SMILES: CN(c1ccc(N)c(F)c1)c1ccccn1]

In embodiments, the compound has the formula:

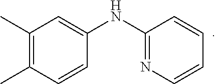

In embodiments, the compound has the formula:

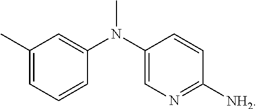 [SMILES: Cc1cccc(N(C)c2ccc(N)nc2)c1]

In embodiments, the compound has the formula:

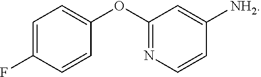 [SMILES: Nc1ccnc(Oc2ccc(F)cc2)c1]

In embodiments, the compound has the formula:

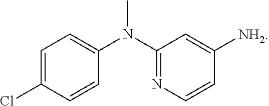 [SMILES: CN(c1ccc(Cl)cc1)c1cc(N)ccn1]

In embodiments, the compound has the formula:

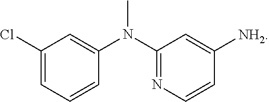 [SMILES: CN(c1cccc(Cl)c1)c1cc(N)ccn1]

In embodiments, the compound has the formula:

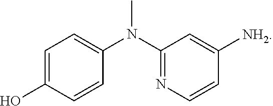 [SMILES: CN(c1ccc(O)cc1)c1cc(N)ccn1]

In embodiments, the compound has the formula:

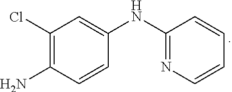 [SMILES: Nc1ccc(Nc2ccccn2)cc1Cl]

In embodiments, the compound has the formula:

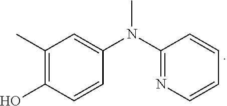 [SMILES: Cc1cc(N(C)c2ccccn2)ccc1O]

In embodiments, the compound has the formula:

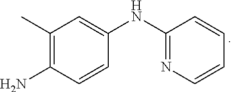

In embodiments, the compound has the formula:

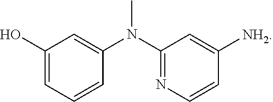 [SMILES: CN(c1cccc(O)c1)c1cc(N)ccn1]

In embodiments, the compound has the formula:

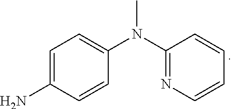

In embodiments, the compound has the formula:

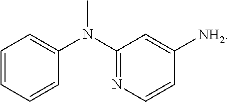

In embodiments, the compound has the formula:

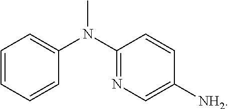

In embodiments, R¹ is not halogen. In embodiments, R¹ is not —F. In embodiments, R¹ is not —Cl. In embodiments, R¹ is not —Br. In embodiments, R¹ is not —I. In embodiments, R¹ is not —NH₂.

In embodiments, R² is not oxo. In embodiments, R² is not halogen. In embodiments, R² is not —F. In embodiments, R² is not —Cl. In embodiments, R² is not —Br.

In embodiments, R² is not —I. In embodiments, R² is not —CF₃. In embodiments, R² is not —CN. In embodiments, R² is not —NR^{2A}R^{2B}. In embodiments, R² is not —NR^{2A}R^{2B}, wherein R^{2A} is substituted or unsubstituted heteroaryl and R^{2B} is hydrogen. In embodiments, R² is not —NH₂. In embodiments, R² is not —NHCH₃. In embodiments, R² is not —NO₂. In embodiments, R² is not —C(O)R^{2C}. In embodiments, R² is not —C(O)—C(O)OH. In embodiments, R² is not —C(O)—C(O)OCH₃. In embodiments, R² is not —NR^{2A}C(O)R^{2C}. In embodiments, R² is not —NR^{2A}C(O)R^{2C}, wherein R^{2A} is hydrogen and R^{2C} is a substituted phenyl. In embodiments, R² is not —SOₙ₂R^{2D}. In embodiments, R² is not —SO₃H. In embodiments, R² is not —C(O)NR^{2A}R^{2B}. In embodiments, R² is not —OR^{2D}. In embodiments, R² is not —OR^{2D}, wherein R^{2D} is a substituted heterocycloalkyl. In embodiments, R² is not —OH. In embodiments, R² is not —OCH₃. In embodiments, R² is not unsubstituted C₁-C₄ alkyl. In embodiments, R² is not unsubstituted methyl. In embodiments, R² is not unsubstituted ethyl. In embodiments, R² is not unsubstituted propyl. In embodiments, R² is not unsubstituted n-propyl. In embodiments, R² is not unsubstituted isopropyl. In embodiments, R² is not unsubstituted butyl. In embodiments, R² is not unsubstituted n-butyl. In embodiments, R² is not unsubstituted isobutyl. In embodiments, R² is not unsubstituted tert-butyl. In embodiments, R² is not substituted or unsubstituted heteroalkyl. In embodiments, R² is not unsubstituted cycloalkyl. In embodiments, R² is not unsubstituted cyclopropyl. In embodiments, R² is not unsubstituted cyclobutyl. In embodiments, R² is not unsubstituted cyclopentyl. In embodiments, R² is not unsubstituted cyclohexyl. In embodiments, R² is not substituted or unsubstituted aryl. In embodiments, R² is not unsubstituted phenyl. In embodiments, R² is not unsubstituted naphthyl. In embodiments, R² is not substituted or unsubstituted heteroaryl. In embodiments, R² is not substituted or unsubstituted pyridyl. In embodiments, R² is not substituted or unsubstituted thiophenyl. In embodiments, R² is not substituted or unsubstituted furanyl. In embodiments, two R² substituents are not joined to form a substituted or unsubstituted aryl. In embodiments, two R² substituents are not joined to form a substituted or unsubstituted heteroaryl.

In embodiments, z1 is not 0. In embodiments, z2 is not 0.

In embodiments, the compound does not have the formula:

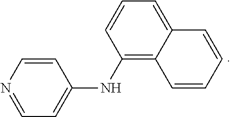 [SMILES: c1ccc2c(Nc3ccncc3)cccc2c1]

In embodiments, the compound does not have the formula:

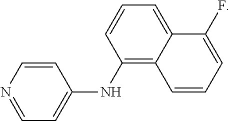 [SMILES: Fc1cccc2c(Nc3ccncc3)cccc12]

In embodiments, the compound does not have the formula:

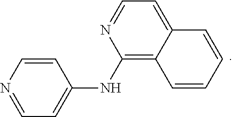 [SMILES: c1ccc2c(Nc3ccncc3)nccc2c1]

In embodiments, the compound does not have the formula:

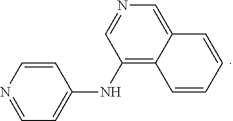 [SMILES: c1ccc2c(Nc3ccncc3)cncc2c1]

In embodiments, the compound does not have the formula:

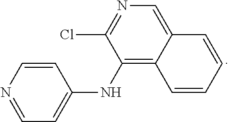 [SMILES: Clc1ncc2ccccc2c1Nc1ccncc1]

In embodiments, the compound does not have the formula:

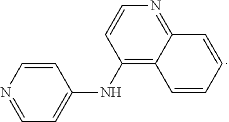 [SMILES: c1ccc2c(Nc3ccncc3)ccnc2c1]

In embodiments, the compound does not have the formula:

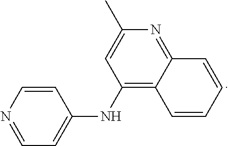 [SMILES: Cc1cc(Nc2ccncc2)c2ccccc2n1]

In embodiments, the compound does not have the formula:

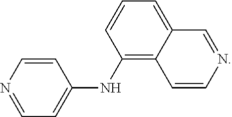 [SMILES: c1cc(Nc2ccncc2)c2ccncc2c1]

In embodiments, the compound does not have the formula:

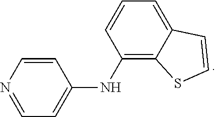 [SMILES: c1cc(Nc2ccncc2)c2sccc2c1]

In embodiments, the compound does not have the formula:

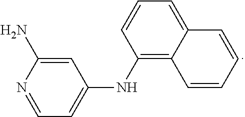 [SMILES: Nc1cc(Nc2cccc3ccccc23)ccn1]

In embodiments, the compound does not have the formula:

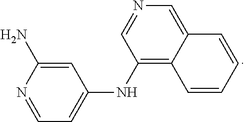 [SMILES: Nc1cc(Nc2cncc3ccccc23)ccn1]

In embodiments, the compound does not have the formula:

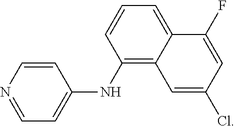 [SMILES: Fc1cc(Cl)cc2c(Nc3ccncc3)cccc12]

In embodiments, the compound does not have the formula:

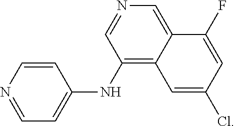 [SMILES: Fc1cc(Cl)cc2c(Nc3ccncc3)cncc12]

In embodiments, the compound is not

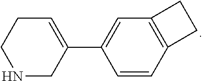 [SMILES: C1=C(c2ccc3c(c2)CC3)CNCC1]

In embodiments, the compound is not

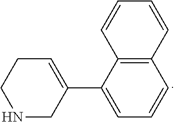 [SMILES: C1=C(c2cccc3ccccc23)CNCC1]

In embodiments, the compound is not

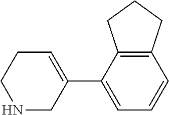 [SMILES: C1=C(c2cccc3c2CCC3)CNCC1]

In embodiments, the compound is not

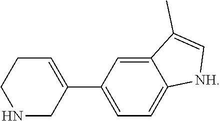 [SMILES: Cc1c[nH]c2ccc(C3=CCCNC3)cc12]

In embodiments, the compound is not

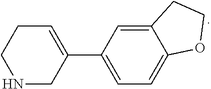 [SMILES: C1=C(c2ccc3c(c2)CCO3)CNCC1]

In embodiments, the compound is not

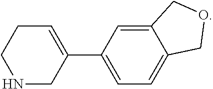 [SMILES: C1=C(c2ccc3c(c2)COC3)CNCC1]

In embodiments, the compound is not

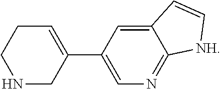 [SMILES: C1=C(c2cnc3[nH]ccc3c2)CNCC1]

In embodiments, the compound is not

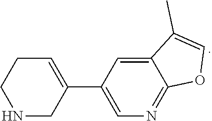 [SMILES: Cc1coc2ncc(C3=CCCNC3)cc12]

In embodiments, the compound is not

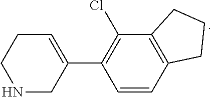 [SMILES: Clc1c(C2=CCCNC2)ccc2c1CCC2]

In embodiments, the compound is not

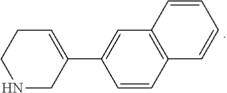 [SMILES: C1=C(c2ccc3ccccc3c2)CNCC1]

In embodiments, the compound is not

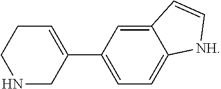 [SMILES: C1=C(c2ccc3[nH]ccc3c2)CNCC1]

In embodiments, the compound is not

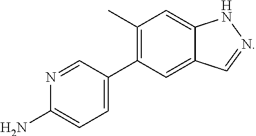 [SMILES: Cc1cc2[nH]ncc2cc1-c1ccc(N)nc1]

In embodiments, the compound is not

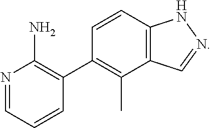 [SMILES: Cc1c(-c2cccnc2N)ccc2[nH]ncc12]

In embodiments, the compound is not

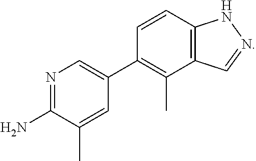 [SMILES: Cc1cc(-c2ccc3[nH]ncc3c2C)cnc1N]

In embodiments, the compound is not

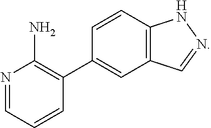 [SMILES: Nc1ncccc1-c1ccc2[nH]ncc2c1]

In embodiments, the compound is not

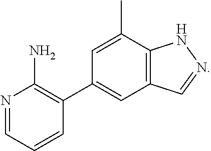 [SMILES: Cc1cc(-c2cccnc2N)cc2cn[nH]c12]

In embodiments, the compound is not

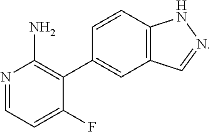 [SMILES: Nc1nccc(F)c1-c1ccc2[nH]ncc2c1]

In embodiments, the compound is not

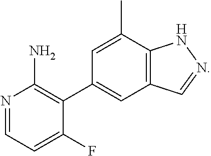 [SMILES: Cc1cc(-c2c(F)ccnc2N)cc2cn[nH]c12]

In embodiments, the compound is not

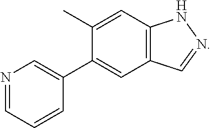 [SMILES: Cc1cc2[nH]ncc2cc1-c1cccnc1]

In embodiments, the compound is not

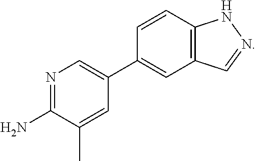 [SMILES: Cc1cc(-c2ccc3[nH]ncc3c2)cnc1N]

In embodiments, the compound is not

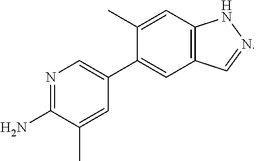 [SMILES: Cc1cc2[nH]ncc2cc1-c1cnc(N)c(C)c1]

In embodiments, the compound is not

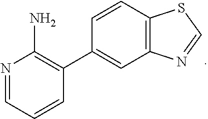 [SMILES: Nc1ncccc1-c1ccc2scnc2c1]

In embodiments, the compound is not

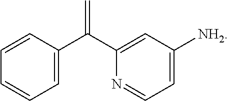

In embodiments, the compound is not

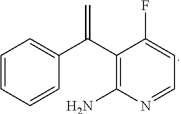 [SMILES: C=C(c1ccccc1)c1c(F)ccnc1N]

In embodiments, the compound is not

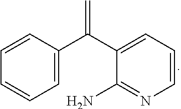

In embodiments, the compound is not

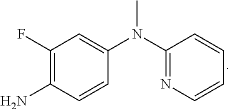 [SMILES: CN(c1ccc(N)c(F)c1)c1ccccn1]

In embodiments, the compound is not

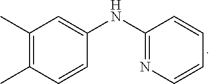

In embodiments, the compound is not

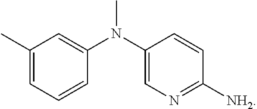 [SMILES: Cc1cccc(N(C)c2ccc(N)nc2)c1]

In embodiments, the compound is not

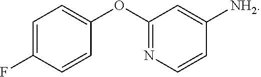 [SMILES: Nc1ccnc(Oc2ccc(F)cc2)c1]

In embodiments, the compound is not

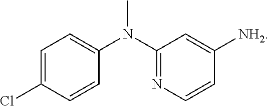 [SMILES: CN(c1ccc(Cl)cc1)c1cc(N)ccn1]

In embodiments, the compound is not

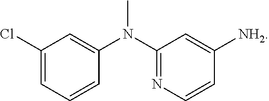 [SMILES: CN(c1cccc(Cl)c1)c1cc(N)ccn1]

In embodiments, the compound is not

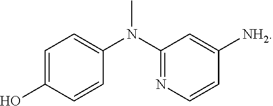 [SMILES: CN(c1ccc(O)cc1)c1cc(N)ccn1]

In embodiments, the compound is not

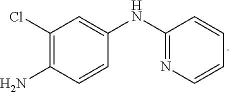 [SMILES: Nc1ccc(Nc2ccccn2)cc1Cl]

In embodiments, the compound is not

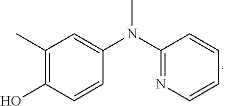 [SMILES: Cc1cc(N(C)c2ccccn2)ccc1O]

In embodiments, the compound is not

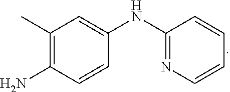

In embodiments, the compound is not

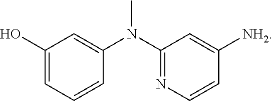 [SMILES: CN(c1cccc(O)c1)c1cc(N)ccn1]

In embodiments, the compound is not

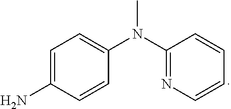

In embodiments, the compound is not

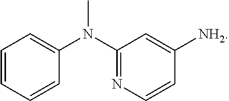

In embodiments, the compound is not

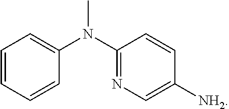

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I), (II), (III), (IV), (V), (VI), (VII), (VIII), (IX), (X), (XI), or (XII).

In embodiments, the compound is a compound of formula (XIII), (XIIIa), (XIIIb), (XIIIc), (XIV), (XIVa), (XIVb), (XIVc), (XIVd), (XV), or (XVa).

In embodiments, the compound is a compound of formula (XVI), (XVIa), (XVII), or (XVIIa).

In embodiments, the compound is a compound of formula (XVIII) or (XIX). In embodiments, the compound is a compound of formula (XVIII).

In embodiments, the pharmaceutical composition further includes a second agent. In embodiments, the second agent is an opioid (e.g., morphine, fentanyl, hydrocodone, methadone, buprenorphine, oxycodone, codeine, tramadol, or tapendatol). In embodiments, the second agent is an anesthetic. In embodiments, the second agent is a local anesthetic (e.g., bupivacaine). In embodiments, the pharmaceutical composition includes an effective amount of the compound and an effective amount of the second agent. In embodiments, the pharmaceutical composition includes a therapeutically effective amount of the compound and a therapeutically effective amount of the second agent.

### IV. Methods of Use

In an aspect is provided a method of treating pain in a subject in need thereof, the method including administering to the subject in need thereof a therapeutically effective amount of a compound described herein, or a pharmaceutically acceptable salt thereof.

In embodiments, the compound is a compound of formula (I), (II), (III), (IV), (V), (VI), (VII), (VIII), (IX), (X), (XI), or (XII).

In embodiments, the compound is a compound of formula (XIII), (XIIIa), (XIIIb), (XIIIc), (XIV), (XIVa), (XIVb), (XIVc), (XIVd), (XV), or (XVa).

In embodiments, the compound is not:

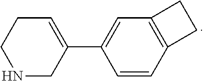 [SMILES: C1=C(c2ccc3c(c2)CC3)CNCC1]

In embodiments, the compound is a compound of formula (XVI), (XVIa), (XVII), or (XVIIa).

In embodiments, the compound is not:

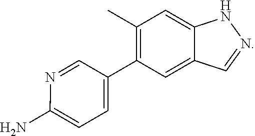 [SMILES: Cc1cc2[nH]ncc2cc1-c1ccc(N)nc1]

In an aspect is provided a method of treating pain in a subject in need thereof, the method including administering to the subject in need thereof a therapeutically effective amount of a compound having the formula:

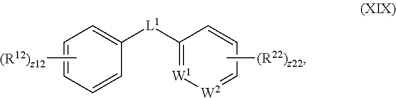

or a pharmaceutically acceptable salt thereof. L¹, R¹², z12, R²², and z22 are as described herein, including in embodiments.

W¹ is N or CR³.

W² is N or CR⁴.

R³ and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

At least one of W¹ or W² is N.

In embodiments, the compound is not:

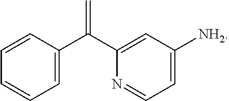

In embodiments, W¹ is N. In embodiments, W¹ is CR³. In embodiments, W¹ is CH. In embodiments, W² is N. In embodiments, W² is CR⁴. In embodiments, W² is CH. In embodiments, W¹ is N and W² is CH. In embodiments, W¹ is CH and W² is N. In embodiments, W¹ and W² are N.

In embodiments, R³ is hydrogen. In embodiments, R³ is halogen. In embodiments, R³ is —F. In embodiments, R³ is —C₁. In embodiments, R³ is —Br. In embodiments, R³ is —I. In embodiments, R³ is —OH. In embodiments, R³ is —NH₂. In embodiments, R³ is —OCF₃. In embodiments, R³ is substituted C₁-C₄ alkyl. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is unsubstituted 2 to 6 membered heteroalkyl. In embodiments, R³ is unsubstituted methoxy. In embodiments, R³ is unsubstituted ethoxy. In embodiments, R³ is unsubstituted propoxy. In embodiments, R³ is unsubstituted n-propoxy. In embodiments, R³ is unsubstituted isopropoxy. In embodiments, R³ is unsubstituted butoxy.

In embodiments, R⁴ is hydrogen. In embodiments, R⁴ is halogen. In embodiments, R⁴ is —F. In embodiments, R⁴ is —C₁. In embodiments, R⁴ is —Br. In embodiments, R⁴ is —I. In embodiments, R⁴ is —OH. In embodiments, R⁴ is —NH₂. In embodiments, R⁴ is —OCF₄. In embodiments, R⁴ is substituted C₁-C₄ alkyl. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted 2 to 6 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy.

In embodiments, the pain is α_{2A}AR-associated pain. In embodiments, the pain is post-operative pain. In embodiments, the post-operative pain is pain after a hysterectomy. In embodiments, the post-operative pain is pediatric post-operative pain. In embodiments, the pain is neuropathic pain. In embodiments, the neuropathic pain is post-traumatic neuropathic pain. In embodiments, the neuropathic pain is diabetic neuropathic pain. In embodiments, the neuropathic pain is post-herpetic neuralgia. In embodiments, the neuropathic pain is chemotherapy-induced pain. In embodiments, the neuropathic pain is phantom limb pain. In embodiments, the pain is inflammatory pain. In embodiments, the inflammatory pain is associated with rheumatoid arthritis, ankylosing spondylitis, osteoarthritis, bursitis, tendinitis, or acute gouty arthritis. In embodiments, the pain is opioid refractory pain. In embodiments, the pain is adiposis dolorosa. In embodiments, the pain is burn pain. In embodiments, the pain is a rebound headache. In embodiments, the pain is migraine pain. In embodiments, the pain is cluster headaches. In embodiments, the pain is central pain conditions following stroke. In embodiments, the pain is chronic musculoskeletal pain.

In embodiments, neuropathic pain is pain caused by various types of nerve damage. Examples of neuropathic pain include post herpetic (or post-shingles) neuralgia, reflex sympathetic dystrophy, components of cancer pain, phantom limb pain, entrapment neuropathy (e.g., carpal tunnel syndrome), and peripheral neuropathy (widespread nerve damage). Neuropathic pain can also be associated with diabetes, as well as chronic alcohol use, exposure to toxins (including many chemotherapeutic agents), and vitamin deficiencies. Additional examples of conditions that can be associated with neuropathic pain include but are not limited to autoimmune disease (e.g., multiple sclerosis), metabolic diseases (e.g., diabetic neuropathy including peripheral, focal, proximal and autonomic), infection (e.g., shingles), vascular disease, trauma, pain resulting from chemotherapy, HIV infection/AIDS, spine or back surgery, post-amputation pain, central pain syndrome, trigeminal neuralgia, reflex sympathetic dystrophy syndrome, nerve compression, stroke, spinal cord injury, herpes zoster, complex regional pain syndrome, neuropathic pain due to chronic disease (e.g., multiple sclerosis, HIV, etc.), neuropathic pain due to trauma (e.g., causalgia), neuropathic pain due to impingement (e.g., sciatica, carpal tunnel, etc.), neuropathic pain due to drug exposure or toxic chemical exposure, neuropathic pain due to impaired organ function, neuropathic low back pain, neuropathic pain due to fibromylagia, glossopharyngeal neuralgia, radiculopathy, dental pain, and cancer. Generally the lesion leading to pain can directly involve the nociceptive pathways. Neuropathic pain can also be idiopathic.

In embodiments, the compound is administered systemically. In embodiments, the compound is administered topically. In embodiments, the compound is administered intrathecally. In embodiments, the compound is administered orally. In embodiments, the compound is administered intravenously.

In embodiments, the method further includes administering a second agent. In embodiments, the second agent is an opioid (e.g., morphine, fentanyl, hydrocodone, methadone, buprenorphine, oxycodone, codeine, tramadol, or tapendatol). In embodiments, the second agent is an anesthetic. In embodiments, the second agent is a local anesthetic (e.g., bupivacaine). In embodiments, the second agent is gabapentin.

In an aspect is provided a method of increasing the level of activity of α_{2A} adrenergic receptor in a cell, said method comprising contacting the cell with an effective of a compound described herein, or a pharmaceutically acceptable salt thereof. In embodiments, the level of activity of α_{2A} adrenergic receptor is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of α_{2A} adrenergic receptor is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the compound binds to D128, V129, T133, 1205, S215, S219, W402, F405, F406, Y409, F427, or Y431 of α_{2A} adrenergic receptor. In embodiments, the compound binds noncovalently to D128, V129, T133, 1205, S215, S219, W402, F405, F406, Y409, F427, or Y431 of α_{2A} adrenergic receptor. In embodiments, the compound binds (e.g., noncovalently) to D128 of α_{2A} adrenergic receptor. In embodiments, the compound binds (e.g., noncovalently) to V129 of α_{2A} adrenergic receptor. In embodiments, the compound binds (e.g., noncovalently) to T133 of α_{2A} adrenergic receptor. In embodiments, the compound binds (e.g., noncovalently) to 1205 of α_{2A} adrenergic receptor. In embodiments, the compound binds (e.g., noncovalently) to S215 of α_{2A} adrenergic receptor. In embodiments, the compound binds (e.g., noncovalently) to S219 of α_{2A} adrenergic receptor. In embodiments, the compound binds (e.g., noncovalently) to W402 of α_{2A} adrenergic receptor. In embodiments, the compound binds (e.g., noncovalently) to F405 of α_{2A} adrenergic receptor. In embodiments, the compound binds (e.g., noncovalently) to F406 of α_{2A} adrenergic receptor. In embodiments, the compound binds (e.g., noncovalently) to Y409 of α_{2A} adrenergic receptor. In embodiments, the compound binds (e.g., noncovalently) to F427 of α_{2A} adrenergic receptor. In embodiments, the compound binds (e.g., noncovalently) to Y431 of α_{2A} adrenergic receptor.

### V. Embodiments

Embodiment P1. A method of treating pain in a subject in need thereof, said method comprising administering to the subject in need thereof a therapeutically effective of a compound, or a pharmaceutically acceptable salt thereof, having the formula:

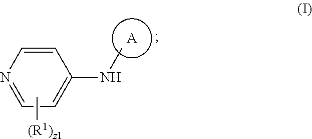

Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹³, —CHX¹², —CH₂X¹, —OCX¹³, —OCH₂X¹, —OCHX¹², —CN, —SOₙ₁R^{1D}—SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P2. The method of embodiment P1, wherein the compound has the formula:

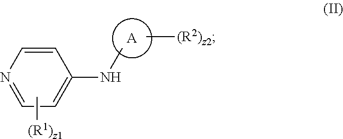

wherein
Ring A is cycloalkyl, heterocycloalkyl, aryl, or heteroaryl;
R² is independently oxo, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and z2 is an integer from 0 to 15.

Embodiment P3. The method of embodiment P2, wherein Ring A is aryl or heteroaryl.

Embodiment P4. The method of embodiment P2, wherein the compound has the formula:

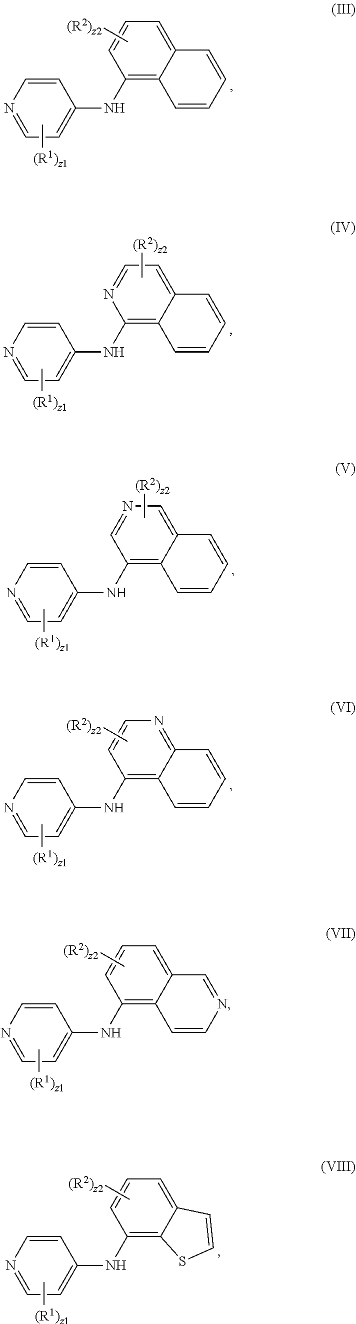

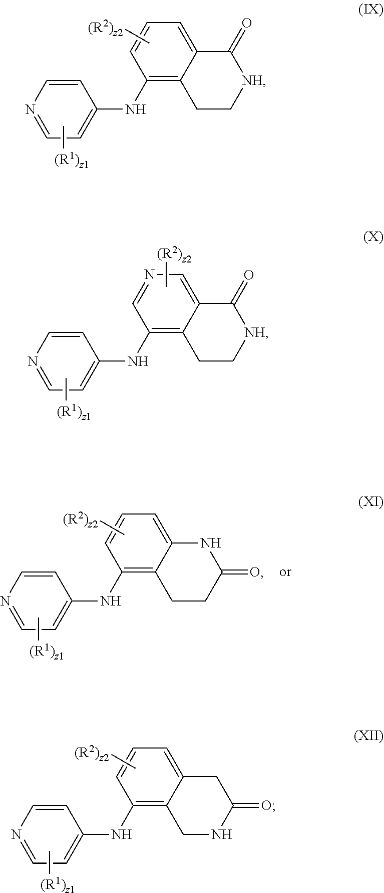

wherein z2 is an integer from 0 to 8.

Embodiment P5. The method of one of embodiments P2 to P4, wherein z2 is 0 or 1.

Embodiment P6. The method of one of embodiments P2 to P5, wherein R² is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P7. The method of one of embodiments P2 to P5, wherein R² is independently oxo, halogen, —CF₃, —OR^{2D}, or unsubstituted C₁-C₄ alkyl.

Embodiment P8. The method of one of embodiments P2 to P5, wherein R² is independently oxo, —F, —Cl, —CF₃, —OH, —OCH₃, or unsubstituted methyl.

Embodiment P9. The method of one of embodiments P1 to P8, wherein z1 is 0.

Embodiment P10. The method of embodiment P1, wherein the compound is:

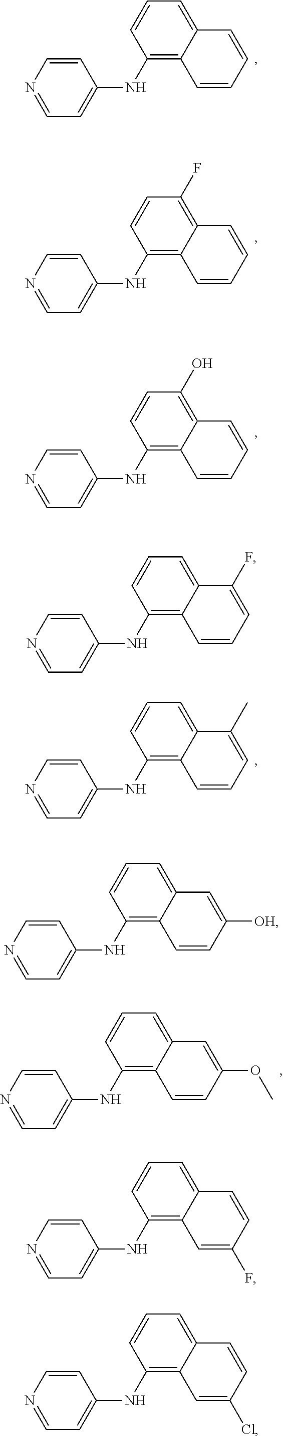

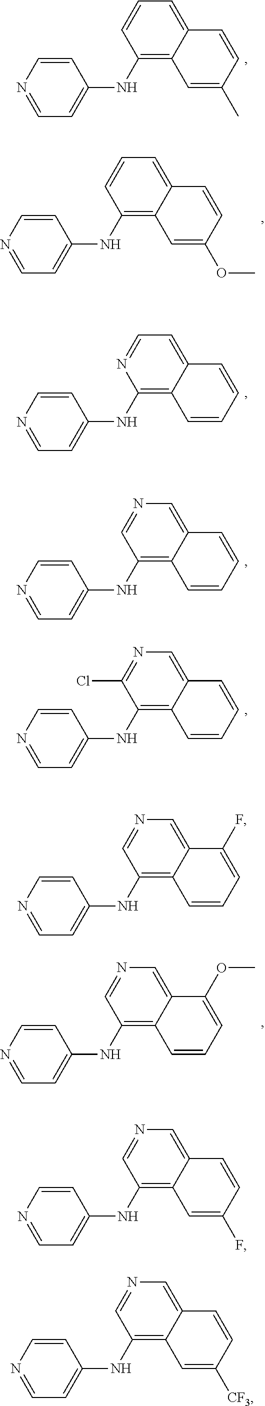

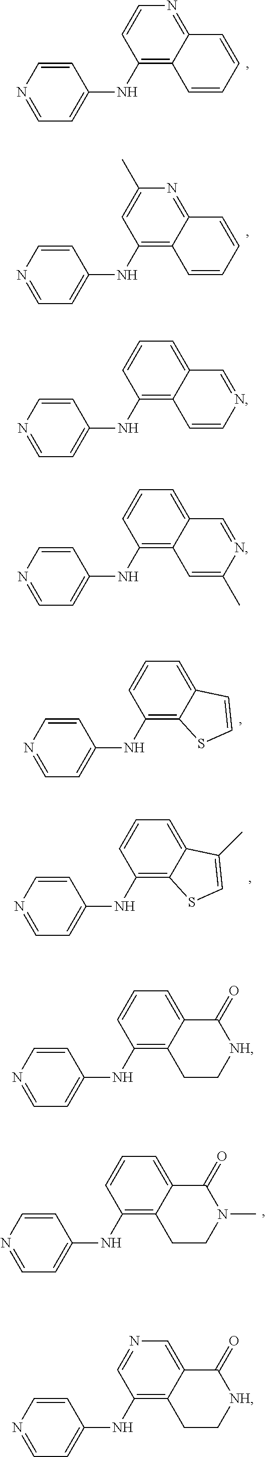

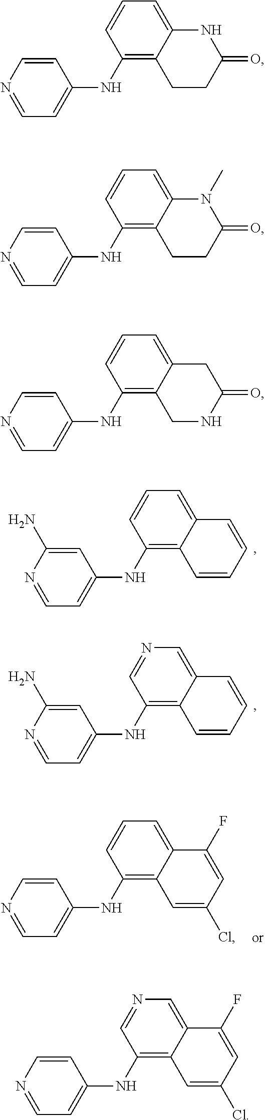

Embodiment P11. The method of one of embodiments P1 to P10, wherein the pain is post-operative pain.

Embodiment P12. The method of embodiment P11, wherein the post-operative pain is pain after a hysterectomy.

Embodiment P13. The method of embodiment P11, wherein the post-operative pain is pediatric post-operative pain.

Embodiment P14. The method of embodiment P11, further comprising administering a second agent.

Embodiment P15. The method of embodiment P14, wherein the second agent is an opioid.

Embodiment P16. The method of embodiment P14, wherein the second agent is bupivacaine.

Embodiment P17. The method of one of embodiments P1 to P10, wherein the pain is neuropathic pain.

Embodiment P18. The method of embodiment P17, wherein the neuropathic pain is post-traumatic neuropathic pain.

Embodiment P19. The method of embodiment P17, wherein the neuropathic pain is diabetic neuropathic pain.

Embodiment P20. The method of embodiment P17, wherein the neuropathic pain is post-herpetic neuralgia.

Embodiment P21. The method of embodiment P17, wherein the neuropathic pain is chemotherapy-induced pain.

Embodiment P22. The method of embodiment P17, wherein the neuropathic pain is phantom limb pain.

Embodiment P23. The method of one of embodiments P1 to P10, wherein the pain is inflammatory pain.

Embodiment P24. The method of one of embodiments P1 to P10, wherein the pain is opioid refractory pain.

Embodiment P25. The method of one of embodiments P1 to P10, wherein the pain is a rebound headache.

Embodiment P26. The method of one of embodiments P1 to P10, wherein the pain is migraine pain.

Embodiment P27. The method of one of embodiments P1 to P26, wherein the compound is administered systemically.

Embodiment P28. The method of one of embodiments P1 to P26, wherein the compound is administered topically.

Embodiment P29. The method of one of embodiments P1 to P26, wherein the compound is administered intrathecally.

Embodiment P30. A method of increasing the level of activity of α_{2A} adrenergic receptor in a cell, said method comprising contacting the cell with an effective of a compound, or a pharmaceutically acceptable salt thereof, having the formula:

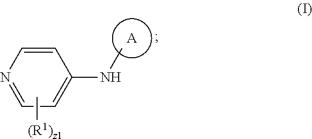

Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P31. The method of embodiment P30, wherein the compound binds to D128, V129, T133, 1205, S215, S219, W402, F405, F406, Y409, F427, or Y431 of α_{2A} adrenergic receptor.

Embodiment P32. The method of embodiment P30, wherein the compound binds noncovalently to D128, V129, T133, 1205, S215, S219, W402, F405, F406, Y409, F427, or Y431 of α_{2A} adrenergic receptor.

Embodiment P33. A pharmaceutical composition comprising a compound, or a pharmaceutically acceptable salt thereof, and a pharmaceutically acceptable excipient, wherein the compound has the formula:

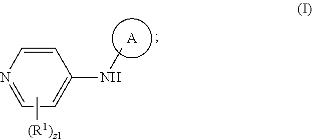

Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹³, —CHX¹², —CH₂X¹, —OCX¹³, —OCH₂X¹, —OCHX¹², —CN, —SOₙ₁R^{1D}—SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment P34. A compound, or a pharmaceutically acceptable salt thereof, having the formula:

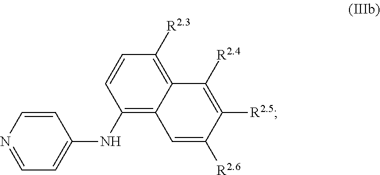

wherein
R^{2.3} is hydrogen, halogen, —OR^{2D}, or unsubstituted alkyl;
R^{2.4} is hydrogen, —OR^{2D}, or unsubstituted alkyl,
R^{2.5} is hydrogen, halogen, —OR^{2D}, or unsubstituted alkyl;
R^{2.6} is hydrogen, halogen, —OR^{2D}, or unsubstituted alkyl; and
R^{2D} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein at least one of R^{2.3}, R^{2.4}, R^{2.5}, and R^{2.6} is not hydrogen.

Embodiment P35. The compound of embodiment P34, wherein R^{2.3} is —F or —OH.

Embodiment P36. The compound of embodiment P34, wherein R^{2.4} is unsubstituted C₁-C₄ alkyl.

Embodiment P37. The compound of embodiment P34, wherein R^{2.5} is —OH or —OCH₃.

Embodiment P38. The compound of embodiment P34, wherein R^{2.6} is —F, —Cl, —OCH₃, or unsubstituted C₁-C₄ alkyl.

Embodiment P39. The compound of embodiment P34, having the formula:

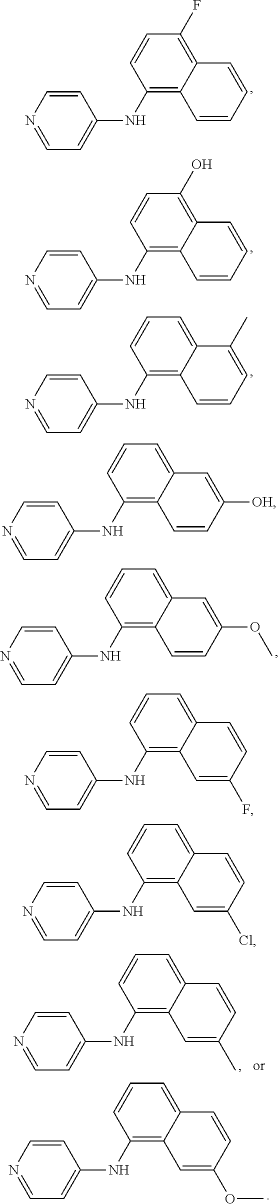

Embodiment P40. A compound, or a pharmaceutically acceptable salt thereof, having the formula:

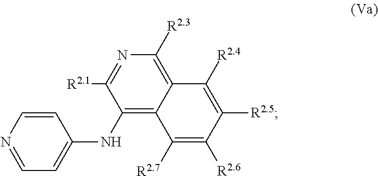

wherein
R^{2.1}, R^{2.3}, R^{2.4}, R^{2.5}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.3}, R^{2.4}, R^{2.5}, R^{2.6}, and R^{2.7} is not hydrogen;
wherein R^{2.1} is not —C₁; and
wherein R^{2.4} is not —C(O)OR^{2C}.

Embodiment P41. The compound of embodiment P40, wherein R^{2.4} is —F or —OCH₃.

Embodiment P42. The compound of embodiment P40, wherein R^{2.6} is —F, —Cl, or —CF₃.

Embodiment P43. The compound of embodiment P40, having the formula:

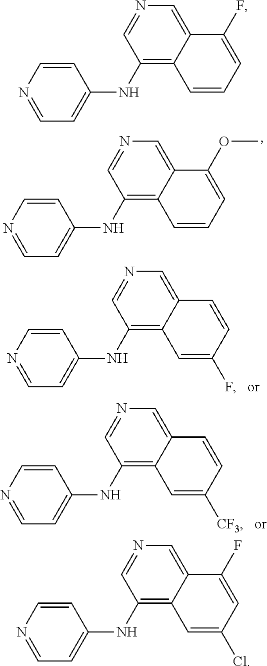

Embodiment P44. A compound, or a pharmaceutically acceptable salt thereof, having the formula:

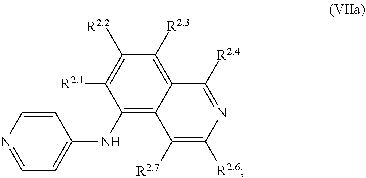

wherein
R^{2.1}, R^{2.2}, R^{2.3}, R^{2.4}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.2}, R^{2.3}, R^{2.4}, R^{2.6}, and R^{2.7} is not hydrogen.

Embodiment P45. The compound of embodiment P44, wherein R^{2.6} is unsubstituted C₁-C₄ alkyl.

Embodiment P46. The compound of embodiment P44, having the formula:

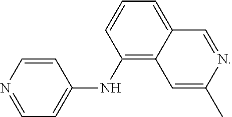 [SMILES: Cc1cc2c(Nc3ccncc3)cccc2cn1]

Embodiment P47. A compound, or a pharmaceutically acceptable salt thereof, having the formula:

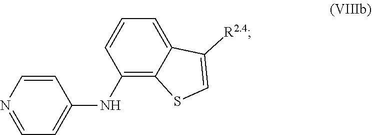

wherein
R^{2.4} is halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

Embodiment P48. The compound of embodiment P47, wherein R^{2.4} is unsubstituted C₁-C₄ alkyl.

Embodiment P49. The compound of embodiment P47, having the formula:

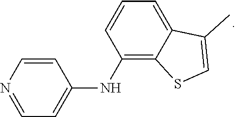 [SMILES: Cc1csc2c(Nc3ccncc3)cccc12]

Embodiment P50. A compound, or a pharmaceutically acceptable salt thereof, having the formula:

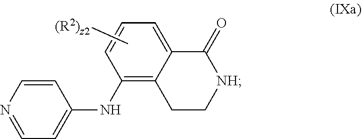

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment P51. The compound of embodiment P50, having the formula:

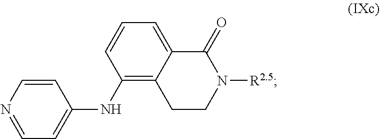

wherein
R^{2.5} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P52. The compound of embodiment P51, wherein R^{2.5} is hydrogen or unsubstituted C₁-C₄ alkyl.

Embodiment P53. The compound of embodiment P50, having the formula:

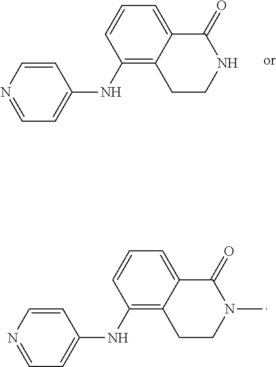 [SMILES: O=C1NCCc2c(Nc3ccncc3)cccc21 ; CN1CCc2c(Nc3ccncc3)cccc2C1=O]

Embodiment P54. A compound, or a pharmaceutically acceptable salt thereof, having the formula:

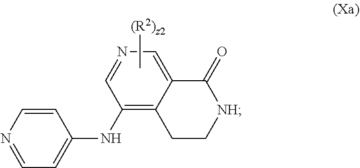

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 7.

Embodiment P55. The compound of embodiment P54, having the formula:

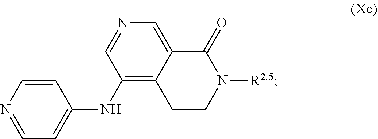

wherein
R^{2.5} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P56. The compound of embodiment P55, wherein R^{2.5} is hydrogen or unsubstituted C₁-C₄ alkyl.

Embodiment P57. The compound of embodiment P54, having the formula:

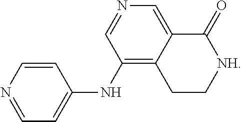 [SMILES: O=C1NCCc2c(Nc3ccncc3)cncc21]

Embodiment P58. A compound, or a pharmaceutically acceptable salt thereof, having the formula:

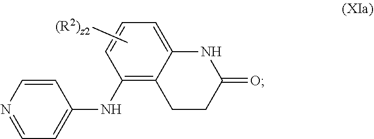

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment P59. The compound of embodiment P58, having the formula:

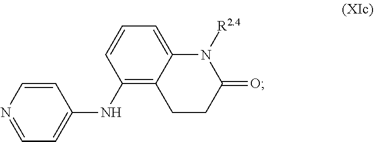

wherein
R^{2.4} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P60. The compound of embodiment P59, wherein R^{2.4} is hydrogen or unsubstituted C₁-C₄ alkyl.

Embodiment P61. The compound of embodiment P58, having the formula:

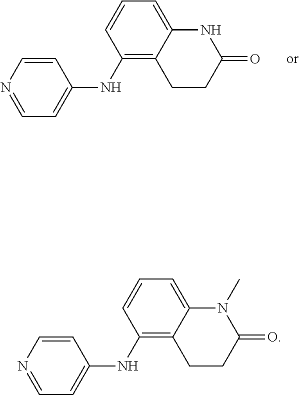 [SMILES: O=C1CCc2c(cccc2Nc2ccncc2)N1 ; CN1C(=O)CCc2c(Nc3ccncc3)cccc21]

Embodiment P62. A compound, or a pharmaceutically acceptable salt thereof, having the formula:

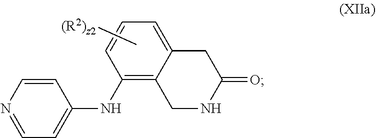

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment P63. The compound of embodiment P62, having the formula:

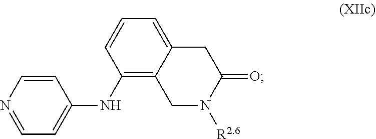

wherein
R^{2.6} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P64. The compound of embodiment P63, wherein R^{2.6} is hydrogen or unsubstituted C₁-C₄ alkyl.

Embodiment P65. The compound of embodiment P62, having the formula:

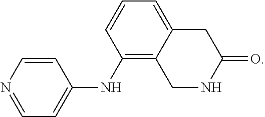 [SMILES: O=C1Cc2cccc(Nc3ccncc3)c2CN1]

Embodiment Q1. A compound, or a pharmaceutically acceptable salt thereof, having the formula:

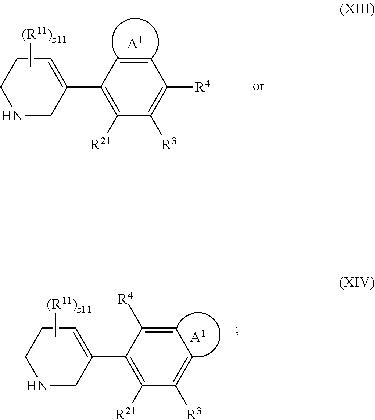

wherein
Ring A¹ is a substituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z11 is an integer from 0 to 8.

Embodiment Q2. The compound of embodiment Q1, wherein ring A¹ is oxo-substituted cycloalkyl.

Embodiment Q3. The compound of embodiment Q1, wherein ring A¹ is a substituted cycloalkyl, wherein the substituent is oxo, halogen, —NH₂, —OH, substituted or unsubstituted C₁-C₄ alkyl, or substituted or unsubstituted 2 to 4 membered heteroalkyl.

Embodiment Q4. The compound of embodiment Q1, wherein ring A¹ is a substituted C₃-C₈ cycloalkyl.

Embodiment Q5. The compound of embodiment Q1, wherein ring A¹ is a substituted C₃-C₆ cycloalkyl.

Embodiment Q6. The compound of embodiment Q1, wherein ring A¹ is a substituted cyclopentyl.

Embodiment Q7. The compound of one of embodiments Q1 to Q6, wherein z11 is 0.

Embodiment Q8. The compound of one of embodiments Q1 to Q7, wherein R²¹ is halogen, —NH₂, —OH, substituted or unsubstituted C₁-C₄ alkyl, or substituted or unsubstituted 2 to 4 membered heteroalkyl.

Embodiment Q9. The compound of one of embodiments Q1 to Q7, wherein R^{2.1} is halogen, —NH₂, —OH, or unsubstituted methyl.

Embodiment Q10. The compound of one of embodiments Q1 to Q9, wherein R³ is halogen, —NH₂, —OH, substituted or unsubstituted C₁-C₄ alkyl, or substituted or unsubstituted 2 to 4 membered heteroalkyl.

Embodiment Q11. The compound of one of embodiments Q1 to Q9, wherein R³ is halogen, —NH₂, —OH, or unsubstituted methyl.

Embodiment Q12. The compound of one of embodiments Q1 to Q11, wherein R⁴ is halogen, —NH₂, —OH, substituted or unsubstituted C₁-C₄ alkyl, or substituted or unsubstituted 2 to 4 membered heteroalkyl.

Embodiment Q13. The compound of one of embodiments Q1 to Q11, wherein R⁴ is halogen, —NH₂, —OH, or unsubstituted methyl.

Embodiment Q14. The compound of embodiment Q1, wherein the compound is:

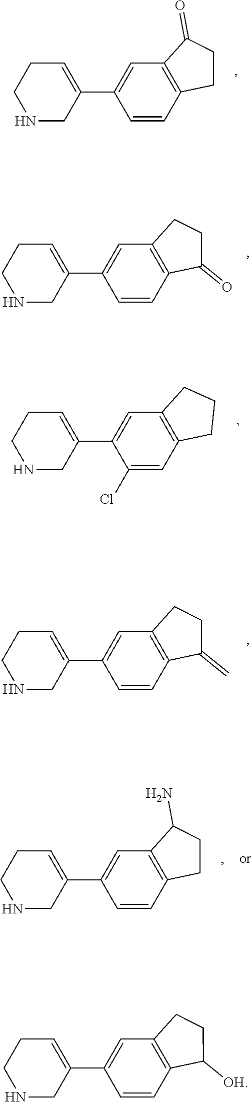 [SMILES: O=C1CCc2ccc(C3=CCCNC3)cc21 ; O=C1CCc2cc(C3=CCCNC3)ccc21 ; Clc1cc2c(cc1C1=CCCNC1)CCC2 ; C=C1CCc2cc(C3=CCCNC3)ccc21 ; NC1CCc2ccc(C3=CCCNC3)cc21 ; OC1CCc2cc(C3=CCCNC3)ccc21]

Embodiment Q15. A pharmaceutical composition comprising the compound of one of embodiments Q1 to Q14 and a pharmaceutically acceptable excipient.

Embodiment Q16. A method of treating pain in a subject in need thereof, said method comprising administering to the subject a therapeutically effective amount of a compound having the formula:

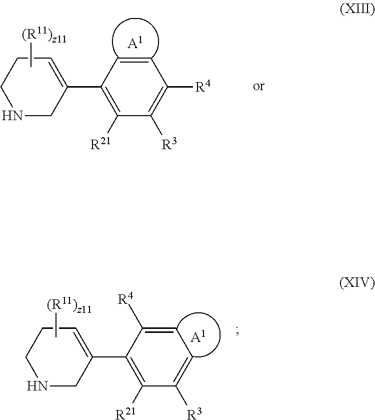

or a pharmaceutically acceptable salt thereof, wherein
Ring A¹ is a substituted or unsubstituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z11 is an integer from 0 to 8.

Embodiment Q17. The method of embodiment Q16, wherein the compound is not:

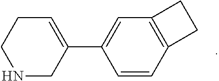 [SMILES: C1=C(c2ccc3c(c2)CC3)CNCC1]

Embodiment Q18. The method of embodiment Q16 or Q17, wherein the pain is post-operative pain.

Embodiment Q19. The method of embodiment Q18, wherein the post-operative pain is pain after a hysterectomy.

Embodiment Q20. The method of embodiment Q18, wherein the post-operative pain is pediatric post-operative pain.

Embodiment Q21. The method of one of embodiments Q18 to Q20, further comprising administering a second agent.

Embodiment Q22. The method of embodiment Q21, wherein the second agent is an opioid.

Embodiment Q23. The method of embodiment Q21, wherein the second agent is bupivacaine or gabapentin.

Embodiment Q24. The method of embodiment Q16 or Q17, wherein the pain is neuropathic pain.

Embodiment Q25. The method of embodiment Q24, wherein the neuropathic pain is post-traumatic neuropathic pain.

Embodiment Q26. The method of embodiment Q24, wherein the neuropathic pain is diabetic neuropathic pain.

Embodiment Q27. The method of embodiment Q24, wherein the neuropathic pain is post-herpetic neuralgia.

Embodiment Q28. The method of embodiment Q24, wherein the neuropathic pain is chemotherapy-induced pain.

Embodiment Q29. The method of embodiment Q24, wherein the neuropathic pain is phantom limb pain.

Embodiment Q30. The method of embodiment Q16 or Q17, wherein the pain is inflammatory pain.

Embodiment Q31. The method of embodiment Q30, wherein the inflammatory pain is associated with rheumatoid arthritis, ankylosing spondylitis, osteoarthritis, bursitis, tendinitis, or acute gouty arthritis.

Embodiment Q32. The method of embodiment Q16 or Q17, wherein the pain is opioid refractory pain.

Embodiment Q33. The method of embodiment Q16 or Q17, wherein the pain is a rebound headache.

Embodiment Q34. The method of embodiment Q16 or Q17, wherein the pain is migraine pain.

Embodiment Q35. The method of embodiment Q16 or Q17, wherein the pain is adiposis dolorosa.

Embodiment Q36. The method of embodiment Q16 or Q17, wherein the pain is a burn pain.

Embodiment Q37. The method of embodiment Q16 or Q17, wherein the pain is cluter headaches.

Embodiment Q38. The method of embodiment Q16 or Q17, wherein the pain is associated with central pain conditions following stroke.

Embodiment Q39. The method of embodiment Q16 or Q17, wherein the pain is a musculoskeletal pain.

Embodiment Q40. The method of one of embodiments Q16 to Q39, wherein the compound is administered systemically.

Embodiment Q41. The method of one of embodiments Q16 to Q39, wherein the compound is administered topically.

Embodiment Q42. The method of one of embodiments Q16 to Q39, wherein the compound is administered intrathecally.

Embodiment Q43. The method of one of embodiments Q16 to Q39, wherein the compound is administered orally.

Embodiment Q44. The method of one of embodiments Q16 to Q39, wherein the compound is administered intravenously.

Embodiment S1. A compound, or a pharmaceutically acceptable salt thereof, having the formula:

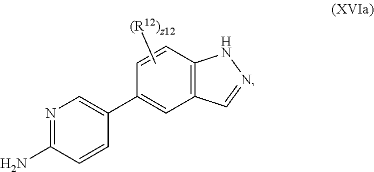

wherein
R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z12 is an integer from 0 to 8;
with the proviso that R¹² is not methyl.

Embodiment S2. The compound of embodiment Si, wherein R¹² is not C₁-C₄-alkyl.

Embodiment S3. The compound of embodiment Si, wherein R¹² is not unsubstituted C₁-C₄-alkyl.

Embodiment S4. The compound of embodiment Si, wherein R¹² is halogen, —CN, —NO₂, substituted C₁-C₄ alkyl, or substituted or unsubstituted 2 to 4 membered heteroalkyl.

Embodiment S5. The compound of embodiment Si, wherein R¹² is halogen, —CN, —NO₂, —CF₃, or —OCH₃.

Embodiment S6. The compound of one of embodiments S1 to S5, wherein z12 is 1.

Embodiment S7. The compound of one of embodiments S1 to S6, wherein the compound is:

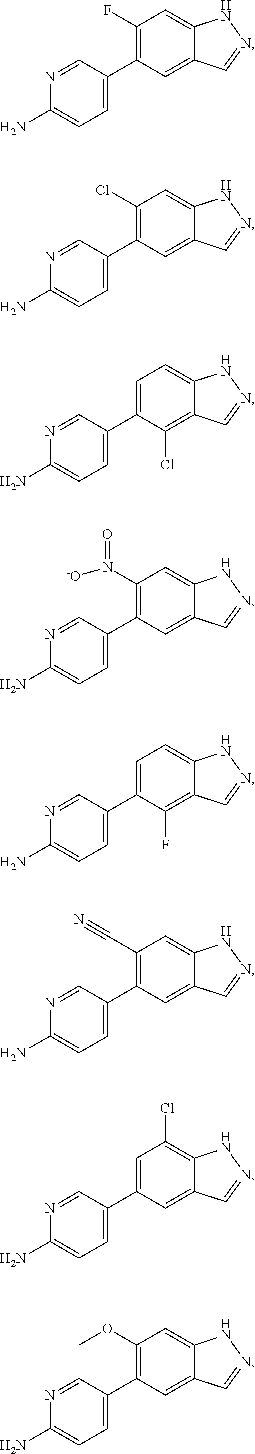

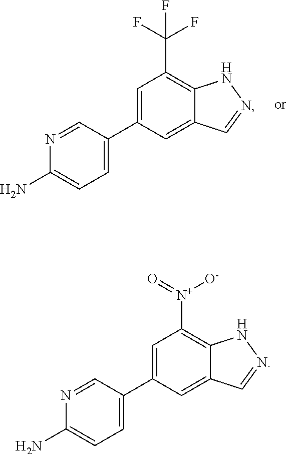 [SMILES: Nc1ccc(-c2cc(C(F)(F)F)c3[nH]ncc3c2)cn1 ; "or" ; Nc1ccc(-c2cc([N+](=O)[O-])c3[nH]ncc3c2)cn1]

Embodiment S8. A pharmaceutical composition comprising the compound of one of embodiments S1 to S7 and a pharmaceutically acceptable excipient.

Embodiment S9. A method of treating pain in a subject in need thereof, said method comprising administering to the subject a therapeutically effective amount of a compound having the formula:

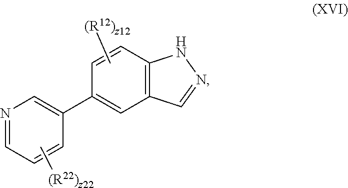

or a pharmaceutically acceptable salt thereof, wherein each R¹² and R²² are independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

Embodiment S10. The method of embodiment S9, wherein the compound is not:

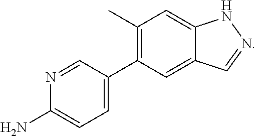 [SMILES: Cc1cc2[nH]ncc2cc1-c1ccc(N)nc1]

Embodiment S11. The method of one of embodiments S9 to S10, wherein the pain is post-operative pain.

Embodiment S12. The method of embodiment S11, wherein the post-operative pain is pain after a hysterectomy.

Embodiment S13. The method of embodiment S11, wherein the post-operative pain is pediatric post-operative pain.

Embodiment S14. The method of one of embodiments S11 to S13, further comprising administering a second agent.

Embodiment S15. The method of embodiment S14, wherein the second agent is an opioid.

Embodiment S16. The method of embodiment S14, wherein the second agent is bupivacaine.

Embodiment S17. The method of one of embodiments S9 to S10, wherein the pain is neuropathic pain.

Embodiment S18. The method of embodiment S17, wherein the neuropathic pain is post-traumatic neuropathic pain.

Embodiment S19. The method of embodiment S17, wherein the neuropathic pain is diabetic neuropathic pain.

Embodiment S20. The method of embodiment S17, wherein the neuropathic pain is post-herpetic neuralgia.

Embodiment 521. The method of embodiment S17, wherein the neuropathic pain is chemotherapy-induced pain.

Embodiment S22. The method of embodiment S17, wherein the neuropathic pain is phantom limb pain.

Embodiment S23. The method of one of embodiments S9 to 510, wherein the pain is inflammatory pain.

Embodiment S24. The method of embodiment S23, wherein the inflammatory pain is associated with rheumatoid arthritis, ankylosing spondylitis, osteoarthritis, bursitis, tendinitis, or acute gouty arthritis.

Embodiment S25. The method of one of embodiments S9 to 510, wherein the pain is opioid refractory pain.

Embodiment S26. The method of one of embodiments S9 to S10, wherein the pain is a rebound headache.

Embodiment S27. The method of one of embodiments S9 to S10, wherein the pain is migraine pain.

Embodiment S28. The method of one of embodiments S9 to S10, wherein the pain is adiposis dolorosa.

Embodiment S29. The method of one of embodiments S9 to S10, wherein the pain is a burn pain.

Embodiment S30. The method of one of embodiments S9 to 510, wherein the pain is cluster headaches.

Embodiment 531. The method of one of embodiments S9 to 510, wherein the pain is associated with central pain conditions following stroke.

Embodiment S32. The method of one of embodiments S9 to S10, wherein the pain is a musculoskeletal pain.

Embodiment S33. The method of one of embodiments S9 to S32, wherein the compound is administered systemically.

Embodiment S34. The method of one of embodiments S9 to S32, wherein the compound is administered topically.

Embodiment S35. The method of one of embodiments S9 to S32, wherein the compound is administered intrathecally.

Embodiment S36. The method of one of embodiments S9 to S32, wherein the compound is administered orally.

Embodiment S37. The method of one of embodiments S9 to S32, wherein the compound is administered intravenously.

Embodiment T1. A compound, or a pharmaceutically acceptable salt thereof, having the formula:

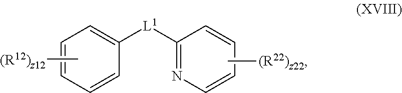

wherein
L¹ is substituted or unsubstituted C₁-C₃ alkylene;
each R¹² and R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

Embodiment T2. The compound of embodiment T1, wherein L¹ is unsubstituted C₁-C₂ alkylene.

Embodiment T3. The compound of embodiment T1, wherein L¹ is unsubstituted ethenylene.

Embodiment T4. The compound of one of embodiments T1 to T3, wherein R¹² is halogen, substituted or unsubstituted C₁-C₄ alkyl, or substituted or unsubstituted 2 to 4 membered heteroalkyl.

Embodiment T5. The compound of one of embodiments T1 to T3, wherein R¹² is —F, —Cl, —Br, —I, or —OCF₃.

Embodiment T6. The compound of one of embodiments T1 to T5, wherein z12 is 1.

Embodiment T7. The compound of one of embodiments T1 to T6, wherein R²² is —NH₂.

Embodiment T8. The compound of one of embodiments T1 to T7, wherein z22 is 1.

Embodiment T9. The compound of embodiment T1, wherein the compound is:

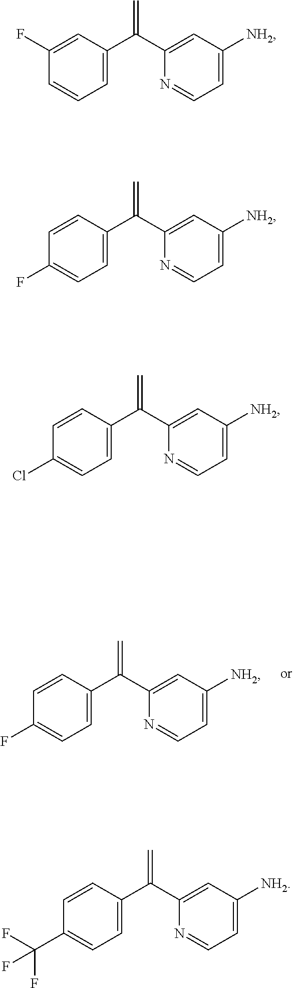 [SMILES: C=C(c1cccc(F)c1)c1cc(N)ccn1 ; C=C(c1ccc(F)cc1)c1cc(N)ccn1 ; C=C(c1ccc(Cl)cc1)c1cc(N)ccn1 ; C=C(c1ccc(F)cc1)c1cc(N)ccn1 ; C=C(c1ccc(C(F)(F)F)cc1)c1cc(N)ccn1]

Embodiment T10. A pharmaceutical composition comprising the compound of one of embodiments T1 to T9 and a pharmaceutically acceptable excipient.

Embodiment T11. A method of treating pain in a subject in need thereof, said method comprising administering to the subject a therapeutically effective amount of a compound having the formula:

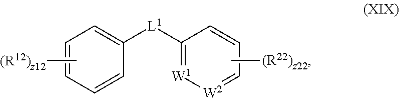

or a pharmaceutically acceptable salt thereof,
wherein
L¹ is —O—, —NR¹⁰—, or substituted or unsubstituted alkylene;
W¹ is N or CR³;
W² is N or CR⁴;
each R¹² and R^{2.2} is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5;
z22 is an integer from 0 to 4;
R³ and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen or unsubstituted C₁-C₄ alkyl;
wherein at least one of W¹ or W² is N.

Embodiment T12. The method of embodiment Ti1, wherein the compound is not:

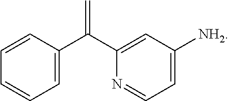

Embodiment T13. The method of one of embodiments T11 to T12, wherein W¹ is N.

Embodiment T14. The method of one of embodiments T11 to T13, wherein W² is CH.

Embodiment T15. The method of one of embodiments T11 to T14, wherein L¹ is substituted or unsubstituted C₁-C₃ alkylene.

Embodiment T16. The method of one of embodiments T11 to T14, wherein L¹ is unsubstituted C₁-C₂ alkylene.

Embodiment T17. The method of one of embodiments T11 to T14, wherein L¹ is unsubstituted ethenylene.

Embodiment T18. The method of one of embodiments T11 to T17, wherein R¹² is independently halogen, substituted or unsubstituted C₁-C₄ alkyl, or substituted or unsubstituted 2 to 4 membered heteroalkyl.

Embodiment T19. The method of one of embodiments T11 to T17, wherein R¹² is independently —F, —Cl, —Br, —I, or —OCF₃.

Embodiment T20. The method of one of embodiments T11 to T19, wherein z12 is 1.

Embodiment T21. The method of one of embodiments T11 to T20, wherein R^{2.2} is —NH₂.

Embodiment T22. The method of one of embodiments T11 to T21, wherein z22 is 1.

Embodiment T23. The method of one of embodiments T11 to T22, wherein R³ and R⁴ are hydrogen.

Embodiment T24. The method of one of embodiments T11 to T23, wherein the pain is post-operative pain.

Embodiment T25. The method of embodiment T24, wherein the post-operative pain is pain after a hysterectomy.

Embodiment T26. The method of embodiment T24, wherein the post-operative pain is pediatric post-operative pain.

Embodiment T27. The method of one of embodiments T24 to T26, further comprising administering a second agent.

Embodiment T28. The method of embodiment T27, wherein the second agent is an opioid.

Embodiment T29. The method of embodiment T27, wherein the second agent is bupivacaine or gabapentin.

Embodiment T30. The method of one of embodiments T11 to T23, wherein the pain is neuropathic pain.

Embodiment T31. The method of embodiment T30, wherein the neuropathic pain is post-traumatic neuropathic pain.

Embodiment T32. The method of embodiment T30, wherein the neuropathic pain is diabetic neuropathic pain.

Embodiment T33. The method of embodiment T30, wherein the neuropathic pain is post-herpetic neuralgia.

Embodiment T34. The method of embodiment T30, wherein the neuropathic pain is chemotherapy-induced pain.

Embodiment T35. The method of embodiment T30, wherein the neuropathic pain is phantom limb pain.

Embodiment T36. The method of one of embodiments T11 to T23, wherein the pain is inflammatory pain.

Embodiment T37. The method of embodiment T36, wherein the inflammatory pain is associated with rheumatoid arthritis, ankylosing spondylitis, osteoarthritis, bursitis, tendinitis, or acute gouty arthritis.

Embodiment T38. The method of one of embodiments T11 to T23, wherein the pain is opioid refractory pain.

Embodiment T39. The method of one of embodiments T11 to T23, wherein the pain is a rebound headache.

Embodiment T40. The method of one of embodiments T11 to T23, wherein the pain is migraine pain.

Embodiment T41. The method of one of embodiments T11 to T23, wherein the pain is adiposis dolorosa.

Embodiment T42. The method of one of embodiments T11 to T23, wherein the pain is a burn pain.

Embodiment T43. The method of one of embodiments T11 to T23, wherein the pain is cluster headaches.

Embodiment T44. The method of one of embodiments T11 to T23, wherein the pain is associated with central pain conditions following stroke.

Embodiment T45. The method of one of embodiments T11 to T44, wherein the compound is administered systemically.

Embodiment T46. The method of one of embodiments T11 to T44, wherein the compound is administered topically.

Embodiment T47. The method of one of embodiments T11 to T44, wherein the compound is administered intrathecally.

Embodiment T48. The method of one of embodiments T11 to T44, wherein the compound is administered orally.

Embodiment T49. The method of one of embodiments T11 to T44, wherein the compound is administered intravenously.

### VI. Additional Embodiments

Embodiment 1. A method of treating pain in a subject in need thereof, said method comprising administering to the subject in need thereof a therapeutically effective of a compound, or a pharmaceutically acceptable salt thereof, having the formula:

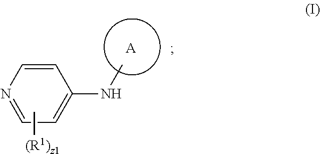

Ring A is substituted or unsubstituted aryl, substituted or unsubstituted heteroaryl, substituted or unsubstituted cycloalkyl, or substituted or unsubstituted heterocycloalkyl;
R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment 2. The method of embodiment 1, wherein the compound has the formula:

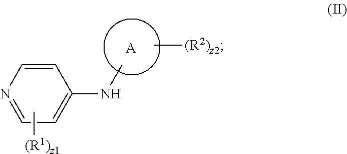

wherein
Ring A is aryl, heteroaryl, cycloalkyl, or heterocycloalkyl;
R² is independently halogen, oxo, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 15.

Embodiment 3. The method of embodiment 2, wherein Ring A is aryl or heteroaryl.

Embodiment 4. The method of embodiment 2, wherein the compound has the formula:

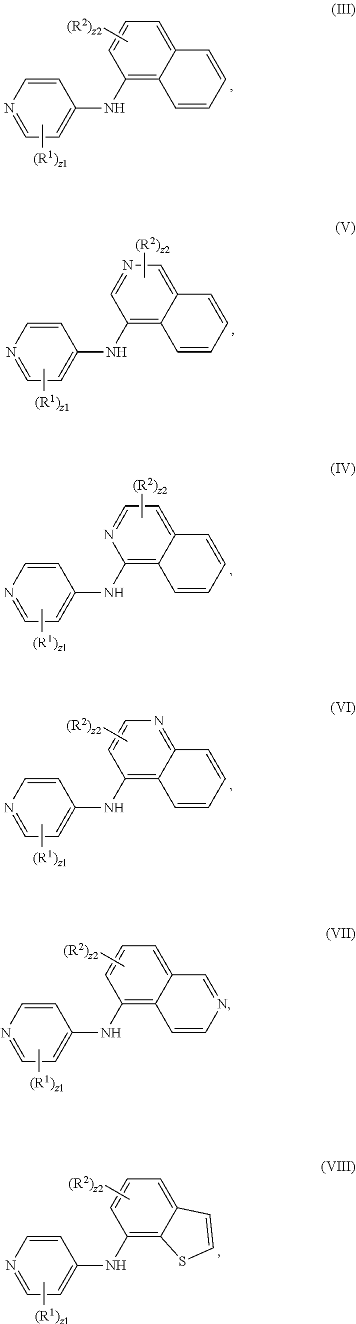

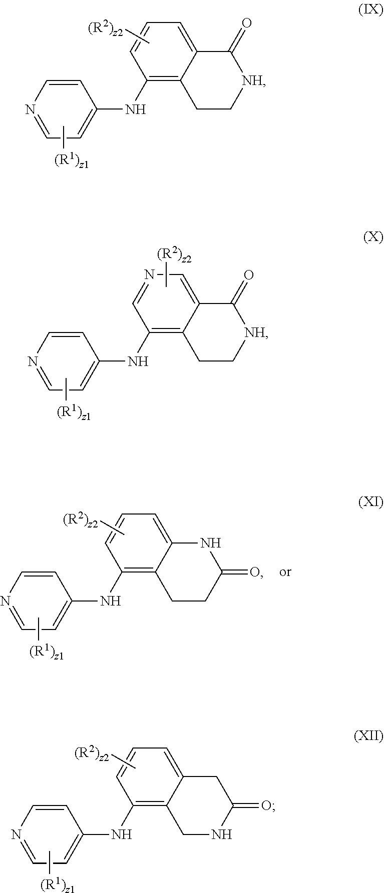

wherein
z2 is an integer from 0 to 8.

Embodiment 5. The method of one of embodiments 2 to 4, wherein z2 is 0 or 1.

Embodiment 6. The method of one of embodiments 2 to 5, wherein R² is independently halogen, oxo, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 7. The method of one of embodiments 2 to 5, wherein R² is independently halogen, oxo, —CF₃, —OR^{2D}, or unsubstituted C₁-C₄ alkyl.

Embodiment 8. The method of one of embodiments 2 to 5, wherein R² is independently —F, —Cl, oxo, —CF₃, —OH, —OCH₃, or unsubstituted methyl.

Embodiment 9. The method of one of embodiments 1 to 8, wherein z1 is 0.

Embodiment 10. The method of embodiment 1, wherein the compound is:

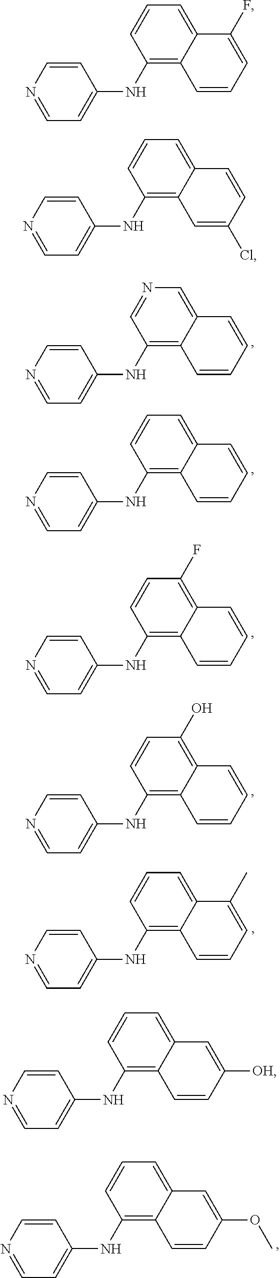

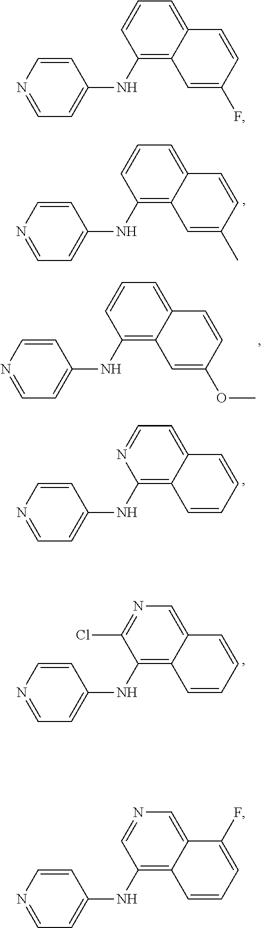 [SMILES: Fc1ccc2cccc(Nc3ccncc3)c2c1 ; Cc1ccc2cccc(Nc3ccncc3)c2c1 ; COc1ccc2cccc(Nc3ccncc3)c2c1 ; c1ccc2c(Nc3ccncc3)nccc2c1 ; Clc1ncc2ccccc2c1Nc1ccncc1 ; Fc1cccc2c(Nc3ccncc3)cncc12]

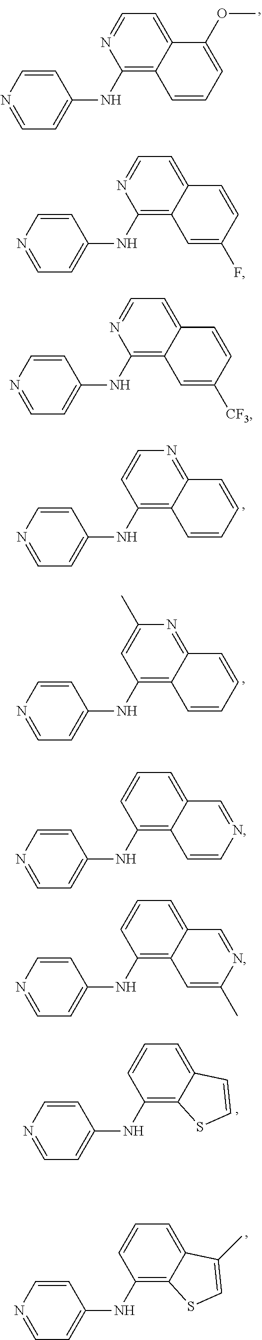

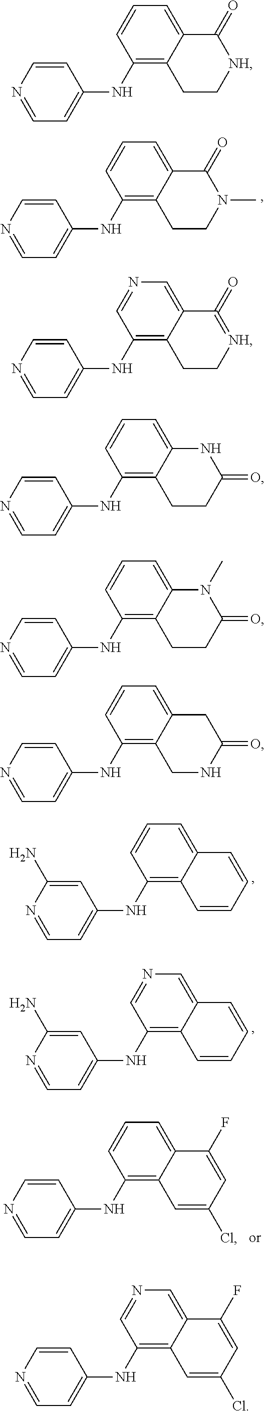

Embodiment 11. The method of one of embodiments 1 to 10, wherein the pain is post-operative pain.

Embodiment 12. The method of embodiment 11, wherein the post-operative pain is pain after a hysterectomy.

Embodiment 13. The method of embodiment 11, wherein the post-operative pain is pediatric post-operative pain.

Embodiment 14. The method of embodiment 11, further comprising administering a second agent.

Embodiment 15. The method of embodiment 14, wherein the second agent is an opioid.

Embodiment 16. The method of embodiment 14, wherein the second agent is bupivacaine.

Embodiment 17. The method of one of embodiments 1 to 10, wherein the pain is neuropathic pain.

Embodiment 18. The method of embodiment 17, wherein the neuropathic pain is post-traumatic neuropathic pain.

Embodiment 19. The method of embodiment 17, wherein the neuropathic pain is diabetic neuropathic pain.

Embodiment 20. The method of embodiment 17, wherein the neuropathic pain is post-herpetic neuralgia.

Embodiment 21. The method of embodiment 17, wherein the neuropathic pain is chemotherapy-induced pain.

Embodiment 22. The method of embodiment 17, wherein the neuropathic pain is phantom limb pain.

Embodiment 23. The method of one of embodiments 1 to 10, wherein the pain is inflammatory pain.

Embodiment 24. The method of one of embodiments 1 to 10, wherein the pain is opioid refractory pain.

Embodiment 25. The method of one of embodiments 1 to 10, wherein the pain is a rebound headache.

Embodiment 26. The method of one of embodiments 1 to 10, wherein the pain is migraine pain.

Embodiment 27. The method of one of embodiments 1 to 26, wherein the compound is administered systemically.

Embodiment 28. The method of one of embodiments 1 to 26, wherein the compound is administered topically.

Embodiment 29. The method of one of embodiments 1 to 26, wherein the compound is administered intrathecally.

Embodiment 30. A method of increasing the level of activity of α_{2A} adrenergic receptor in a cell, said method comprising contacting the cell with an effective of a compound, or a pharmaceutically acceptable salt thereof, having the formula:

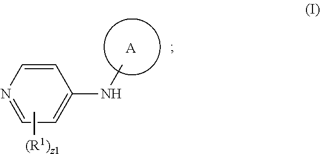

Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹³, —CHX¹², —CH₂X¹, —OCX¹³, —OCH₂X¹, —OCHX¹², —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment 31. The method of embodiment 30, wherein the compound binds to D128, V129, T133, 1205, S215, S219, W402, F405, F406, Y409, F427, or Y431 of α_{2A} adrenergic receptor.

Embodiment 32. The method of embodiment 30, wherein the compound binds noncovalently to D128, V129, T133, 1205, S215, S219, W402, F405, F406, Y409, F427, or Y431 of α_{2A} adrenergic receptor.

Embodiment 33. A pharmaceutical composition comprising a compound, or a pharmaceutically acceptable salt thereof, and a pharmaceutically acceptable excipient, wherein the compound has the formula:

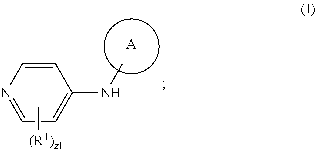

Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹³, —CHX¹², —CH₂X¹, —OCX¹³, —OCH₂X¹, —OCHX¹², —CN, —SOₙ₁R^{1D}, SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

Embodiment 34. A compound, or a pharmaceutically acceptable salt thereof, having the formula:

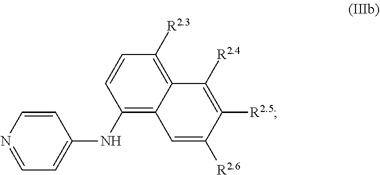

wherein
R^{2.3} is hydrogen, halogen, —OR^{2D}, or unsubstituted alkyl;
R^{2.4} is hydrogen, —OR^{2D}, or unsubstituted alkyl,
R^{2.5} is hydrogen, halogen, —OR^{2D}, or unsubstituted alkyl;
R^{2.6} is hydrogen, halogen, —OR^{2D}, or unsubstituted alkyl; and
R^{2D} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein at least one of R^{2.3}, R^{2.4}, R^{2.5}, and R^{2.6} is not hydrogen.

Embodiment 35. The compound of embodiment 34, wherein R^{2.3} is —F or —OH.

Embodiment 36. The compound of embodiment 34, wherein R^{2.4} is unsubstituted C₁-C₄ alkyl.

Embodiment 37. The compound of embodiment 34, wherein R^{2.5} is —OH or —OCH₃.

Embodiment 38. The compound of embodiment 34, wherein R^{2.6} is —F, —Cl, —OCH₃, or unsubstituted C₁-C₄ alkyl.

Embodiment 39. The compound of embodiment 34, having the formula:

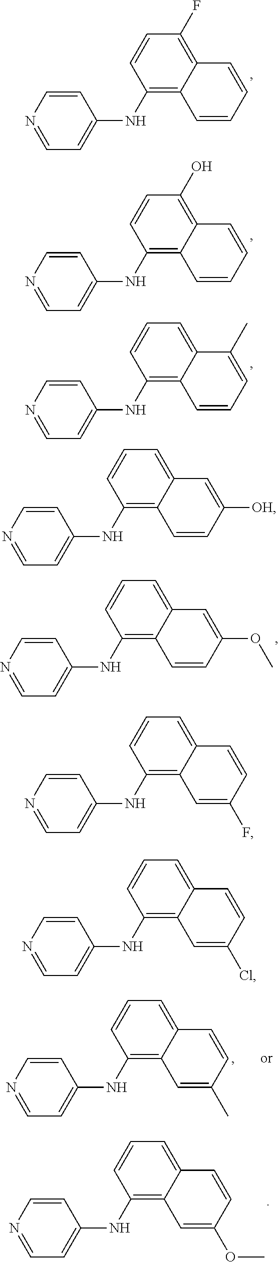

Embodiment 40. A compound, or a pharmaceutically acceptable salt thereof, having the formula:

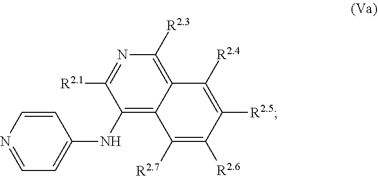

wherein
R^{2.1}, R^{2.3}, R^{2.4}, R^{2.5}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.3}, R^{2.4}, R^{2.5}, R^{2.6}, and R^{2.7} is not hydrogen;
wherein R^{2.1} is not —C₁; and
wherein R^{2.4} is not —C(O)OR^{2C}.

Embodiment 41. The compound of embodiment 40, wherein R^{2.4} is —F or —OCH₃.

Embodiment 42. The compound of embodiment 40, wherein R^{2.6} is —F, —Cl, or —CF₃.

Embodiment 43. The compound of embodiment 40, having the formula:

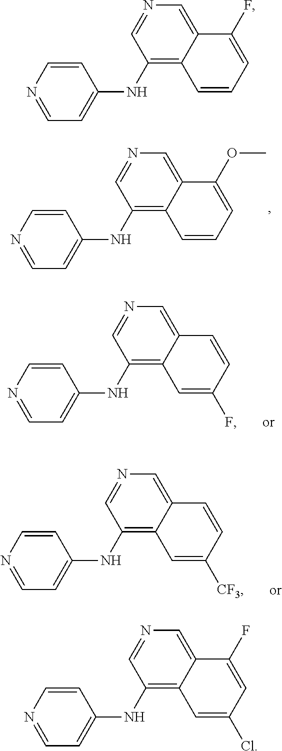 [SMILES: Fc1cccc2c(Nc3ccncc3)cncc12 ; COc1cccc2c(Nc3ccncc3)cncc12 ; Fc1ccc2cncc(Nc3ccncc3)c2c1 ; FC(F)(F)c1ccc2cncc(Nc3ccncc3)c2c1 ; Fc1cc(Cl)cc2c(Nc3ccncc3)cncc12]

Embodiment 44. A compound, or a pharmaceutically acceptable salt thereof, having the formula:

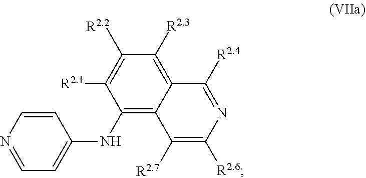

wherein
R^{2.1}, R^{2.2}, R^{2.3}, R^{2.4}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.2}, R^{2.3}, R^{2.4}, R^{2.6}, and R^{2.7} is not hydrogen.

Embodiment 45. The compound of embodiment 44, wherein R^{2.6} is unsubstituted C₁-C₄ alkyl.

Embodiment 46. The compound of embodiment 44, having the formula:

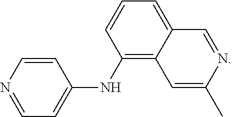 [SMILES: Cc1cc2c(Nc3ccncc3)cccc2cn1]

Embodiment 47. A compound, or a pharmaceutically acceptable salt thereof, having the formula:

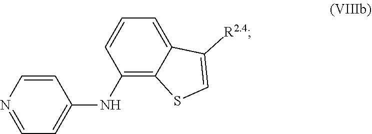

wherein
R^{2.4} is halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

Embodiment 48. The compound of embodiment 47, wherein R^{2.4} is unsubstituted C₁-C₄ alkyl.

Embodiment 49. The compound of embodiment 47, having the formula:

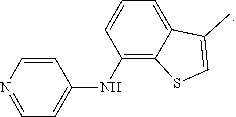 [SMILES: Cc1csc2c(Nc3ccncc3)cccc12]

Embodiment 50. A compound, or a pharmaceutically acceptable salt thereof, having the formula:

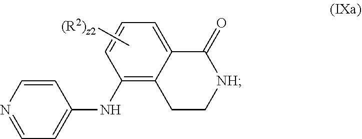

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment 51. The compound of embodiment 50, having the formula:

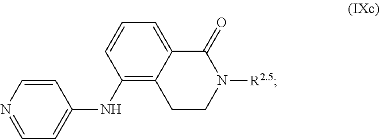

wherein
R^{2.5} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 52. The compound of embodiment 51, wherein R^{2.5} is hydrogen or unsubstituted C₁-C₄ alkyl.

Embodiment 53. The compound of embodiment 50, having the formula:

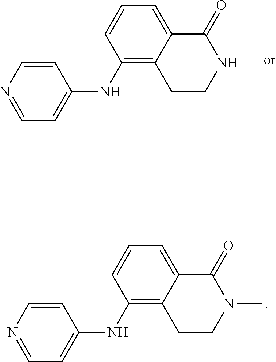 [SMILES: O=C1NCCc2c(Nc3ccncc3)cccc21 ; CN1CCc2c(Nc3ccncc3)cccc2C1=O]

Embodiment 54. A compound, or a pharmaceutically acceptable salt thereof, having the formula:

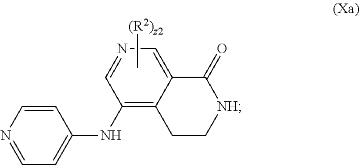

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 7.

Embodiment 55. The compound of embodiment 54, having the formula:

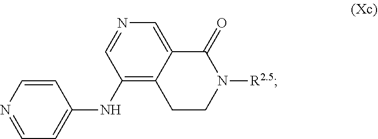

wherein
R^{2.5} is hydrogen, halogen, —CX²₃, —CHX²₂, —CH₂X², —OCX²₃, —OCH₂X², —OCHX²₂, —CN, —SOₙ₂R^{2D}—SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 56. The compound of embodiment 55, wherein R^{2.5} is hydrogen or unsubstituted C₁-C₄ alkyl.

Embodiment 57. The compound of embodiment 54, having the formula:

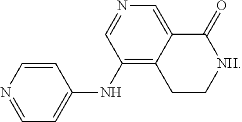 [SMILES: O=C1NCCc2c(Nc3ccncc3)cncc21]

Embodiment 58. A compound, or a pharmaceutically acceptable salt thereof, having the formula:

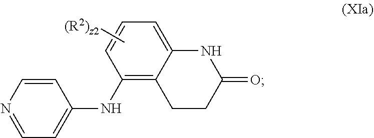

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment 59. The compound of embodiment 58, having the formula:

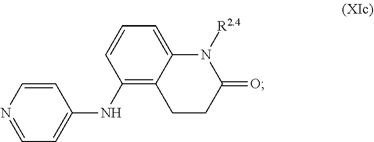

wherein
R^{2.4} is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 60. The compound of embodiment 59, wherein R^{2.4} is hydrogen or unsubstituted C₁-C₄ alkyl.

Embodiment 61. The compound of embodiment 58, having the formula:

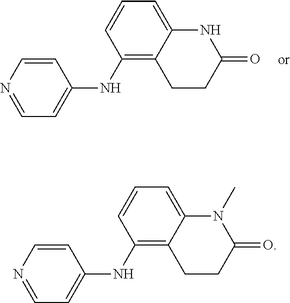 [SMILES: O=C1CCc2c(cccc2Nc2ccncc2)N1 ; CN1C(=O)CCc2c(Nc3ccncc3)cccc21]

Embodiment 62. A compound, or a pharmaceutically acceptable salt thereof, having the formula:

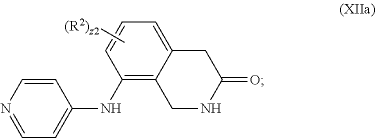

wherein
R² is independently halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

Embodiment 63. The compound of embodiment 62, having the formula:

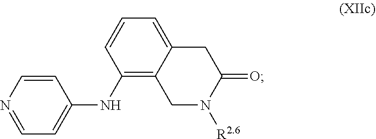

wherein
R^{2.6} is hydrogen, halogen, —CX²₃, —CHX²₂, —CH₂X², —OCX²₃, —OCH₂X², —OCHX²₂, —CN, —SOₙ₂R^{2D}—SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 64. The compound of embodiment 63, wherein R^{2.6} is hydrogen or unsubstituted C₁-C₄ alkyl.

Embodiment 65. The compound of embodiment 62, having the formula:

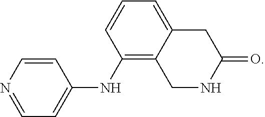 [SMILES: O=C1Cc2cccc(Nc3ccncc3)c2CN1]

Embodiment 66. A compound, or a pharmaceutically acceptable salt thereof, having the formula:

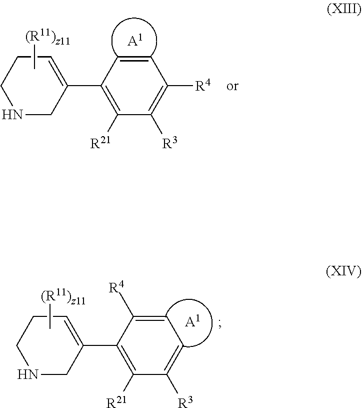

wherein
Ring A¹ is a substituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and z11 is an integer from 0 to 8.

Embodiment 67. The compound of embodiment 66, wherein ring A¹ is oxo-substituted cycloalkyl.

Embodiment 68. The compound of embodiment 66, wherein ring A¹ is a substituted cycloalkyl, wherein the substituent is oxo, halogen, —NH₂, —OH, substituted or unsubstituted C₁-C₄ alkyl, or substituted or unsubstituted 2 to 4 membered heteroalkyl.

Embodiment 69. The compound of embodiment 66, wherein ring A¹ is a substituted C₃-C₈ cycloalkyl.

Embodiment 70. The compound of embodiment 66, wherein ring A¹ is a substituted C₃-C₆ cycloalkyl.

Embodiment 71. The compound of embodiment 66, wherein ring A¹ is a substituted cyclopentyl.

Embodiment 72. The compound of one of embodiments 66 to 71, wherein z11 is 0.

Embodiment 73. The compound of one of embodiments 66 to 72, wherein R²¹ is halogen, —NH₂, —OH, substituted or unsubstituted C₁-C₄ alkyl, or substituted or unsubstituted 2 to 4 membered heteroalkyl.

Embodiment 74. The compound of one of embodiments 66 to 72, wherein R^{2.1} is halogen, —NH₂, —OH, or unsubstituted methyl.

Embodiment 75. The compound of one of embodiments 66 to 74, wherein R³ is halogen, —NH₂, —OH, substituted or unsubstituted C₁-C₄ alkyl, or substituted or unsubstituted 2 to 4 membered heteroalkyl.

Embodiment 76. The compound of one of embodiments 66 to 74, wherein R³ is halogen, —NH₂, —OH, or unsubstituted methyl.

Embodiment 77. The compound of one of embodiments 66 to 76, wherein R⁴ is halogen, —NH₂, —OH, substituted or unsubstituted C₁-C₄ alkyl, or substituted or unsubstituted 2 to 4 membered heteroalkyl.

Embodiment 78. The compound of one of embodiments 66 to 76, wherein R⁴ is halogen, —NH₂, —OH, or unsubstituted methyl.

Embodiment 79. The compound of embodiment 66, wherein the compound is:

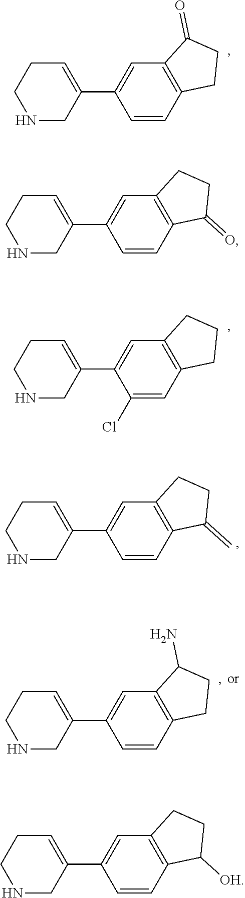 [SMILES: O=C1CCc2ccc(C3=CCCNC3)cc21 ; O=C1CCc2cc(C3=CCCNC3)ccc21 ; Clc1cc2c(cc1C1=CCCNC1)CCC2 ; C=C1CCc2cc(C3=CCCNC3)ccc21 ; NC1CCc2ccc(C3=CCCNC3)cc21 ; OC1CCc2cc(C3=CCCNC3)ccc21]

Embodiment 80. A pharmaceutical composition comprising the compound of one of embodiments 66 to 79 and a pharmaceutically acceptable excipient.

Embodiment 81. A method of treating pain in a subject in need thereof, said method comprising administering to the subject a therapeutically effective amount of a compound having the formula:

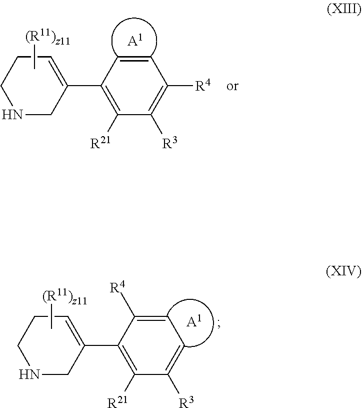

or a pharmaceutically acceptable salt thereof, wherein
Ring A¹ is a substituted or unsubstituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z11 is an integer from 0 to 8.

Embodiment 82. The method of embodiment 81, wherein the compound is not:

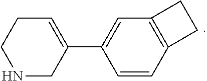 [SMILES: C1=C(c2ccc3c(c2)CC3)CNCC1]

Embodiment 83. The method of embodiment 81 or 82, wherein the pain is post-operative pain.

Embodiment 84. The method of embodiment 83, wherein the post-operative pain is pain after a hysterectomy.

Embodiment 85. The method of embodiment 83, wherein the post-operative pain is pediatric post-operative pain.

Embodiment 86. The method of one of embodiments 83 to 85, further comprising administering a second agent.

Embodiment 87. The method of embodiment 86, wherein the second agent is an opioid.

Embodiment 88. The method of embodiment 86, wherein the second agent is bupivacaine or gabapentin.

Embodiment 89. The method of embodiment 81 or 82, wherein the pain is neuropathic pain.

Embodiment 90. The method of embodiment 89, wherein the neuropathic pain is post-traumatic neuropathic pain.

Embodiment 91. The method of embodiment 89, wherein the neuropathic pain is diabetic neuropathic pain.

Embodiment 92. The method of embodiment 89, wherein the neuropathic pain is post-herpetic neuralgia.

Embodiment 93. The method of embodiment 89, wherein the neuropathic pain is chemotherapy-induced pain.

Embodiment 94. The method of embodiment 89, wherein the neuropathic pain is phantom limb pain.

Embodiment 95. The method of embodiment 81 or 82, wherein the pain is inflammatory pain.

Embodiment 96. The method of embodiment 95, wherein the inflammatory pain is associated with rheumatoid arthritis, ankylosing spondylitis, osteoarthritis, bursitis, tendinitis, or acute gouty arthritis.

Embodiment 97. The method of embodiment 81 or 82, wherein the pain is opioid refractory pain.

Embodiment 98. The method of embodiment 81 or 82, wherein the pain is a rebound headache.

Embodiment 99. The method of embodiment 81 or 82, wherein the pain is migraine pain.

Embodiment 100. The method of embodiment 81 or 82, wherein the pain is adiposis dolorosa.

Embodiment 101. The method of embodiment 81 or 82, wherein the pain is a burn pain.

Embodiment 102. The method of embodiment 81 or 82, wherein the pain is cluter headaches.

Embodiment 103. The method of embodiment 81 or 82, wherein the pain is associated with central pain conditions following stroke.

Embodiment 104. The method of embodiment 81 or 82, wherein the pain is a musculoskeletal pain.

Embodiment 105. The method of one of embodiments 81 to 104, wherein the compound is administered systemically.

Embodiment 106. The method of one of embodiments 81 to 104, wherein the compound is administered topically.

Embodiment 107. The method of one of embodiments 81 to 104, wherein the compound is administered intrathecally.

Embodiment 108. The method of one of embodiments 81 to 104, wherein the compound is administered orally.

Embodiment 109. The method of one of embodiments 81 to 104, wherein the compound is administered intravenously.

Embodiment 110. A compound, or a pharmaceutically acceptable salt thereof, having the formula:

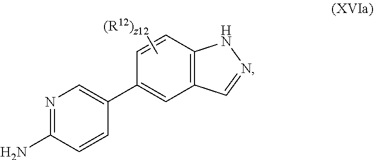

wherein
R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z12 is an integer from 0 to 8;
with the proviso that R¹² is not methyl.

Embodiment 111. The compound of embodiment 110, wherein R¹² is not C₁-C₄-alkyl.

Embodiment 112. The compound of embodiment 110, wherein R¹² is not unsubstituted C₁-C₄-alkyl.

Embodiment 113. The compound of embodiment 110, wherein R¹² is halogen, —CN, —NO₂, substituted C₁-C₄ alkyl, or substituted or unsubstituted 2 to 4 membered heteroalkyl.

Embodiment 114. The compound of embodiment 110, wherein R¹² is halogen, —CN, —NO₂, —CF₃, or —OCH₃.

Embodiment 115. The compound of one of embodiments 110 to 114, wherein z12 is 1.

Embodiment 116. The compound of embodiment 110, wherein the compound is:

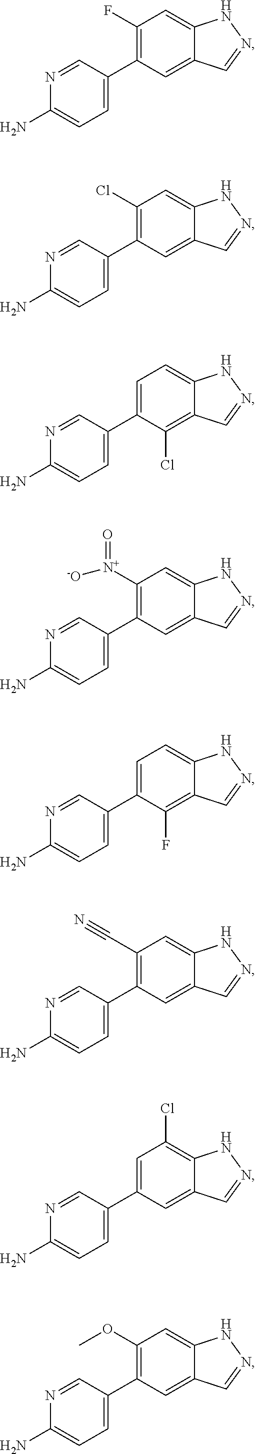

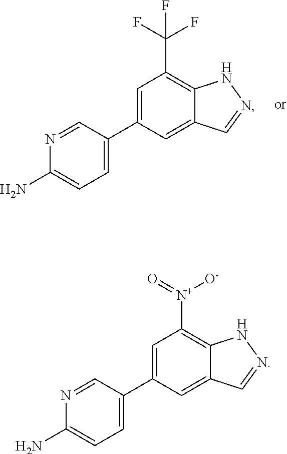 [SMILES: Nc1ccc(-c2cc(C(F)(F)F)c3[nH]ncc3c2)cn1 ; "or" ; Nc1ccc(-c2cc([N+](=O)[O-])c3[nH]ncc3c2)cn1]

Embodiment 117. A pharmaceutical composition comprising the compound of one of embodiments 110 to 116 and a pharmaceutically acceptable excipient.

Embodiment 118. A method of treating pain in a subject in need thereof, said method comprising administering to the subject a therapeutically effective amount of a compound having the formula:

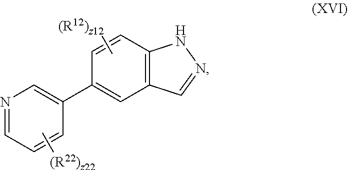

or a pharmaceutically acceptable salt thereof, wherein each R¹² and R²² are independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

Embodiment 119. The method of embodiment 118, wherein the compound is not:

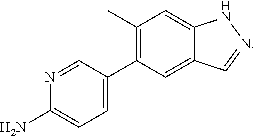 [SMILES: Cc1cc2[nH]ncc2cc1-c1ccc(N)nc1]

Embodiment 120. The method of one of embodiments 118 to 119, wherein the pain is post-operative pain.

Embodiment 121. The method of embodiment 120, wherein the post-operative pain is pain after a hysterectomy.

Embodiment 122. The method of embodiment 120, wherein the post-operative pain is pediatric post-operative pain.

Embodiment 123. The method of one of embodiments 120 to 122, further comprising administering a second agent.

Embodiment 124. The method of embodiment 123, wherein the second agent is an opioid.

Embodiment 125. The method of embodiment 123, wherein the second agent is bupivacaine.

Embodiment 126. The method of one of embodiments 118 to 119, wherein the pain is neuropathic pain.

Embodiment 127. The method of embodiment 126, wherein the neuropathic pain is post-traumatic neuropathic pain.

Embodiment 128. The method of embodiment 126, wherein the neuropathic pain is diabetic neuropathic pain.

Embodiment 129. The method of embodiment 126, wherein the neuropathic pain is post-herpetic neuralgia.

Embodiment 130. The method of embodiment 126, wherein the neuropathic pain is chemotherapy-induced pain.

Embodiment 131. The method of embodiment 126, wherein the neuropathic pain is phantom limb pain.

Embodiment 132. The method of one of embodiments 118 to 119, wherein the pain is inflammatory pain.

Embodiment 133. The method of embodiment 132, wherein the inflammatory pain is associated with rheumatoid arthritis, ankylosing spondylitis, osteoarthritis, bursitis, tendinitis, or acute gouty arthritis.

Embodiment 134. The method of one of embodiments 118 to 119, wherein the pain is opioid refractory pain.

Embodiment 135. The method of one of embodiments 118 to 119, wherein the pain is a rebound headache.

Embodiment 136. The method of one of embodiments 118 to 119, wherein the pain is migraine pain.

Embodiment 137. The method of one of embodiments 118 to 119, wherein the pain is adiposis dolorosa.

Embodiment 138. The method of one of embodiments 118 to 119, wherein the pain is a burn pain.

Embodiment 139. The method of one of embodiments 118 to 119, wherein the pain is cluster headaches.

Embodiment 140. The method of one of embodiments 118 to 119, wherein the pain is associated with central pain conditions following stroke.

Embodiment 141. The method of one of embodiments 118 to 119, wherein the pain is a musculoskeletal pain.

Embodiment 142. The method of one of embodiments 118 to 141, wherein the compound is administered systemically.

Embodiment 143. The method of one of embodiments 118 to 141, wherein the compound is administered topically.

Embodiment 144. The method of one of embodiments 118 to 141, wherein the compound is administered intrathecally.

Embodiment 145. The method of one of embodiments 118 to 141, wherein the compound is administered orally.

Embodiment 146. The method of one of embodiments 118 to 141, wherein the compound is administered intravenously.

Embodiment 147. A compound, or a pharmaceutically acceptable salt thereof, having the formula:

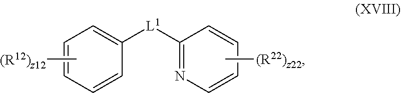

wherein
L¹ is substituted or unsubstituted C₁-C₃ alkylene;
each R¹² and R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

Embodiment 148. The compound of embodiment 147, wherein L¹ is unsubstituted C₁-C₂ alkylene.

Embodiment 149. The compound of embodiment 147, wherein L¹ is unsubstituted ethenylene.

Embodiment 150. The compound of one of embodiments 147 to 149, wherein R¹² is halogen, substituted or unsubstituted C₁-C₄ alkyl, or substituted or unsubstituted 2 to 4 membered heteroalkyl.

Embodiment 151. The compound of one of embodiments 147 to 149, wherein R¹² is —F, —Cl, —Br, —I, or —OCF₃.

Embodiment 152. The compound of one of embodiments 147 to 151, wherein z12 is 1.

Embodiment 153. The compound of one of embodiments 147 to 152, wherein R^{2.2} is —NH₂.

Embodiment 154. The compound of one of embodiments 147 to 153, wherein z22 is 1.

Embodiment 155. The compound of embodiment 147, wherein the compound is:

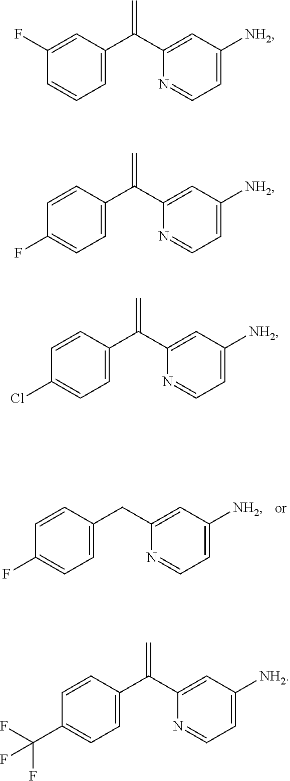 [SMILES: C=C(c1cccc(F)c1)c1cc(N)ccn1 ; C=C(c1ccc(F)cc1)c1cc(N)ccn1 ; C=C(c1ccc(Cl)cc1)c1cc(N)ccn1 ; Nc1ccnc(Cc2ccc(F)cc2)c1 ; C=C(c1ccc(C(F)(F)F)cc1)c1cc(N)ccn1]

Embodiment 156. A pharmaceutical composition comprising the compound of one of embodiments 147 to 155 and a pharmaceutically acceptable excipient.

Embodiment 157. A method of treating pain in a subject in need thereof, said method comprising administering to the subject a therapeutically effective amount of a compound having the formula:

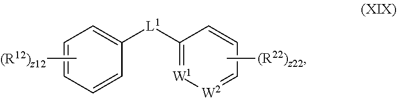

or a pharmaceutically acceptable salt thereof,
wherein
L¹ is —O—, —NR¹⁰—, or substituted or unsubstituted alkylene;
W¹ is N or CR³;
W² is N or CR⁴;
each R¹² and R^{2.2} is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5;
z22 is an integer from 0 to 4;
R³ and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen or unsubstituted C₁-C₄ alkyl;
wherein at least one of W¹ or W² is N.

Embodiment 158. The method of embodiment 157, wherein the compound is not:

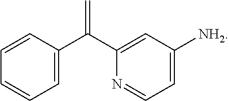

Embodiment 159. The method of one of embodiments 157 to 158, wherein W¹ is N.

Embodiment 160. The method of one of embodiments 157 to 159, wherein W² is CH.

Embodiment 161. The method of one of embodiments 157 to 160, wherein L¹ is substituted or unsubstituted C₁-C₃ alkylene.

Embodiment 162. The method of one of embodiments 157 to 160, wherein L¹ is unsubstituted C₁-C₂ alkylene.

Embodiment 163. The method of one of embodiments 157 to 160, wherein L¹ is unsubstituted ethenylene.

Embodiment 164. The method of one of embodiments 157 to 163, wherein R¹² is independently halogen, substituted or unsubstituted C₁-C₄ alkyl, or substituted or unsubstituted 2 to 4 membered heteroalkyl.

Embodiment 165. The method of one of embodiments 157 to 163, wherein R¹² is independently —F, —Cl, —Br, —I, or —OCF₃.

Embodiment 166. The method of one of embodiments 157 to 165, wherein z12 is 1.

Embodiment 167. The method of one of embodiments 157 to 166, wherein R^{2.2} is —NH₂.

Embodiment 168. The method of one of embodiments 157 to 167, wherein z22 is 1.

Embodiment 169. The method of one of embodiments 157 to 168, wherein R³ and R⁴ are hydrogen.

Embodiment 170. The method of one of embodiments 157 to 169, wherein the pain is post-operative pain.

Embodiment 171. The method of embodiment 170, wherein the post-operative pain is pain after a hysterectomy.

Embodiment 172. The method of embodiment 170, wherein the post-operative pain is pediatric post-operative pain.

Embodiment 173. The method of one of embodiments 170 to 172, further comprising administering a second agent.

Embodiment 174. The method of embodiment 173, wherein the second agent is an opioid.

Embodiment 175. The method of embodiment 173, wherein the second agent is bupivacaine or gabapentin.

Embodiment 176. The method of one of embodiments 157 to 169, wherein the pain is neuropathic pain.

Embodiment 177. The method of embodiment 176, wherein the neuropathic pain is post-traumatic neuropathic pain.

Embodiment 178. The method of embodiment 176, wherein the neuropathic pain is diabetic neuropathic pain.

Embodiment 179. The method of embodiment 176, wherein the neuropathic pain is post-herpetic neuralgia.

Embodiment 180. The method of embodiment 176, wherein the neuropathic pain is chemotherapy-induced pain.

Embodiment 181. The method of embodiment 176, wherein the neuropathic pain is phantom limb pain.

Embodiment 182. The method of one of embodiments 157 to 169, wherein the pain is inflammatory pain.

Embodiment 183. The method of embodiment 182, wherein the inflammatory pain is associated with rheumatoid arthritis, ankylosing spondylitis, osteoarthritis, bursitis, tendinitis, or acute gouty arthritis.

Embodiment 184. The method of one of embodiments 157 to 169, wherein the pain is opioid refractory pain.

Embodiment 185. The method of one of embodiments 157 to 169, wherein the pain is a rebound headache.

Embodiment 186. The method of one of embodiments 157 to 169, wherein the pain is migraine pain.

Embodiment 187. The method of one of embodiments 157 to 169, wherein the pain is adiposis dolorosa.

Embodiment 188. The method of one of embodiments 157 to 169, wherein the pain is a burn pain.

Embodiment 189. The method of one of embodiments 157 to 169, wherein the pain is cluster headaches.

Embodiment 190. The method of one of embodiments 157 to 169, wherein the pain is associated with central pain conditions following stroke.

Embodiment 191. The method of one of embodiments 157 to 190, wherein the compound is administered systemically.

Embodiment 192. The method of one of embodiments 157 to 190, wherein the compound is administered topically.

Embodiment 193. The method of one of embodiments 157 to 190, wherein the compound is administered intrathecally.

Embodiment 194. The method of one of embodiments 157 to 190, wherein the compound is administered orally.

Embodiment 195. The method of one of embodiments 157 to 190, wherein the compound is administered intravenously.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Discovery of Novel α_{2A}AR Analgesics from Molecular Docking

The high prevalence of pain (1) and the opioid use disorder epidemic (2, 3) have inspired a search for non-opioid analgesic targets (4-7). Among the rare ones validated as an effective therapeutic is the α_{2A}-adrenergic receptor (α_{2A}AR), the target of dexmedetomidine (8). While this drug has many advantages in emergency room and intensive care settings, its strong sedative effects and its lack of an oral formulation have limited its broad use as an analgesic.

Most α_{2A}AR analgesics are chemically related, and the relationship of their sedative to their analgesic properties is unclear. Thus, it seemed attractive to look for new chemotypes, topologically unrelated to known α_{2A}AR agonists, which nevertheless are potent on-target. With the determination of the structure of the highly-related α_{2B}-adrenergic receptor (α_{2B}AR) (9), this seemed possible via structure-based docking. Docking computationally screens libraries of molecules for those that well-complement a binding site. The advent of readily accessible make-on-demand molecules (10-12) ranging from hundreds-of-millions (10, 13, 14) to over a billion (15) molecules has vastly increased chemotype coverage, making the discovery of novel ligands for even well-studied targets plausible. Often, these new chemotypes are accompanied by new pharmacology (10, 16, 17). This approach has revealed new ligands with 30-60% hit rates (13, 14, 16, 18) and sometimes nanomolar potencies for a growing range of targets (10, 13, 14, 18-21). Therefore, we targeted the α_{2B}AR with an ultra-large library docking screen seeking novel α_{2A}AR agonists with diverse chemotypes. Such new chemotypes—unrelated to known α_{2A}AR agonists, including dexmedetomidine and clonidine—may confer analgesia without the sedative and dosing liabilities of the current drugs.

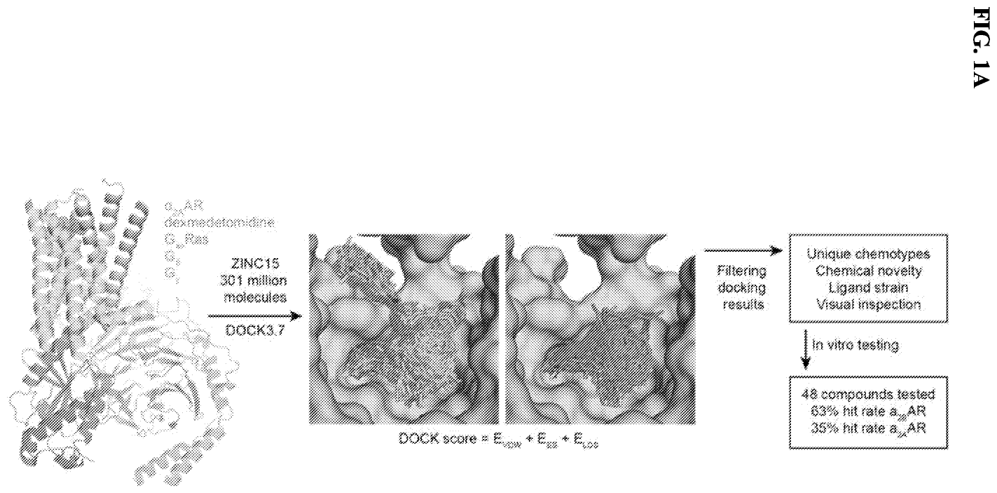

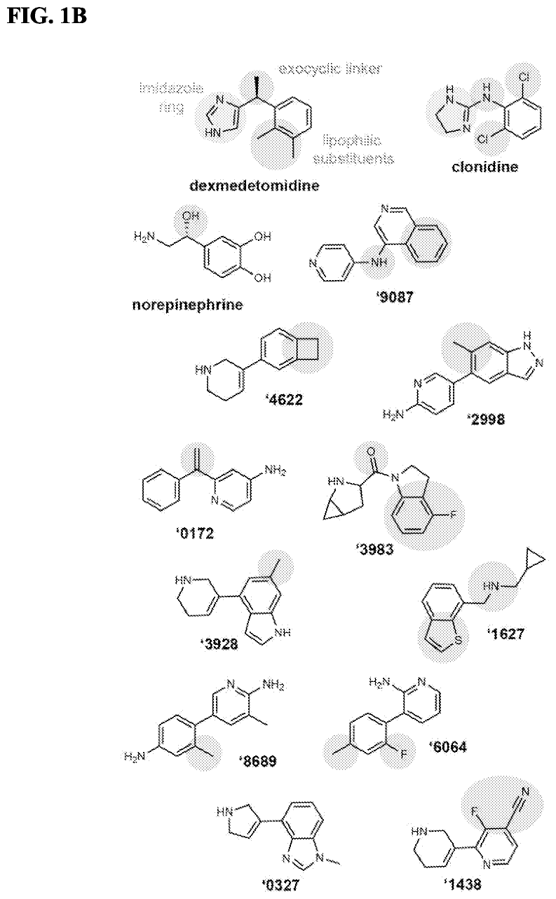

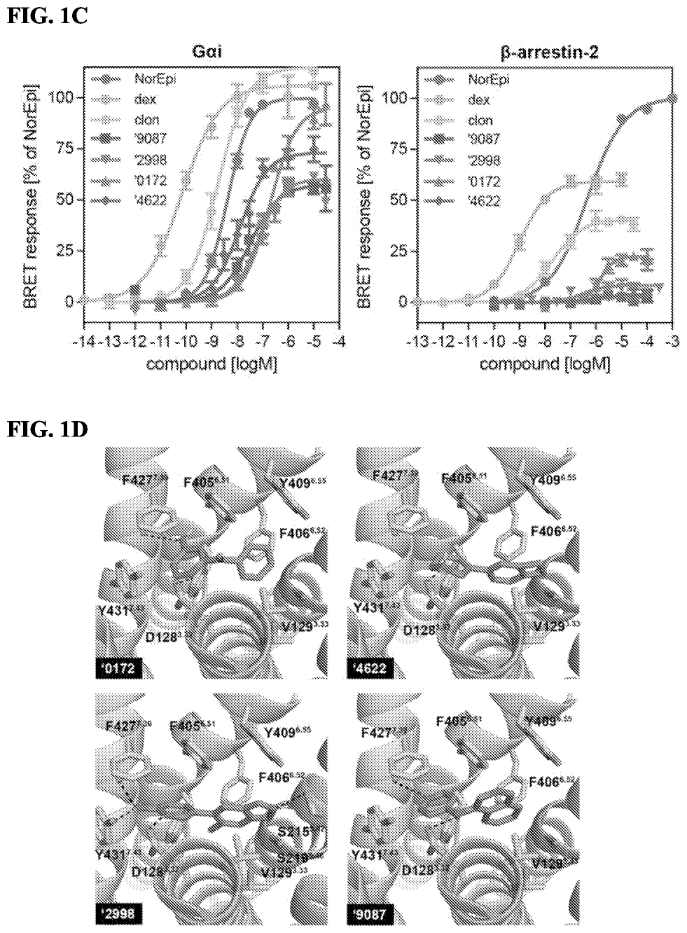

Docking 301 million molecules versus the α_{2B}AR. Given the small size of the α_{2B}AR/α_{2A}AR orthosteric sites, we docked both the 20 million ‘fragment-like’ (<250 amu, calculated Log P (c Log P)≤2.5) and 281 million ‘lead-like’ (250-350 amu, c Log P<3.5) molecules (11) (FIG. 1A). Over 234 trillion receptor-molecule complexes were sampled by DOCK3.7 and scored with its physics-based energy function (22) across three separate screens (see Example 2). For each screen, the top 300,000 docking-ranked compounds were clustered for topological similarity, filtered to identify scaffolds dissimilar to agonists from the IUPHAR database and literature (23-26) using ECFP4 fingerprints, and ligands with internal torsional strain were removed (27). An additional novelty filter was performed removing molecules similar to annotated ChEMBL29 α_{2A}AR compounds (23). The remaining top-ranked molecules were visually evaluated for key polar and nonpolar interactions with α_{2B}AR (9), including D128^{3.32}, F427^{7.39}, F405^{6.51}, Y409^{6.55}, and F406^{6.52} (superscripts use Ballesteros-Weinstein and GPCRdb nomenclature (28, 29). Most α_{2A}AR agonists, and certainly the clinically used dexmedetomidine and clonidine, are fragments (30), and the docking results reflected this. The docked fragment molecules fit in the orthosteric site making key contacts with the receptor, while molecules in the lead-like screen struggled to fit in the small cavity (FIG. 1A). Accordingly, most selected ligands came from the fragment docking screens.

Of the 64 compounds prioritized for synthesis and testing, 48 were successfully synthesized, 44 fragments and 4 lead-like molecules (a synthetic fulfillment rate of 75%). Compounds were first tested for binding to the α_{2B}AR receptor, the structure used in docking screens. Thirty molecules had Kᵢ values less than 10 μM, a hit rate of 63% (30 actives/48 compounds tested), among the highest for a docking campaign to date. In radioligand displacement assays compound ‘9087 had a Kᵢ of 1.7 nM, while 29 others had Kᵢ values ranging from 60 nM to 9.4 μM, which is relatively potent for initial docking hits. The compounds were then tested for binding to the murine α_{2A}AR, again by radioligand displacement. Of these, 17 had Kᵢ better than 10 μM, a hit rate of 35% with affinities ranging from 72 nM to 9.4 μM. Against the human α_{2A}AR compounds had affinities as low as 12 nM (Table 1).

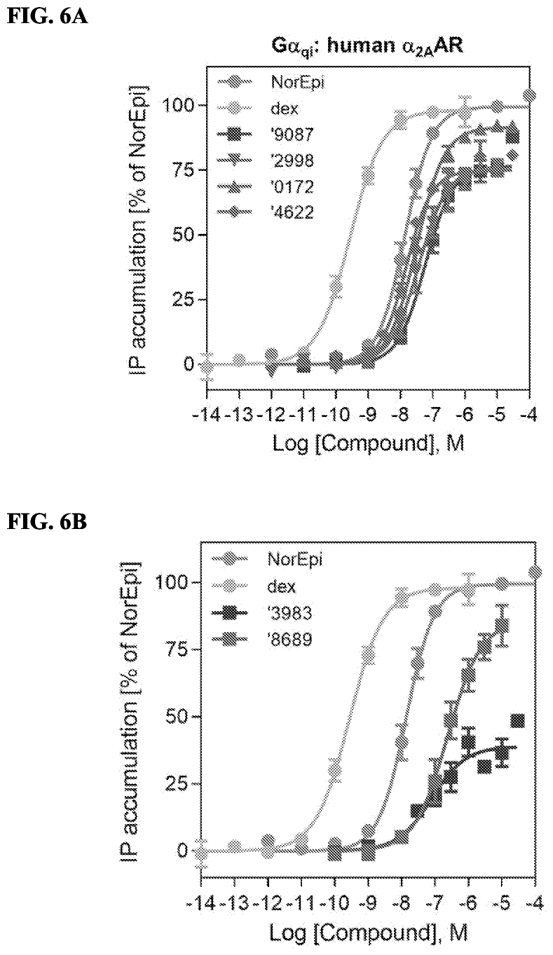

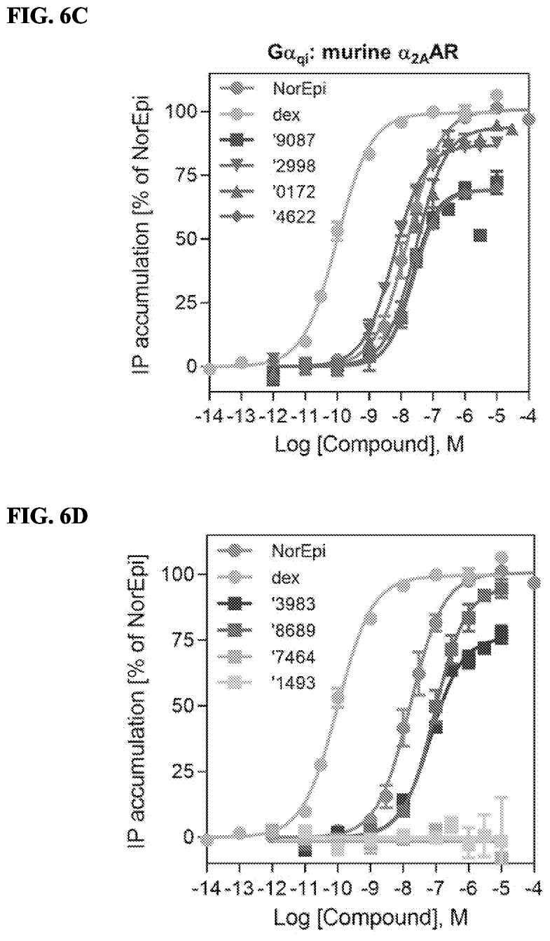

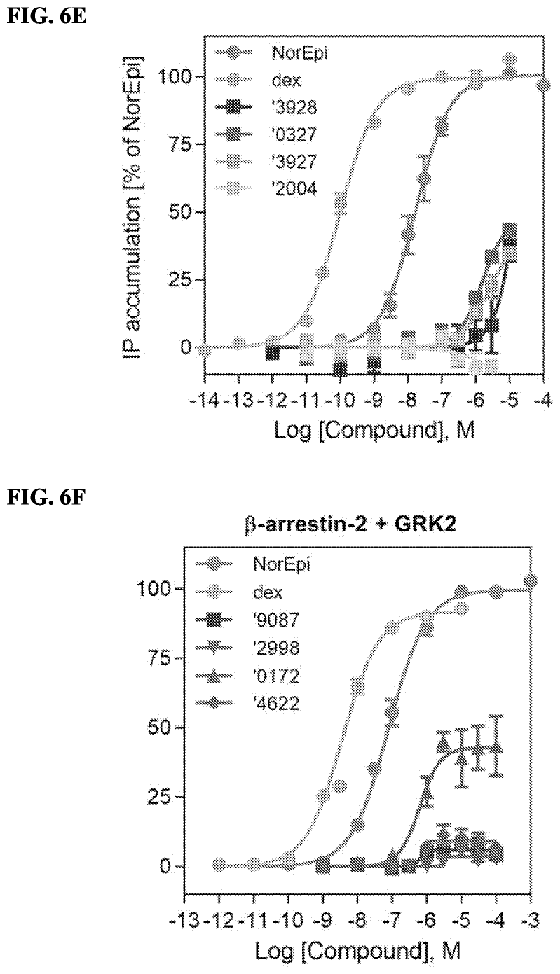

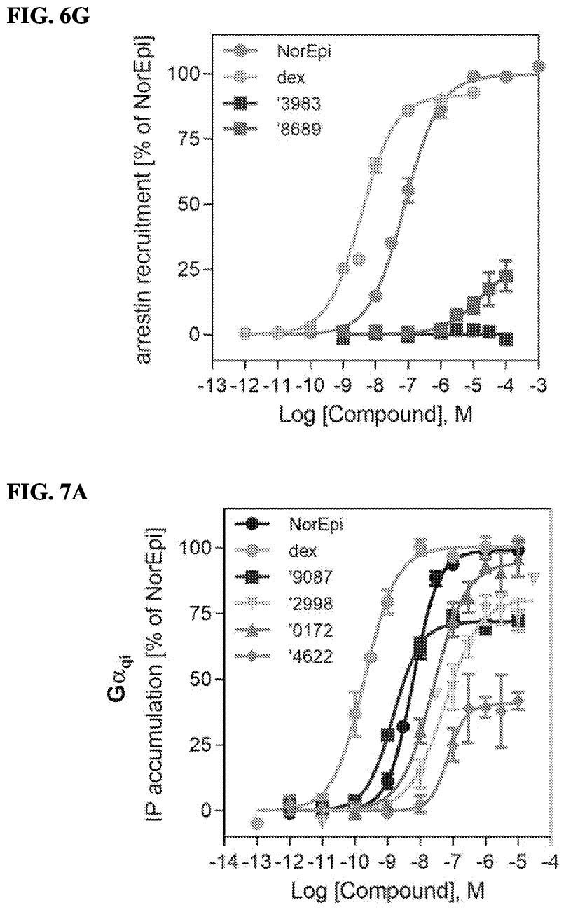

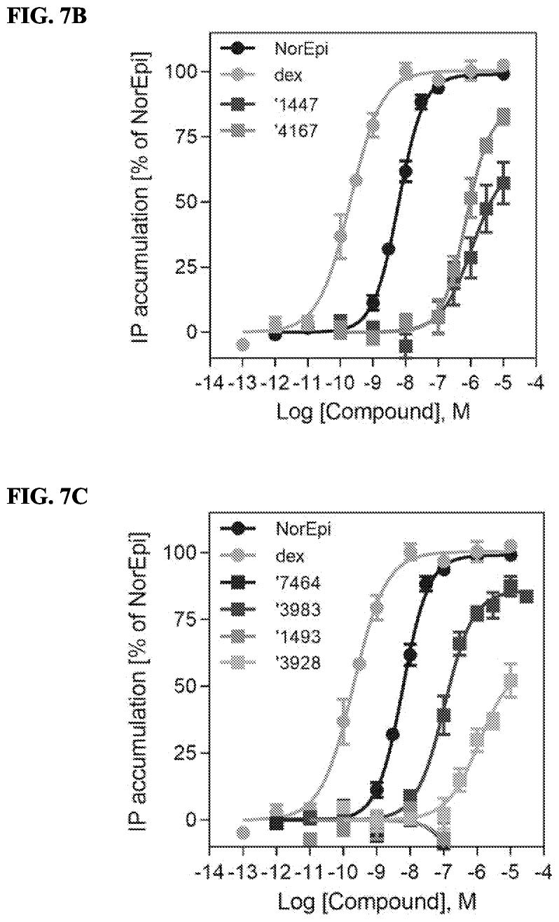

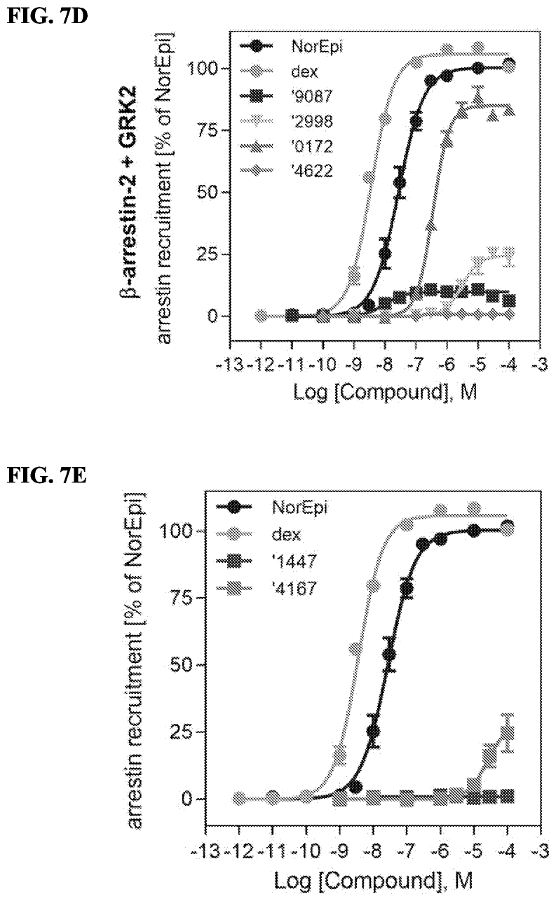

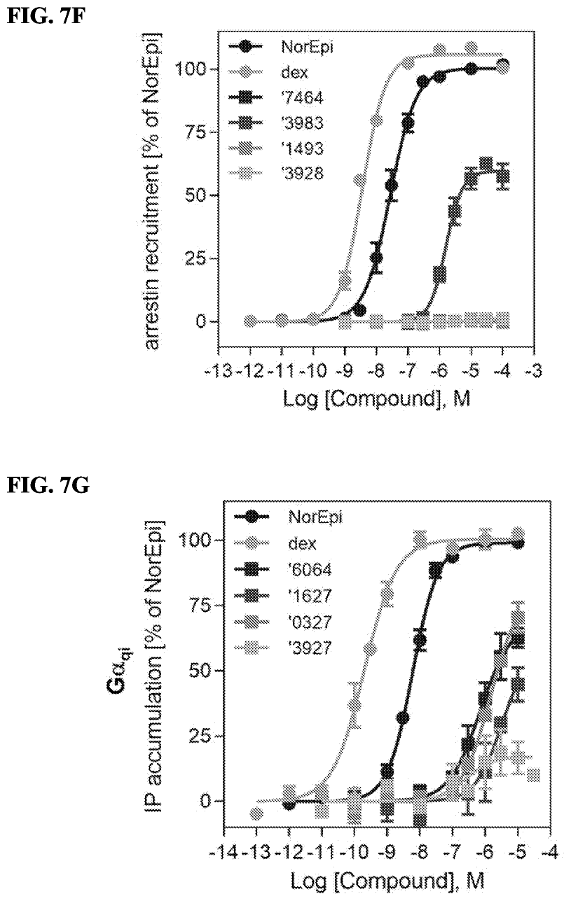

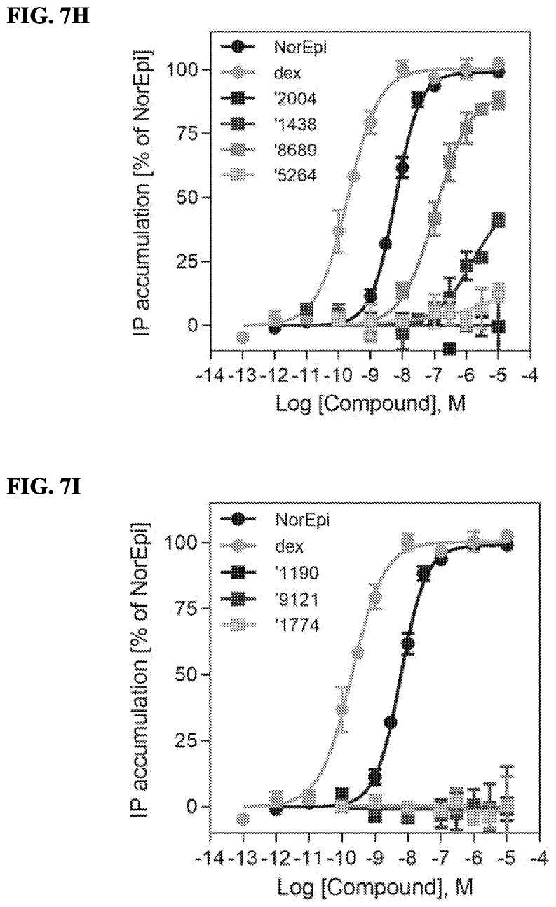

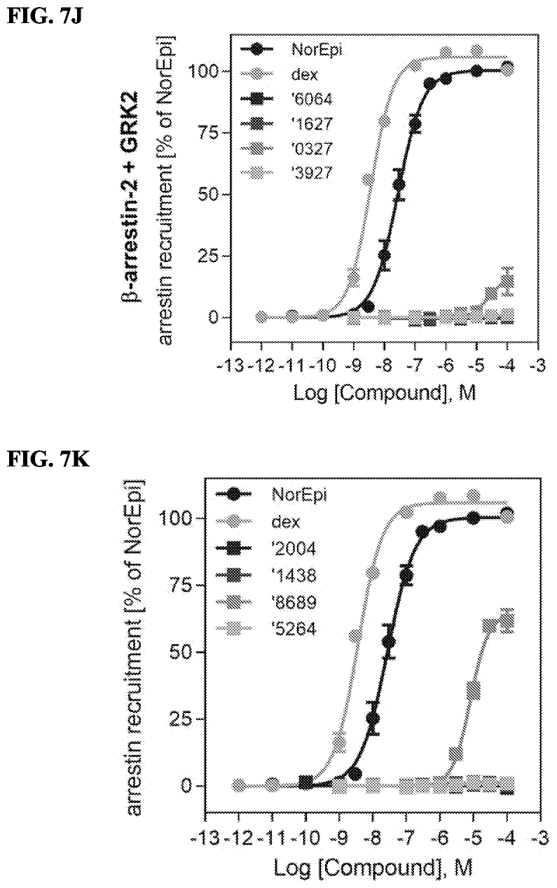

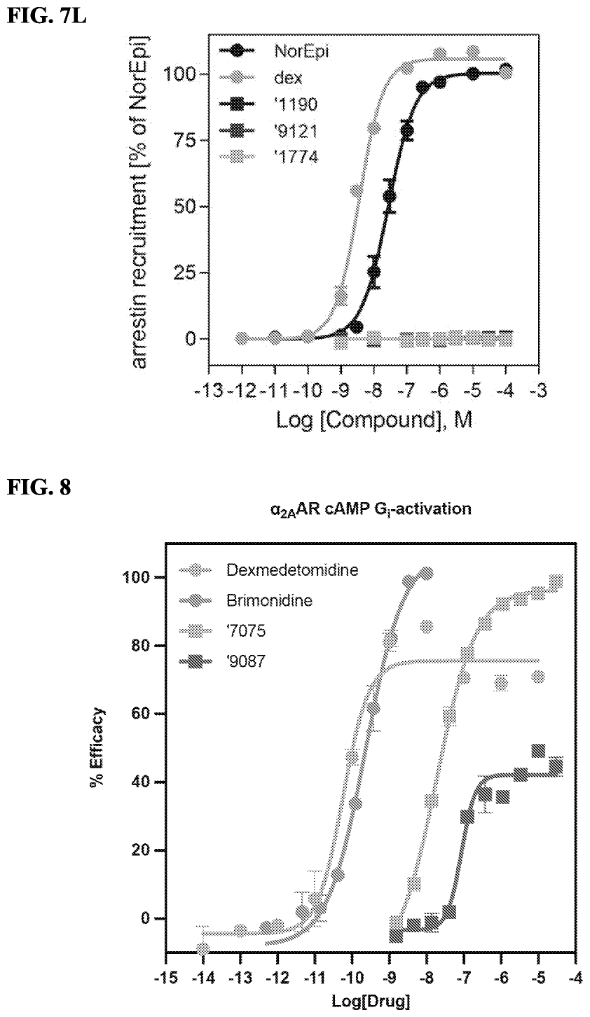

Discovery of novel, G-protein-biased agonists. In functional assays, most of the potent binders were agonists for α_{2A}AR and α_{2B}AR (FIGS. 1B-1D, FIGS. 6A-6G, FIGS. 7A-7L, FIG. 8); few antagonists were found among the more potent docking hits. This reflects the targeting of the activated state of the receptor and was a goal of the screen. More surprising was the strong bias of most of the new agonists for Gᵢ activation versus arrestin recruitment. The best agonists from the docking screen include ZINC1173879087, ZINC1240664622, ZINC1242282998, and ZINC001242890172 (from here on referred to as ‘9087, ‘4622, ‘2998, and ‘0172, respectively), with the α_{2A}AR-mediated Gᵢ activation Eₘax ranging from 60-95% of norepinephrine response and EC₅₀ values of 9.7 to 210 nM in Gαi BRET assays. To confirm these results we tested ‘9087 (and ultimately, an optimized analog PS75, below) in a second assay, conducted by a third-party CRO (Eurofins) using the DiscoverX HitHunter cAMP assay. Here, ‘9087 had an EC₅₀ of 87 nM and Eₘₐₓ of 42%, which is broadly consistent with our own observations in the BRET assay (from here on Gᵢ activities are the Gαi BRET assay values unless otherwise noted) (FIG. 1C, FIG. 8, Table 1). Of the four agonists, ‘0172 recruited β-arrestin-2 with the highest Eₘax, but even so with only 22% efficacy versus norepinephrine and a weaker potency (EC₅₀=1.47 PM) (FIG. 1C), while for the other three β-arrestin recruitment was negligible (FIG. 1C, Table 1).

Comparing the new docking-derived agonists to dexmedetomidine, clonidine, norepinephrine, and to a previously described pharmacophore model for α_{AR} selective agonists (9, 34), both similar and unique features emerge (FIG. 1B). The model for known agonists and the new docking compounds both have basic, nitrogen-containing rings. However, known agonists are dominated by imidazoles (unsaturated or partially saturated) while the docking compounds have diverse, non-imidazole rings. Both sets of compounds contain additional moieties off of a second aryl ring, however for the docking actives these vary from bulky hydrophobic rings, to hydrophilic rings, to single substituents, to having no substituents off of the aryl ring. Not all of the docking compounds have an exocyclic linker as described in the pharmacophore model. The imidazole of known agonists in its protonated form forms a salt bridge with D128^{3.32} and hydrogen bonds to the backbone of F427^{7.39} (9, 33). Although several of the docking actives also interacted with both D128^{3.32} and F427^{7.39}, they did so with different heterocyclic rings (FIG. 1D).

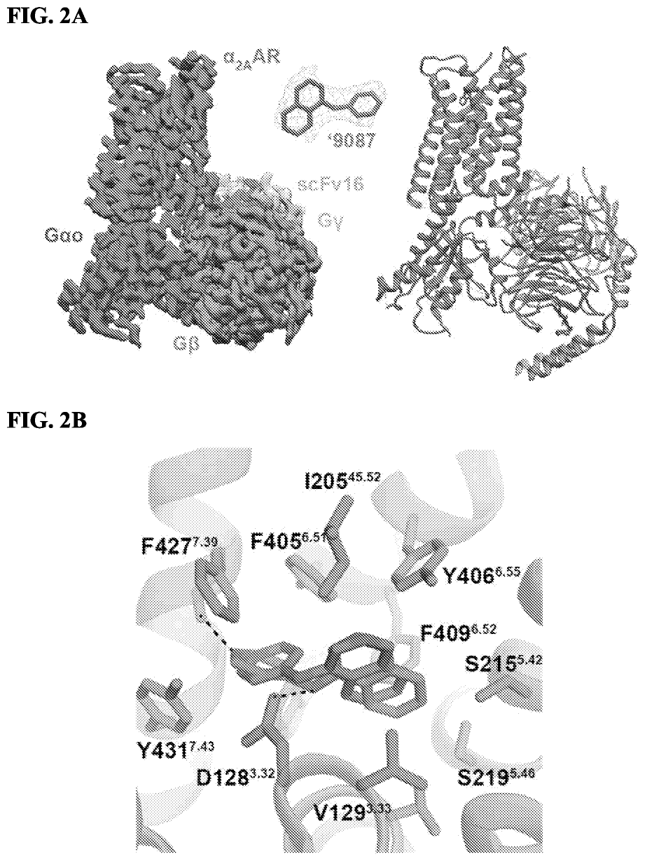

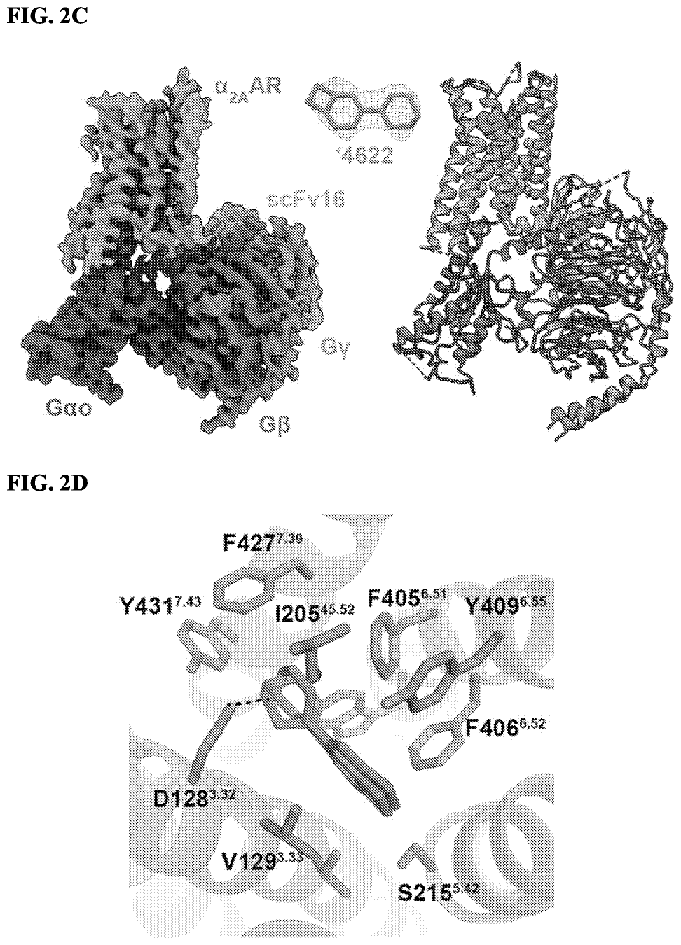

To test the docking model and to template structure-based optimization, we determined the structure of the ‘9087-α_{2A}AR-Goa and ‘4622 α_{2A}AR-Goα signaling complexes at a nominal resolution of 3.47 Å and 3.38 Å, respectively, using single particle cryo-electron microscopy (cryo-EM) (FIGS. 2A-2D). The predicted docked pose superimposes on the cryo-EM result of ‘9087 with a 1.14 Å all-atom RMSD of the agonist; the docking-predicted interactions are recapitulated in the experimental structure (FIG. 2B). The interactions between ‘9087 and α_{2A}AR differ from that of norepinephrine, but resemble those of imidazoline-containing agonists, despite the different chemotype of the new agonist (9, 33). ‘9087 interacts with α_{2A}AR mainly through van der Waals and aromatic interactions to transmembrane helices (TM) 3, 5, 6 and 7 as well as with I205^{45.52} of extracellular loop 2 (ECL2). It also forms an ionic interaction with the conserved D128^{3.32}, although the interaction is relatively distant at 4 Å, compared to those of norepinephrine (33) and dexmedetomidine (9) that are 3.5 Å or less from the aspartate (FIG. 2B). Notably, there is no polar interaction between ‘9087 and Y431^{7.43}, instead, ‘9087 hydrogen-bonds with the backbone carbonyl of F427^{7.39}, which is not observed for norepinephrine but is seen with dexmedetomidine (9). We note that, as in the docking prediction, the basic, formally cationic nitrogen of ‘9087 is not oriented toward D128^{3.32} to form a salt bridge (FIG. 2B), but instead it hydrogen-bonds with the backbone carbonyl of F427^{7.39}, while it is the bridging exocyclic and formally neutral amine of ‘9087 that ion pairs with D128^{3.32}. Intuitively, one might expect the stronger base to make the conserved hydrogen bond with D128^{3.32} (9, 33-36). In fact, the formal charge of ‘9087 after protonation of the pyridine moiety is almost equally shared between the two nitrogens, as calculated by semi-empirical quantum mechanics and as reflected in the docking model. For ‘4622 the docked pose is also in good agreement with the cryo-EM result with an all-atom RMSD of 1.14 Å all-atom RMSD; ‘4622 hydrogen bonds to D128^{3.32} in 3.4 Å and forms many hydrophobic interactions (FIG. 2D).

The weaker polar interaction network with D128^{3.32} and Y431^{7.43} may relate to the unusual lack of β-arrestin recruitment for ‘9087 (FIG. 1C). Consistent with this possibility, similarly weak interactions with D128³³⁴/Y431^{7.43} were observed in the oxymetazoline-α_{2A}AR structure (33); oxymetazoline also has partial β-arrestin activity. Since ‘9087, oxymetazoline, and dexmedetomidine lack polar interactions with TM5 and TM6 which are in turn observed for norepinephrine, these interactions may contribute to β-arrestin recruitment for α_{2A}AR agonists (FIG. 1C, FIG. 2B). Recent studies on dopamine and serotonin receptors show the polar interactions with TM5 and TM6 are key for signaling bias (37). Returning to the overall structure, when comparing the ‘9087-α_{2A}AR complexes to that of other α_{2A}AR agonists (33), Goα appears to couple tighter to α_{2A}AR indicated by a ˜1.5 Å shift of Goα towards α_{2A}AR and a ˜1.5 Å closer arrangement of the conserved α_{2A}AR residues Y^{5.58} and Y^{7.53}, which may stabilize a more occluded active confirmation of α_{2A}AR.

The interactions observed in the ‘9087 receptor complex were further investigated by residue substitution. Consistent with the ion-pair with D128^{3.32} observed in their complexes, norepinephrine and dexmedetomidine are highly sensitive to substitutions to D128^{3.32}, with a loss of Gᵢ activation in most mutations and EC₅₀ values increasing (worsening) by 200,000-fold with D128^{3.32}A for dexmedetomidine. While the activity of ‘9087 is also diminished in the D128^{3.32} mutant receptors, the potency only falls by about 300 to 900 fold, consistent with the more distant interaction with this aspartate seen the in the ‘9087 complex (FIG. 2B). In contrast, the activity of ‘9087 is eliminated by a substitution to F427^{7.39}, with whose backbone carbonyl ‘9087 hydrogen-bonds with in the cryo-EM structure, whereas dexmedetomidine and norepinephrine are less affected by this mutant (FIG. 2B). This interaction is not seen in the norepinephrine-α_{2A}AR structure (33) though it is observed in the dexmedetomidine-α_{2A}AR structure (9). For mutations of Y409^{6.55}, with which norepinephrine hydrogen bonds (33), norepinephrine is most affected with increased EC₅₀ s by 260 to 6,200-fold, whereas the activity of ‘9087 is only modestly affected. Substitutions to Y431^{7.43} strongly affected all three agonists: Y431^{7.43}F essentially eliminated Gᵢ signaling by ‘9087, while Y431^{7.43}A did the same for norepinephrine and dexmedetomidine. Taken together, the differential response to these substitutions supports suggestions from the structures that the new agonists, while binding in the same overall site as the canonical agonists, interact in meaningfully different ways, with potential implications for differential receptor signaling.

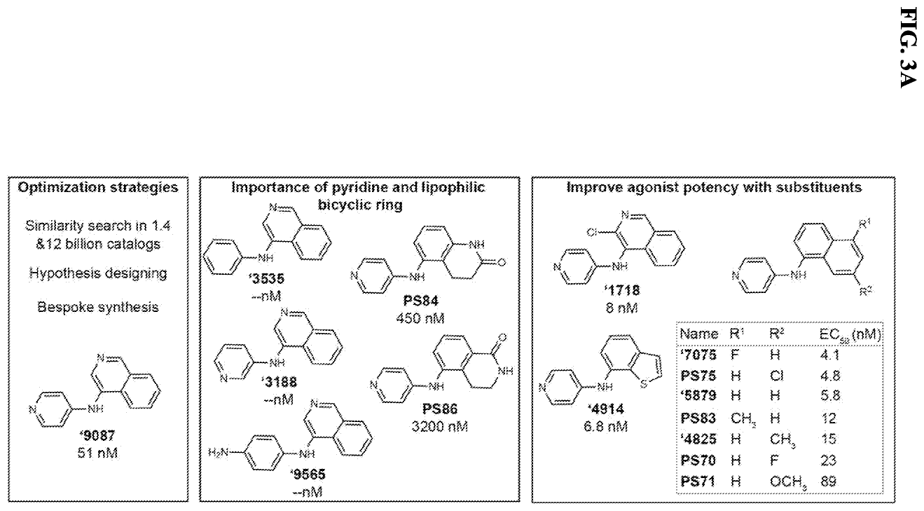

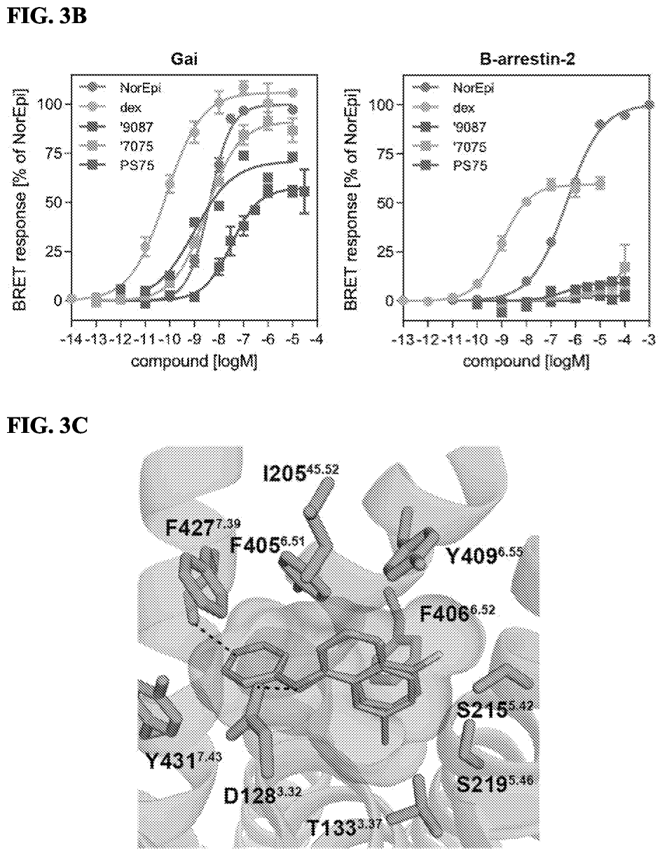

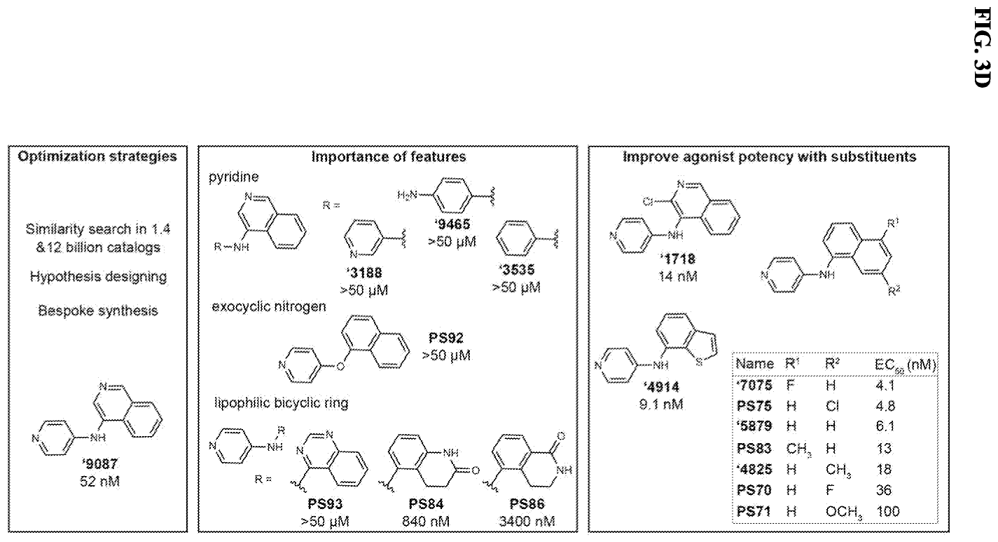

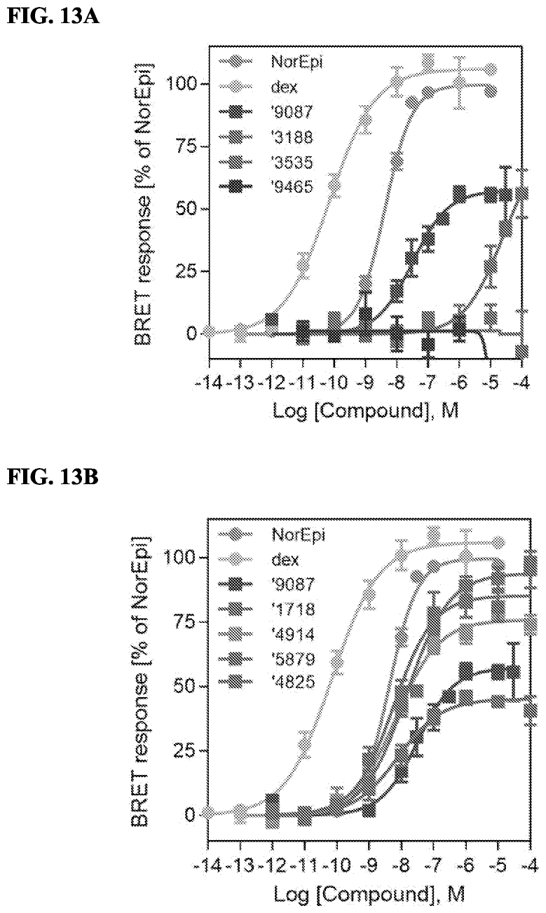

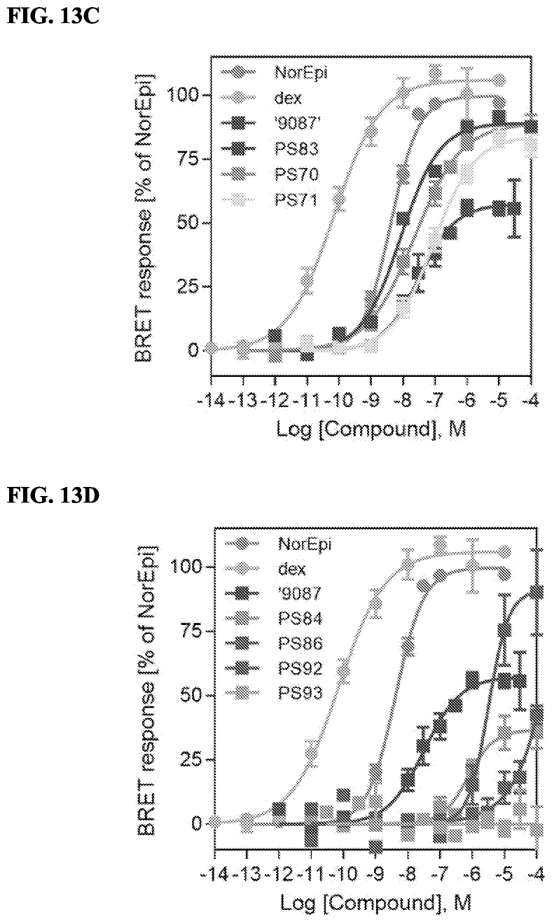

To optimize the new ligand, we adopted two strategies. Initially, we used classic medicinal chemistry hypothesis testing and analoging to investigate and improve key recognition features. An innovation was initially looking for possible analogs by similarity searching among 1.4 billion and 12 billion make-on-demand molecules using Arthor and SmallWorld (12) (NextMove Software, England) and docking prioritized subsets, which returned 13 of the 19 ‘9087 analogs prioritized in this strategy (FIG. 3A). Another six molecules were designed to probe particular interactions; five of these were also available in ZINC15 (11) or in the 12 billion set, except one compound that was designed for bespoke synthesis (Example 2, FIG. 3A). Analogs were also investigated around compounds ‘2998, '4622, and ‘0172. In the ‘9087 series, convering the pyridine to a phenyl eliminated activity, confirmed the importance of the cationic character, ion pair with Asp128, and the importance of the hydrogen bond with F427^{7.39} (FIG. 3A, FIGS. 13A-13C). The most potent analogs emerged from variations of the isoquinoline ring in ‘9087. This group is complemented by hydrophobic residues like F405^{6.51}, F406^{6.52}, Y409^{6.55}, and I205^{45.52} without obvious polar interactions with the isoquinoline nitrogen of ‘9087 (FIG. 2B, FIG. 3A). Accordingly, we added small non-polar groups, like the chlorine in ‘1718, we changed the isoquinoline to a benzothiophene in ‘4914, we replaced it for a naphthalene, or a combination of these modifications as in ‘4825, resulting in five more potent agonists (EC₅₀ 4.1 nM to 15 nM) (FIGS. 3A-3B, FIGS. 13A-13C, Table 1). ‘7075 showed 13-fold increased potency (EC₅₀ 4.1 nM) and higher Eₘₐₓ of 93% for Gᵢ activation in the BRET assay and the cAMP assay (EC₅₀ 18 nM, Eₘₐₓ 96%), and nearly the same weak 0-arrestin-2 recruitment as ‘9087 (FIG. 3B, FIG. 8, Table 1). The modeled ‘7075 complex suggests it maintains the orientation adopted by ‘9087, with its new fluorine oriented towards open space in the site between residues Y409^{6.55} and S215^{5.42} and with its naphthalene ring making the same interactions as the original isoquinoline, though without the desolvation penalty incurred by the more polar ring (FIG. 2B, FIG. 3C). The effect of mutations of putative α_{2A}AR recognition residues were largely consistent with this structure and Gᵢ activation of ‘9087 except for S215^{5.42}A in which ‘7075 potency improved 11-fold.

The determination of the ‘9087-α_{2A}AR complex also afforded us the chance to use a purely structure-based strategy for ligand optimization. New analogs were designed using structure-activity relationships (SAR) from the first round of ‘9087 optimization, as well as introducing new perturbations to the molecule (FIG. 3A). Molecules were prioritized for their favorable docked pose in the ligand-free α_{2A}AR-‘9087 structure, or simply for hypothesis testing, and were acquired through bespoke synthesis, resulting in 6 compounds (FIG. 3A). Assuming the same binding mode for ‘9087 and its naphthalene derivative ‘5879, unexploited space between the ligand and the receptor in the orthosteric site is revealed in positions 5 and 7 of the bicyclic moiety of ‘5879 (R¹ and R² in FIG. 3A, respectively; FIG. 2B, FIG. 3C). To probe the available space in these positions, substituents of different size were docked or modeled in silico to the scaffold of ‘5879.

In position 7 of ‘5879, multiple substituents were investigated to increase the contact area between the ligands and the receptor. Our model suggested introducing a chlorine substituent at position 7 in PS75 would increase of the contact area by 20 Å² and have high complementarity between the receptor and ligand, whereas a fluorine substituent in PS70 was predicted to minimally increase the contact area (FIG. 3A, FIG. 3C). The potency of PS75 for Gᵢ activation was similar to ‘5879 (EC₅₀ s of 4.8 nM and 5.8 nM, respectively), however PS70 (EC₅₀=23 nM) did not improve activity (FIGS. 3A-3B, FIGS. 13A-13C, Table 1). Adding a methoxy in the 7 position in analog PS71 reduced activity 15-fold compared to ‘5879 (FIG. 3A, FIGS. 13A-13C); this is likely the result of entropic and desolvation penalties, and a repulsive interactions predicted in the in silico model for the bulky methoxy substituent. Position 5 of the bicyclic moiety revealed less space to be exploited by spatially demanding substituents. The calculated contact area between the ligands and the receptor increased for H<F<CH₃ substituents (those for ‘5879, ‘7075, PS83, respectively) indicating the analog PS83 could be favorable (FIG. 3A). However, a clash of the methyl of PS83 to the receptor was predicted in the models and was reflected in the worsened Gᵢ activity of 12 nM compared to ‘5879 and ‘7075 (EC₅₀ s of 5.8 nM and 4.8 nM, respectively; FIGS. 3A-3B, FIGS. 13A-13C, Table 1).

We also tried to take advantage of two proximal serine residues S215^{5.42} and S219^{5.46} in TM5 to increase hydrogen bonding by introducing a lactam group into the bicyclic moiety of ‘9087 (FIG. 2B, FIG. 3A). Analog PS84 and its regioisomer PS86 were less potent for both EC₅₀ and Eₘₐₓ of Gᵢ activity (FIG. 3A, FIGS. 13A-13C). These findings highlight the importance of the lipophilic and aromatic properties of the bicyclic moiety for α_{2A}AR binding and activation, facilitating favorable interactions with the aromatic residues F405^{6.51}, F406^{6.52} and Y409^{6.55} (FIG. 2B).

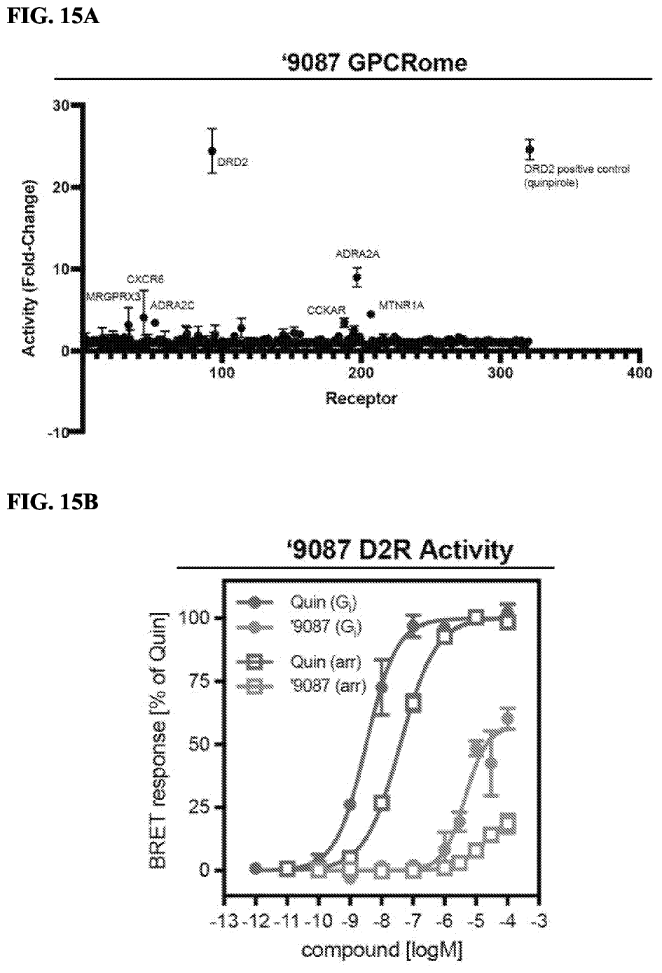

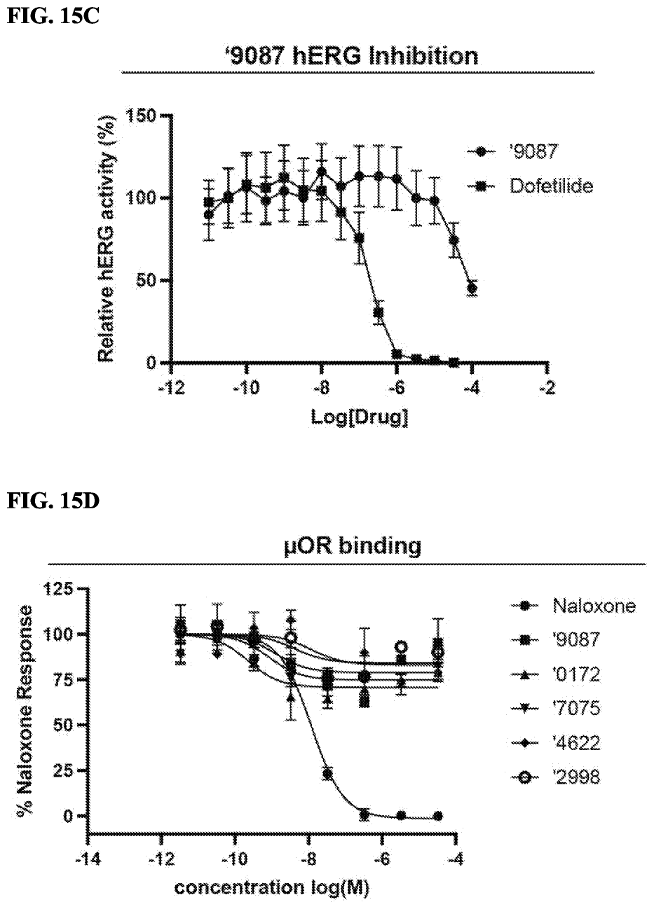

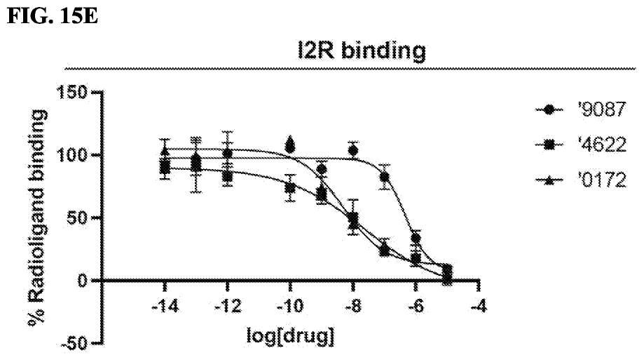

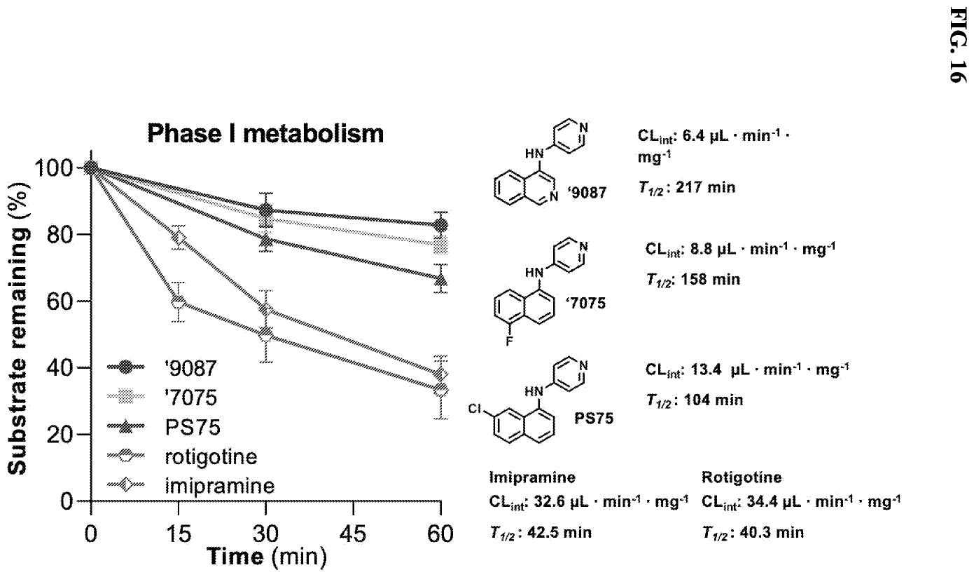

Novel α_{2A}AR agonists are analgesic with reduced side effects. In preparation for in vivo studies, we investigated the selectivity and pharmacokinetic properties of our most potent agonists. ‘9087 activated only a few of 320 G protein coupled receptors (GPCRs) screened (38) (FIGS. 15A-15E). Only the dopamine D₂ receptor (D2R) had weak reproducible activity in secondary assays, with EC₅₀ values of 4.5 μM and 16 μM in G-protein signaling and β-arrestin recruitment, respectively. ‘9087 did not measurably activate the μ-opioid receptor (OR) nor did it inhibit hERG at concentrations below 10 μM (FIGS. 15A-15E). In binding experiments to other adrenergic receptors, ‘9087 bound to the α_{2C}-subtype at mid-nM concentration and to other al-subtypes in the 1 to 10 μM range (Table 4). The molecule had no measurable binding for β-subtypes up to 10 PM. Against the imidazoline-2 receptor (I2R), a common off-target of α_{2A}AR agonists, ‘9087 bound with a Kᵢ of 300 nM, showing a modest 6-fold selectivity for the α_{2A}AR, while a few docking actives actually had higher affinities for I2R than for the α_{2A}AR (FIGS. 15A-15E).

Computational models suggested that ‘9087, ‘4622, ‘7075, and ‘2998 would all have good blood-brain barrier permeability, consistent with their small size, low topological polar surface area, and weakly basic character. Consistent with this prediction, on 10 mg/kg intraperitoneal (i.p.) injection in mice, the first three compounds, especially, had high brain and cerebral spinal fluid (CSF) exposure, indicating the compounds are likely to reach centrally acting α_{2A}ARs (Table 5). Encouragingly, ‘9087 reached high brain exposure after both intravenous (i.v.) and oral administration and had high oral bioavailability.

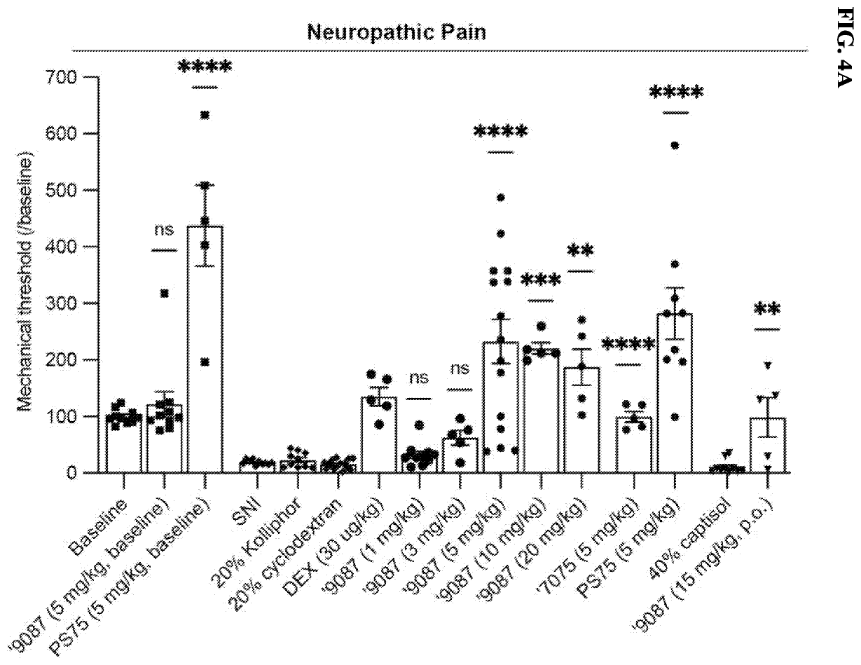

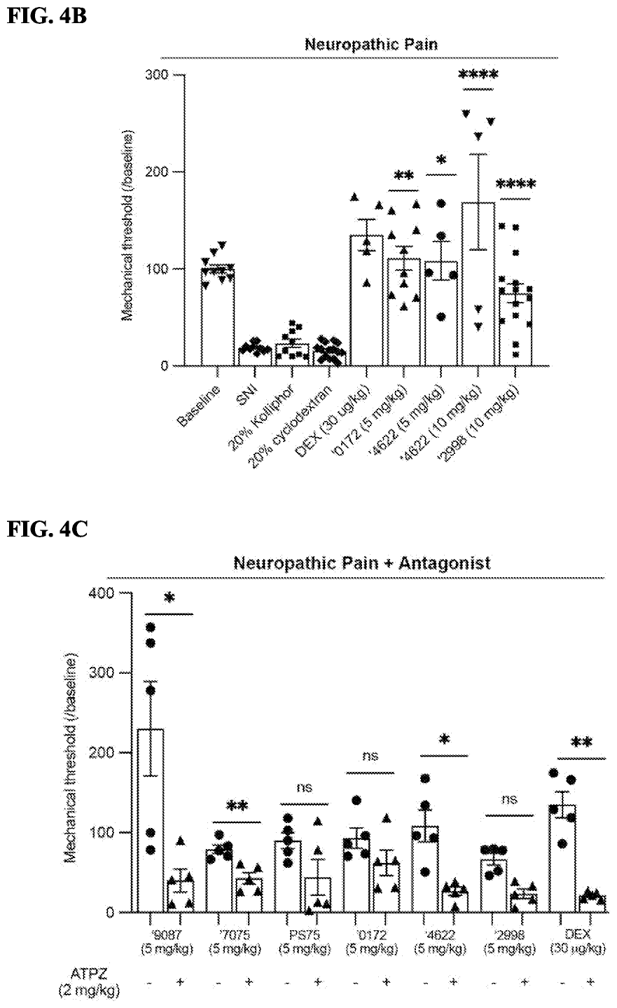

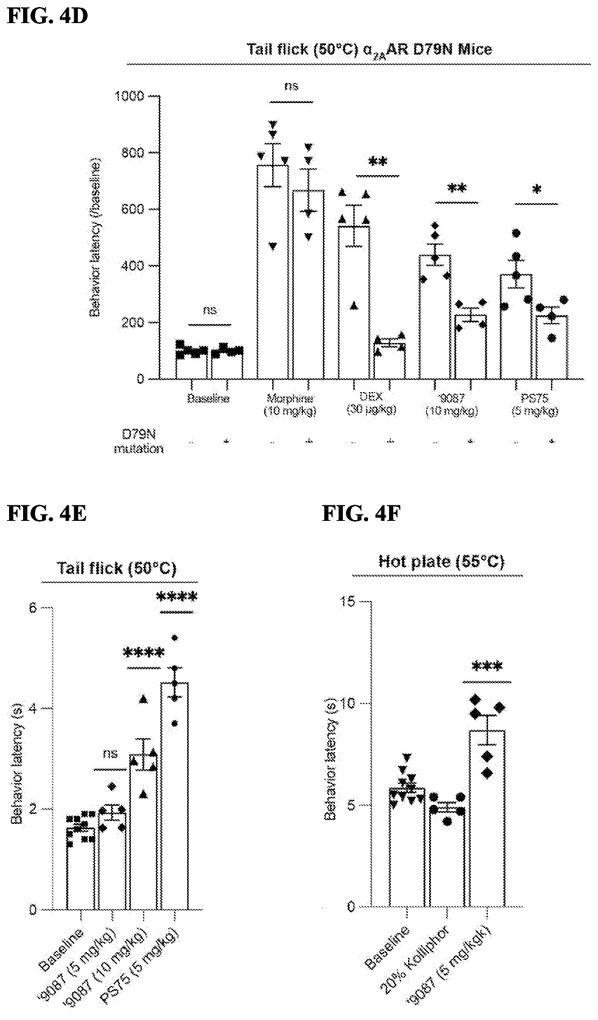

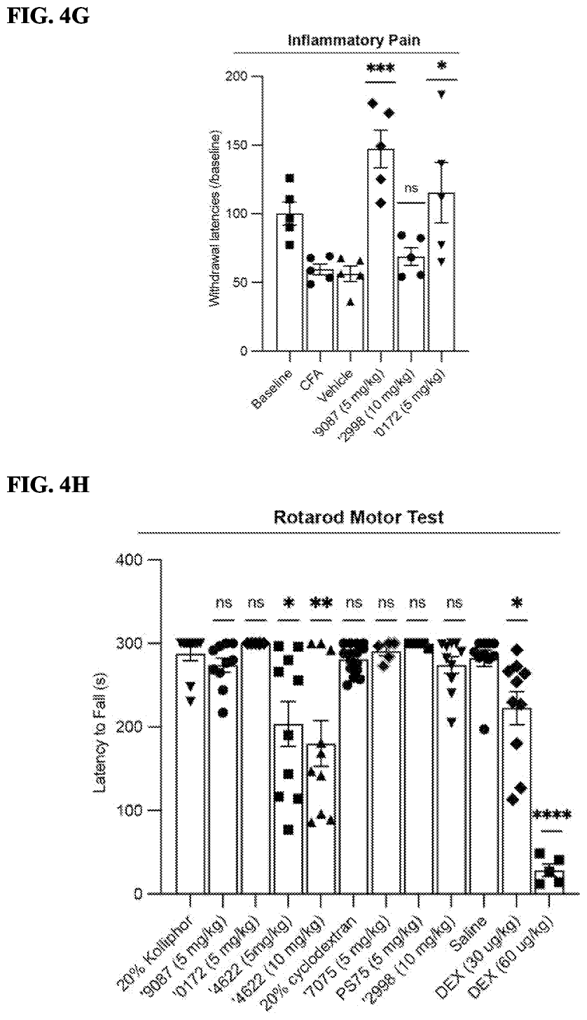

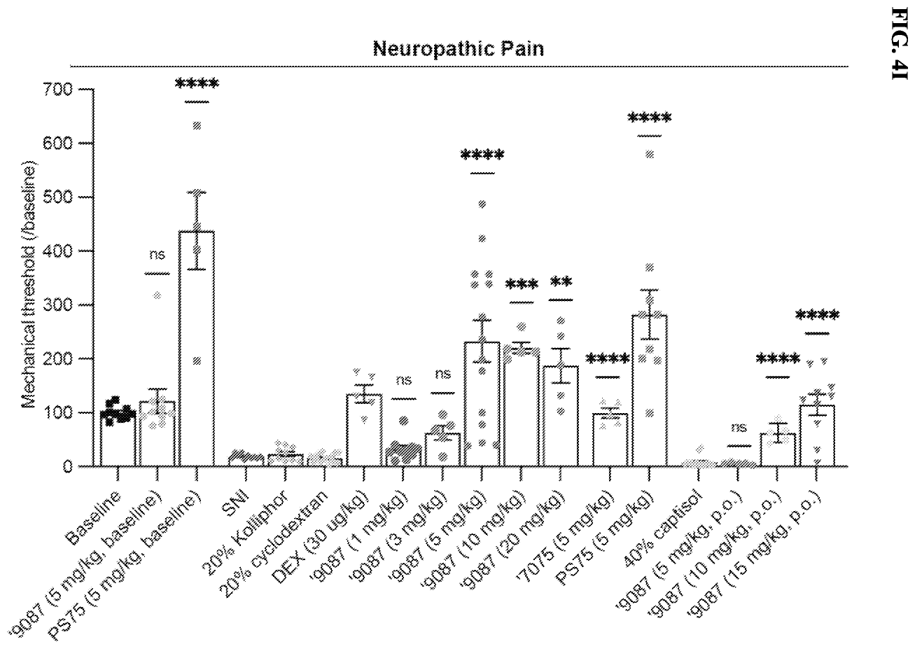

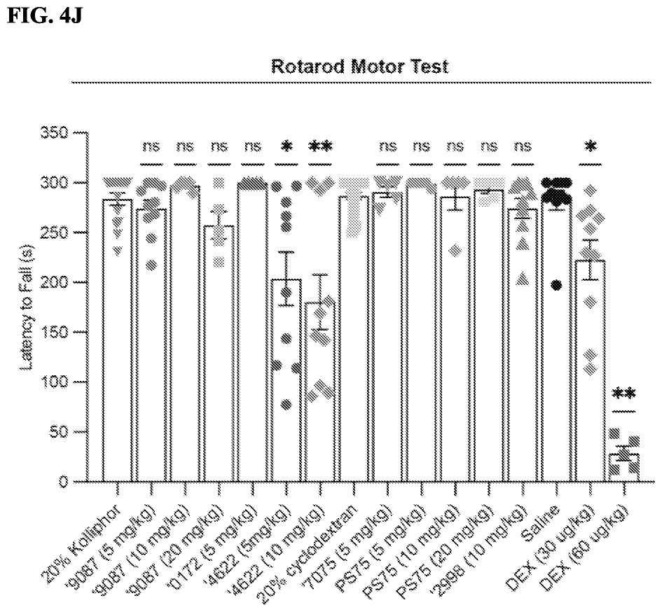

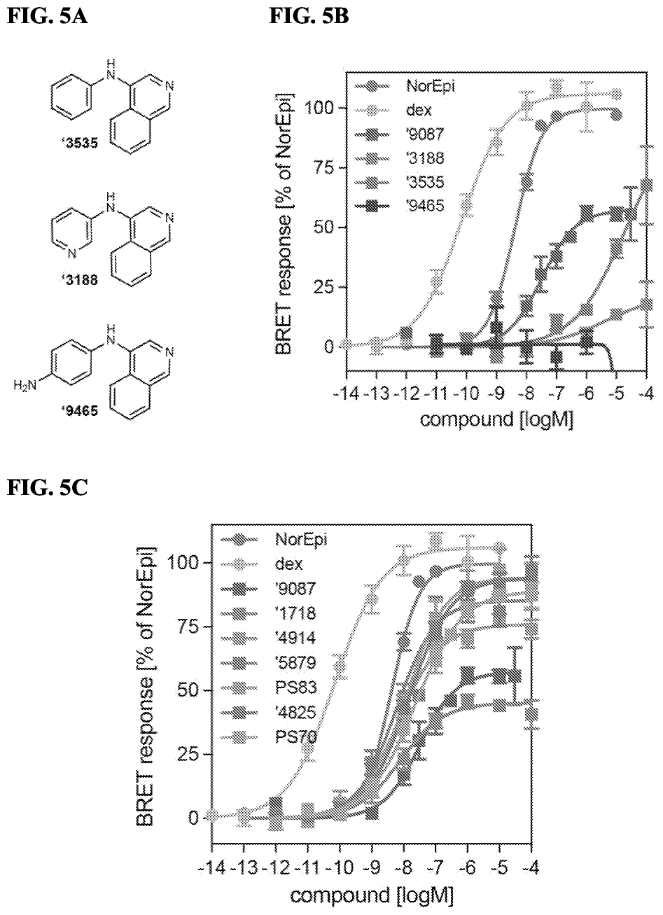

Given their selectivity and high brain exposures, we were motivated to test the more potent agonists for pain-relieving properties (FIGS. 4A-4G). We first investigated their activity in a mouse model of neuropathic pain, in which partial peripheral nerve injury invokes profound mechanical hypersensitivity (39). Systemic i.p. injections of the α_{2A}AR agonist ‘9087 dose-dependently increased the mechanical thresholds of spared nerve injured (SNI) mice, with a sharp increase in activity from 3 mg/kg to 5 mg/kg, at which points the effects plateaued (FIG. 4A). Lower doses were also anti-allodynic, returning mechanical thresholds to pre-injury levels, however, the higher doses were also analgesic, generating mechanical thresholds substantially higher than baseline, pre-injury levels. ‘9087 also increased thermal latencies in the complete Freund's adjuvant (CFA)-mediated inflammatory pain model, indicating that the molecule is effective both tissue and nerve injury-induced pain models (FIG. 4G). Furthermore, as for the analgesic action of the higher ‘9087 doses in the neuropathic pain model, ‘9087 also increased withdrawal latencies in the hot plate (55° C.) and tail flick (50° C.) assays of acute thermal (heat) pain (FIGS. 4E-4F). Of particular interest and consistent with its relatively high exposure on oral dosing, this molecule also conferred an anti-allodynic effect when delivered orally in the SNI neuropathic pain model (FIG. 4A). Crucially, while dexmedetomidine was completely sedating at 60 μg/kg, equi-analgesic doses of ‘9087 were not produce sedating in the rotarod test (FIG. 4H). This finding is an important differentiator for the new series, and also indicates that the analgesic effects of ‘9087 are not due to motor impairment.

We also investigated the mechanistic bases for the analgesia of the new α_{2A}AR agonists, both pharmacologically and genetically. Pharmacologically, the analgesic effect of ‘9087 was reversed by a systemic injection of the well-known α₂AR antagonist atipamezole (2 mg/kg; administered 15 minutes prior to ‘9087) (FIG. 4C). Because atipamezole has broad activity against the α₂AR receptor subtypes and imidazoline receptors (40), we also tested ‘9087 in mice that express an inactive form of the α_{2A}AR (point mutation D79N) (8, 41). D79N mutant mice were tested in the tail flick (50° C.) assays. As previously reported, dexmedetomidine no longer induced analgesia in the mutant mice (42), and importantly the analgesia conferred by a 10 mg/kg dose of morphine was not altered by the mutation (FIG. 4D). On the other hand, and consistent with an action via the α_{2A}AR receptor, the analgesia conferred by ‘9087 in the D79N mutant mice was significantly reduced by 70% compared to wild-type mice (FIG. 4D).

Five other novel α_{2A}AR agonists (‘2998, ‘4622, ‘0172, ‘7075, PS75) also exhibited anti-allodynic effects in the SNI mice. The ‘9087 analogs, ‘7075 and PS75, completely reversed the mechanical hypersensitivity in the neuropathic pain model. Although both analogs are 13-fold more potent for receptor activation, only PS75 was more effective than ‘9087 (FIG. 4B). This finding may reflect the different pharmacokinetic exposure levels of the two molecules, with the CSF exposure of ‘9087 being 13-fold higher than that of ‘7075 (Table 5). In contrast to ‘9087, PS75 did increase the mechanical thresholds of naïve (uninjured) mice (FIG. 4A). The anti-allodynic effects of ‘4622 and ‘7075 were reversed by atipamezole (40), however there was not significant reversal for PS75, ‘2998, and ‘0172 (FIG. 4C). PS75 also increased withdrawal latencies in the tail flick (50° C.) acute thermal pain assay, and when tested in the D79N mutant mice, its analgesic effect was reduced by 60% compared to wild-type mice (FIGS. 4D-4E). Furthermore, ‘0172 and ‘4622 exhibited anti-hyperalgesic effects in the CFA inflammatory pain model (FIG. 4G); ‘2998 did not, which may reflect the reduced brain penetration of this particular molecule (Table 5). Only ‘4622 caused slight motor impairment at its equi-analgesic dose in the rotarod test, however, the effect did not reach the full sedation observed with dexmedetomidine (FIG. 4G).

Three key observations emerge from this study. First, multiple compounds discovered directly from large-library docking are efficacious in neuropathic, inflammatory, and acute pain models via α_{2A}AR agonism (FIGS. 4A-4F). Second, functional assays reveal strong G-protein-bias of the most potent docking hits (FIG. 1C, Table 1). Biased ligands towards G-protein signaling or β-arrestin recruitment is not a novel concept for GPCR pharmacology (10, 16, 17, 43-46) or for docking screens of ultra-large chemical libraries (16, 44), but was unexpected to this degree. Third, unlike dexmedetomidine and clonidine (47, 48), ‘9087 does not cause sedation or motor impairment at analgesic doses, potentially enabling broader applications to pain treatment and attesting to the ability to differentiate these two effects with α_{2A}AR agonists (FIG. 4H).

How exactly the new chemotypes lead to new signaling and new physiology is uncertain. As in earlier studies (10, 13, 16, 44, 49), the new ligands engage many of the same receptor residues as do canonical ligands, in this case the agonists norepinephrine and dexmedetomidine (9, 33). Still, the new agonists did make non-canonical interations. For ‘9087 these included apparently weaker interactions with the key polar residues D128^{3.32} and Y431^{7.43}, and apparently stronger interactions with F427^{7.39} (FIG. 1B, FIG. 1D, FIG. 2B). Such differential engagement may contribute to the partial and G protein biased agonism of the new ligands versus norepinephrine and dexmedetomidine, which in turn may play a role in their lack of sedation. As both bias and partial agonism derive from engagement of transducing proteins 35 Å away from the orthosteric site, other mechanisms may be considered. Moreover, their physiological impact will be entangled with the pharmacokinetics of the molecules. What should be clear is that the analgesic potential of α_{2A}AR agonists may be disentangled from their sedative effect, something important for subsequent drug development and flowing from the exploration of new chemotypes.

‘9087 and its analogs are not as potent as dexmedetomidine, something reflected in the lower dose of 30 μg/kg of the latter needed for analgesia (FIG. 4A). The action of ‘9087 in vivo is blocked by the α_{2A}AR antagonist atipamezole (40, 50), and much reduced in D79N α_{2A}AR mice indicating the α_{2A}AR is the primary receptor mediating activity in vivo (FIGS. 4C-4D). Especially for the mutant mice, we do note that while most efficacy above baseline was reduced, it was not fully reversed, and other targets may also play a role including the I2R and other α₂AR-subtypes (FIGS. 4C-4D, FIGS. 7A-7L, FIGS. 15A-15E, Table 4).

From an ultra-large library docking screen emerged low nM α_{2A}AR agonists, topologically unrelated to previously known ligands, making new interactions with the receptor that appear to confer new pharmacology (FIGS. 1A-1C, Table 1). Several of the new agonists, were anti-allodynic and analgesic in neuropathic and inflammatory pain models, and against nociception in naïve animals (FIGS. 4A-4G). ‘9087 and PS75 are promising, both of which are strongly analgesic without the sedative effects of dexmedetomidine, while ‘9087 is also orally bioavailable (FIG. 4A, FIG. 4H). These properties make these compounds plausible therapeutic leads for new pain therapeutics without the liabilities of classic α_{2A}AR receptor drugs.

### Example 2: Experimental Methods and Characterization Data

Molecular docking. The α_{2B}AR receptor with dexmedetomidine and Goα (PDB 6K41) (9) was used for docking calculations. Three screens of the ZINC15 database (11) were run, two for fragment molecules (less than 250 amu, c Log P≤3.5) and one for lead-like (250-350 amu, c Log P≤3.5). Docking was performed with DOCK3.7 (22). For the first screen, 45 matching spheres (22) were used, 15 from the docked-pose of dexmedetomidine and 30 from SPHGEN-generated spheres (51). The receptor structure was protonated using REDUCE (52) and AMBER united atom charges were assigned (53). Control calculations (54) using 15 known agonists generated from IUPHAR (30) and the literature (24-26) and 1800 property matched decoys (55) were used to optimize docking parameters based on logAUC (54) and on ligand interactions with residues D128^{3.32}, F427^{7.39}, F405^{6.51}, Y409^{6.55}, and F406^{6.52} of the receptor. An “extrema” set was used to evaluate cationic charge preference, as described (18, 55). The protein low dielectric and desolvation regions were extended as previously described (56), based on control calculations, by a radius of 1 Å and 0.3 Å, respectively. SPHGEN (51) was used to generate pseudo-atoms to define the extended low protein dielectric and desolvation region (10, 57). Energy potential grids were calculated using CHEMGRID (58) for AMBER-based van der Waals potential, QNIFFT (59) for Poisson-Boltzmann-based electrostatic potentials, and SOLVMAP (60) for context-dependent ligand desolvation. In the second and third screens, differences included modified matching spheres (added rigid fragments of xylazine docked-pose, only used 40 matching spheres) and extension of the desolvation by a radius of 0.2 Å.

For the first screen, 20 million molecules from the ZINC15 (http://zinc15.docking.org/) fragments subset were docked in 3,008 core hours or about 6 wall-clock hours on a 500-core cluster. Almost 5 trillion complexes were sampled, on average each molecule sampled 2,405 orientations and 202 conformations. Only about 8 of 20 million could be sterically accommodated in the orthosteric site, reflecting its small size. For the second screen, the same 20 million fragments were docked in 3830 core hours or 7.7 hours on 500-core cluster, sampling over 6 trillion complexes; on average each molecule sampled 3,122 orientations and 203 conformations. About 9 million molecules were accommodated in the site. For the third screen, 281 million molecules from ZINC15 lead-like subset were screened in 71,625 core hours or about 1 week on 500 cores. Over 222 trillion complexes were sampled with an average of 4,553 orientations and 469 conformations per molecule, though ultimately only 13.5 million could sterically fit in the site.

For the first and second screens, the top 161,055 scored compounds were clustered by ECFP4-based Tanimoto coefficient (Tc) of 0.5 to identify unique chemotypes, resulting in 37,150 and 33,378 clusters. For the third screen, the top 300,000 scored compounds were clustered in the same manner resulting in 57,168 clusters. Molecules were filtered for novelty, removing those with Tc>0.35 to 15 α_{2A}AR agonists used in control calculations. The top 5,000 ranked molecules remaining were visually filtered for interactions at residues D128^{3.32}, F427^{7.39}, F405^{6.51}, Y409^{6.55}, and F406^{6.52} for the first and second screens; for the third screen, the top 20,000 molecules were examined by the same criteria. Lastly, prioritized molecules were also filtered for internal torsional strain; this was done visually for the first screen, while the second and third screens used a method drawing on CSD torsion populations cutting off at a total energy of 2 Torsion Energy Units (27). An additional novelty filter was performed removing molecules with Tc>0.35 to CHEMBL29 (23) α_{2A}AR compounds. Sixty-four molecules were selected for purchasing: 33, 26, and 5 from the first, second and third screens, respectively. Nine were sourced from WuXi and another 54 from Enamine, of which 7 and 37 were successfully synthesized, respectively. Most of these compounds have not previously been synthesized before, to the best of our knowledge, except for some of the smaller fragments, which have been previously used as building blocks.

Make-on-demand synthesis. Forty-eight molecules prioritized for purchasing were synthesized by Enamine and Wuxi for a total fulfilment rate of 75%. Compounds were sourced from the WuXi GalaXi Virtual library or Enamine REAL database (https://enamine.net/compound-collections/real-compounds). The purities of active molecules synthesized by Enamine and WuXi were at least 90% and typically above 95%. The purity of compounds tested in vivo were >95% and typically above 98% (see Appendix I).

Ligand optimization. Analogs for four docking hits (‘9087, ‘2998, ‘0172, ‘4622) were queried in Arthor and SmallWorld 1.4 and 12 Billion make-on-demand libraries (https:/sw.docking.org/, https://arthor.docking.org), the latter primarily containing Enamine REAL Space compounds (https://enamine.net/compound-collections/real-compounds/real-space-navigator). Results from SmallWorld, Bemis-Murcko framework, and substructure queries were pooled, docked into the α_{2B}AR site (prior to ‘9087-α_{2A}AR structure being determined). Compounds with favorable interactions in the orthosteric site were prioritized, leading to 13 analogs for ‘9087. Also, for the four docking hits, analogs were designed by modifying the 2D chemical structure to test specific hypotheses, adding another six analogs for ‘9087. The second round of analogs for ‘9087 were designed and prioritized for bespoke synthesis. Some were docked to the ‘9087-α_{2A}AR structure, while several were designed and synthesized regardless of docked pose to test specific hypotheses; in total 15 of these were synthesized and tested. Calculation of the contact areas was performed by means of UCSF Chimera (61).

Molecular modeling of ‘7075 and PS75. Maestro (v. 2019-4, Schradinger, LLC) was used to manually change the chemical structure of ‘9087 to ‘7075 or PS75 in the ‘9087-α_{2A}AR complex cryo-EM structure. The isoquinoline nitrogen was changed to a carbon and the fluorine or chlorine substituent was added to the naphthalene ring for ‘7075 and PS75, respectively. The resulting complex of ‘7075 or PS75 and α_{2A}AR coupled to the G-protein but without scFv16 was energetically minimized following the Protein Preparation Wizard protocol using the OPLS3e force field (62). The maximum heavy-atom deviation from the initial model was 0.3 Å.

Passive-membrane permeability prediction. Ligand structures were converted from SMILES strings to three-dimensional structures using LigPrep (v. 53013, Schrodinger, New York). For the passive-membrane permeability prediction (63, 64), we retained only neutral form for each ligand. Passive-membrane permeability of a ligand is predicted from the free-energy of insertion (ΔG_{I}), i.e., from the energy difference between a conformer in low and high dielectric media. Therefore, we generated conformations of each ligand using ConfGen software (v. 5.1, Schrodinger, New York). We then minimized each conformer in a low dielectric medium (chloroform) to mimic the membrane dielectric using Protein Local Optimization Program (PLOP) (65). After finding the lowest energy conformer in the low dielectric medium, we calculated the energy of that energy-minimized conformer in water.

We subtracted the energy of the ligand in the high-dielectric water from the low-dielectric medium. We further added a deionization penalty term to account for transforming ionized form of the ligand in water to its neutral form in membrane. We computed the deionization penalty energy using the empirical pKa prediction software Epik (v. 5.1013, Schrodinger Inc.). We then rank-ordered the ligands based on their free-energy of insertion.

Radioligand binding experiments. Receptor binding affinities for the α_{2A}AR receptor and to α_{2B}AR as well as the related adrenergic subtypes α_{1A}, α_{1B}, α_{2C}, β₁ and β₂ were determined according to methods as described previously (66, 67). In brief, membranes were prepared from HEK293T cells transiently transfected with the cDNA for human α_{2A}AR, murine α_{2A}AR (provided by D. Calebiro, Birmingham, UK), human α_{2B}AR (obtained from the cDNA resource center, www.cdna.org) or with the cDNAs for the human α_{1A}, α_{1B}, α_{2C}, β₂ (all from cDNA resource center) and β₁ (provided by R. Sunahara, UCSD). Receptor densities (Bₘₐₓ value) and specific binding affinities (K_{D} value) for the radioligand [³H]RX82,1002 (specific activity 52 Ci/mmol, Novandi, Södertälje, Sweden) were determined as 1,400±210 fmol/mg protein and 0.54±0.024 nM for human α_{2A}AR, 4,000±720 fmol/mg protein and 1.8±0.61 nM for murine α_{2A}AR, and 3,400±580 fmol/mg protein and 2.3±0.52 nM for α_{2B}AR, respectively. Further values are 3,200±1,900 fmol/mg protein and 0.58±0.11 nM for α_{2C}, 2,000±950 fmol/mg protein and 0.70±0.13 nM for α_{1A}, and 4,000 fmol/mg protein and 0.11 nM for α_{1B}, both determined with [³H]prazosin (51 Ci/mmol, PerkinElmer, Rodgau, Germany), respectively and 1,400±360 fmol/mg protein and 0.070±0.006 nM for β₁, and 1,300±230 fmol/mg protein and 0.074±0.012 nM for β₂, both determined with [³H]CGP12,188 (52 Ci/mmol, PerkinElmer).

Competition binding experiments with α₂AR subtypes were performed by incubating membranes in buffer A (50 mM TRIS at pH 7.4) at final protein concentrations of 3-10 μg/well with the radioligand (final concentration 0.5-2.0 nM according to the appropriate K_{D} and Bₘₐₓ) and varying concentrations of the competing ligands for 60 minutes at 37° C. Binding to α_{1A} and α_{1B} was done with buffer B (50 mM TRIS, 5 mM MgCl₂, 1 mM EDTA, 100 μg/mL bacitracin and 5 μg/mL soybean trypsin inhibitor at pH 7.4) at 2-6 μg/well (radioligand at 0.2-0.3 nM) and to β₁ and β₂ with buffer C (25 mM HEPES, 5 mM MgCl₂, 1 mM EDTA, and 0.006% bovine serum albumin at pH 7.4) at 4-8 μg/well (radioligand 0.2 nM). Non-specific binding was determined in the presence of unlabeled ligand at 10 PM. Protein concentration was measured using the method of Lowry (68).

The resulting displacement curves were analyzed by nonlinear regression using the algorithms implemented in PRISM 8.0 (GraphPad Software, San Diego, CA) to provide IC₅₀ values, which were subsequently transformed into a Kᵢ values applying the equation of Cheng and Prusoff (69). Mean Kᵢ values (±s.d.) were derived from 2-7 experiments each performed in triplicates.

Functional assays. The human wild type α_{2A}AR, its respective receptor mutants (70) and the murine α_{2A}AR, all carrying an N-terminal HA-signal sequence and a FLAG-tag, as well as the human adrenergic receptor subtypes α_{1A}, α_{1B}, α_{2C}, β₁ and β₂ and the dopamine receptor D_{2long} were cloned to pCDNA3.1 for G protein activation assays. Human α_{2A}AR and α_{2B}AR were fused to the ARMS2-PK2 sequence and cloned to pCMV (DiscoverX, Eurofins) for β-arrestin-2 recruitment assays, respectively, using polymerase chain reaction and Gibson Assembly (New England Biolabs) (67). Sequence integrity was verified by DNA sequencing (Eurofins Genomics).

Bioluminescense resonance energy transfer. G protein mediated activation of human α_{2A}AR and D_{2long} was monitored with Gαᵢ₁-RLucII (71, 72) together with Gβ₁ and Gγ₁-GFP₁₀. Arrestin recruitment was performed by enhanced bystander BRET using CAAX-rGFP and β-arrestin-2-RLucII as biosensors (all biosensors are provided from M. Bouvier, Université de Montréal, Canada) (71, 73) in the presence of GRK2 as described (66, 74). In brief, HEK293T cells were transfected with a total amount of 3.3 μg DNA per 10⁶ cells using linear polyethyleneimine (PEI, Polysciences, 3:1 PEI:DNA ratio) and transferred into 96-well half-area plates (Greiner, Frickenhausen, Germany) at a density of 10,000 cells per well and incubated for 48 hrs. Medium was exchanged with PBS (Hank's balanced salt solution) and cells were stimulated with ligands at 37° C. for 10 min for Gαᵢ stimulation or arrestin recruitment, respectively. Coelenterazine 400a at a final concentration of 2.5 PM) was added 5 min before measurement. BRET was monitored on a Clariostar plate reader (BMG, Ortenberg, Germany) with the appropriate filter sets (donor 410±80 nm, acceptor 515±30 nm) and was calculated as the ratio of acceptor emission to donor emission. BRET ratio was normalized to the effect of buffer (0%) and the maximum effect of norepinephrine (100%) for adrenergic receptors and quinpirole (100%) for D_{2long}. For each compound 3 to 17 individual experiments were performed each done in duplicates.

Investigating the sensitivity of selected ligands to the receptor mutants α_{2A}AR-D128^{3.32}A, α_{2A}AR-D128^{3.32}T, α_{2A}AR-D128^{3.32}L, α_{2A}AR-S215^{5.42}A, α_{2A}AR-Y409^{6.55}A, α_{2A}AR-Y409^{6.55}T, α_{2A}AR-Y409^{6.55}F, α_{2A}AR-F427^{7.39}F, α_{2A}AR-Y431^{7.43}A, and α_{2A}AR-Y431^{7.43}F was determined by monitored G protein stimulation as described above transfecting the appropriate receptor together with Gαᵢ₁-RLucII and Gβ₁/Gγ₁-GFP₁₀. Normalization was done according to the effect of buffer (0%) and norepinephrine (100%) with the exception of α_{2A}AR-D128^{3.32}A (dexmedetomidine=100%), α_{2A}AR-D128^{3.32}T and α_{2A}AR-D128^{3.32}L (‘7075=100%). Three to ten experiments were done in duplicate.

IP accumulation assay. Determination of G protein mediated signaling by human α_{2A}AR, murine α_{2A}AR, and human α_{2B}AR was performed applying an IP accumulation assay (IP-One HTRF®, Cisbio, Codolet, France) according to the manufacturer's protocol and in analogy to previously described protocols (75, 76). In brief, HEK 293T cells were co-transfected with the cDNA for a receptor and the hybrid G-protein Gα_{qi} (Gα_{q} protein with the last five amino acids at the C-terminus replaced by the corresponding sequence of Gαᵢ(gift from The J. David Gladstone Institutes, San Francisco, CA), respectively in a ratio of 1:2. After one day cells were transferred into 384 well micro plates (Greiner) and incubated for further 24 hrs. On the day of the experiment cells were incubated with test compounds for 90 min ((α_{2A}AR) or 120 min ((α_{2B}AR) and accumulation of second messenger was stopped by adding detection reagents (IP1-d2 conjugate and Anti-IP1cryptate TB conjugate). After 60 min TR-FRET was monitored with a Clariostar plate reader. FRET-signals were normalized to buffer (0%) and the maximum effect of norepinephrine (100%). Three to nine (murine α_{2A}AR, α_{2B}AR) or 4-11 repeats (human α_{2A}AR), respectively in duplicate were performed for each test compound all done in duplicate.

Arrestin recruitment assay. Investigation of α_{2A}AR and α_{2B}AR stimulated (3-arrestin-2 recruitment was performed applying an assay which is based on fragment complementation of β-galactosidase (PathHunter assay, DiscoverX, Birmingham, U.K.) as described (77). In detail, HEK293T cells stably expressing the enzyme acceptor (EA) tagged β-arrestin-2 were co-transfected with human α_{2A}AR or α_{2B}AR each fused to the ProLink-ARMS2-PKS2 fragment for enzyme complementation and GRK2 (cDNA Resource Center) at equal amounts and subsequently transferred into 384 well micro plates(Greiner) after 1 day. After incubation for further 24 hrs cells were incubated with test compounds for 60 min ((α_{2A}AR) or 90 min (α_{2B}AR), arrestin recruitment was stopped by adding detection regent and the resulting chemoluminescence was monitored with a Clariostar plate microreader. Data was normalized relative to buffer (0%) and the maximum effect of norepinephrine (100%). Three to nine repeats for α_{2A}AR (3-6 for (α_{2B}AR) in duplicate were measured.

DiscoverX HitHunter cAMP G-protein activation assay. Dexmedetomidine, brimonidine, ‘9087, and ‘7075 were tested by DiscoverX (Eurofins; CA, USA) in their HitHunter XS+ assay. Freezer stock cAMP Hunter cell lines were expanded, then seeded in a total volume of 20 uL into white walled, 384-well microplates and incubated at 37° C. prior to testing. For agonist determination, cells were incubated with compound samples in the presence of EC80 forskolin to induce response. Media was aspirated from cells and replaced with 15 uL 2:1 HBSS/10 mM Hepes: cAMP XS+Ab reagent. Intermediate dilution of sample stocks was performed to generate 4× sample in assay buffer containing 4× EC80 forskolin. 5 μL of 4× sample was added to cells and incubated at 37° C. or room temperature for 30 to 60 minutes. Finally assay vehicle concentration was 1%. After sample incubation assay signal was generated through incubation with 20 uL cAMP XS+ED/CL lysis cocktail for one hour followed by incubation with 20 uL cAMP XS+EA reagent for three hours at room temperature. Microplates were read following signal generation with a PerkinElmer Envision Instrument for chemiluminescent signal detection. Compound activity was analyzed using CBIS data analysis suite. For Gᵢ agonist mode, percentage activity is calculated using the following formula: % Activity=100%×(1−(mean RLU of test sample−mean RLU of MAX control)/(mean RLU of vehicle control−mean RLU of MAX control)). Brimonidine was used as the control agonist. Each measurement was done in duplicate.

Preparation of the ‘9087-α_{2A}AR-Goα complex. The human wild type α_{2A}AR was cloned to pFastBac vector with a N-terminal FLAG tag and a C-terminal histidine Tag. This construct was expressed in Sf9 insect cells using the pfastBac baculovirus system (Expression Systems). Cells were infected at a density of 4×10⁶ cells per mL and then incubated for 48 hours at 27° C. Receptor was extracted and purified following the protocol described previously for α_{2B}AR (9). Briefly, receptor was purified by Ni-NTA chromatography, Flag affinity chromatography and size exclusion chromatography in the presence of 100 μM ‘9087. The monomeric peak fractions of receptor were collected and concentrated to −20 mg/mL. The fresh purified ‘9087-bound α_{2A}AR was used for complex formation without frozen. Goα heterotrimer were expressed and purified as previously described with minor modifications (76). Briefly, Hi5 cells were grown to a density of 3 million per m1 and then infected with Goα and Goβ1γ2 baculovirus at a ratio of 10-20 mL/L and 1-2 ml/L respectively and then incubated for 48 hours at 27° C. Cells were solubilized with 1% (w/v) sodium cholate and 0.05% (w/v) DDM. After centrifugation, the supernatant was loaded onto Ni-NTA column and then exchanged to 0.05% DDM. The eluted Goα heterotrimer was dephosphorylated by lambda phosphatase (homemade) and further purified through ion exchange using a Mono Q 10/100 GL column (GE Healthcare) and the peak fractions were collected and flash frozen in liquid nitrogen until use. The scFv16 (78) protein was expressed in insect Sf9 cells and purified with Ni-NTA column followed by the Superdex 200 Increase 10/300GL column (GE Healthcare) with a buffer composed of 20 mM HEPEs, pH 7.5 and 100 mM NaCl. The monomeric peak fractions of receptor were collected and concentrated and stored at −80° C. until use. The complex formation process is same as described (35). Briefly, the complex of α_{2A}AR with heterotrimeric Goα was formed in a buffer containing 20 mM HEPEs pH 7.5, 100 mM NaCl, 0.1% DDM, 1 mM MgCl₂, 10 μM GDP and 100 μM ‘9087. The α_{2A}AR-Goα complex was then treated with 50 units of apyrase (NEB) on ice overnight, and exchanged on an anti-Flag M1 column into a buffer containing 20 mM HEPES, pH 7.5, 100 mM NaCl, 0.0075% lauryl maltose neopentyl glycol (MNG, NG310 Anatrace), 0.0025% GDN (GDN101, Anatrace), and 0.001% CHS, 100 μM ‘9087 and 2 mM CaCl₂) in a stepwise manner. After elution by adding 5 mM EDTA and 0.2 mg/mL Flag peptide, the complex was concentrated and incubated with excess scFv16 for 1 hour on ice, then further purified using Superdex 200 Increase 10/300GL column (GE Healthcare) with a running buffer of 20 mM HEPES, pH 7.5, 100 mM NaCl, 0.00075% MNG, 0.00025% GDN and 0.0001% CHS, 100 μM ‘9087. The monomeric peak fraction of α_{2A}AR-Goα complex was collected and concentrated to −5 mg/mL for cryo-EM.

Cryo-EM data collection, processing and model building. 3 μL purified complex sample was applied onto the grid (CryoMatrix nickel titanium alloy film, R1.2/1.3, Zhenjiang Lehua Electronic Technology Co., Ltd.) (79) glow discharged at Tergeo-EM plasma cleaner and then blotted for 3 see with blotting force of 0 and quickly plunged into liquid ethane cooled by liquid nitrogen using Vitrobot Mark IV (Thermo Fisher Scientific, USA) at 10° C. and with 95% humidity. Cryo-EM data was collected on a 300 kV Titan Krios Gᵢ3 microscope. The raw movies were recorded by Gatan K3 BioQuantum Camera at the magnification of 105,000 and the corresponding pixel size is 0.85 Å. Inelastically scattered electrons were excluded by a GIF Quantum energy filter (Gatan, USA) using a slit width of 20 eV. The movie stacks were acquired with the defocus range of −1.0 to −1.6 micron with total exposure time 2.5 s fragmented into 50 frames (0.05 s/frame) with the dose rate of 22.0 e/pixel/s. The imaging mode is super resolution with 2-time hardware binning. The semi-automatic data acquisition was performed using SerialEM (80). Raw movie frames were aligned with MotionCor2 (81) using a 9×7 patch and the contrast transfer function (CTF) parameters were estimated using Gctf and ctf in JSPR (82). Micrographs with consistent CTF values including defocus and astigmatism parameter were kept for the following image processing, which kept 3768 micrographs from 4217 raw movies. Templates for particle auto-picking were generated by projecting the 3D volume of norepinephrine-bound α_{2A}AR-Goα complex (33). The 2,137,146 particles picked from template picking was subjected 2D classification in cryoSPARC (83) and 3D-classication in Relion (84). The sorted 321,762 particles were then subjected to Homogeneous reconstruction in cryoSPARC, yielding a 3.57 Å map. Further 3D Ab-initio reconstruction reduced the particles number to 287,431, which was subjected to CTF refinement and non-uniform refinement after extracting with larger particle box size, and finally yield the 3.47 Å map. The norepinephrine-α_{2A}AR-GoA complex structure (PDB 7EJ0) (33) was used as the initial template for model building. The model was docked into the cryo-EM density map using Chimera (61), followed by iterative manual building in Coot (85) and real space refinement in Phenix (86). The statistics of the final model was validated by Molprobity (87). The ligand symmetry accounted RMSD between the docked pose and cryo-EM pose of ‘9087 was calculated by the Hungarian algorithm in DOCK6 (88).

pKa determination for ‘9087. The pKa of ‘9087 (2.90 mg, 0.013 mmol) was determined by potentiometric titration using a Metrohm pH Meter 632 equipped with a glass electrode (Metrohm 6.0259.100). The compound was dissolved in 15 mL of 10% methanol aqueous solution, at an ionic strength of I=0.15 M using KCl. The resulting solution was stirred throughout the experiment using a magnetic stir bar and a magnetic agitator. The compound was titrated with 0.01 M HCl (Titrisol®) using an automatic burette (Metrohm Dosimat Plus 876). The titrant was added to the analyte stepwise (0.024-2.87 mL). The resulting graph for pKa-determination is presented in dependence of τ and pH(τ). The pKa value was then determined using a simplified Henderson-Hasselbalch equation. The data from the titration experiment was evaluated with Origin 9.60.

### Off-Target Activity

GPCRome. 10 μM ‘9087 was tested for off-target activity at a panel of 320 non-olfactory GPCRs using PRESTO-Tango GPCRome arresin-recruitment assay as described (38). Receptors with at least three-fold increased relative luminescence over corresponding basal activity are potential positive hits. Screening was performed by the National Institutes of Mental Health Psychoactive Drug Screen Program (PDSP) (89). Detailed experimental protocols are available on the NIMH PDSP website at https://pdsp.unc.edu/pdspweb/content/PDSP %20Protocols %20II %202013-03-28.pdf.

D2R Activation. D2R was selected following the GPCRome panel and ‘9087 was re-tested for full dose-response to determine G-protein and arrestin recruitment.

I2R Binding. Top docking compounds (‘9087, ‘2998, ‘4622, ‘0172) were tested for I2R binding, performed by Eurofins Cerep (France; catalog #81) as described (78). For compound ‘2998, no binding was seen in a single point radioligand displacement experiment tested at 500 nM and the compound is not shown.

μOR competition binding. Equilibrium [3H] Diprenorphine competition and saturation binding were carried out in membranes prepared from Chinese Hamster Ovary (CHO-K1) cells stably expressing human μ-Opioid receptor, as previously described (90-92). Briefly, binding was performed at 25° C. for 90 min in the dark. Binding in μOR/CHO-K1 cells was carried out in a buffer consisting of 50 mM HEPES-base pH 7.4 (pH adjusted with KOH), 10 mM MgCl₂, 0.1 mM EDTA, and 0.1% (w/v) Bovine Serum Albumin with membranes containing approximately 40 μg/mL protein. Following incubation with radioligand (1 μM to 10 nM for saturation, 500 μM for competition), drugs (33 μM to 3.3 μM) and/or 20 μM cold competitor naloxone, the reaction was rapidly filtered onto GF/B (PerkinElmer #1450-521) glass fiber filtermats which were equilibrated for 1 hour in binding buffer supplemented with 0.3% (v/v) polyethyleneimine. The filtermats were washed 5 times in ice-cold 50 mM HEPES-base pH 7.4 using a Perkin Elmer semi-automated cell harvester (Perkin Elmer FilterMate Harvester). The filtermats were dried and Meltilex solid scintillant (Perkin Elmer #1450-442) was melted onto the mats for 10 min at 60° C. The scintillant was allowed to re-solidify before disintegrations were quantified with a Wallac MicroBeta Scintillation counter using an integration time of 1 min. Non-specific binding, total binding, the number of receptor binding sites, and the K_{d} of the radiotracer were determined from saturation binding experiments. Protein concentrations were determined using the microBCA method with BSA as the standard. Kᵢ values were calculated by non-linear regression analysis and application of the Cheng-Prusoff correction in GraphPad Prism 9.0.

hERG inhibition assays. ‘9087 was tested for hERG inhibition in the FluxOR assay as described (93). hERG experiments used the National Institutes of Mental Health Psychoactive Drug Screening Program.

### In Vivo Methods

Animals. Animal experiments were approved by the UCSF Institutional Animal Care and Use Committee and were conducted in accordance with the NIH Guide for the Care and Use of Laboratory animals. Adult (8-10 weeks old) male C56BL/6 mice (strain #664) were purchased from the Jackson Laboratory. Mice were housed in cages on a standard 12:12 hour light/dark cycle with food and water ad libitum. The α_{2A}AR D79N mutant mice were purchased from Jackson (stock #002777), and 7-8 week old females were used.

Compound preparation. All ligands were synthesized by Enamine (‘2998, ‘7075) or WuXi (‘9087, ‘4622, ‘0172) or in house (PS75) and dissolved 30 minutes prior to testing. ‘9087, ‘4622 and ‘0172 were resuspended in 20% Kolliphor. ‘2998, ‘7075, and PS75 were resuspended in 20% cyclodextran. Atipamezole (Cayman Chemical Company; cat. #9001181) and Dexmedetomidine (Cayman Chemical Company; cat. #15581) were resuspended with NaCl 0.9%.

Behavioral analyses. For all behavioral tests, the experimenter was always blind to treatment. Animals were first habituated for 1 hour in Plexiglas cylinders and then tested 30 minutes after subcutaneous injection of the α_{2A}AR compounds. The α_{2A}AR antagonist atipamezole (2 mg/kg) was intraperitoneally injected 15 minutes prior to subcutaneous injection of the α_{2A}AR agonists. The mechanical (Von Frey), thermal (Hargreaves, hotplate and tail flick) and ambulatory (rotarod) tests were conducted as described previously (94). Hindpaw mechanical thresholds were determined with von Frey filaments using the updown method (95). Hindpaw thermal sensitivity was measured with a radiant heat source (Hargreaves) or a 55° C. hotplate. For the tail flick assay, sensitivity was measured by immersing the tail into a 50° C. water bath for both WT and D79N mutant mice. For the ambulatory (rotarod) test, mice were first trained on an accelerating rotating rod, 3 times for 5 min, before testing with any compound.

Spared-nerve injury (SNI) model of neuropathic pain. Under isoflurane anesthesia, two of the three branches of the sciatic nerve were ligated and transected distally, leaving the sural nerve intact. Behavior was tested 7 to 14 days after injury and in situ hybridization was performed one week post-injury.

Complete Freund's Adjuvant (CFA). The CFA model of chronic inflammation was induced as described previously (96). Briefly, CFA (Sigma) was diluted 1:1 with saline and vortexed for 30 minutes. When fully suspended, we injected 20 μL of CFA into one hindpaw. Heat thresholds were measured before the injection (baseline) and 3 days after using the Hargreaves test.

Pharmacokinetics. Pharmacokinetic experiments were performed by Bienta (Enamine Biology Services) in accordance with Enamine pharmacokinetic study protocols and Institutional Animal Care and Use Guidelines (protocol number 1-2/2020). Plasma pharmacokinetics and brain distribution for ‘9087, ‘2998, ‘4622, ‘7075, PS75, and CSF distribution for ‘7075, PS75, ‘9087, and ‘4622, were measured following a 10 mg/kg (i.p.) dose. Plasma and brain samples were also collected for ‘9087 following 10 mg/kg i.v. and 30 mg/kg p.o. (oral) dose to determine oral bioavailability. In each compound study, nine time points (5, 15, 30, 60, 120, 240, 360, 480 and 1440 min) were collected, each of the time point treatment group included 3 animals. There was also a control group of one animal. In the ‘9087, ‘7075, ‘4622 studies, male C57BL/6N mice were used, for PS75 CD-1 mice, and for ‘2998 male Balb/cAnN mice. For all compound studies the animals were randomly assigned to the treatment groups before the pharmacokinetic study; all animals were fasted for 4 h before dosing. For injections, ‘9087 was dissolved in Captisol—water (40%:60%, w/v), ‘4622 was dissolved in a 20% Kolliphor HS—physiological saline solution, and ‘7075, PS75, and ‘2998 were dissolved in a 20% 2-HPBCD—aqueous solution. The batches of working formulations were prepared 10 minutes prior to the in vivo study.

Mice were injected i.p. with 2,2,2-tribromoethanol at 150 mg/kg prior to drawing CSF and blood. CSF was collected under a stereomicroscope from cisterna magna using 1 mL syringes. Blood collection was performed from the orbital sinus in microtainers containing K3EDTA. Animals were sacrificed by cervical dislocation after the blood samples collection. Blood samples were centrifuged 10 min at 3000 rpm. Brain samples (right lobe) were weighed and transferred into 1.5 ml tubes. All samples were immediately processed, flash-frozen and stored at −70° C. until subsequent analysis.

Plasma samples (40 μL) were mixed with 200 μL of internal standard (IS) solution. After mixing by pipetting and centrifuging for 4 min at 6000 rpm, 4 μL of each supernatant was injected into the LC-MS/MS system. Solutions of internal standards were used to quantify compounds in the plasma samples. Brain samples (weight 200 mg±1 mg) were homogenized with 800 μl of an internal stock solution using zirconium oxide beads (115 mg±5 mg) in a Bullet Blender® homogenizer for 30 seconds at speed 8. After this, the samples were centrifuged for 4 min at 14,000 rpm, and supernatant was injected into LC-MS/MS system. CSF samples (2 μL) were mixed with 40 μL of an internal stock solution. After mixing by pipetting and centrifuging for 4 min at 6,000 rpm, 5 μL of each supernatant was injected into LC-MS/MS system.

Analyses of plasma, brain and CSF samples were conducted at Enamine/Bienta. The concentrations of compounds in plasma and brain samples were determined using high performance liquid chromatography/tandem mass spectrometry (HPLC-MS/MS). Data acquisition and system control was performed using Analyst 1.5.2 software (AB Sciex, Canada). The concentrations of the test compound below the lower limit of quantitation (LLOQ=10 ng/ml for plasma, 20 ng/g for brain and 5 ng/ml for CSF samples) were designated as zero. Pharmacokinetic data analysis was performed using noncompartmental, bolus injection or extravascular input analysis models in WinNonlin 5.2 (PharSight). Data below LLOQ were presented as missing to improve validity of T/2 calculations.

Statistical analyses. Data from functional experiments of adrenergic and D2long receptors were analyzed applying the algorithms for four parameter non-linear regression implemented in Prism 8.0 (GraphPad, San Diego, CA) to get dose-response curves representing EC₅₀ and Eₘₐₓ values. Mean values were derived by summarizing the results from each individual experiment to provide EC₅₀±s.e.m. and Eₘₐₓ±s.e.m. Additional statistical analyses for FIGS. 4A-4H, FIG. 8, FIG. 15A, and FIGS. 15C-15E were performed with GraphPad Prism 9.0 (GraphPad Software Inc., San Diego). Data reported are means±s.e.m. or in FIGS. 4A-4H, single data points with mean s±s.e.m. Experiments of the compounds in the in vivo neuropathic, inflammatory, hot-plate, tailflick, and rotarod models were evaluated using unpaired two-tailed Student's t-test (20% kolliphor vs W9686), and rotarod experiments were analyzed with unpaired two-tailed Student's t-test or one-way ANOVA with Dunnett's multiple comparison post-hoc test to determine differences between groups.

### General Synthetic Routes

Method 5. Amine (1 eq), halogenide (1.1 eq), and caesium carbonate (Cs₂CO₃) (2.5 eq) were mixed in dry dioxane (approx. 0.5 mL per 100 mg of product). Then RuPhos Pd G4 (0.05 eq) (as a stock solution in dioxane approx. 0.05 mL per 100 mg of product), and RuPhos (0.05 eq) (as a stock solution in dioxane approx. 0.05 mL per 100 mg of product) were added in one portion under inert atmosphere. The reaction mixture was sealed and heated with stirring for 16 hours at 100° C. Then the mixture was cooled to ambient temperature and the Cleavage cocktail (CC) (Trifluoroacetic acid, triisopropylsilane, water (93:5:2; v/v), approx. 1 mL per 100 mg of product) was added in one portion. The mixture was stirred for 6 hours at ambient temperature and the solvent was evaporated under reduced pressure and the residue was dissolved in DMSO (approx. 1 mL up to 300 mg of product). The DMSO solution was treated with scavenger SiliaMetS DMT, filtered, analyzed by LC/MS, and transferred for HPLC purification.

Method 7. Amine (1.2 eq), halogenide 2 (1 eq), and Cs₂CO₃ (2.5 eq) were mixed in dry dioxane (apprpx. 0.7 mL per 100 mg of product). Then RuPhos Pd G4 (0.05 eq) (as a stock solution in dioxane approx. 0.05 mL per 100 mg of product), and RuPhos (0.05 eq) (as a stock solution in dioxane approx. 0.05 mL per 100 mg of product) were added in one portion under inert atmosphere. The reaction mixture was sealed and heated with shaking for 16 hours at 100° C. Then the mixture was cooled and trifluoroacetic acid (TFA) was added dropwise until neutral pH and the solvent was evaporated under reduced pressure, and the residue was dissolved in DMSO (approx. 1 mL per 100 mg of product). The DMSO solution was treated with Scavenger SiliaMetS DMT and filtered, analyzed by LC/MS, and transferred for HPLC purification.

Method 20. 1-Benzothiophen-7-amine hydrochloride (1 eq), 4-bromopyridine hydrochloride (1.2 eq), and caesium carbonate (Cs₂CO₃) (5 eq) were mixed in dry dioxane (0.7 mL). Then XantPhos Pd G4 (0.05 eq), and XantPhos (0.05 eq) were added in one portion in an inert atmosphere. The reaction mixture was sealed and heated with shaking for 24 hours at 100° C. Then the mixture was cooled and trifluoroacetic acid (TFA) was added dropwise until neutral pH, and the solvent was evaporated under reduced pressure, and the residue was dissolved in the DMSO (1 mL). DMSO solution was treated with Scavenger SiliaMetS DMT and filtered, analyzed by LC/MS, and transferred for HPLC purification.

General (Methods 21 and 22). Chemicals, building blocks and solvents were purchased from Carl Roth, Merck, Life Chemicals, Fluorochem, BLDpharm, Sigma Aldrich, abcr, AboChem, Acros, fisher chemical, Iris Biotech GmbH and Enamine. Analytical LC/MS was performed on a Thermo Scientific Dionex Ultimate 3000 HPLC system using DAD detection (230 nm; 254 nm) equipped with a Zorbax Eclipse XDB-C8 (4.6×150 mm, 5 μm) HPLC column, using mass detection on a BRUKER amaZon SL mass spectrometer using ESI ionization source with the following binary eluent system LC/MS(HPLC): 25% for 0.25 min, 25% to 100% in 5.75 min, 100% for 2.5 min, 100% to 25% in 0.5 min, 25% for 3 min, flow 0.4 mL/min (methanol/water+0.1% (v/v) formic acid). HPLC purity analyses were performed with an Agilent binary gradient system using UV detection (λ=210, 220, 230, 254, 280 nm) in combination with ChemStation software on an Agilent 1200 system. A Zorbax Eclipse XDB-C8 (4.6 mm×150 mm, 5 μm) column was used with a flow rate of 0.5 mL/min. For purity determination two binary systems were used: System A: 10% for 3 min, 10-95% in 15 min, 95% for 6 min, 95-10% in 3 min, 10% for 3 min (methanol/water+0.1% (v/v) formic acid) and System B: 10% for 3 min, 10-95% in 15 min, 95% for 6 min, 95-10% in 3 min, 10% for 3 min (acetonitrile/water+0.1% (v/v) trifluoroacetic acid). Preparative HPLC was performed on an Agilent 1260 infinity system using an Agilent Zorbax XDB-C8 21.2×150 mm, 5 μm column (Column 1), Macharey Nagel VP 250/21 Nucleodur C18 Pyramid, 5 μm column (Column 2) or Macharey Nagel VP 250/32 Nucleodur C18 HTec, 5 μm (Column 3) with the solvent systems indicated. Yields were not optimized. ¹H NMR and ¹³C DEPTQ NMR spectra were recorded on a Bruker Avance 400 (¹H: 400 MHz, ¹³C: 101 MHz) or a Bruker Avance 600 (¹H: 600 MHz, ¹³C: 151 MHz) NMR spectrometer. Chemical shifts were calculated as δ (ppm) relative to residual solvent signals. High resolution mass spectra were measured with a timsTOF Pro Mass Spectrometer from Bruker Daltonics using ESI as ionization source. Analytical data marked with a # was determined by the respective vendor.

Method 21. Primary aromatic amine, Pd₂(dba)₃ (0.1 eq), t-Bu XPhos (0.1 eq), sodium tert-butylate (3.0 eq), 4-bromopyridine hydrochloride (1 eq) and DMF (4 mL) were put into a microwave tube. The tube was sealed and purged with nitrogen. The reaction was stirred at 80° C. for two days. After cooling to room temperature, thiourea resin (50 mg, QuadraPure®) was added and the mixture was stirred for 24 hours. DMF was evaporated under reduced pressure and the residue was extracted 3 times with dichloromethane, then dried with sodium sulfate and filtered over celite. The solvent was evaporated under reduced pressure. The obtained residue was taken up in acetonitrile, filtered (CHROMAFIL® Xtra PTFE 0.2) and purified by preparative HPLC. The obtained fractions were collected and freeze-dried.

Method 22. Primary aromatic amine, sodium tert-butylate (3.0 eq), 4-bromopyridine hydrochloride (1 eq) and DMF (4 mL) were put into a microwave tube. The tube was sealed and purged with nitrogen. The reaction was stirred at 120° C. for two days. After cooling to room temperature the DMF was evaporated under reduced pressure and the residue was extracted 3 times with dichloromethane, then dried with sodium sulfate and filtered. The solvent was evaporated under reduced pressure. The obtained residue was taken up in acetonitrile, filtered (CHROMAFIL® Xtra PTFE 0.2) and purified by preparative HPLC. The obtained fractions were collected and freeze-dried.

Method 23. Pyridin-4-amine (1.2 eq), 4-bromoquinoline (1 eq), and caesium carbonate (Cs₂CO₃) (3 eq) were mixed in dry dioxane (0.7 mL). Then XantPhos Pd G4 (0.05 eq), and XantPhos (0.05 eq) were added in one portion in an inert atmosphere. The reaction mixture was sealed and heated with shaking for 24 hours at 100° C. Then the mixture was cooled and trifluoroacetic acid (TFA) was added dropwise until neutral pH, and the solvent was evaporated under reduced pressure, and the residue was dissolved in the DMSO (1 mL). DMSO solution was treated with Scavenger SiliaMetS DMT and filtered, analyzed by LC/MS, and transferred for HPLC purification.

### N-(pyridin-4-yl)isoquinolin-4-amine (Compound 9087, ZINC001173879087)

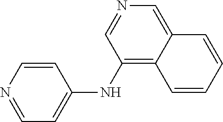 [SMILES: c1ccc2c(Nc3ccncc3)cncc2c1]

Isoquinolin-4-amine (0.15 g, 1.04 mmol, 1 eq) was dissolved in DMA (2 mL) in 40 mL vial at 25° C. 4-Bromopyridine (163.34 mg, 1.04 mmol, 1 eq) was added to the vial at 25° C. t-BuONa (199.97 mg, 2.08 mmol, 2 eq) was added to the vial at 25° C. Pd₂(dba)₃ (59.83 mg, 104.04 μmol, 0.1 eq) and t-Bu Xphos (44.18 mg, 104.04 μmol, 0.1 eq) were added to the vial in one portion at 25° C. under N₂. The mixtures were shaken at 80° C. for 16 hours under N₂ and turned to brown suspension. LC-MS showed the desired product mass was detected. To the reaction mixture was added thiourea resin (0.5 g), and the mixture was stirred at 25° C. for another 16 hours. Then the mixture was filtered through a Celite pad, and the filtrate was concentrated to give the crude product. The residue was purified by prep-HPLC (column: Phenomenex Gemini-NX C18 75*30 mm*3 um; mobile phase: water (10 mM NH₄HCO₃)-ACN]; B %: 14%-34%, 6 min). QC LCMS showed the compound N-(4-pyridyl)isoquinolin-4-amine (99.1 mg, 437.15 μmol, 42.8% yield) was obtained as a yellow solid. ¹H NMR (400 MHz, DMSO-d₆) δ 9.17 (s, 1H), 8.93 (s, 1H), 8.50 (s, 1H), 8.19 (d, J=8.0 Hz, 1H), 8.17-8.14 (m, 2H), 7.98 (dd, J=8.4, 1.0 Hz, 1H), 7.81 (ddd, J=8.4, 6.9, 1.4 Hz, 1H), 7.74 (ddd, J=8.1, 6.8, 1.2 Hz, 1H), 6.77-6.71 (m, 2H). ¹³C NMR (101 MHz, DMSO-d₆) δ 151.86, 150.07, 149.03, 137.58, 131.13, 130.98, 130.52, 128.92, 128.03, 127.90, 121.97, 109.20. LC/MS (ESI) m/z [M+H] calculated 221.3, found: 222.0.

### 3-chloro-N-(pyridin-4-yl)isoquinolin-4-amine (Compound 1718, ZINC001173881718)

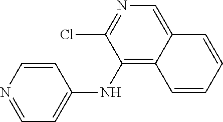 [SMILES: Clc1ncc2ccccc2c1Nc1ccncc1]

Synthesized according to Method 7. ¹H NMR (400 MHz, DMSO-d₆) 5=9.21 (s, 1H), 8.98 (s, 1H), 8.30-8.25 (m, 1H), 8.19-8.05 (m, 2H), 7.90-7.83 (m, 2H), 7.76 (ddd, J=8.1, 5.8, 2.2 Hz, 1H), 6.51-6.35 (m, 2H). ¹³C NMR (151 MHz, DMSO-d₆) δ 151.67, 150.63, 149.72, 142.97, 135.01, 131.96, 128.42, 128.39, 128.11, 127.32, 122.44, 108.65. LC/MS (ESI) m/z [M+H]+ calculated 255.7 found 256.1.

### N-(5-fluoronaphthalen-1-yl)pyridin-4-amine (Compound 7075, Z4767467075)

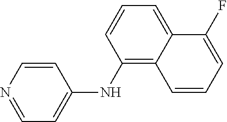 [SMILES: Fc1cccc2c(Nc3ccncc3)cccc12]

Synthesized according to Method 7. ¹H NMR (400 MHz, DMSO-d₆) δ 8.97 (s, 1H), 8.20-8.11 (m, 2H), 7.93-7.84 (m, 2H), 7.66-7.56 (m, 2H), 7.53 (ddd, J=8.6, 7.7, 5.7 Hz, 1H), 7.39 (ddd, J=10.9, 7.7, 0.9 Hz, 1H), 6.87-6.68 (m, 2H). ¹³C NMR (101 MHz, DMSO-d₆) δ 158.21 (d, ¹J_{C-F}=249.4 Hz), 151.74, 149.94, 136.29 (d, ⁴J_{C-F}=4.0 Hz), 129.99 (d, ³J_{C-F}=4.8 Hz), 126.97, 125.91 (d, ³J_{C-F}=8.6 Hz), 124.24 (d, ²J_{C-F}=16.9 Hz), 120.75, 119.23 (d, ⁴J_{C-F}=3.9 Hz), 116.37 (d, ³J_{C-F}=5.7 Hz), 110.26 (d, ²J_{C-F}=19.6 Hz), 109.31. LC/MS (ESI) m/z [M+H]+ calculated 238.3, found 239.0.

### N-(naphthalen-1-yl)pyridin-4-amine (Compound 5879, ZINC000082715879)

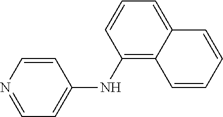 [SMILES: c1ccc2c(Nc3ccncc3)cccc2c1]

Synthesized according to Method 7. ¹H NMR (400 MHz, DMSO-d₆) δ 8.87 (s, 1H), 8.17-8.10 (m, 2H), 8.03-7.94 (m, 2H), 7.77 (d, J=8.2, 1H), 7.59-7.49 (m, 3H), 7.46 (dd, J=7.4, 1.2 Hz, 1H), 6.78-6.69 (m, 2H). ¹³C NMR (101 MHz, DMSO-d6) δ 152.11, 149.94, 135.94, 134.37, 128.61, 128.34, 126.39, 126.05, 125.96, 124.86, 122.84, 120.19, 109.03. LC/MS (ESI) m/z [M+H]+ calculated 220.3, found 220.9.

### N-(7-methylnaphthalen-1-yl)pyridin-4-amine (Compound 4825, ZINC001173884825)

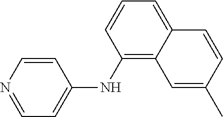 [SMILES: Cc1ccc2cccc(Nc3ccncc3)c2c1]

Synthesized according to Method 7. ¹H NMR (400 MHz, DMSO-d₆) δ 8.79 (s, 1H), 8.16-8.09 (m, 2H), 7.87 (d, J=8.4 Hz, 1H), 7.81-7.78 (m, 1H), 7.75-7.68 (m, 1H), 7.47-7.42 (m, 2H), 7.40 (dd, J=8.2, 1.6 Hz, 1H), 6.76-6.71 (m, 2H), 2.46 (s, 3H). ¹³C NMR (101 MHz, DMSO-d6) δ 152.15, 149.94, 135.30, 135.25, 132.67, 128.85, 128.51, 128.27, 125.05, 124.64, 121.61, 120.30, 108.96, 21.66. LC/MS (ESI) m/z [M+H]+ calculated 234.3 found 234.9.

### N4-(naphthalen-1-yl)pyridine-2.4-diamine (Compound 9835, ZINC000156409835)

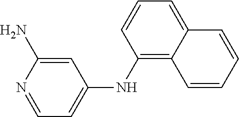 [SMILES: Nc1cc(Nc2cccc3ccccc23)ccn1]

Synthesized according to Method 5. ¹H NMR DMSO-d₆, 600 MHz): δ (ppm) 5.47 (s, 2H), 5.78 (d, 1H), 6.11 (dd, 1H), 7.38 (d, 1H), 7.49 (m, 3H), 7.57 (d, 1H), 7.70 (d, 1H), 7.92 (d, 1H), 8.00 (d, 1H), 8.42 (s, 1H).

### N-(4-fluoronaphthalen-1-yl)pyridin-4-amine (Compound 4487, ZINC001173884487)

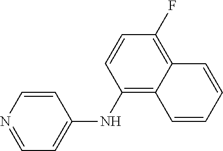 [SMILES: Fc1ccc(Nc2ccncc2)c2ccccc12]

Synthesized according to Method 7. ¹H NMR (DMSO-d₆, 500 MHz): δ (ppm) 6.62 (d, 2H), 7.34 (dd, 1H), 7.41 (dd, 1H), 7.64 (m, 2H), 7.98 (d, 1H), 8.09 (m, 3H), 8.77 (s, 1H).

### N-(pyridin-4-yl)isoquinolin-5-amine (Compound 2813, ZINC001173882813)

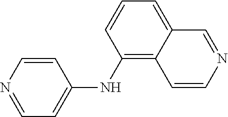 [SMILES: c1cc(Nc2ccncc2)c2ccncc2c1]

Synthesized according to Method 7. ¹H NMR (DMSO-d₆, 500 MHz): δ (ppm) 6.81 (d, 2H), 7.67 (t, 1H), 7.71 (d, 1H), 7.84 (d, 1H), 7.92 (d, 1H), 8.17 (d, 2H), 8.51 (d, 1H), 9.02 (s, 1H), 9.33 (s, 1H).

### 2-methyl-N-(pyridin-4-yl)quinolin-4-amine (Compound 9506, ZINC001173879506)

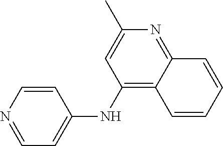 [SMILES: Cc1cc(Nc2ccncc2)c2ccccc2n1]

Synthesized according to Method 7. ¹H NMR (DMSO-d₆, 500 MHz): δ (ppm) 2.55 (s, 3H), 7.23 (d, 2H), 7.28 (s, 1H), 7.50 (t, 1H), 7.69 (t, 1H), 7.85 (d, 1H), 8.20 (d, 1H), 8.35 (d, 2H), 9.23 (s, 1H).

### N-(pyridin-4-yl)isoquinolin-1-amine (Compound 9204, ZINC001173879204)

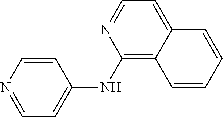 [SMILES: c1ccc2c(Nc3ccncc3)nccc2c1]

Synthesized according to Method 7. ¹H NMR (DMSO-d₆, 500 MHz): δ (ppm) 7.35 (d, 1H), 7.67 (t, 1H), 7.75 (t, 1H), 7.88 (d, 1H), 7.91 (d, 2H), 8.12 (d, 1H), 8.36 (d, 2H), 8.53 (d, 1H), 9.54 (s, 1H).

### Compound 4914, ZINC000150124914

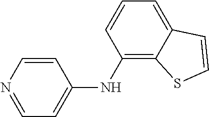 [SMILES: c1cc(Nc2ccncc2)c2sccc2c1]

Synthesized according to Method 20. ¹H NMR (400 MHz, DMSO-d6) δ 6.70-6.67 (m, 2H).7.25-7.33 (m, 1H), 7.42 (t, J=7.7 Hz, 1H), 7.51 (d, J=5.4 Hz, 1H), 7.73 (dd, J=7.9, 1.0 Hz, 1H), 7.76 (dd, J=5.4, 0.5 Hz, 1H), 8.12-8.19 (m, 2H), 8.88 (s, 1H). ¹³C NMR (101 MHz, DMSO) δ 109.37, 118.27, 120.20, 124.62, 125.38, 127.44, 134.43, 134.67, 141.39, 149.95, 150.63.

### Compound 3084, ZINC143573084

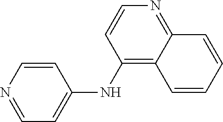 [SMILES: c1ccc2c(Nc3ccncc3)ccnc2c1]

Synthesized according to Method 23. ¹H NMR (400 MHz, DMSO-d6) δ 7.23-7.29 (m, 2H), 7.39 (d, J=5.1 Hz, 1H), 7.60 (ddd, J=8.3, 6.8, 1.3 Hz, 1H), 7.76(ddd, J=8.4, 6.8, 1.4 Hz, 1H), 7.97 (dd, J=8.5, 1.2 Hz, 1H), 8.30 (dd, J=8.5, 0.8 Hz, 1H), 8.36-8.41 (m, 2H), 8.67 (d, J=5.1 Hz, 1H), 9.37 (s, 1H). ¹³C NMR (101 MHz, DMSO-d6) δ 106.81, 112.64, 121.22, 122.50, 125.51, 129.39, 129.71, 136.84, 144.64, 149.13, 150.50, 150.79.

### Compound PS86

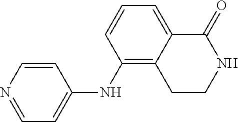 [SMILES: O=C1NCCc2c(Nc3ccncc3)cccc21]

Synthesized according to Method 21. ¹H NMR (400 MHz, DMSO-d6) δ 1H NMR (400 MHz, DMSO-d6) δ 2.51-2.57 (m, 2H), 2.74 (t, J=6.5 Hz, 2H), 6.86 (s, 2H), 7.46-7.54 (m, 2H), 7.91 (dd, J=6.5, 2.5 Hz, 1H), 8.11 (s, 1H), 8.18-8.30 (m, 2H), 10.07-10.41 (m, 1H), 13.49 (s, 1H). ¹³C NMR (101 MHz, DMSO-d6) δ 23.45, 38.50, 126.56, 127.75, 129.47, 131.48, 133.97, 135.72, 141.05, 157.18, 163.89.

### Compound PS77

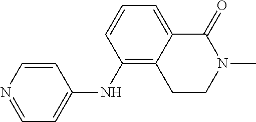 [SMILES: CN1CCc2c(Nc3ccncc3)cccc2C1=O]

Synthesized according to Method 21. ¹H NMR (400 MHz, DMSO-d6): δ 2.82 (t, J=6.6 Hz, 2H), 3.03 (s, 3H), 3.53 (t, J=6.7 Hz, 2H), 6.88 (s(br), 2H), 7.46-7.54 (m, 2H), 7.93 (dd, J=6.6, 2.5 Hz, 1H), 8.12-8.42 (m, 2H), 10.35 (s, 1H), 13.69 (s(br), 1H). ¹³C NMR (101 MHz, DMSO-d6): 22.96, 34.61, 46.59, 126.72, 127.78, 129.19, 131.14, 133.80, 134.99, 140.83, 157.28, 162.88.

### Compound PS85

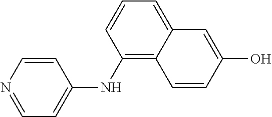 [SMILES: Oc1ccc2c(Nc3ccncc3)cccc2c1]

Synthesized according to Method 21. ¹H NMR (400 MHz, DMSO-d6): δ 6.90 (s (br), 2H), 7.16 (dd, J=9.0, 2.4 Hz, 1H), 7.25 (d, J=2.4 Hz, 1H), 7.30 (d, J=7.2 Hz, 1H), 7.49 (dd, J=8.3, 7.3 Hz, 1H), 7.75 (dd, J=9.1, 9.1 Hz, 2H), 8.18-8.32 (m, 2H), 9.91-10.17 (m, 1H), 10.43-10.76 (m, 1H), 13.56 (s(br), 1H). ¹³C NMR (101 MHz, DMSO-d6): δ 109.59, 119.62, 119.95, 123.13, 124.05, 126.28, 126.38, 132.80, 136.19, 140.69, 156.17, 158.01.

### Compound PS84

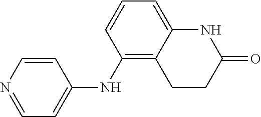 [SMILES: O=C1CCc2c(cccc2Nc2ccncc2)N1]

Synthesized according to Method 21. ¹H NMR (400 MHz, DMSO-d6): δ 2.45-2.48 (m, 2H), 2.85-2.95 (m, 2H), 6.78 (d, J=2.2 Hz, 1H), 6.88 (dd, J=8.0, 2.2 Hz, 1H), 7.02-7.10 (m, 2H), 7.27 (d, J=8.0 Hz, 1H), 8.21-8.31 (m, 2H), 10.22 (s, 1H), 10.35 (s, 1H), 13.51 (s(br), 1H). ¹³C NMR (101 MHz, DMSO-d6): δ 24.38, 30.30, 109.56, 116.35, 121.71, 129.01, 136.09, 139.61, 141.20, 156.00, 170.21.

### Compound PS62

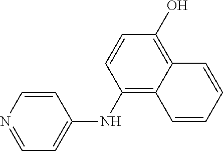 [SMILES: Oc1ccc(Nc2ccncc2)c2ccccc12]

Synthesized according to Method 21.

### Compound PS70

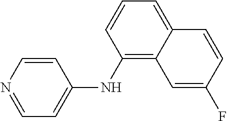 [SMILES: Fc1ccc2cccc(Nc3ccncc3)c2c1]

Synthesized according to Method 21. ¹H NMR (400 MHz, DMSO-d6): δ 6.96 (s (br), 2H), 7.56 (ddd, J=8.8, 8.8, 2.6 Hz, 1H), 7.60-7.71 (m, 3H), 8.01-8.11 (m, 1H), 8.19 (dd, J=9.1, 5.8 Hz, 1H), 8.24-8.32 (m, 2H), 10.64 (s(br), 1H), 13.74 (s(br), 1H). ¹³C NMR (101 MHz, DMSO-d6): δ 106.09 (d, 2J_{C-F}=22.5 Hz), 117.16 (d, 2J_{C-F}=25.3 Hz), 124.43, 125.57 (d, 4J_{C-F}=2.6 Hz), 127.93, 129.75 (d, 3J_{C-F}=8.8 Hz), 131.56, 131.92 (d, 3J_{C-F}=9.2 Hz), 132.57 (d, 4J_{C-F}=5.4 Hz), 140.79, 157.88, 160.74 (d, 1J_{C-F}=245.4 Hz).

### Compound PS78

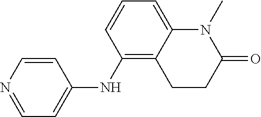 [SMILES: CN1C(=O)CCc2c(Nc3ccncc3)cccc21]

Synthesized according to Method 21. ¹H NMR (400 MHz, DMSO-d6): δ 2.50-2.55 (m, 2H), 2.66-2.78 (m, 2H), 3.29 (s, 3H), 6.87 (s(br), 2H), 7.04 (d, J=7.8 Hz, 1H), 7.17 (d, J=8.2 Hz, 1H), 7.41 (dd, J=8.1 Hz, 1H), 8.20-8.31 (m, 2H), 10.26-10.40 (m, 1H), 13.61 (s(br), 1H). ¹³C NMR (101 MHz, DMSO-d6): δ 19.96, 29.46, 30.26, 114.37, 120.17, 122.64, 128.29, 134.07, 140.72, 142.14, 157.31, 169.15.

### Compound PS71

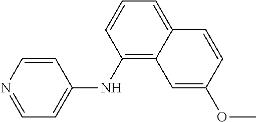 [SMILES: COc1ccc2cccc(Nc3ccncc3)c2c1]

Synthesized according to Method 21. ¹H NMR (400 MHz, DMSO-d6): δ 3.82 (s, 3H), 6.95 (s(br), 2H), 7.19 (s(br), 1H), 7.30 (ddd, J=9.0, 2.6, 1.0 Hz, 1H), 7.47 (dd, J=7.7, 7.7 Hz, 1H), 7.55 (dd, J=7.4, 1.5 Hz, 1H), 7.94 (d, J=8.1 Hz, 1H), 8.00 (d, J=9.0 Hz, 1H), 8.24-8.29 (m, 2H), 10.55-10.64 (m, 1H), 13.58 (s(br), 1H). ¹³C NMR (101 MHz, DMSO-d6): δ 55.32, 100.98, 119.17, 123.55, 123.78, 127.72, 129.79, 130.00, 130.47, 131.66, 140.71, 157.87, 158.13.

### Compound PS72

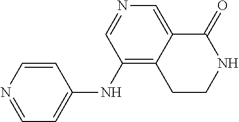 [SMILES: O=C1NCCc2c(Nc3ccncc3)cncc21]

Synthesized according to Method 21. ¹H NMR (400 MHz, DMSO-d6): δ 2.85-2.95 (m, 2H), 4.08-4.21 (m, 2H), 6.88-6.99 (m, 1H), 7.10-7.19 (m, 1H), 7.27-7.36 (m, 1H), 7.98-8.05 (m, 2H), 8.74-8.86 (m, 2H). ¹³C NMR (101 MHz, DMSO-d6): δ 22.15, 46.80, 117.02, 118.93, 119.03, 123.24, 127.13, 128.40, 143.19, 144.74, 155.24, 164.36.

### Compound PS74

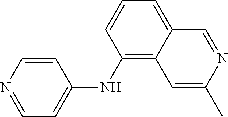 [SMILES: Cc1cc2c(Nc3ccncc3)cccc2cn1]

Synthesized according to Method 21. ¹H NMR (400 MHz, DMSO-d6): δ 2.65 (s, 3H), 6.99 (s(br), 2H), 7.67-7.75 (m, 1H), 7.75-7.83 (m, 1H), 7.86-7.93 (m, 1H), 8.17-8.24 (m, 1H), 8.26-8.35 (m, 2H), 9.47 (s, 1H), 10.69 (s, 1H), 13.66 (s(br), 1H). ¹³C NMR (101 MHz, DMSO-d6): δ 23.25, 113.89, 127.29, 127.41, 127.79, 128.32, 131.72, 132.50, 140.86, 151.76, 151.81, 157.81.

### Compound PS79

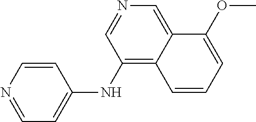 [SMILES: COc1cccc2c(Nc3ccncc3)cncc12]

Synthesized according to Method 21. ¹H NMR (400 MHz, DMSO-d6): δ 4.06 (s, 3H), 6.98 (s(br), 2H), 7.26 (d, J=7.9 Hz, 1H), 7.43 (dd, J=8.6, 2.4 Hz, 1H), 7.71-7.87 (m, 1H), 8.16-8.38 (m, 2H), 8.63 (s, 1H), 9.54 (s, 1H), 10.62-10.71 (m, 1H), 13.70 (s, 1H). ¹³C NMR (101 MHz, DMSO-d6): δ 56.22, 107.29, 113.12, 120.67, 127.95, 128.02, 132.90, 140.53, 140.65, 140.84, 146.16, 156.39, 158.18.

### Compound PS76

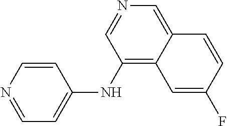 [SMILES: Fc1ccc2cncc(Nc3ccncc3)c2c1]

Synthesized according to Method 21. ¹H NMR (400 MHz, DMSO-d6): δ 7.03 (s (br), 2H), 7.68-7.72 (m, 1H), 7.75 (ddd, J=8.9, 8.9, 2.5 Hz, 1H), 8.32 (d, J=6.8 Hz, 2H), 8.44 (dd, J=9.0, 5.6 Hz, 1H), 8.65 (s, 1H), 9.42 (s, 1H), 10.71 (s(br), 1H), 13.86 (s(br), 1H). ¹³C NMR (101 MHz, DMSO-d6): δ 105.72 (d, 2J_{C-F}=23.1 Hz), 109.30, 118.93 (d, 2J_{C-F}=25.5 Hz), 126.42, 128.39 (d, 4J_{C-F}=4.8 Hz), 132.53 (d, 3J_{C-F}=10.1 Hz), 133.41 (d, 3J_{C-F}=10.1 Hz), 140.53, 140.91, 151.54, 158.04, 163.43 (d, 1J_{C-F}=251.7 Hz).

### Compound PS73

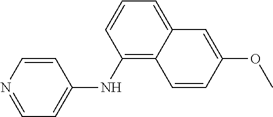 [SMILES: COc1ccc2c(Nc3ccncc3)cccc2c1]

Synthesized according to Method 21. ¹H NMR (400 MHz, DMSO-d6): δ 3.90 (s, 3H), 6.92 (s(br), 2H), 7.25 (dd, J=9.2, 2.6 Hz, 1H), 7.36-7.44 (m, 1H), 7.48 (d, J=2.6 Hz, 1H), 7.58 (dd, J=8.3, 7.3 Hz, 1H), 7.80 (d, J=9.2 Hz, 1H), 7.90 (d, J=8.3 Hz, 1H), 8.17-8.33 (m, 2H), 10.68 (s, 1H), 13.73 (s(br), 1H). ¹³C NMR (101 MHz, DMSO-d6): δ 55.41, 106.86, 119.60, 120.86, 123.97, 124.00, 126.75, 126.88, 132.84, 135.96, 140.71, 157.91, 158.02.

### N-(7-Chloronaphthalen-1-yl)pyridin-4-amine x TFA (Compound PS75)

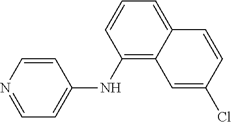 [SMILES: Clc1ccc2cccc(Nc3ccncc3)c2c1]

Synthesized according to Method 22. ¹H NMR (400 MHz, DMSO-d6): δ 6.99 (s (br), 2H), 7.63-7.70 (m, 3H), 7.99 (d, J=2.1 Hz, 1H), 8.05 (dd, J=5.5, 4.0 Hz, 1H), 8.14 (d, J=8.8 Hz, 1H), 8.23-8.31 (m, 2H), 10.58 (s, 1H), 13.58 (s(br), 1H); ¹³C NMR (101 MHz, DMSO-d6): δ 99.56, 121.20, 124.32, 126.73, 127.51, 127.77, 129.46, 131.02, 131.96, 132.30, 132.76, 141.00, 157.73; LC/MS (ESI+) m/z: 254.9 [M+H]+; HRMS (ESI+) m/z calcd for C₁₅H₁₁ClN₂+H+: 255.0684 [M+H]+, found: 255.0681 [M+H]+; HPLC System A: t_{R}=16.5 min, purity: 98% (254 nm), System B: t_{R}=14.5 min, purity: 98% (254 nm).

### Compound PS83

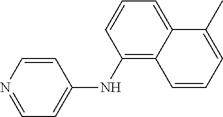 [SMILES: Cc1cccc2c(Nc3ccncc3)cccc12]

Synthesized according to Method 21. ¹H NMR (400 MHz, DMSO-d6): δ 2.71 (s, 3H), 6.90 (s(br), 2H), 7.45-7.53 (m, 2H), 7.60 (d, J=7.3 Hz, 1H), 7.68 (dd, J=8.5, 7.3 Hz, 1H), 7.76 (ddd, J=6.4, 3.0, 1.2 Hz, 1H), 8.11 (dd, J=8.5, 1.1 Hz, 1H), 8.21-8.29 (m, 2H), 10.71 (s, 1H), 13.70 (s(br), 1H). ¹³C NMR (101 MHz, DMSO-d6): δ 19.33, 120.55, 123.38, 124.38, 126.00, 126.79, 127.60, 129.01, 133.19, 133.45, 135.10, 140.69, 158.14.

### Compound PS82

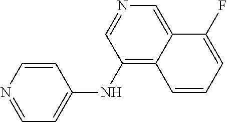 [SMILES: Fc1cccc2c(Nc3ccncc3)cncc12]

Synthesized according to Method 21. ¹H NMR (400 MHz, DMSO-d6): δ 6.95-7.11 (m, 2H), 7.63 (ddd, J=10.7, 7.8, 0.8 Hz, 1H), 7.78 (d, J=8.5, 1H), 7.89 (ddd, J=8.6, 7.8, 5.5 Hz, 1H), 8.27-8.40 (m, 2H), 8.74 (s, 1H), 9.52 (s, 1H), 10.86 (s, 1H), 14.05 (s(br), 1H). ¹³C NMR (101 MHz, DMSO-d6): δ 109.3, 112.65 (d, 2J_{C-F}=18.7 Hz), 118.14 (d, 4J_{C-F}=4.3 Hz), 119.20 (d, 2J_{C-F}=16.0 Hz), 128.32 (d, 4J_{C-F}=3.6 Hz), 132.55 (d, 3J_{C-F}=8.9 Hz), 133.04 (d, 4J_{C-F}=3.4 Hz), 141.00, 141.08, 144.95 (d, 4J_{C-F}=4.5 Hz), 158.13, 158.60 (d, 1J_{C-F}=255.2 Hz).

### References for Examples 1 and 2

1. J. Dahlhamer, J. Lucas, C. Zelaya, R. Nahin, S. Mackey, L. DeBar, R. Kerns, M. Von Korff, L. Porter, C. Helmick, MMWR Morb. Mortal. Wkly. Rep. 67, 1001-1006 (2018).
2. L. Manchikanti, S. Helm, B. Fellows, J. W. Janata, V. Pampati, J. S. Grider, M. V. Boswell, Pain Physician. 15, ES9-38 (2012).
3. J. Strang, N. D. Volkow, L. Degenhardt, M. Hickman, K. Johnson, G. F. Koob, B. D. L. Marshall, M. Tyndall, S. L. Walsh, Nat. Rev. Dis. Primer. 6, 3 (2020).
4. K. Rasmussen, D. A. White, J. B. Acri, Neuropsychopharmacology. 44, 657-659 (2019).
5. T. Grosser, C. J. Woolf, G. A. FitzGerald, Science. 355, 1026-1027 (2017).
6. S. W. Hohmann, C. Angioni, S. Tunaru, S. Lee, C. J. Woolf, S. Offermanns, G. Geisslinger, K. Scholich, M. Sisignano, Sci. Rep. 7, 446 (2017).
7. C.-R. Lin, F. Amaya, L. Barrett, H. Wang, J. Takada, T. A. Samad, C. J. Woolf, J. Pharmacol. Exp. Ther. 319, 1096-1103 (2006).
8. A. B. Malmberg, L. R. Hedley, J. R. Jasper, J. C. Hunter, A. I. Basbaum, Br. J. Pharmacol. 132, 1827-1836 (2001).
9. D. Yuan, Z. Liu, J. Kaindl, S. Maeda, J. Zhao, X. Sun, J. Xu, P. Gmeiner, H.-W. Wang, B. K. Kobilka, Nat. Chem. Biol. 16, 507-512 (2020).
10. J. Lyu, S. Wang, T. E. Balius, I. Singh, A. Levit, Y. S. Moroz, M. J. O'Meara, T. Che, E. Algaa, K. Tolmachova, A. A. Tolmachev, B. K. Shoichet, B. L. Roth, J. J. Irwin, Nature. 566, 224-229 (2019).
11. T. Sterling, J. J. Irwin, J. Chem. Inf Model. 55, 2324-2337 (2015).
12. J. J. Irwin, K. G. Tang, J. Young, C. Dandarchuluun, B. R. Wong, M. Khurelbaatar, Y. S. Moroz, J. Mayfield, R. A. Sayle, J. Chem. Inf Model. 60, 6065-6073 (2020).
13. R. M. Stein, H. J. Kang, J. D. McCorvy, G. C. Glatfelter, A. J. Jones, T. Che, S. Slocum, X.-P. Huang, O. Savych, Y. S. Moroz, B. Stauch, L. C. Johansson, V. Cherezov, T. Kenakin, J. J. Irwin, B. K. Shoichet, B. L. Roth, M. L. Dubocovich, Nature. 579, 609-614 (2020).
14. A. Alon, J. Lyu, J. M. Braz, T. A. Tummino, V. Craik, M. J. O'Meara, C. M. Webb, D. S. Radchenko, Y. S. Moroz, X.-P. Huang, Y. Liu, B. L. Roth, J. J. Irwin, A. I. Basbaum, B. K. Shoichet, A. C. Kruse, (preprint, Pharmacology and Toxicology, 2021), doi:10.1101/2021.04.29.441652.
15. C. Gorgulla, A. Boeszoermenyi, Z.-F. Wang, P. D. Fischer, P. W. Coote, K. M. Padmanabha Das, Y. S. Malets, D. S. Radchenko, Y. S. Moroz, D. A. Scott, K. Fackeldey, M. Hoffmann, I. Iavniuk, G. Wagner, H. Arthanari, Nature. 580, 663-668 (2020).
16. A. Manglik, H. Lin, D. K. Aryal, J. D. McCorvy, D. Dengler, G. Corder, A. Levit, R. C. Kling, V. Bernat, H. Hubner, X.-P. Huang, M. F. Sassano, P. M. Giguère, S. Löber, Da Duan, G. Scherrer, B. K. Kobilka, P. Gmeiner, B. L. Roth, B. K. Shoichet, Nature. 537, 185-190 (2016).
17. J. A. Allen, J. M. Yost, V. Setola, X. Chen, M. F. Sassano, M. Chen, S. Peterson, P. N. Yadav, X.-P. Huang, B. Feng, N. H. Jensen, X. Che, X. Bai, S. V. Frye, W. C. Wetsel, M. G. Caron, J. A. Javitch, B. L. Roth, J. Jin, Proc. Natl. Acad. Sci. 108, 18488-18493 (2011).
18. M. Schuller, G. J. Correy, S. Gahbauer, D. Fearon, T. Wu, R. E. Díaz, I. D. Young, L. Carvalho Martins, D. H. Smith, U. Schulze-Gahmen, T. W. Owens, I. Deshpande, G. E. Merz, A. C. Thwin, J. T. Biel, J. K. Peters, M. Moritz, N. Herrera, H. T. Kratochvil, QCRG Structural Biology Consortium, A. Aimon, J. M. Bennett, J. Brandao Neto, A. E. Cohen, A. Dias, A. Douangamath, L. Dunnett, O. Fedorov, M. P. Ferla, M. R. Fuchs, T. J. Gorrie-Stone, J. M. Holton, M. G. Johnson, T. Krojer, G. Meigs, A. J. Powell, J. G. M. Rack, V. L. Rangel, S. Russi, R. E. Skyner, C. A. Smith, A. S. Soares, J. L. Wierman, K. Zhu, P. O'Brien, N. Jura, A. Ashworth, J. J. Irwin, M. C. Thompson, J. E. Gestwicki, F. von Delft, B. K. Shoichet, J. S. Fraser, I. Ahel, Sci. Adv. 7, eabf8711 (2021).
19. C. de Graaf, A. J. Kooistra, H. F. Vischer, V. Katritch, M. Kuijer, M. Shiroishi, S. Iwata, T. Shimamura, R. C. Stevens, I. J. P. de Esch, R. Leurs, J. Med. Chem. 54, 8195-8206 (2011).
20. R. Kiss, B. Kiss, Á. Könczöl, F. Szalai, I. Jelinek, V. László, B. Noszál, A. Falus, G. M. Keserü, J. Med. Chem. 51, 3145-3153 (2008).
21. V. Katritch, V.-P. Jaakola, J. R. Lane, J. Lin, A. P. IJzerman, M. Yeager, I. Kufareva, R. C. Stevens, R. Abagyan, J. Med. Chem. 53, 1799-1809 (2010).
22. R. G. Coleman, M. Carchia, T. Sterling, J. J. Irwin, B. K. Shoichet, PLoS ONE. 8, e75992 (2013).
23. A. Gaulton, L. J. Bellis, A. P. Bento, J. Chambers, M. Davies, A. Hersey, Y. Light, S. McGlinchey, D. Michalovich, B. Al-Lazikani, J. P. Overington, Nucleic Acids Res. 40, D1100-D1107 (2012).
24. H. S. Blaxall, T. J. Murphy, J. C. Baker, C. Ray, D. B. Bylund, J. Pharmacol. Exp. Ther. 259, 323-329 (1991).
25. P. B. M. W. M. Timmermans, P. A. Van Zwieten, J. Med. Chem. 25, 1389-1401 (1982).
26. B. V. Clineschmidt, L. M. Flataker, E. P. Faison, D. R. Haubrich, Arch. Int. Pharmacodyn. Ther. 244, 231-243 (1980).
27. S. Gu, M. S. Smith, Y. Yang, J. J. Irwin, B. K. Shoichet, (preprint, Bioinformatics, 2021), doi:10.1101/2021.04.06.438722.
28. Márton Vass, A. J. Kooistra, S. Verhoeven, D. Gloriam, I. J. P. de Esch, C. de Graaf, in Computational Methods for GPCR Drug Discovery, pp. 73-113.
29. V. Isberg, C. de Graaf, A. Bortolato, V. Cherezov, V. Katritch, F. H. Marshall, S. Mordalski, J.-P. Pin, R. C. Stevens, G. Vriend, D. E. Gloriam, Trends Pharmacol. Sci. 36, 22-31 (2015).
30. K. Altosaar, P. Balaji, R. A. Bond, D. B. Bylund, S. Cotecchia, D. Devost, V. A. Doze, D. C. Eikenburg, S. Gora, E. Goupil, R. M. Graham, T. Hébert, J. P. Hieble, R. Hills, S. Kan, G. Machkalyan, M. C. Michel, K. P. Minneman, S. Parra, D. Perez, R. Sleno, R. Summers, P. Zylbergold, Adrenoceptors (version 2019.4) in the IUPHAR/BPS Guide to Pharmacology Database. IUPHARBPS Guide Pharmacol. CITE. 2019 (2019), doi:10.2218/gtopdb/F4/2019.4.
31. S. Wang, T. Che, A. Levit, B. K. Shoichet, D. Wacker, B. L. Roth, Nature. 555, 269-273 (2018).
32. R. R. Ruffolo, W. Bondinell, J. P. Hieble, J. Med. Chem. 38, 3681-3716 (1995).
33. J. Xu, S. Cao, H. Hübner, D. Weikert, G. Chen, Q. Lu, D. Yuan, P. Gmeiner, Z. Liu, Y. Du, Sci. Adv. (Under review).
34. D. Wacker, G. Fenalti, M. A. Brown, V. Katritch, R. Abagyan, V. Cherezov, R. C. Stevens, J. Am. Chem. Soc. 132, 11443-11445 (2010).
35. K. Kim, T. Che, O. Panova, J. F. DiBerto, J. Lyu, B. E. Krumm, D. Wacker, M. J. Robertson, A. B. Seven, D. E. Nichols, B. K. Shoichet, G. Skiniotis, B. L. Roth, Cell. 182, 1574-1588.e19 (2020).
36. K. H. Wang, A. Penmatsa, E. Gouaux, Nature. 521, 322-327 (2015).
37. T. M. Stepniewski, A. Mancini, R. Ågren, M. Torrens-Fontanals, M. Semache, M. Bouvier, K. Sahlholm, B. Breton, J. Selent, Chem. Sci., in press, doi:10.1039/D1SC00749A.
38. W. K. Kroeze, M. F. Sassano, X.-P. Huang, K. Lansu, J. D. McCorvy, P. M. Giguère, N. Sciaky, B. L. Roth, Nat. Struct. Mol. Biol. 22, 362-369 (2015).
39. S. D. Shields, W. A. Eckert, A. I. Basbaum, J. Pain. 4, 465-470 (2003).
40. R. Virtanen, J. M. Savola, V. Saano, Arch. Int. Pharmacodyn. Ther. 297, 190-204 (1989).
41. L. B. MacMillan, L. Hein, M. S. Smith, M. T. Piascik, L. E. Limbird, Science. 273, 801-803 (1996).
42. J. C. Hunter, D. J. Fontana, L. R. Hedley, J. R. Jasper, R. Lewis, R. E. Link, R. Secchi, J. Sutton, R. M. Eglen, Br. J. Pharmacol. 122, 1339-1344 (1997).
43. C. J. Langmead, S. P. Andrews, M. Congreve, J. C. Errey, E. Hurrell, F. H. Marshall, J. S. Mason, C. M. Richardson, N. Robertson, A. Zhukov, M. Weir, J. Med. Chem. 55, 1904-1909 (2012).
44. S. Wang, D. Wacker, A. Levit, T. Che, R. M. Betz, J. D. McCorvy, A. J. Venkatakrishnan, X.-P. Huang, R. O. Dror, B. K. Shoichet, B. L. Roth, Science. 358, 381-386 (2017).
45. L. M. Wingler, M. A. Skiba, C. McMahon, D. P. Staus, A. L. W. Kleinhenz, C.-M. Suomivuori, N. R. Latorraca, R. O. Dror, R. J. Lefkowitz, A. C. Kruse, Science. 367, 888-892 (2020).
46. M. T. Drake, J. D. Violin, E. J. Whalen, J. W. Wisler, S. K. Shenoy, R. J. Lefkowitz, J. Biol. Chem. 283, 5669-5676 (2008).
47. P. P. Lakhlani, L. B. MacMillan, T. Z. Guo, B. A. McCool, D. M. Lovinger, M. Maze, L. E. Limbird, Proc. Natl. Acad. Sci. 94, 9950-9955 (1997).
48. M. Maze, W. Tranquilli, Anesthesiology. 74, 581-605 (1991).
49. R. A. Powers, F. Morandi, B. K. Shoichet, Structure. 10, 1013-1023 (2002).
50. S. Karhuvaara, A. Kallio, M. Salonen, J. Tuominen, M. Scheinin, Br. J. Clin. Pharmacol. 31, 160-165 (1991).
51. I. D. Kuntz, J. M. Blaney, S. J. Oatley, R. Langridge, T. E. Ferrin, J. Mol. Biol. 161, 269-288 (1982).

52. J. M. Word, S. C. Lovell, J. S. Richardson, D. C. Richardson, J. Mol. Biol. 285, 1735-1747 (1999).
53. S. J. Weiner, P. A. Kollman, D. A. Case, U. C. Singh, C. Ghio, G. Alagona, S. Profeta, P. Weiner, J. Am. Chem. Soc. 106, 765-784 (1984).
54. B. J. Bender, S. Gahbauer, A. Luttens, J. Lyu, C. M. Webb, R. M. Stein, E. A. Fink, T. E. Balius, J. Carlsson, J. Irwin, B. K. Shoichet, Nat Protoc (2021), doi:10.1038/s41596-021-00597-z.
55. R. M. Stein, Y. Yang, T. E. Balius, M. J. O'Meara, J. Lyu, J. Young, K. Tang, B. K. Shoichet, J. J. Irwin, J. Chem. Inf Model. 61, 699-714 (2021).
56. M. M. Mysinger, D. R. Weiss, J. J. Ziarek, S. Gravel, A. K. Doak, J. Karpiak, N. Heveker, B. K. Shoichet, B. F. Volkman, Proc. Natl. Acad. Sci. 109, 5517-5522 (2012).
57. B. K. Shoichet, A. R. Leach, I. D. Kuntz, Proteins. 34, 4-16 (1999).
58. E. C. Meng, B. K. Shoichet, I. D. Kuntz, J. Comput. Chem. 13, 505-524 (1992).
59. K. Gallagher, K. Sharp, Biophys. J. 75, 769-776 (1998).
60. M. M. Mysinger, B. K. Shoichet, J. Chem. Inf Model. 50, 1561-1573 (2010).
61. E. F. Pettersen, T. D. Goddard, C. C. Huang, G. S. Couch, D. M. Greenblatt, E. C. Meng, T. E. Ferrin, J. Comput. Chem. 25, 1605-1612 (2004).
62. G. Madhavi Sastry, M. Adzhigirey, T. Day, R. Annabhimoju, W. Sherman, J. Comput. Aided 30 Mol. Des. 27, 221-234 (2013).
62. T. Rezai, J. E. Bock, M. V. Zhou, C. Kalyanaraman, R. S. Lokey, M. P. Jacobson, J. Am. Chem. Soc. 128, 14073-14080 (2006).
63. T. Rezai, J. E. Bock, M. V. Zhou, C. Kalyanaraman, R. S. Lokey, M. P. Jacobson, J. Am. Chem. Soc. 128, 14073-14080 (2006).
64. S. S. F. Leung, J. Mijalkovic, K. Borrelli, M. P. Jacobson, J. Chem. Inf. Model. 52, 1621-1636 (2012).
65. M. P. Jacobson, G. A. Kaminski, R. A. Friesner, C. S. Rapp, J. Phys. Chem. B. 106, 11673-11680 (2002).
66. M. Stanek, L.-P. Picard, M. F. Schmidt, J. M. Kaindl, H. Hubner, M. Bouvier, D. Weikert, P. Gmeiner, J. Med. Chem. 62, 5111-5131 (2019).
67. D. G. Gibson, L. Young, R.-Y. Chuang, J. C. Venter, C. A. Hutchison, H. O. Smith, Nat. Methods. 6, 343-345 (2009).
68. O. H. Lowry, N. J. Rosebrough, A. L. Farr, R. J. Randall, J. Biol. Chem. 193, 265-275 (1951).
69. Y. Cheng, W. H. Prusoff, Biochem. Pharmacol. 22, 3099-3108 (1973).
70. X. M. Guan, T. S. Kobilka, B. K. Kobilka, J. Biol. Chem. 267, 21995-21998 (1992).
71. J. Quoyer, J. M. Janz, J. Luo, Y. Ren, S. Armando, V. Lukashova, J. L. Benovic, K. E. Carlson, S. W. Hunt, M. Bouvier, Proc. Natl. Acad. Sci. U.S.A 110, E5088-5097 (2013).
72. C. Galds, J. J. J. Van Durm, S. Schaak, S. Pontier, Y. Percherancier, M. Audet, H. Paris, M. Bouvier, Nat. Struct. Mol. Biol. 13, 778-786 (2006).
73. Y. Namkung, C. Le Gouill, V. Lukashova, H. Kobayashi, M. Hogue, E. Khoury, M. Song, M. Bouvier, S. A. Laporte, Nat. Commun. 7, 12178 (2016).
74. D. Möller, A. Banerjee, T. C. Uzuneser, M. Skultety, T. Huth, B. Plouffe, H. Hubner, C. Alzheimer, K. Friedland, C. P. Müller, M. Bouvier, P. Gmeiner, J. Med. Chem. 60, 2908-2929 (2017).
75. H. Liu, J. Hofmann, I. Fish, B. Schaake, K. Eitel, A. Bartuschat, J. Kaindl, H. Rampp, A. Banerjee, H. Hubner, M. J. Clark, S. G. Vincent, J. T. Fisher, M. R. Heinrich, K. Hirata, X. Liu, R. K. Sunahara, B. K. Shoichet, B. K. Kobilka, P. Gmeiner, Proc. Natl. Acad. Sci. U.S.A 115, 12046-12050 (2018).
76. J. Xu, Y. Hu, J. Kaindl, P. Risel, H. Hubner, S. Maeda, X. Niu, H. Li, P. Gmeiner, C. Jin, B. K. Kobilka, Mol. Cell. 75, 53-65.e7 (2019).
77. X. Liu, J. Kaindl, M. Korczynska, A. Stößel, D. Dengler, M. Stanek, H. Hubner, M. J. Clark, J. Mahoney, R. A. Matt, X. Xu, K. Hirata, B. K. Shoichet, R. K. Sunahara, B. K. Kobilka, P. Gmeiner, Nat. Chem. Biol. 16, 749-755 (2020).
78. S. Maeda, A. Koehl, H. Matile, H. Hu, D. Hilger, G. F. X. Schertler, A. Manglik, G. Skiniotis, R. J. P. Dawson, B. K. Kobilka, Nat. Commun. 9, 3712 (2018).
79. X. Huang, L. Zhang, Z. Wen, H. Chen, S. Li, G. Ji, C. Yin, F. Sun, Prog. Biophys. Mol. Biol. 156, 3-13 (2020).
80. D. N. Mastronarde, J. Struct. Biol. 152, 36-51 (2005).
81. S. Q. Zheng, E. Palovcak, J.-P. Armache, K. A. Verba, Y. Cheng, D. A. Agard, Nat. Methods. 14, 331-332 (2017).
82. W. Jiang, F. Guo, Z. Liu, J. Struct. Biol. 180, 343-351 (2012).
83. A. Punjani, J. L. Rubinstein, D. J. Fleet, M. A. Brubaker, Nat. Methods. 14, 290-296 (2017).
84. S. H. W. Scheres, J. Struct. Biol. 180, 519-530 (2012).
85. P. Emsley, B. Lohkamp, W. G. Scott, K. Cowtan, Acta Crystallogr. D Biol. Crystallogr. 66, 486-501 (2010).
86. P. D. Adams, P. V. Afonine, G. Bunkóczi, V. B. Chen, I. W. Davis, N. Echols, J. J. Headd, L.-W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger, P. H. Zwart, Acta Crystallogr. D Biol. Crystallogr. 66, 213-221 (2010).
87. V. B. Chen, W. B. Arendall, J. J. Headd, D. A. Keedy, R. M. Immormino, G. J. Kapral, L. W. Murray, J. S. Richardson, D. C. Richardson, Acta Crystallogr. D Biol. Crystallogr. 66, 12-21 (2010).
88. W. J. Allen, R. C. Rizzo, J. Chem. Inf. Model. 54, 518-529 (2014).
89. J. Besnard, G. F. Ruda, V. Setola, K. Abecassis, R. M. Rodriguiz, X.-P. Huang, S. Norval, M. F. Sassano, A. I. Shin, L. A. Webster, F. R. C. Simeons, L. Stojanovski, A. Prat, N. G. Seidah, D. B. Constam, G. R. Bickerton, K. D. Read, W. C. Wetsel, I. H. Gilbert, B. L. Roth, A. L. Hopkins, Nature. 492, 215-220 (2012).
90. C. A. Flanagan, in Methods in Cell Biology (Elsevier, 2016; https://linkinghub.elsevier.com/retrieve/pii/S0091679X15002058), vol. 132, pp. 191-215.
91. R. N. DeHaven, D. L. DeHaven-Hudkins, Curr. Protoc. Pharmacol. 8 (2000), doi:10.1002/0471141755.ph0104 s08.
92. E. C. Hulme, M. A. Trevethick, Br. J. Pharmacol. 161, 1219-1237 (2010).
92. E. C. Hulme, M. A. Trevethick, Br. J. Pharmacol. 161, 1219-1237 (2010).
82. X.-P. Huang, T. Mangano, S. Hufeisen, V. Setola, B. L. Roth, Assay Drug Dev. Technol. 8, 727-742 (2010).
93. X.-P. Huang, T. Mangano, S. Hufeisen, V. Setola, B. L. Roth, ASSAY Drug Dev. Technol. 8, 727-742 (2010).
94. C. G. Alvaro, J. M. Braz, M. Bernstein, K. A. Hamel, V. Craik, H. Yamanaka, A. I. Basbaum, PLOS ONE. 15, e0226289 (2020).
95. S. R. Chaplan, F. W. Bach, J. W. Pogrel, J. M. Chung, T. L. Yaksh, J. Neurosci. Methods. 53, 55-63 (1994).
96. Y. Q. Cao, P. W. Mantyh, E. J. Carlson, A.-M. Gillespie, C. J. Epstein, A. I. Basbaum, Nature. 392, 390-394 (1998).

### Example 3: Structure-Based Discovery of Nonopioid Analgesics Acting Through the α_{2A}-Adrenergic Receptor

As non-opioid analgesics are much sought, we computationally docked over 301 million virtual molecules against a validated pain target, the α_{2A}-adrenergic receptor ((α_{2A}AR), seeking new chemotypes that lack the sedation conferred by known α_{2A}AR drugs, e.g., dexmedetomidine. We identified 17 new ligands with potencies as low as 12 nM, many with partial agonism and preferential G_{i/o} signaling. Experimental structures of α_{2A}AR complexed with two of the new agonists confirmed the docking predictions and templated further optimization. Several compounds, including the initial docking hit ‘9087 (EC₅₀ 52 nM), and two analogs, ‘7075 and PS75 (EC₅₀ 4.1 and 4.8 nM) exerted on-target analgesic activity in multiple in vivo pain models, without sedation. These new agonists are interesting as therapeutic leads that lack the liabilities of opioids and the sedation of dexmedetomidine.

Epidemics in pain (1) and in opioid use disorder (2, 3) have inspired a search for non-opioid analgesics (1, 4). The α_{2A}-adrenergic receptor (α_{2A}AR) is a non-opioid receptor targeted by dexmedetomidine, a sedative that also has strong analgesic activity (5). While dexmedetomidine has many advantages in emergency room and intensive care settings, its strong sedative effects (6, 7) and its lack of an oral formulation (8) have limited its broad use as an analgesic. These properties are barriers for future therapeutics targeting this receptor.

Most α_{2A}AR analgesics are chemically related, and the relationship of their sedative to their analgesic properties is unclear. To find therapeutics with new pharmacology we sought new chemotypes, topologically unrelated to known α_{2A}AR agonists. The α_{2B}-adrenergic receptor ((α_{2B}AR) active-state structure (9) became available and its binding site is highly-conserved versus α_{2A}AR, therefore it should be possible to identify new α_{2A}AR agonists by structure-based docking. Meanwhile, the advent of readily accessible make-on-demand (“tangible”) molecules (10-12) ranging from hundreds-of-millions (10, 13, 14) to over a billion molecules (15, 16) has vastly increased chemotypes available for ligand discovery. Docking these libraries has revealed new ligands with 20-60% hit rates (13, 14, 17-20) and sometimes nanomolar potencies for a growing range of targets (10, 13, 14, 18, 21-24), often with new pharmacology (10, 13, 17, 25). Therefore, we targeted the α_{2B}AR with an ultra-large library docking screen.

### Docking 301 Million Molecules Versus the α_{2B}AR

The ZINC15/20 virtual library is comprised of millions to billions of tangible molecules, depending on the molecular property range targeted, and is accessed by combining hundreds of thousands of diverse building blocks through hundreds of well-characterized reactions (10-12). Most of the molecules have not previously been synthesized, and range in mass, calculated Log P (c Log P) values (a measure of hydrophobicity), and formal charge. Given the small size of the α_{2B}AR orthosteric site, we docked both the 20 million ‘fragment-like’ (compounds with smaller masses of <250 amu) and 281 million ‘lead-like’ (compounds with larger masses of 250-350 amu) molecules from the ZINC15 library (both sets having c Log P≤3.5) (11) (FIG. 1A). Over 233 trillion complexes, an average of 452,000 per molecule, were sampled by DOCK3.7 and scored with its physics-based energy function (26) across three separate screens (two fragment screens with different variables and one lead-like screen). For each screen, the top 300,000 docking-ranked compounds were clustered for topological similarity and then filtered to identify scaffolds dissimilar to known agonists using an Extended Connectivity Fingerprint (ECFP4). These agonists were drawn from the International Union of Basic and Clinical Pharmacology (IUPHAR)/British Pharmacological Society (BPS) database (27) and from the literature (28-31). Ligands with internal torsional strain were deprioritized (32). An additional novelty filter removed molecules similar to annotated α_{2A}AR compounds in CHEMBL29 (28). Of the remaining top-ranked cluster representatives, 5,000 from each fragment screen and 20,000 for the lead-like screen were manually evaluated in UCSF Chimera (https://rbvi.ucsf.edu/chimera) for key polar and nonpolar interactions with α_{2B}AR (9), including with D92^{3.32}, F412^{7.39}, F387^{6.51}, Y391^{6.55}, and F388^{6.52} (residues conserved in α_{2A}AR: D128^{3.32}, F427^{7.39}, F405^{6.51}, Y409^{6.55}, and F406^{6.52}; superscripts use Ballesteros-Weinstein and the G Protein-Coupled Receptor database (GPCRdb) nomenclature (33)). Most α_{2A}AR agonists, and certainly the clinically used dexmedetomidine and clonidine, are fragments (27), and the docking results reflected this. The docked fragment molecules fit in the orthosteric site, making key contacts with the receptor, while molecules in the lead-like screen generally did not to fit in the small cavity (FIG. 1A). Accordingly, most selected ligands came from the fragment docking screens.

From the 64 high-ranking docked compounds prioritized by visual inspection and purchased for in vitro testing, 48 were successfully synthesized: 44 fragments and 4 lead-like molecules. Compounds were first tested for binding to the human α_{2B}AR receptor, the structure used in docking screens. Thirty molecules of the 48 tested had Kᵢ values less than 10 μM. This 63% hit rate is among the highest for a docking campaign to date (10, 14, 21, 23, 34). In radioligand competition assays, compound ZINC1173879087 (from here on referred to as ‘9087) had a Kᵢ of 1.7 nM; the remaining 29 had Kᵢ values ranging from 60 nM to 9.4 μM, which is relatively potent for initial docking hits. Ten compounds (21%) had Kᵢ values below 1 μM. The compounds were then tested for binding to the murine α_{2A}AR, again by radioligand competition. Of these, 17 (35%) had a Kᵢ better than 10 μM, with affinities ranging from 72 nM to 9.4 μM; five compounds (10%) had Kᵢ values below 1 μM. Against human α_{2A}AR, the highest affinity was 12 nM (Table 1).

### Discovery of Novel, Partial G_{i/o} Agonists

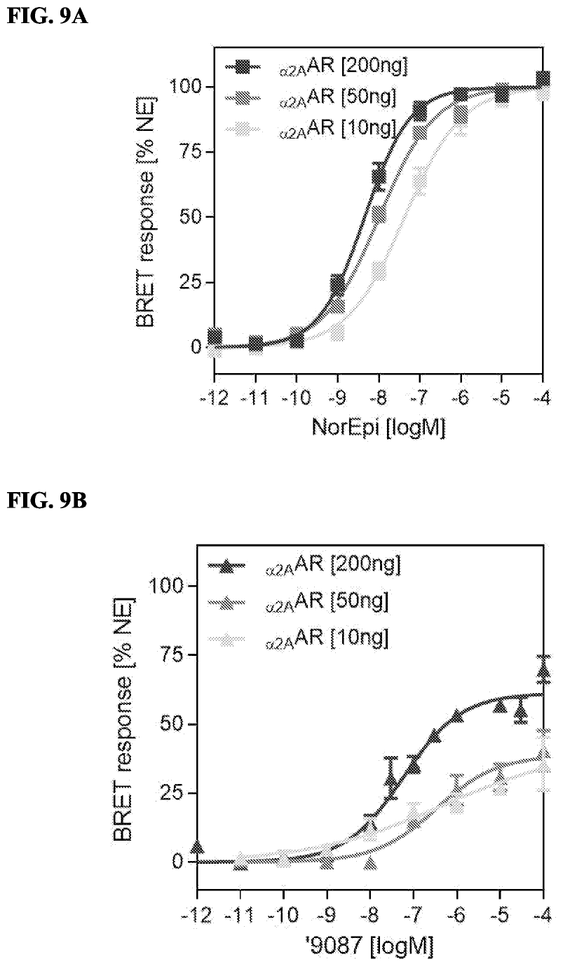

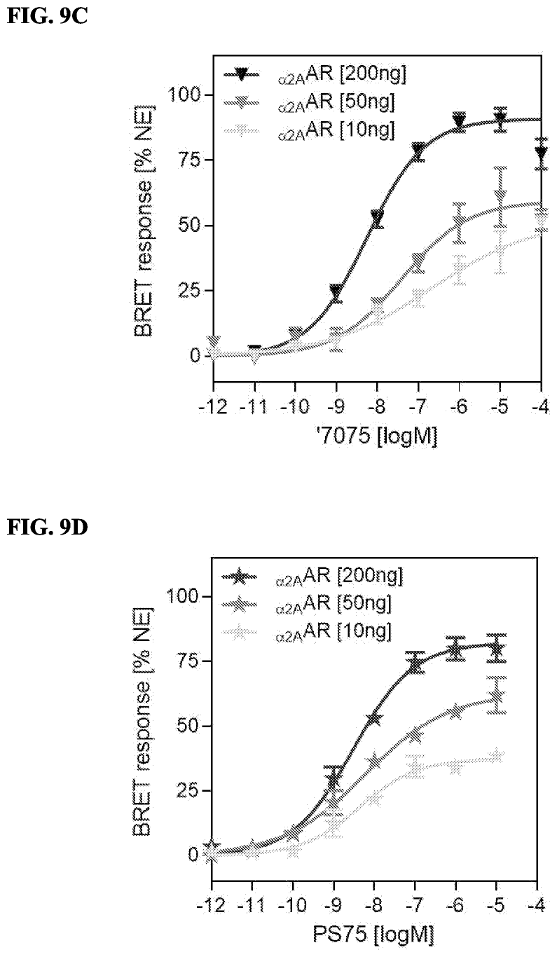

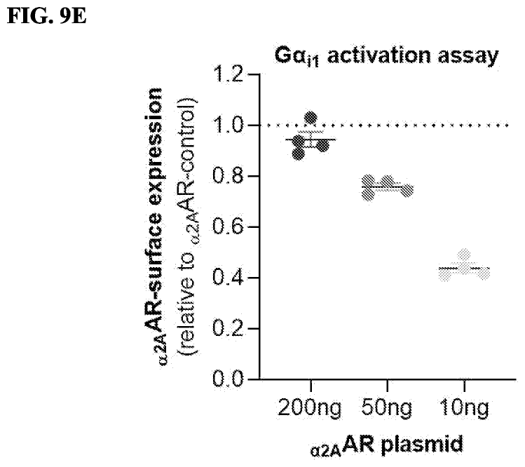

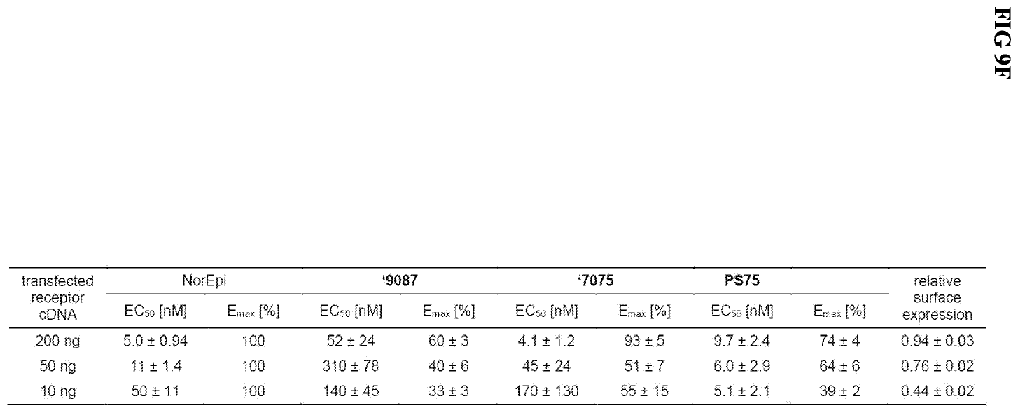

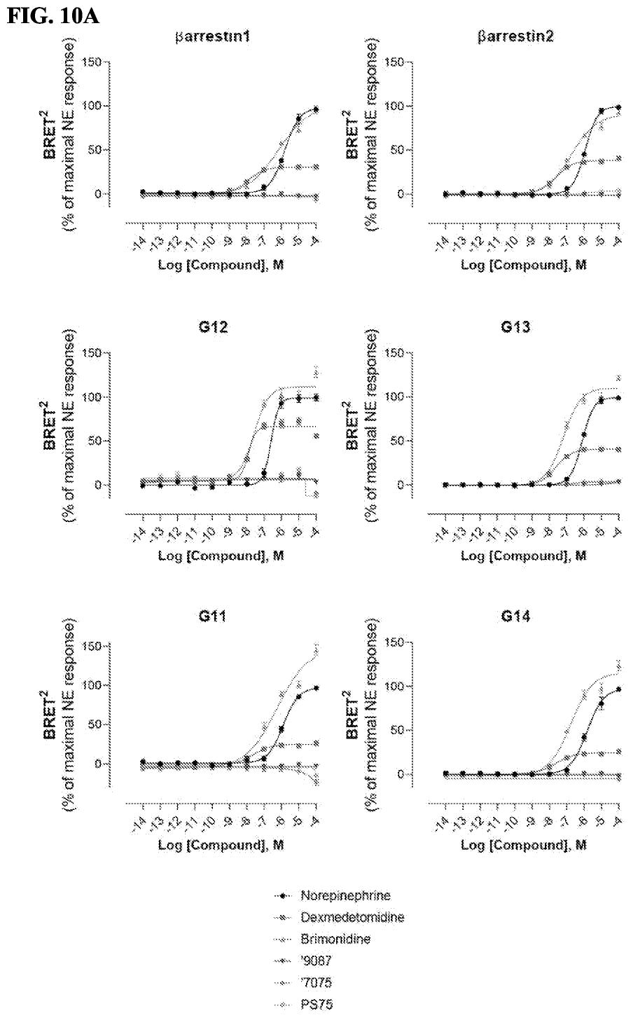

Gratifyingly, in functional assays most of the potent binders were partial or full agonists for α_{2A}AR and α_{2B}AR (FIGS. 1B-1D, FIGS. 6A-6G, FIGS. 7A-7L, FIG. 8, Table 1); few antagonists were found among the more potent docking hits. This reflects the targeting of the activated state of the receptor (35, 36) and was a goal of the screen. The best four agonists from the docking screen include ‘9087, ZINC1240664622, ZINC1242282998, and ZINC001242890172 (from here on referred to as ‘4622, ‘2998, and ‘0172, respectively), with the α_{2A}AR-mediated Gᵢ activation Eₘₐₓ ranging from 60-95% of norepinephrine response and EC₅₀ values of 9.7 to 210 nM in Gαᵢ BRET assays (FIG. 1C, Table 1). We tested the effect of receptor expression in cells on the functional properties of the partial agonist, ‘9087, and ultimately of two optimized analogs, ‘7075 and PS75; all three remained potent Gᵢ partial agonists, with Eₘₐₓ decreasing with receptor expression (FIGS. 9A-9F). In an orthogonal cAMP assay, ‘9087 was a partial agonist with an EC₅₀ of 87 nM and Eₘₐₓ of 42%, which is broadly consistent with the BRET assay (from here on Gᵢ activities are the Gαi BRET assay values unless otherwise noted) (FIG. 1C, FIG. 8, Table 1).

The new agonists had strong differential activity for Gᵢ activation compared to recruitment of β-arrestin-2. Although this was also true of the known agonists, dexmedetomidine and clonidine, for the new agonists the difference was accentuated so that arrestin recruitment was almost completely eliminated at relevant concentrations. Indeed, of the four best new agonists, only ‘0172 had a measurable efficacy for β-arrestin-2 recruitment, but even here only with 22% of the Eₘₐₓ of norepinephrine, and with weak potency (EC₅₀=1.7 μM); for the other three, β-arrestin-2 recruitment was negligible (FIG. 1C, Table 1). We note that this lack of arrestin recruitment could reflect the partial agonism of the new agonists combined with the weaker coupling of the arrestin pathway versus the well-coupled Gᵢ pathway, as indicated by the differences in potency and efficacy of the reference agonists dexmedetomidine and clonidine.

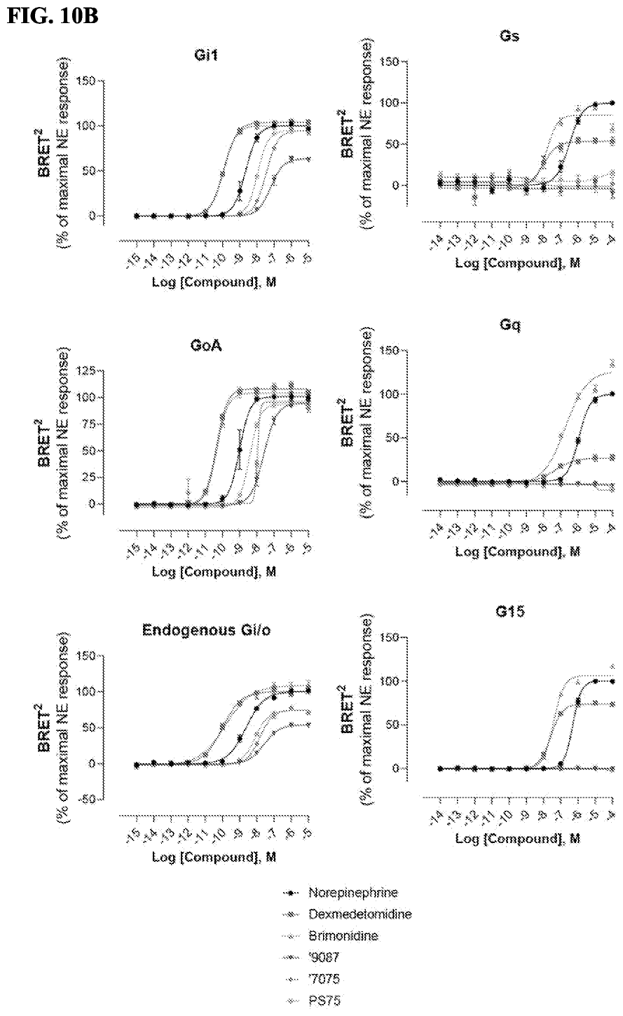

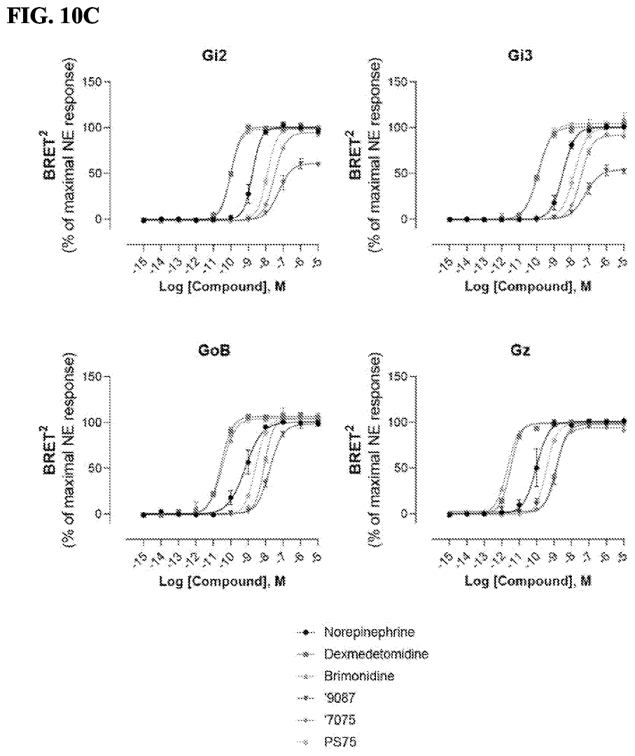

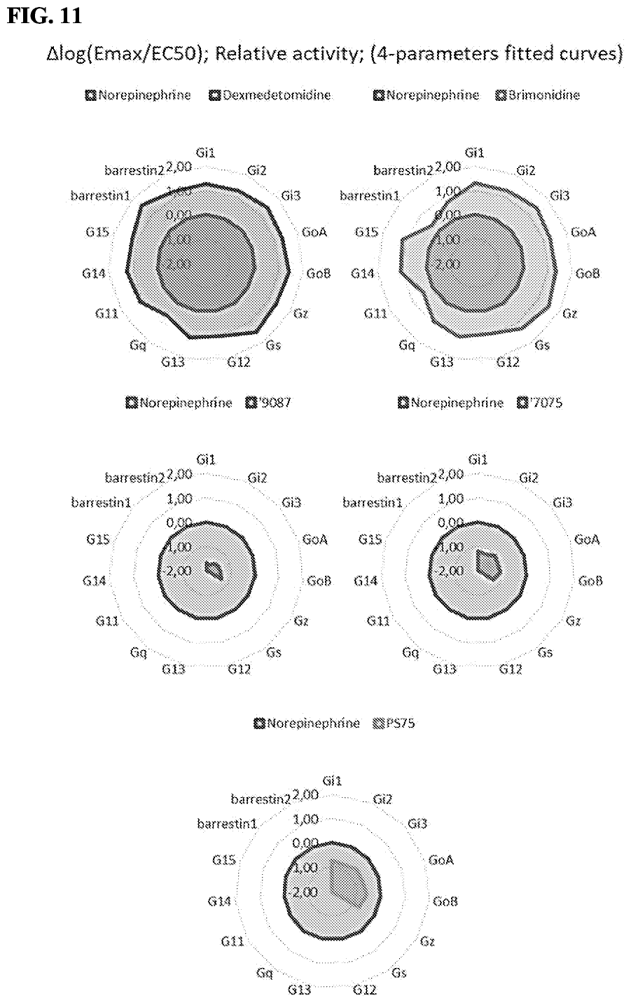

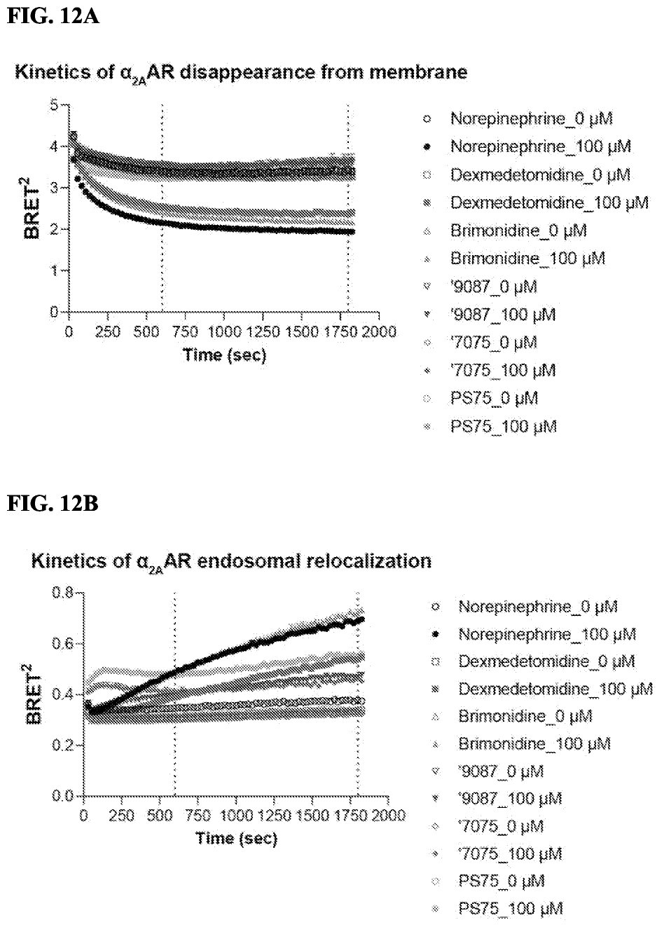

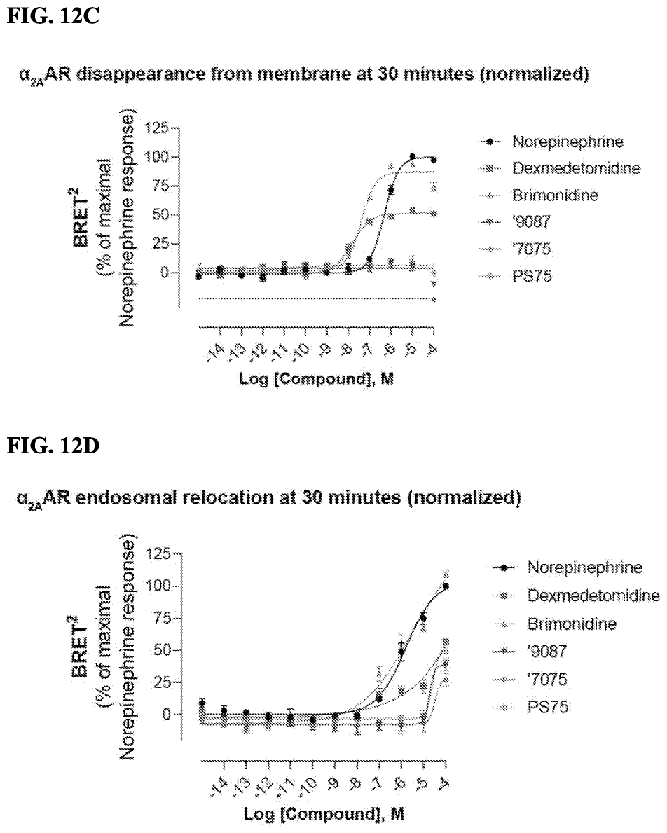

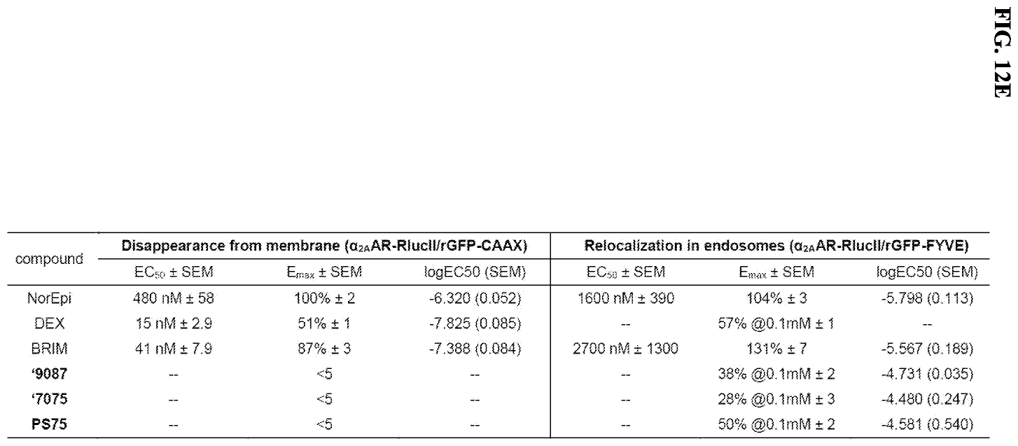

Agonists of α_{2A}AR, including its endogenous ligand, norepinephrine, also activate other G protein pathways (37). Accordingly, we used the Enhanced bystander BRET (ebBRET)-based effector Membrane Translocation Assay (EMTA) (38) to test ‘9087 and its analogs, ‘7075 and PS75, against a more expansive panel of G protein and β-arrestin subtypes. The docking compounds preferentially activated G_{i/o/z} signaling, while known agonists, norepinephrine, dexmedetomidine, and brimonidine strongly activated multiple additional G proteins (FIGS. 10A-10C, FIG. 11, Table 2). Receptor internalization following treatment with compound was also investigated by monitoring disappearance of α_{2A}ARs from the plasma membrane ((α_{2A}AR-RlucII/rGFP-CAAX biosensor) and relocalization of the receptors in endosomes ((α_{2A}AR-RlucII/rGFP-FYVE biosensor) (39). Known agonists brimonidine and norepinephrine show comparable responses for both biosensors, while dexmedetomidine has about half of this response. Consistent with their absence of β-arrestin recruitment, we found no effect of ‘9087, ‘7075, and PS75 on disappearance from the plasma membrane and marginal effect at the highest concentrations on endosomal relocalization (FIGS. 12A-12E). Although such functional selectivity was not explicitly modeled in the docking, it likely results from the novel chemistry, which was explicitly required (13, 14, 17).

Comparing the new agonists to dexmedetomidine, clonidine, norepinephrine, and to a previously described pharmacophore model for αAR selective agonists (9), both similar and distinct features emerge (FIG. 1B). The pharmacophore model for known agonists and the new docking compounds both have basic, nitrogen-containing rings. However, known agonists are dominated by imidazoles (unsaturated or partially saturated) while the docking compounds have diverse, non-imidazole rings. Both sets of compounds contain additional moieties off of a second aryl ring, typically two substituents for the known agonists; however, for the docking-derived compounds these vary from bulky hydrophobic rings, to hydrophilic rings, to single substituents, to having no substituents off of the aryl ring at all. Not all of the docking compounds have an exocyclic linker as described in the pharmacophore model. The protonated imidazole of known agonists ion pairs with D92^{3.32} and hydrogen bonds to the backbone of F412^{7.39} of α_{2B}AR (9, 40). Although several of the docking-derived compounds also interacted with both D92^{3.32} and F412^{7.39}, they did so with different heterocyclic rings (FIG. 1D).

To test the docking model and to template structure-based optimization, we determined the structure of the ‘9087-α_{2A}AR-Goα and ‘4622-α_{2A}AR-Goα complexes at a nominal resolution of 3.47 Å and 3.38 Å, respectively, using single particle cryo-electron microscopy (cryo-EM) (FIGS. 2A-2D). The predicted docked pose superimposes on the cryo-EM result of ‘9087 with a 1.14 Å all-atom RMSD of the agonist; the docking-predicted interactions are recapitulated in the experimental structure (FIG. 2B). The interactions between ‘9087 and α_{2A}AR differ from that of norepinephrine, but resemble those of imidazoline-containing agonists (9, 40). ‘9087 interacts with α_{2A}AR mainly through van der Waals and aromatic interactions to transmembrane helices (TM) 3, 5, 6 and 7 and I205^{45.52} of extracellular loop 2 (ECL2). It also forms an ionic interaction with the conserved D128^{3.32}, and although this interaction is relatively distant at 3.6 Å, it is similar to those of norepinephrine (40) and dexmedetomidine (9) that are 3.0 Å and 3.7 Å from D128^{3.32}, respectively (FIG. 2B). As in the docking prediction, the basic, formally cationic nitrogen of ‘9087 is not oriented toward D128^{3.32} to form a salt bridge (FIG. 2B), as seen in norepinephrine, but instead hydrogen bonds with the backbone carbonyl of F427^{7.39}, as does dexmedetomidine (9). The bridging exocyclic and formally neutral amine of ‘9087 ion pairs with D128^{3.32}. Typically for aminergic G protein-coupled receptors (GPCRs), the conserved hydrogen bond with D128^{3.32} would be made by the stronger base (9, 17, 20, 40). In fact, the formal charge of ‘9087 after protonation of the pyridine moiety is almost equally shared between the two nitrogens, as calculated by semi-empirical quantum mechanics and as reflected in the docking model. For ‘4622 the docked pose is also in good agreement with the cryo-EM result with an all-atom RMSD of 1.14 Å; ‘4622 forms a 3.4 Å hydrogen bond to D128^{3.32} and makes several hydrophobic interactions (FIG. 2D). Both ‘4622 and ‘9087-bound structures have similar receptor-GoA interfaces to other ligand-bound α₂AR-G protein complexes.

The interactions observed in the ‘9087 and ‘4622 receptor complexes, and in the modeled pose of analog ‘7075, were tested by residue substitution for impacts on Gᵢ activation and β-arrestin recruitment. Consistent with the observed ion pair with D128^{3.32}, norepinephrine and dexmedetomidine are highly sensitive to substitutions to D128^{3.32}, with an almost complete loss of Gᵢ activation and β-arrestin recruitment. For dexmedetomidine, the Gᵢ EC₅₀ is 170,000-fold higher for activation of D128^{3.32}A. While the Gᵢ activity of ‘9087 and ‘7075 is also diminished in the D128^{3.32} mutant receptors, potency only falls by about 200 to 1600-fold. In contrast, the Gᵢ activity of ‘9087, ‘7075 and ‘4622 is eliminated in the F427^{7.39}A mutant. The backbone carbonyl of F427^{7.39} hydrogen-bonds with ‘9087, while its aromatic side chain stacks with the agonist in the cryo-EM structure, perhaps indicating formation of a cation-pi interaction between the pyridine of ‘9087 and F427^{7.39} as previously suggested for agonist-induced α₂AR activation (41). Meanwhile, dexmedetomidine and especially norepinephrine, which lack these interactions, are less affected by this mutant (FIG. 2B). Mutations of Y409^{6.55} greatly affect norepinephrine, increasing (weakening) Gᵢ EC₅₀ values 500 to 10,000-fold, likely disrupting a key hydrogen bond (40, 41); the importance of position 6.55 was previously observed in agonist-induced β₂AR activation (42-44). The potencies of ‘9087 and dexmedetomidine are only modestly worse in Y409^{6.55} mutants and for ‘9087 the Gᵢ Eₘₐₓ is even slightly increased. For ‘4622 and ‘7075, most substitutions diminished activity, with the exception of S215^{5.42}A, which slightly increased the agonist activity of ‘7075 and ‘4622 in the Gᵢ activation and β-arrestin recruitment assays, and hardly influenced ‘9087 and dexmedetomidine. In contrast, S215^{5.42}A negatively affected norepinephrine-induced receptor activation, consistent with previous reports on direct interactions of full agonists and S215^{5.42} (40, 41). The Y431^{7.43}A/F mutations overall influenced β-arrestin recruitment of norepinephrine more than Gᵢ signaling. This has been previously observed, leading to the proposal that direct hydrogen bonding between the agonist and the residue at position 7.43 could more tightly couple TM7 and thereby play a role in β-arrestin signaling (40). Taken together, the differential responses to the residue substitutions supports suggestions from the structures that the new agonists, while binding in the same overall site as the canonical agonists, interact in meaningfully different ways, with potential implications for differential receptor signaling.

### Optimization of Docking Hit ‘9087

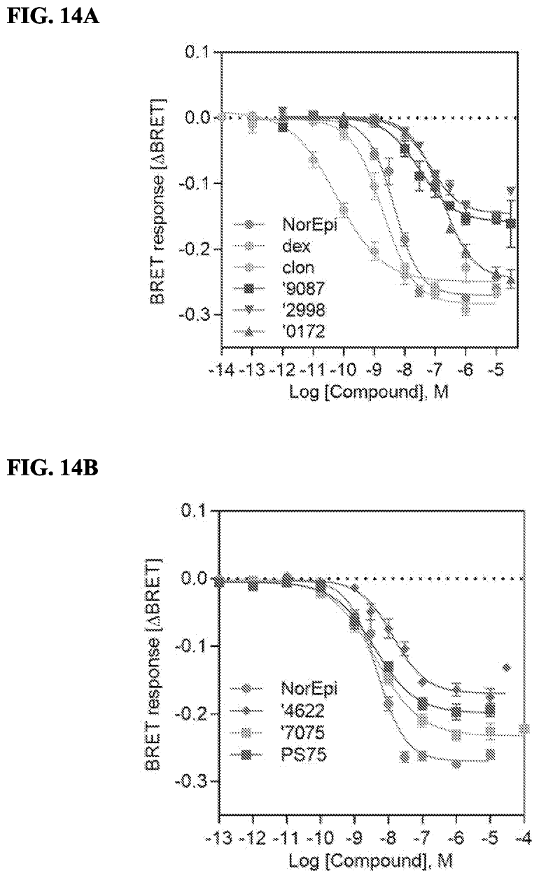

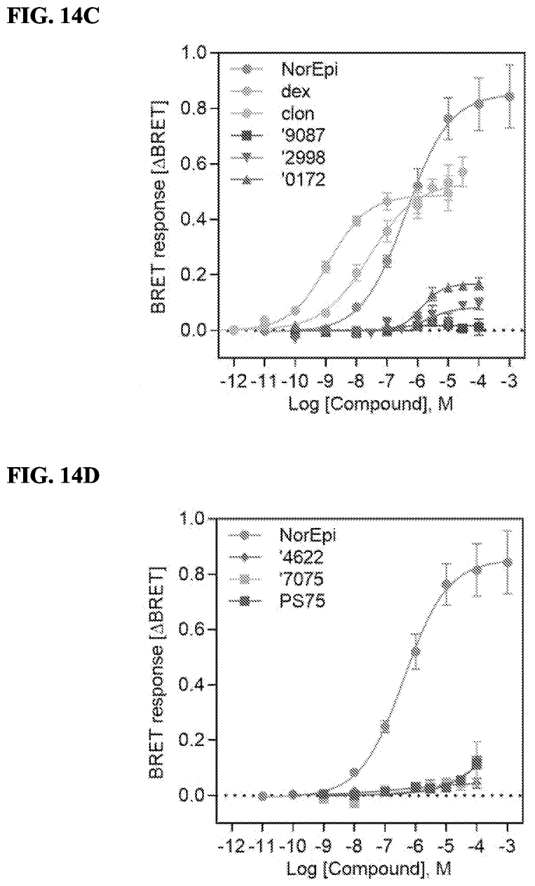

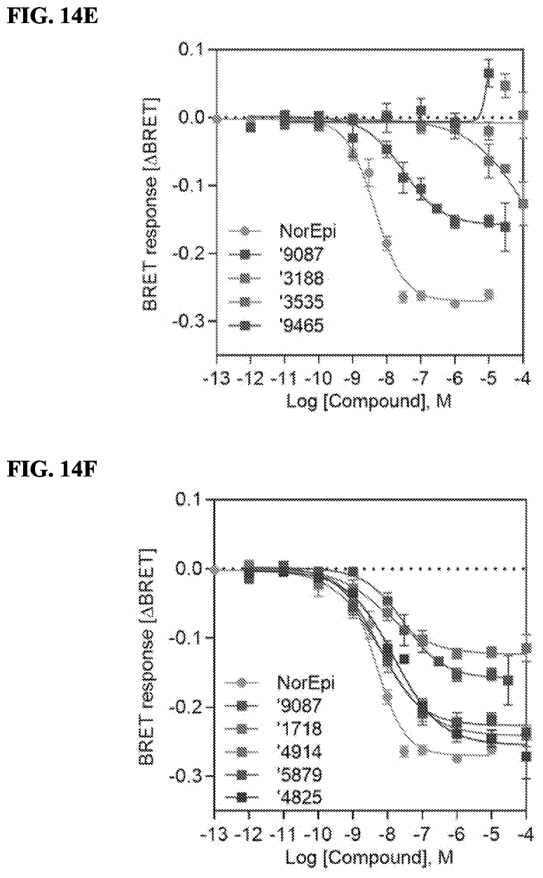

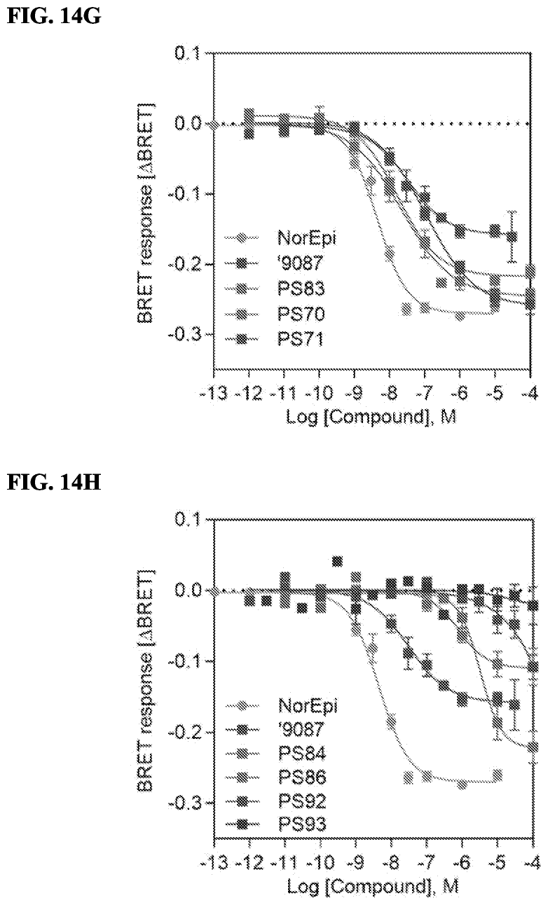

To optimize ‘9087, we adopted two strategies. First, we used classic medicinal chemistry hypothesis-testing and generation of analogs to investigate and improve key recognition features. We looked for possible analogs by similarity-searching among 1.4 billion and 12 billion tangible molecules using the Arthor and SmallWorld programs (12) (NextMove Software, England). From these searches, we docked prioritized subsets, leading to 13 of the 19 ‘9087 analogs we ultimately selected (FIG. 3D). Another six analogs were designed to probe particular protein-ligand interactions. Analogs were also investigated around compounds ‘2998, ‘4622, and ‘0172. In the ‘9087 series, perturbing the pyridine eliminated activity and confirmed the importance of the cationic character and the importance of the hydrogen bond with F427^{7.39} (FIG. 3D, FIGS. 13A-13D, FIGS. 14A-14H, Table 3).

The most potent analogs emerged from variations of the isoquinoline ring in ‘9087. Proximal hydrophobic residues F405^{6.51}, F406^{6.52}, Y409^{6.55}, and I205^{45.52} do not make obvious polar interactions with the isoquinoline nitrogen of ‘9087 (FIG. 2B). Accordingly, we added small non-polar groups, like the chlorine in ‘1718, or changed the isoquinoline to a different bicyclic system, like the benzothiophene in ‘4914 or naphthalene in ‘5879. A set of analogs also had the isoquinoline to naphthalene change, but with a single substituent added at two different positions of the naphthalene, as in ‘4825 and PS83. Overall, this set of analogs resulted in five potent agonists (EC₅₀ 4.1 nM to 18 nM) (Data supporting the ‘9087 optimization are summarized in FIG. 3B, FIG. 3D, FIGS. 13A-13D, FIGS. 14A-14H, Table 1, Table 3). ‘7075 was the most potent full agonist in the ‘9087 series with 13-fold increased potency in the BRET Gᵢ activation (EC₅₀ 4.1 nM, Eₘₐₓ 93%) and cAMP assays (EC₅₀ 18 nM, Eₘₐₓ 96%) (FIG. 3B, FIG. 8, Table 1). It preferentially activated G_{i/o} signaling over other G protein subtypes and β-arrestins, and caused no receptor internalization (FIGS. 10A-10C, FIG. 11, FIGS. 12A-12E, Table 2).

Our second strategy for ligand optimization was purely structure-based, using the newly-determined ‘9087-α_{2A}AR complex. Molecules were prioritized for their favorable docked pose in a ligand-free version of the α_{2A}AR-‘9087 structure, or designed to improve protein-ligand interactions based on the ‘9087-alpha2a cryoEM structure, leading us to synthesize eight further analogs. Two derivatives of ‘9087 (PS84 and PS86) confirm the importance of the lipophilic and aromatic properties of the bicyclic moiety for α_{2A}AR binding and activation, facilitating favorable interactions with the aromatic residues F405^{6.51}, F406^{6.52} and Y409^{6.55} (FIG. 2B). Other substitutions confirmed the importance of the ion-pair to D128^{3.32} and of the interaction between the protonated pyridine of ‘9087 and the backbone carbonyl atom of F427^{7.39} (PS92, PS93), as also shown by the mutational analysis.

Seeking more potent analogs, we focused on derivatizing the lipophilic substituents of ‘9087, building off analogs ‘5879 and ‘7075. Assuming the same binding mode for the naphthalene derivative ‘5879 as for ‘9087 in the cryo-EM structure, unexploited space between the ligand and the receptor in the orthosteric site was revealed in positions 5 and 7 of the bicyclic moiety of ‘5879 (R¹ and R² in FIG. 3D). We first tried to fill available space with substituents of different size at the R¹ position of ‘5879. From largest to smallest substituents were methoxy (PS71), chlorine (PS75), and fluorine (PS70). The potency of PS75 for Gᵢ activation was similar to ‘5879 (EC₅₀ of 4.8 nM and 6.1 nM, respectively), PS70 (EC₅₀ 36 nM) did not improve activity, and PS71 had 16-fold worse activity compared to ‘5879; for PS71 this may reflect entropic and desolvation penalties, and a repulsive interaction for the bulky methoxy substituent that the receptor was unable to accommodate. Similar to ‘7075 with a substituent at the R² position of the bicyclic moiety, addition of a methyl (PS83) had a similar EC₅₀ of 13 nM to ‘7075 and ‘5879 (EC₅₀ s of 4.1 nM and 6.1 nM, respectively).

PS75 was the most potent analog to arise from the second round of optimization. The molecule was a full agonist with 11-fold improved potency (EC₅₀ 4.8 nM, Eₘₐₓ 82%) for Gᵢ activation than ‘9087, and more potently activated G_{i/o/z} subtypes than did ‘9087. Meanwhile, PS75 still retained the preferential signaling through the G_{i/o} versus other G protein families and β-arrestins, and again led to negligible receptor internalization (FIGS. 9A-9F, FIGS. 10A-10C, FIG. 11, FIGS. 12A-12E, Table 2). In the modeled pose of PS75, the chlorine substituent is oriented towards the open space below its naphthalene ring towards T133^{3.37} (FIG. 3C). Its potency and efficacy make PS75 a lead molecule for treatment in pain.

Novel α_{2A}AR Agonists are Analgesic with Reduced Side Effects

In preparation for in vivo studies, we investigated the selectivity and pharmacokinetic properties of our most potent agonists. ‘9087 activated only a few of 320 GPCRs screened (45) (FIG. 15A). Only the dopamine D₂ receptor (D2R) had weak activity in secondary assays, with EC₅₀ values of 4.5 μM and 16 μM in G protein signaling and β-arrestin recruitment, respectively (FIG. 15B). ‘9087 did not measurably activate the μ-opioid receptor (OR) nor did it inhibit the human Ether-á-go-go-Related Gene (hERG) at concentrations below 10 μM (FIGS. 15C-15D). In binding experiments to other adrenergic receptors, ‘9087 bound to the α_{2C}-subtype at mid-nM concentration and to other α₁-subtypes in the 1 to 10 μM range (Table 4). The molecule had no measurable binding for β-adrenergic receptors up to 10 μM. Against the imidazoline-2 receptor (I2R), a common off-target of α_{2A}AR agonists, ‘9087 bound with a Kᵢ of 300 nM, showing a modest 6-fold selectivity for the α_{2A}AR, while a few docking-derived compounds actually had higher affinities for I2R than for the α_{2A}AR (FIG. 15E).

Computational models suggested that ‘9087, ‘4622, ‘7075 and ‘2998 would all have good physiologic permeability, consistent with their small size, low topological polar surface area, and weakly basic character (Table 5). Consistent with this prediction, on 10 mg/kg intraperitoneal (i.p.) injection in mice the first three compounds, especially, had high brain and cerebral spinal fluid (CSF) exposure, indicating the compounds are likely to reach centrally acting α_{2A}ARs (Table 5). ‘9087 reached a similar maximum concentration (Cₘₐₓ) in the CSF as did ‘7075, both of which were 4-fold greater than the Cₘₐₓ of PS75, and ‘9087 had a 12 to 20-fold higher area under the concentration-time curve (AUC) in the CSF than either ‘7075 and PS75; CSF concentrations are often used as a proxy for fraction unbound in the brain (46). Encouragingly, ‘9087 reached high brain exposure after both intravenous (i.v.) and oral (p.o.) administration, with AUC values of 420,000 ng*min/mL and 2,540,000 ng*min/mL (Table 5). The oral bioavailability was higher than 100%, which may reflect metabolic saturation at non-equal i.v. and p.o. doses, or entero-hepatic recirculation (Table 5); this merits further investigation. PS75 was fully bioavailable, though as an analog of ‘9087 the same caveats apply. We also investigated the metabolic stability of ‘9087, ‘7075 and PS75 in male rat liver microsomes. All three compounds remain largely unmodified after 1 hour, with ‘9087 having lower clearance and a higher half-life than its two analogs (FIG. 16).

Given their selectivity and high brain exposures, we tested the more potent agonists for pain-relief following systemic dosing (FIGS. 4B-4G and FIG. 4I). Initial doses were chosen to be less than the 10 mg/kg dose used in pharmacokinetics studies due to favorable CSF and brain properties. We started with ‘9087, the initial docking hit, and evaluated analogs as they emerged from compound optimization. With naïve (uninjured) mice, ‘9087 did not increase baseline mechanical withdrawal thresholds, something observed with many anti-pain medications, which often only have an anti-nociceptive effect in the presence of pain. We then investigated the activity of ‘9087 in a mouse model of neuropathic pain, in which partial peripheral nerve injury invokes profound mechanical hypersensitivity (47). Systemic subcutaneous (s.c.) injections of ‘9087 dose-dependently increased the mechanical thresholds of spared nerve injured (SNI) mice, with a sharp increase in activity from 3 mg/kg to 5 mg/kg, at which points the effects plateaued (FIG. 4I). Lower doses were anti-allodynic, returning mechanical thresholds to pre-injury levels, while the higher doses were genuinely analgesic, generating mechanical thresholds substantially higher than baseline, pre-injury levels. ‘9087 also increased thermal latencies in the complete Freund's adjuvant (CFA)-mediated inflammatory pain model, indicating that the molecule is effective in both tissue and nerve injury-induced pain models (FIG. 4G). ‘9087 also increased withdrawal latencies in the hot plate (55° C.) and tail flick (50° C.) assays of acute thermal (heat) pain (FIGS. 4E-4F). Consistent with its relatively high exposure on oral dosing, this molecule also conferred a dose-dependent anti-allodynic effect when delivered orally in the SNI neuropathic pain model (FIG. 4I). Doses up to 20 mg/kg of ‘9087 did not reduce the ability of the mice to perform in the rotarod test, which contrasts with the complete sedation of a dexmedetomidine dose of 60 μg/kg (FIG. 4J). This finding is an important differentiator for the new series, and also indicates that the analgesic effects of ‘9087 are not due to motor impairment.

We also investigated the mechanistic bases for the analgesia of the new α_{2A}AR agonists, both pharmacologically and genetically. Pharmacologically, the analgesic effect of ‘9087 was reversed by a systemic injection of the well-known α₂AR antagonist, atipamezole (2 mg/kg; administered 15 minutes prior to ‘9087) (FIG. 4C). Because atipamezole has broad activity against the α₂AR receptor subtypes and imidazoline receptors (48), we also tested ‘9087 in mice that express an inactive form of the α_{2A}AR (point mutation D79N) (5, 49-51). D79N mutant mice were tested in the tail flick (50° C.) assays. As previously reported, dexmedetomidine no longer induced analgesia in the mutant mice (52), and as a control the analgesia conferred by a 10 mg/kg dose of morphine was not significantly altered by the mutation (FIG. 4D). Consistent with activity through α_{2A}AR, the analgesia conferred by ‘9087 was reduced by over 50% back to baseline in the D79N mutant mice (FIG. 4D). Whereas most of the anti-nociceptive activity appears to derive from activity at the α_{2A}AR, we cannot discount contributions from other receptors.

Five other of the novel α_{2A}AR agonists (‘2998, ‘4622, ‘0172, ‘7075, PS75) also exhibited anti-allodynic effects in the SNI mice (FIG. 4B, FIG. 4I). The ‘9087 analogs, ‘7075 and PS75, completely reversed the mechanical hypersensitivity in the neuropathic pain model, with PS75 being more effective than ‘9087 (FIG. 4I). In contrast to ‘9087, PS75 did increase the mechanical thresholds of naïve (uninjured) mice (FIG. 4I). The anti-allodynic effects of ‘4622 and ‘7075 were reversed by atipamezole (48); while this antagonist also partially reversed the anti-allodynia of PS75, ‘2998, and ‘0172, these effects did not reach statistical significance at the small numbers of mice tested (FIG. 4C). PS75 also increased withdrawal latencies in the tail flick (50° C.) acute thermal pain assay, and when tested in the D79N mutant mice, its analgesic effect was reduced by over 50% (FIGS. 4D-4E). Compounds ‘0172 and ‘4622 also exhibited anti-hyperalgesic effects in the CFA inflammatory pain model (FIG. 4G); ‘2998 did not, which may reflect the reduced brain penetration of this molecule (Table 5). Only ‘4622 caused slight motor impairment at its equi-analgesic dose in the rotarod test, however, the effect did not reach the full sedation observed with dexmedetomidine (FIG. 4J). As with ‘9087, increased dosing up to 20 mg/kg PS75 did not have an effect on the rotarod test (FIG. 4J). Taken together, these pharmacological and chemical-genetic epistasis experiments support a mechanism of action primarily through the α_{2A}AR receptor, though a lesser contribution of other α₂AR subtypes cannot be ruled out.

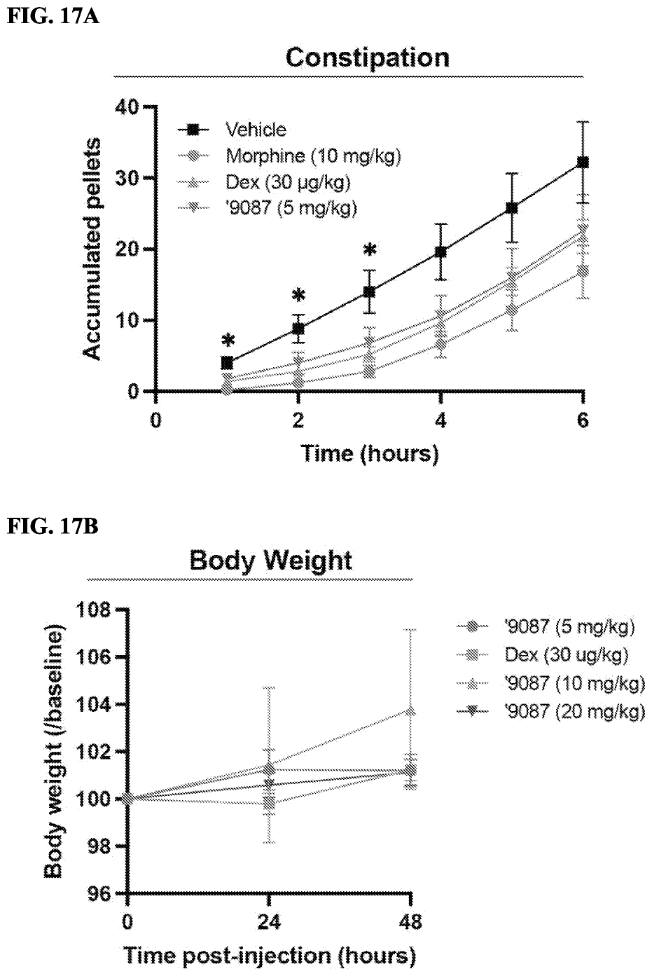

Some α_{2A}AR agonists can produce changes in feeding, weight gain and hyperglycemia (53, 54) as side effects. Accordingly, we evaluated the effect of compound treatment on body weight over 48 hours post-injection while allowing the mice to freely feed. We found no effect on body weight for ‘9087 dosed at 5 mg/kg, 10 mg/kg or 20 mg/kg, nor for dexmedetomidine dosed at 30 μg/kg (FIGS. 17A-17B). We also asked whether ‘9087 induced constipation, a side effect well-known for opioids and other classes of analgesics, comparing it to dexmedetomidine and to morphine tested at analgesic doses (30 μg/kg and 10 mg/kg, respectively). The number of accumulated pellets over 6 hours following vehicle or compound injection was measured. As expected, morphine induced constipation when compared to vehicle at the 1-, 2-, and 3-hour marks. In contrast, although pellet number did decrease modestly with ‘9087 and dexmedetomidine, neither effect differed significantly from vehicle (FIGS. 17A-17B). We recognize that other possible side effects remain untested in this study; we return to these in the Discussion.

Three key observations emerge from this study. First, multiple chemotypes, unrelated to known agonists, discovered directly from large-library docking are efficacious in neuropathic, inflammatory, and acute pain models through α_{2A}AR agonism (FIGS. 4B-4F and FIG. 4I). In docking, as in other target-based screens, the initial goal is to identify molecules with in vitro activity; these are then optimized for in vivo activity through extensive structure-activity optimization (13, 14, 17, 20). While it may be rare that direct hits from a docking screen are themselves in vivo active, such activity of the direct docking hits here does speak to the strengths of interrogating vast virtual libraries (11, 12). Second, functional assays reveal preferential G_{i/o/z} activation versus other G protein subtypes, no (3-arrestin activity and no receptor internalization for ‘9087 and its analogs, ‘7075 and PS75, compared to the established therapeutic α_{2A}AR agonists, like dexmedetomidine and brimonidine, that also activate multiple other G protein and β-arrestin signaling pathways (FIG. 1C, FIG. 3B, FIGS. 10A-10C, FIG. 11, FIGS. 12A-12E, Table 1). Although the high rate of agonist discovery was an intended outcome of docking against the activated state of the α_{2B}AR, the functional selectivity was not designed and can be attributed to the novel chemotypes they explore and by extension their use of both canonical and non-canonical receptor interactions (FIGS. 2A-2D). Third, unlike dexmedetomidine and clonidine (6, 7, 55), ‘9087 and its analogs, PS75 and ‘7075, do not cause sedation or motor impairment at analgesic doses, potentially enabling broader applications to pain treatment and attesting to the ability to differentiate these two effects with α_{2A}AR agonists (FIG. 4J).

The new chemotypes explored (FIG. 1B) reflect the size and diversity of the docked libraries. Most of the actives emerged from the fragment-like library in ZINC, which covers a much greater portion of the chemical universe in its size range than do the lead-like or drug-like libraries. This is akin to physical fragment libraries, which typically might include about 1,500 molecules (56) but are thought to cover more chemotypes than high-throughput screening libraries that are 1,000-fold larger. Meanwhile, the virtual fragment library in ZINC enumerates over 20 million molecules (11, 12), about 10,000-fold more than in most physical fragment libraries. Indeed, with over 800,000 Bemis-Murcko scaffolds (18), there are 500-fold more fragment scaffolds in the docked library than there are molecules in most physical fragment libraries. From this great chemotype diversity springs opportunities for ligands with new pharmacology.

From the cryo-EM co-complex with ‘9087 and substitution of binding site residues, ‘9087 and ‘7075 appear to make weaker interactions with D128^{3.32}, and an apparently stronger interaction with F427⁷³⁹, than do the canonical agonists (FIG. 2B). These structural differences may contribute to the unusual functional G_{i/o/z} selectivity over other G protein subtypes and β-arrestins as compared to known agonists, and to the lack of receptor internalization (FIG. 3B, FIGS. 10A-10C, FIG. 11, FIGS. 12A-12E). In turn, this unusual signaling may play a role in conferring analgesia without sedation (FIGS. 4B-4G and FIGS. 4I-4J). As the engagement of transducing G proteins and β-arrestins occurs 35 Å away from the orthosteric site, other mechanisms may be involved (57). Moreover, the physiological impact of the selective signaling will be entangled with the pharmacokinetics of the molecules. Irrespective, what should be clear is that the analgesic potential of α_{2A}AR agonists may be disentangled from their sedative effect, which is important for subsequent drug development.

From an ultra-large library docking screen emerged low nM (α_{2A}AR partial agonists, topologically unrelated to previously known ligands, making new interactions with the receptor that appear to confer new pharmacology (FIGS. 1A-1D, FIG. 2B, FIGS. 10A-10C, FIG. 11, FIGS. 12A-12E, Table 1). Several of the new agonists were anti-allodynic and analgesic in neuropathic and inflammatory pain models, and against acute nociception in naïve animals (FIGS. 4B-4G and FIG. 4I). Among the most promising are ‘9087 and PS75, both of which are strongly analgesic without the sedative effects of dexmedetomidine (FIGS. 30 4B-4G and FIGS. 4I-4J) and also orally bio-available (Table 5). These properties make the compounds plausible therapeutic leads for new non-opioid pain therapeutics without the sedation of classic α_{2A}AR drugs.

### Materials and Methods

Molecular docking. The α_{2B}AR receptor with dexmedetomidine and GOA (PDB 6K41) (9) was used for docking calculations prior to the determination of the α_{2B}AR dexmedetomidine-bound structure (PDB 7EJA). Three screens of the ZINC15 database were run, two for fragment molecules (less than 250 amu, c Log P<3.5) and one for lead-like (250-350 amu, c Log P<3.5). Docking was performed with DOCK3.7 (26). For the first screen, 45 matching spheres (26) were used, 15 from the docked pose of dexmedetomidine and 30 from SPHGEN-generated spheres (58). The receptor structure was protonated using REDUCE (59) and AMBER united atom charges were assigned (60). In control calculations (61) with 15 known agonists from the IUPHAR/BPS database (27) and from the literature (29-31), balanced against 1800 property matched decoys (62), docking parameters were optimized based on adjusted logAUC (61) and based on recapitulation of ligand interactions with residues α_{2B}AR D92^{3.32}, F412^{7.39}, F387^{6.51}, Y391^{6.55}, and F388^{6.52} (residues conserved in α_{2A}AR: D128^{3.32}, F427^{7.39}, F405^{6.51}, Y409^{6.55}, and F406^{6.52}). An “extrema” set was used to evaluate cationic charge preference, as described (18, 62). The protein low dielectric and desolvation regions, defined by pseudo-atoms calculated with SPHGEN (58), were extended as previously described (63), based on the control calculations, by a radius of 1 Å and 0.3 Å, respectively (10, 64). Energy potential grids were calculated using CHEMGRID for AMBER-based van der Waals potential, QNIFFT (65) for Poisson-Boltzmann-based electrostatic potentials, and SOLVMAP (66) for context-dependent ligand desolvation. In the second and third docking screens, differences included modified matching spheres (added rigid fragments of xylazine docked-pose, only used 40 matching spheres) and extension of the desolvation pseudo-atoms by a radius of 0.2 Å.

For the first screen, 20 million molecules from the ZINC15 (http://zinc15.docking.org/) fragments subset were docked in 3,008 core hours or about 6 wall-clock hours on a 500-core cluster. Almost 5 trillion complexes were sampled, on average each molecule sampled 2,405 orientations and 202 conformations. Only about 8 of 20 million could be sterically accommodated in the orthosteric site, reflecting its small size. For the second screen, the same 20 million fragments were docked in 3830 core hours or 7.7 hours on 500-core cluster, sampling over 6 trillion complexes; on average each molecule sampled 3,122 orientations and 203 conformations. About 9 million molecules were accommodated in the site. For the third screen, 281 million molecules from ZINC15 lead-like subset were screened in 71,625 core hours or about 1 week on 500 cores. Over 222 trillion complexes were sampled with an average of 4,553 orientations and 469 conformations per molecule, though ultimately only 13.5 million could sterically fit in the site.

For the first and second screens, the top 161,055 scored compounds were clustered by ECFP4-based Tanimoto coefficient (Tc) of 0.5 to identify unique chemotypes, resulting in 37,150 and 33,378 clusters. For the third screen, the top 300,000 scored compounds were clustered in the same manner resulting in 57,168 clusters. Molecules were filtered for novelty, removing those with Tc>0.35 to 15 α_{2A}AR agonists used in control calculations. The top 5,000 ranked molecules remaining were visually filtered for interactions at α_{2B}AR residues D92^{3.32}, F412^{7.39}, F387^{6.51}, Y391^{6.55}, and F388^{6.52} for the first and second screens; for the third screen, the top 20,000 molecules were examined by the same criteria. Lastly, prioritized molecules were also filtered for internal torsional strain; this was done visually for the first screen, while the second and third screens used a method drawing on CSD torsion populations cutting off at a total energy of 2 Torsion Energy Units (32). An additional novelty filter was performed removing molecules with TC>0.35 to CHEMBL29 (28) α_{2A}AR compounds. Sixty-four molecules were selected for purchasing: 33, 26, and 5 from the first, second and third screens, respectively. Ten were sourced from WuXi and another 54 from Enamine, of which 8 and 40 were successfully synthesized, respectively. Most of these compounds have not previously been synthesized before, to the best of our knowledge, except for some of the smaller fragments, which have been previously used as building blocks.

Synthesis of tangible molecules. Forty-eight molecules prioritized for purchasing were synthesized by Enamine and Wuxi for a total fulfilment rate of 75%. Compounds were sourced from the Enamine REAL database (https://enamine.net/compound-collections/real-compounds) or the WuXi GalaXi Virtual library. The purities of active molecules synthesized by Enamine and WuXi were at least 90% and typically above 95%. For bespoke compound synthesized in house purities were at least 96% and typically above 99%. The purity of compounds tested in vivo were >95% and typically above 98%.

Ligand optimization. Analogs for four docking hits (‘9087, ‘2998, ‘0172, ‘4622) were queried in Arthor and SmallWorld 1.4 and 12 Billion tangible libraries (https:/sw.docking.org/, https://arthor.docking.org), the latter primarily containing Enamine REAL Space compounds (https://enamine.net/compound-collections/real-compounds/real-space-navigator). Results from SmallWorld, Bemis-Murcko framework, and substructure queries were pooled, docked into the α_{2B}AR site prior to ‘9087-α_{2A}AR structure being determined. Compounds with favorable interactions in the orthosteric site were prioritized, leading to 13 analogs for ‘9087. Also, for the 4 docking hits, analogs were designed by modifying the 2D chemical structure to test specific hypotheses, adding another 6 analogs for ‘9087. The second round of analogs for ‘9087 were designed and prioritized for bespoke synthesis. Some were docked to a preliminary cryo-EM model of the ‘9087-α_{2A}AR structure, while several were designed and synthesized regardless of docked pose to test specific hypotheses; in total 8 of these were synthesized and tested. Calculation of the contact areas was performed by means of UCSF Chimera (https://rbvi.ucsf.edu/chimera).

Bespoke synthesis. ‘7075 was designed for hypothesis testing and was bespoke synthesized by Enamine.

Molecular modeling of ‘7075 and PS75. Maestro (v. 2019-4, Schradinger, LLC) was used to manually change the chemical structure of ‘9087 to ‘7075 or PS75 in a preliminary model of the ‘9087-α_{2A}AR complex cryo-EM structure. The isoquinoline nitrogen was changed to a carbon and the fluorine or chlorine substituent was added to the naphthalene ring for ‘7075 and PS75, respectively. The resulting complex of ‘7075 or PS75 and α_{2A}AR coupled to the G protein but without scFv16 was energy minimized following the Protein Preparation Wizard protocol using the OPLS3e force field. The maximum heavy atom deviation from the initial model was 0.3 Å.

Passive-membrane permeability prediction. Ligand structures were converted from SMILES strings to three-dimensional structures using LigPrep (v. 53013, Schrodinger, New York). For the passive-membrane permeability prediction (67, 68), we retained only neutral form for each ligand. Passive-membrane permeability of a ligand is predicted from the free-energy of insertion (ΔG_{I}), i.e., from the energy difference between a conformer in low and high dielectric media. Therefore, we generated conformations of each ligand using ConfGen software (v. 5.1, Schrodinger, New York). We minimized each conformer in a low dielectric medium (chloroform) to mimic the membrane dielectric using Protein Local Optimization Program (PLOP) (69). After finding the lowest energy conformer in the low dielectric medium, we calculated the energy of that energy-minimized conformer in water. We subtracted the energy of the ligand in the high-dielectric water from the low-dielectric medium. We further added a deionization penalty term to account for transforming ionized form of the ligand in water to its neutral form in membrane. We computed the deionization penalty energy using the empirical pKa prediction software Epik (v. 5.1013, Schrodinger Inc.). We rank-ordered the ligands based on their free-energy of insertion.

Radioligand binding experiments. Receptor binding affinities for the α_{2A}AR receptor and to α_{2B}AR as well as the related adrenergic subtypes α_{1A}, +_{1B}, α_{2C}, β₁ and β₂ were determined as described previously (44, 70). In brief, membranes were prepared from HEK293T cells transiently transfected with the cDNA for human α_{2A}AR, murine α_{2A}AR (provided by D. Calebiro, Birmingham, UK), human α_{2B}AR (obtained from the cDNA resource center, www.cdna.org) or with the cDNAs for the human α_{1A}, α_{1B}, α_{2C}, β₂ (all from cDNA resource center) and β₁ (provided by R. Sunahara, UCSD). Receptor densities (Bₘₐₓ value) and specific binding affinities (K_{D} value) for the radioligand [³H]RX82,1002 (specific activity 52 Ci/mmol, Novandi, Södertälje, Sweden) were determined as 1,400±210 fmol/mg protein and 0.54±0.024 nM for human α_{2A}AR, 4,000±720 fmol/mg protein and 1.8±0.61 nM for murine α_{2A}AR, and 3,400±580 fmol/mg protein and 2.3±0.52 nM for α_{2B}AR, respectively. Further values are 3,200±1,900 fmol/mg protein and 0.58±0.11 nM for α_{2C}, 2,000±950 fmol/mg protein and 0.70±0.13 nM for α_{1A}, and 4,000 fmol/mg protein and 0.11 nM for α_{1B}, both determined with [³H]prazosin (51 Ci/mmol, PerkinElmer, Rodgau, Germany), respectively and 1,400±360 fmol/mg protein and 0.070±0.006 nM for 1, and 1,300±230 fmol/mg protein and 0.074±0.012 nM for 12, both determined with [³H]CGP12,188 (52 Ci/mmol, PerkinElmer).

Competition binding with α₂AR subtypes were performed by incubating membranes in buffer A (50 mM TRIS at pH 7.4) at final protein concentrations of 3-10 g/well with the radioligand (final concentration 0.5-2.0 nM according to the appropriate K_{D} and Bₘₐₓ) and varying concentrations of the competing ligands for 60 minutes at 37° C. Binding to α_{1A} and α_{1B} was measured with buffer B (50 mM TRIS, 5 mM MgCl₂, 1 mM EDTA, 100 μg/mL bacitracin and 5 μg/mL soybean trypsin inhibitor at pH 7.4) at 2-6 g/well (radioligand at 0.2-0.3 nM) and binding to 1 and 12 was measured with buffer C (25 mM HEPES, 5 mM MgCl₂, 1 mM EDTA, and 0.006% bovine serum albumin at pH 7.4) at 4-8 μg/well (radioligand 0.2 nM). Non-specific binding was determined in the presence of unlabeled ligand at 10 μM. Protein concentration was measured using the method of Lowry (71).

The resulting competition curves were analyzed by nonlinear regression using the algorithms implemented in Prism 8.0 (GraphPad Software, San Diego, CA) to provide IC₅₀ values, which were subsequently transformed into a Kᵢ values applying the equation of Cheng and Prusoff (72). Mean Kᵢ values (±s.e.m. for n≥3, or ±s.d. for n=2) were derived from 2-7 experiments each performed in triplicates.

### Functional Assays

Plasmids. The human wild type α_{2A}AR, its respective receptor mutants (73) and the murine α_{2A}AR, all carrying an N-terminal HA-signal sequence and a FLAG-tag, as well as the human adrenergic receptor subtypes α_{1A}, α_{1B}, α_{2C}, β₁ and β₂ and the dopamine receptor D_{2long} were cloned to pCDNA3.1 for G protein activation assays (BRET, IP accumulation). Human α_{2A}AR and α_{2B}AR were fused to the ARMS2-PK2 sequence and cloned to pCMV (DiscoverX, Eurofins) for β-arrestin-2 recruitment assays, respectively, using polymerase chain reaction and Gibson Assembly (New England Biolabs) (70). Sequence integrity was verified by DNA sequencing (Eurofins Genomics).

Bioluminescence resonance energy transfer. G protein activation by human α_{2A}AR and D_{2long} was monitored with Gαᵢ₁-RLucII (74, 75) together with Gβ₁ and Gγ₂-GFP₁₀. Assessment of arrestin recruitment was performed by enhanced bystander BRET using CAAX-rGFP and β-arrestin-2-RLucII as biosensors (39, 74) in the presence of GRK2 as described (44, 76). In brief, HEK293T cells (gift from the Chair of Physiology, FAU Erlangen-Nurnberg) were transfected with 200 ng receptor plasmid for G protein activation (receptor:Gα:Gβ:Gγratio 2:0.5:1:4) or 100 ng receptor plasmid for β-arrestin recruitment (receptor:β-arrestin:GRK2:CAAX ratio 1:0.2:1:3) using linear polyethyleneimine (PEI, Polysciences, 3:1 PEI:DNA ratio). The DNA was complemented to a total amount of 1 μg DNA per 3-10⁵ cells with ssDNA (Sigma Aldrich) and 10,000 cells per well were transferred into 96-well half-area plates (Greiner, Frickenhausen, Germany). Additional experiments were performed using the same amount of G protein plasmids as described above but 50 ng or 10 ng α_{2A}AR plasmid instead. 48 h after transfection, the cell medium was exchanged with PBS (phosphate buffered saline) and cells were stimulated with ligands at 37° C. for 10 min. Coelenterazine 400a (abcr GmbH, Karlsruhe, Germany) at a final concentration of 2.5 μM was added 5 min before measurement. BRET was monitored on a Clariostar plate reader (BMG, Ortenberg, Germany) with the appropriate filter sets (donor 410/80 nm, acceptor 515/30 nm) and was calculated as the ratio of acceptor emission to donor emission. BRET ratio was normalized to the effect of buffer (0%) and the maximum effect of norepinephrine (100%) for adrenergic receptors and quinpirole (100%) for D_{2long}. For each compound 3 to 17 individual experiments were performed each done in duplicates.

Surface expression of the α_{2A}AR in the G protein activation assays was monitored applying an enzyme-linked immunosorbent assay (ELISA) directed against the N-terminal FLAG tag. HEK293T cells were transfected with the cDNAs encoding α_{2A}AR, Gαᵢ₁-RLucII, Gβ₁, Gγ₂-GFP₁₀ and ssDNA as described above. As a control, cells transfected with only α_{2A}AR or mock pcDNA3.1 plasmid and ssDNA were used. Immediately after transfection, 50,000 cells/well were transferred to a 48-well plate (Greiner) pretreated with poly-D-lysine (Sigma Aldrich) and incubated at 37° C. and 5% CO₂ for 48 h. The medium was removed, cells were treated with 4% paraformaldehyde for 10 min, washed once (wash buffer, 150 mM NaCl, 25 mM Tris, pH 7.5), and blocked for 60 min (30 g·L−1 skim milk powder in wash buffer, all steps carried out at room temperature). After incubation with anti-FLAG M2 mouse IgG (F3165, Sigma Aldrich, 1:4,000 in blocking solution) for 60 min, cells were washed twice, blocked again for 60 min and incubated with anti-mouse rabbit IgG-HPR (A9044, Sigma Aldrich, 1:20,000 in blocking solution) for 60 min. Cells were washed thrice, before 200 μL substrate buffer was added (2.8 mM o-phenylenediamine, 35 mM citric acid, 66 mM Na₂HPO₄, pH 5.0). Reactions were kept in the dark for 5-15 min and stopped by addition of 1 M H₂SO₄ (200 μL). Resulting mixtures were transferred to a 96-well plate and absorption was determined with the Clariostar microplate reader at 492 nm. Data were normalized using cells transfected with only α_{2A}AR (100%) and mock pcDNA3.1 (0%), respectively. N=4 independent experiments were performed with each condition in triplicate.

The sensitivity of selected ligands to the receptor mutants α_{2A}AR-D128^{3.32}A, α_{2A}AR-D128^{3.32}T, α_{2A}AR-D128^{3.32}L, α_{2A}AR-S215^{5.42}A, α_{2A}AR-Y409^{6.55}A, α_{2A}AR-Y409^{6.55}T, α_{2A}AR-Y409^{6.55}F, α_{2A}AR-F427^{7.39}F, α_{2A}AR-Y431^{7.43}A, and α_{2A}AR-Y431^{7.43}F was monitored by G protein activation as described above transfecting the appropriate receptor together with Gαᵢ₁-RLucII and Gβ₁/Gγ₂-GFP₁₀. Data was analyzed as ligand induced changes in BRET compared to vehicle (deltaBRET) and additionally normalization was done according to the effect of buffer (0%) and norepinephrine (100%) with the exception of α_{2A}AR-D128^{3.32}A (dexmedetomidine=100%), α_{2A}AR-D128^{3.32}T and α_{2A}AR-D128^{3.32}L (‘7075=100%). Similarly, the effect of the α_{2A}AR-D128^{3.32}A, α_{2A}AR-S215^{5.42}A, α_{2A}AR-Y409^{6.55}A, α_{2A}AR-Y409^{6.55}F, α_{2A}AR-F427^{7.39}F, α_{2A}AR-Y431^{7.43}A, and α_{2A}AR-Y431^{7.43}F mutations on arrestin recruitment was evaluated as described above transfecting the appropriate receptor together with CAAX-rGFP, GRK2 and β-arrestin-2-RLucII. Data was analyzed as ligand induced changes in BRET compared to vehicle (deltaBRET). Three to seven experiments were done in duplicate.

IP accumulation assay. Determination of G protein mediated signaling by human α_{2A}AR, murine α_{2A}AR, and human α_{2B}AR was performed applying an IP accumulation assay (IP-One HTRF®, Cisbio, Codolet, France) according to the manufacturer's protocol and in analogy to previously described protocols (77, 78). In brief, HEK 293T cells were co-transfected with the cDNA for a receptor and the hybrid G-protein Gα_{qi} (Gα_{q} protein with the last five amino acids at the C-terminus replaced by the corresponding sequence of Gαᵢ(gift from The J. David Gladstone Institutes, San Francisco, CA), respectively in a ratio of 1:2. After one day cells were transferred into 384 well micro plates (Greiner) and incubated for further 24 hrs. On the day of the experiment cells were incubated with test compounds for 90 min ((α_{2A}AR) or 120 min ((α_{2B}AR) and accumulation of second messenger was stopped by adding detection reagents (IP1-d2 conjugate and Anti-IP1cryptate TB conjugate). After 60 min TR-FRET was monitored with a Clariostar plate reader. FRET-signals were normalized to buffer (0%) and the maximum effect of norepinephrine (100%). Three to nine (murine α_{2A}AR, α_{2B}AR) or 4-11 repeats (human α_{2A}AR), respectively in duplicate were performed for each test compound all done in duplicate.

PathHunter arrestin recruitment assay. Investigation of α_{2A}AR and α_{2B}AR stimulated β-arrestin-2 recruitment was performed applying an assay which is based on fragment complementation of β-galactosidase (PathHunter assay, DiscoverX, Birmingham, U.K.) as described (79). In detail, HEK293T cells stably expressing the enzyme acceptor (EA) tagged β-arrestin-2 were co-transfected with human α_{2A}AR or α_{2B}AR each fused to the ProLink-ARMS2-PKS2 fragment for enzyme complementation and GRK2 (cDNA Resource Center) at equal amounts and subsequently transferred into 384 well micro plates (Greiner) after 1 day. After incubation for further 24 hrs cells were incubated with test compounds for 60 min ((α_{2A}AR) or 90 min (α_{2A}R), arrestin recruitment was stopped by adding detection regent and the resulting chemoluminescence was monitored with a Clariostar plate microreader. Data was normalized relative to buffer (0%) and the maximum effect of norepinephrine (100%). Three to nine repeats for α_{2A}AR (3-6 for α_{2B}AR) in duplicate were measured.

DiscoverX HitHunter cAMP G-protein activation assay. Dexmedetomidine, brimonidine, ‘9087, and ‘7075 were tested by DiscoverX (catalog item 86-0007P-2270AG; Eurofins; CA, USA) in their HitHunter XS+ assay. Freezer stock cAMP Hunter cell lines were expanded, then seeded in a total volume of 20 μL into white walled, 384-well microplates and incubated at 37° C. prior to testing. For agonist determination, cells were incubated with compound samples in the presence of EC₅₀ forskolin to induce response. Media was aspirated from cells and replaced with 15 μL 2:1 HBSS/10 mM Hepes: cAMP XS+Ab reagent. Intermediate dilution of sample stocks was performed to generate 4× sample in assay buffer containing 4× EC₈₀ forskolin. 5 μL of 4× sample was added to cells and incubated at 37° C. or room temperature for 30 to 60 minutes. Finally assay vehicle concentration was 1%. After sample incubation assay signal was generated through incubation with 20 μL cAMP XS+ED/CL lysis cocktail for one hour followed by incubation with 20 μL cAMP XS+EA reagent for three hours at room temperature. Microplates were read following signal generation with a PerkinElmer Envision Instrument for chemiluminescent signal detection. Compound activity was analyzed using CBIS data analysis suite. For Gᵢ agonist mode, percentage activity is calculated using the following formula: % Activity=100%×(1−(mean RLU of test sample−mean RLU of MAX control)/(mean RLU of vehicle control−mean RLU of MAX control)). Brimonidine was used as the control agonist. Each measurement was done in duplicate.

### EMTA Coupling Panel for α_{2A}AR

The ebBRET-based effector membrane translocation assay (EMTA) allows detection of each Ga protein subunit activation. Upon receptor activation, G protein-effector proteins fused at their C-terminus to Renilla luciferase (RlucII) translocate from cytoplasm to the plasma membrane to selectively bind activated Gα proteins (p63-RhoGEF-RlucII with G_{q/11} family, Rap1GAP-RlucII with G_{i/o} family and PDZ-RhoGEF-RlucII with G_{12/13} family), thus leading to an increase in ebBRET by becoming in close proximity to the plasma membrane targeted energy acceptor, Renilla green fluorescent protein (rGFP-CAAX). The heterologous co-expression of each Ga subunits allow to identify which specific members of each G protein families (i.e., Gᵢ₁, Gᵢ₂, Gᵢ₃, G_{oA}, G_{oB}, G_{z}, G_{q}, G₁₁, G₁₄, G₁₅, G₁₂ and G₁₃) is activated by a receptor. The assay is also sensitive enough to detect responses elicited by endogenous G_{i/o} protein families in the absence of heterologously expressed G protein. The same plasma membrane translocation principle is used to measure β-arrestin-1 or -2 recruitment (39) using β-arrestin-RlucII/rGFP-CAAX biosensors.

Cell Culture. HEK293 clonal cell line (HEK293SL cells), hereafter referred as HEK293 cells, were a gift from S. Laporte (McGill University, Montreal, Quebec, Canada) and previously described (39). Cells were cultured in DMEM medium (Wisent; St-Jean-Baptiste, QC, Canada) supplemented with 10% newborn calf serum iron fortified (NCS; Wisent). Cells were passaged weekly and incubated at 37° C. in a humidified atmosphere with 5% CO₂ and checked for mycoplasma contamination.

Transfection. HEK293 cells (1.2 mL at 3.5×10⁵ cells per mL) were transfected with a fixed final amount of pre-mixed biosensor-encoding DNA (0.57 μg, adjusted with salmon sperm DNA; Invitrogen) and human α_{2A}AR DNA for Gₛ, G_{i/o}, G_{q/11} and β-arrestins experiments. For G_{12/13} experiments, cells were transfected with 1 μg of total DNA (adjusted with salmon sperm DNA; Invitrogen), including empty pCDNA3.1 vector or human α_{2A}AR DNA. Transfections were performed using linear polyethylenimine (PEI, 1 mg/mL; Polysciences) diluted in NaCl (150 mM, pH 7.0) as a transfecting agent (3:1 PEI/DNA ratio). Cells were immediately seeded (3.5×10⁴ cells/well) into 96-well white microplates (Perkin Elmer), maintained in culture for the next 48 h and BRET experiments carried out. ebBRET (38) was used to monitor the activation of each Ga protein, as well as β-arrestin-1 and -2 recruitment to the plasma membrane. Gas protein engagement was measured between the plasma membrane marker rGFP-CAAX and human Gas-RlucII in presence of human Gβ₁, Gγ₉ and α_{2A}AR. Gα₁₂ or Gα₁₃ protein family activation was assessed using the selective-G_{12/13} effector PDZ-RhoGEF-RlucII and rGFP-CAAX co-expressed with Gβ₁, Gγ₁ and either Gα₁₂ or Gα₁₃, in presence of α_{2A}AR. Gα_{q/11} protein family activation was followed using the selective-G_{i/o} effector Rap1GAP-RlucII and rGFP-CAAX along with the human Gαᵢ₁, Gαᵢ₂, Gα_{oA}, Gα_{oB} or Gα_{z} subunits and α_{2A}AR. Gα_{q/11} protein family activation was determined using the selective-G_{q/11} effector p63-RhoGEF-RlucII and rGFP-CAAX along with the human Gα_{q}, Gα₁₁, Gα₁₄ or Gα₁₅ subunits and α_{2A}AR. β-arrestin recruitment to the plasma membrane was determined using DNA mix containing rGFP-CAAX and β-arrestin-1-RlucII or β-arrestin-2-RlucII in presence of α_{2A}AR.

Bioluminescence Resonance Energy Transfer Measurement. The day of the experiment, cells were washed with phosphate-buffered saline (PBS) and incubated in Tyrode Hepes buffer (137 mM NaCl, 0.9 mM KCl, 1 mM MgCl₂, 11.9 mM NaHCO₃, 3.6 mM NaH₂PO₄, 25 mM HEPES, 5.5 mM D-Glucose and 1 mM CaCl₂), pH 7.4) for 1 h at 37° C. Cells were then treated with increasing concentrations of compounds for 10 min at 37° C. The luciferase substrate Prolume purple (1 μM, NanoLight Technologies) was added during the last 6 min before the reading. Plates were read on the TriStar² LB 942 Multimode Microplate Reader (Berthold Technologies) with the energy donor filter (410±80 nm; RlucII) and energy acceptor filter (515±40 nm; GFP10 and rGFP CAAX). BRET signal (BRET²) was determined by calculating the ratio of the light intensity emitted by the acceptor (515 nm) over the light intensity emitted by the donor (410 nm) and data were normalized in percentage of the maximal response elicited by the reference compound Norepinephrine. The data were analyzed in GraphPad Prism 9.1 using “dose-response-stimulation log(agonist) vs response (four parameters)” and data were presented as mean±s.e.m. of at least 3 different experiments each done in simplicate. Eₘₐₓ and EC₅₀ values were determined from dose-response curves to calculate the log(Eₘₐₓ/EC₅₀) value for each pathway and each compound.

To determine the relative efficacy of the compounds to activate the different signaling pathways, the difference between the log(Eₘₐₓ/EC₅₀) values was calculated using the following equation:

$$Δ Log ( \frac{Emax}{EC 50} ) = {Log ( \frac{Emax}{EC 50} )}_{compund} - {Log ( \frac{Emax}{EC 50} )}_{Norepinephrine}$$

The compounds' efficacy toward each pathway, relative to norepinephrine, were calculated as the inverse logarithm of the Δ log(Eₘₐₓ/EC₅₀) using the following equation:

$$Δ Log ( \frac{Emax}{EC 50} ) = {Log ( \frac{Emax}{EC 50} )}_{compund} - {Log ( \frac{Emax}{EC 50} )}_{Norepinephrine}$$

The SEM was calculated for the log(Eₘₐₓ/EC₅₀) ratios using the following equation:

$$SEM = \frac{σ}{\sqrt{n}}$$

where σ is the standard deviation, and n is the number of experiments.

The SEM was calculated for the Δ log(Eₘₐₓ/EC₅₀) ratios using the following equation:

$${SEM}_{( Δ Log ( \frac{Emax}{EC 50} ) )} = \sqrt{( {SEM}_{compound )}^{2} + ( {SEM}_{Norepinephrine )}^{2}}$$

Statistical analysis was performed using a two-tailed unpaired t test on the Δ log(Eₘₐₓ/EC₅₀) ratios to make pairwise comparisons between tested compounds and norepinephrine for a given pathway, where p<0.05 was considered to be statistically significant.

Internalization assay with rGFP-CAAX and rGFP-FYVE biosensors.

Plasmids. Human α_{2A}AR sequence was fused to RlucII by cloning between the NheI and BamHI sites of pCDNA3.1/Zeo(+)-RlucII vector, using polymerase chain reaction (Q5 Hot Start High-Fidelity DNA Polymerase from NEB), enzymatic digestion (NEB) and ligation (Anza™ T4 DNA Ligase Master Mix; Invitrogen).

Transfection. The protocol used for transfection is the same as for G_{12/13} EMTA experiments (i.e., cells were transfected with 1 μg of total DNA (adjusted with salmon sperm DNA; Invitrogen)). Transfections were performed using linear polyethylenimine (PEI, 1 mg/mL; Polysciences) diluted in NaCl (150 mM, pH 7.0) as a transfecting agent (3:1 PEI/DNA ratio). Cells were immediately seeded (3.5×10⁴ cells/well) into 96-well white microplates (Perkin Elmer), maintained in culture for the next 48 h and BRET experiments carried out. Human α_{2A}AR internalization was evaluated by measuring the disappearance of hα_{2A}AR-RlucII from the plasma membrane labeled with rGFP-CAAX and its relocalization in endosome labeled with rGFP-FYVE (39).

Bioluminescence Resonance Energy Transfer Measurement. The day of the experiment, cells were washed with phosphate-buffered saline (PBS) and incubated in Tyrode Hepes buffer for 1 h at 37° C. Cells were incubated during 6 min with the luciferase substrate Prolume purple (1 μM, NanoLight Technologies) before addition of the indicated compound (0 or 100 μM) and kinetics were recorded during 30 min. For concentration-response curves, BRET signal was measured after 30 min incubation. Plates were read on a Spark microplate reader (Tecan; Mannedorf, Switzerland) using the BRET2 manufacturer settings. BRET signal (BRET²) was determined by calculating the ratio of the light intensity emitted by the acceptor (515 nm) over the light intensity emitted by the donor (410 nm) and for concentration-response curves, data were normalized in percentage of the maximal response elicited by the reference compound norepinephrine. The data were analyzed in GraphPad Prism 9.1 using “log(agonist) vs. response—Variable slope (four parameters)” and data were presented as mean±s.e.m. of 3 experiments performed in triplicate for kinetics or in simplicate for concentration-response curves.

### Cryo-EM Sample Preparation and Structure Determination

Preparation of the ‘9087-α_{2A}AR-G_{oA}-scFv16 and ‘4622-α_{2A}AR-G_{oA}-scFv16 complexes. The human wild type α_{2A}AR was cloned to pFastBac vector with a N-terminal FLAG tag and a C-terminal histidine Tag. This construct was expressed in Sf9 insect cells using the pfastBac baculovirus system (Expression Systems). Cells were infected at a density of 4×10⁶ cells per m1 and then incubated for 48 hours at 27° C. Receptor was extracted and purified following the protocol described previously for α_{2B}AR (9). Briefly, receptor was purified by Ni-NTA chromatography, Flag affinity chromatography and size exclusion chromatography in the presence of 100 μM ‘9087 or ‘4622. The monomeric peak fractions of receptor were collected and concentrated to ˜20 mg/mL. The freshly purified ‘9087-bound or ‘4622-bound α_{2A}AR was used for complex formation with the G protein. G_{oA} heterotrimers were expressed and purified as previously described with minor modifications (78). Briefly, Hi5 cells were grown to a density of 3 million per mL and then infected with Gαₒ and Gβ₁γ₂ baculovirus at a ratio of 10-20 mL/L and 1-2 mL/L, respectively, and then incubated for 48 hours at 27° C. Cells were solubilized with 1% (w/v) sodium cholate and 0.05% (w/v) DDM. After centrifugation, the supernatant was loaded onto Ni-NTA column and then exchanged to 0.05% DDM. The eluted G_{oA} heterotrimer was dephosphorylated by lambda phosphatase (homemade) and further purified through ion exchange using a Mono Q 10/100 GL column (GE Healthcare) and the peak fractions were collected and flash frozen in liquid nitrogen until use. The scFv16 (80) protein was expressed in insect Sf9 cells and purified with Ni-NTA column followed by the Superdex 200 Increase 10/300GL column (GE Healthcare) with a buffer composed of 20 mM HEPEs, pH 7.5 and 100 mM NaCl. The monomeric peak fractions of receptor were collected and concentrated and stored at −80° C. until use. The complex formation process is same as described. Briefly, the complex of α_{2A}AR with heterotrimeric G_{oA} was formed in a buffer containing 20 mM HEPEs pH 7.5, 100 mM NaCl, 0.1% DDM, 1 mM MgCl₂, 10 μM GDP and 100 μM ‘9087 or ‘4622. The α_{2A}AR-G_{oA} complex was then treated with 50 units of apyrase (NEB) on ice overnight, and exchanged on an anti-Flag M1 column into a buffer containing 20 mM HEPES, pH 7.5, 100 mM NaCl, 0.0075% lauryl maltose neopentyl glycol (MNG, NG310 Anatrace), 0.0025% GDN (GDN101, Anatrace), and 0.001% CHS, 100 μM ‘9087 or ‘4622 and 2 mM CaCl₂) in a stepwise manner. After elution by adding 5 mM EDTA and 0.2 mg/mL Flag peptide, the complex was concentrated and incubated with 1.5× molar excess scFv16 for 1 hour on ice, then further purified using Superdex 200 Increase 10/300GL column (GE Healthcare) with a running buffer of 20 mM HEPES, pH 7.5, 100 mM NaCl, 0.00075% MNG, 0.00025% GDN and 0.0001% CHS, 100 μM ‘9087 or ‘4622. The monomeric peak fraction of α_{2A}AR-G_{oA} complex was collected and concentrated to ˜5 mg/mL for cryo-EM.

Cryo-EM data collection, processing, and model building. 3 μL of purified complex sample was applied onto the grid (CryoMatrix nickel titanium alloy film, R1.2/1.3, Zhenjiang Lehua Electronic Technology Co., Ltd.) (81) glow discharged at Tergeo-EM plasma cleaner and then blotted for 3 see with blotting force of 0 and quickly plunged into liquid ethane cooled by liquid nitrogen using Vitrobot Mark IV (Thermo Fisher Scientific, USA) at 10° C. and with 95% humidity. Cryo-EM data was collected on a 300 kV Titan Krios Gi3 microscope. The raw movies were recorded by Gatan K3 BioQuantum Camera at the magnification of 105,000 and the corresponding pixel size is 0.85 Å. Inelastically scattered electrons were excluded by a GIF Quantum energy filter (Gatan, USA) using a slit width of 20 eV. The movie stacks were acquired with the defocus range of −1.0 to −1.6 micron with total exposure time 2.5 s fragmented into 50 frames (0.05 s/frame) with the dose rate of 22.0 e/pixel/s. The imaging mode is super resolution with 2-time hardware binning. The semi-automatic data acquisition was performed using SerialEM (82).

For the ‘9087-α_{2A}AR-G_{oA}-scFv16 complex, raw movie frames were aligned with MotionCor2 (83) using a 9×7 patch and the contrast transfer function (CTF) parameters were estimated using Gctf and ctf in JSPR (84). Micrographs with consistent CTF values including defocus and astigmatism parameter were kept for the following image processing, which kept 3,768 micrographs from 4,217 raw movies. Templates for particle auto-picking were generated by projecting the 3D volume of norepinephrine-bound α_{2A}AR-G_{oA} complex (40). The 2,137,146 particles picked from template picking was subjected 2D classification in cryoSPARC (85) and 3D-classication in Relion3.1 (86). The sorted 321,762 particles were then subjected to homogeneous reconstruction in cryoSPARC, yielding a 3.57 Å map. Further 3D Ab-initio reconstruction reduced the particles number to 287,431, which was subjected to CTF refinement and non-uniform refinement after extracting with larger particle box size, and finally yield the 3.47 Å map.

For the ‘4622-α_{2A}AR-Goa-scFv16 complex, 6,983 raw movies were collected and subjected for motion correction using MotionCor2 (83). Contrast transfer function parameters were estimated by CTFFIND4, implemented in Relion3.1 (86). 2,593,747 particles were auto-picked using the templates in RELION3.1 and then subjected to 2D classification using cryoSPARC. Selected particles with appropriate 2D average from 2D classification were further subjected to Ab-initio reconstruction. Particles with appropriate initial model were selected from Ab-initio followed by heterogeneous refinement in cryoSPARC. The particles kept to 563,506 particles were subjected to non-uniform refinement and local refinement and yield a 3.38 Å reconstruction determined by gold standard Fourier shell correlation using the 0.143 criterion.

The norepinephrine-α_{2A}AR-Goα complex structure (PDB 7EJ0) (40) was used as the initial template for model building. The model was docked into the cryo-EM density map using UCSF Chimera (https://rbvi.ucsf.edu/chimera), followed by iterative manual building in Coot (87) and real space refinement in Phenix. The statistics of the final models were validated by Molprobity. The ligand symmetry accounted RMSDs between the docked pose and cryo-EM pose of ‘9087 and ‘4622 were calculated by the Hungarian algorithm in DOCK6 (88).

pKa determination for ‘9087. The pKa of ‘9087 (2.90 mg, 0.013 mmol) was determined by potentiometric titration using a Metrohm pH Meter 632 equipped with a glass electrode (Metrohm 6.0259.100). The compound was dissolved in 15 mL of 10% methanol aqueous solution, at an ionic strength of I=0.15 μM using KCl. The resulting solution was stirred throughout the experiment using a magnetic stir bar and a magnetic agitator. The compound was titrated with 0.01 M HCl (Titrisol®) using an automatic burette (Metrohm Dosimat Plus 876). The titrant was added to the analyte stepwise (0.024-2.87 mL). The resulting graph for pKa-determination is presented in dependence of t and pH(t). The pKa value was then determined using a simplified Henderson-Hasselbalch equation. The data from the titration experiment was evaluated with Origin 9.60.

### Off-Target Activity

GPCRome. 10 μM ‘9087 was tested for off-target activity at a panel of 320 non-olfactory GPCRs using PRESTO-Tango GPCRome arrestin-recruitment assay as described (45). Receptors with at least three-fold increased relative luminescence over corresponding basal activity are potential positive hits. Screening was performed by the National Institutes of Mental Health Psychoactive Drug Screen Program (PDSP). Detailed experimental protocols are available on the NIMH PDSP website at https://pdsp.unc.edu/pdspweb/content/PDSP %20Protocols %20II %202013-03-28.pdf.

D2R Activation. D2R was selected following the GPCRome panel and ‘9087 was re-tested for full dose-response to determine G protein and arrestin recruitment (see above).

I2R Binding. Top docking compounds (‘9087, ‘2998, ‘4622, ‘0172) were tested for I2R binding, performed by Eurofins Cerep (France; catalog #81) as described (78). For compound ‘2998, no binding was seen in a single point radioligand competition experiment tested at 500 nM and the compound is not shown.

μOR competition binding. Equilibrium [3H] Diprenorphine competition and saturation binding were carried out in membranes prepared from Chinese Hamster Ovary (CHO-K1) cells stably expressing human μ-opioid receptor, as previously described (89-91). Briefly, binding was performed at 25° C. for 90 min in the dark. Binding in OR/CHO-K1 cells was carried out in a buffer consisting of 50 mM HEPES-base pH 7.4 (pH adjusted with KOH), 10 mM MgCl₂, 0.1 mM EDTA, and 0.1% (w/v) Bovine Serum Albumin with membranes containing approximately 40 μg/mL protein. Following incubation with radioligand (1 μM to 10 nM for saturation, 500 μM for competition), drugs (33 μM to 3.3 μM) and/or 20 μM cold competitor naloxone, the reaction was rapidly filtered onto GF/B (PerkinElmer #1450-521) glass fiber filtermats which were equilibrated for 1 hour in binding buffer supplemented with 0.3% (v/v) polyethyleneimine. The filtermats were washed 5 times in ice-cold 50 mM HEPES-base pH 7.4 using a Perkin Elmer semi-automated cell harvester (Perkin Elmer FilterMate Harvester). The filtermats were dried and Meltilex solid scintillant (Perkin Elmer #1450-442) was melted onto the mats for 10 min at 60° C. The scintillant was allowed to re-solidify before disintegrations were quantified with a Wallac MicroBeta Scintillation counter using an integration time of 1 min. Non-specific binding, total binding, the number of receptor binding sites, and the K_{d} of the radiotracer were determined from saturation binding experiments. Protein concentrations were determined using the microBCA method with BSA as the standard. Kᵢ values were calculated by non-linear regression analysis and application of the Cheng-Prusoff correction in GraphPad Prism 9.0.

hERG inhibition assays. ‘9087 was tested for hERG inhibition in the FluxOR assay as described (92). hERG experiments used the National Institutes of Mental Health (NIMH) Psychoactive Drug Screening Program (PDSP). Experimental protocols are available on the NIMH PDSP website at https://pdsp.unc.edu/pdspweb/content/PDSP %20Protocols %20II %202013-03-28.pdf.

Metabolic stability studies. Metabolic stability of the test compounds was assessed using male pooled rat liver microsomes (Sprague Dawley, Sigma Aldrich) as previously described (93, 94). The reactions were carried out in 2 mL polyethylene tubes on a rotator carousel (Stuart™ SB3) in an incubator at 37° C. The incubation mixture contained ‘9087, '7075, PS75, or the positive controls rotigotine or imipramine (final concentration 20 μM), and pooled rat liver microsomes (0.25 mg protein/tube) in Tris-MgCl₂ buffer (50 mM Tris, 5 mM MgCl₂, pH 7.4, final volume 500 μL). Transformation reactions were initiated by the addition of 50 μL of cofactor solution (NADPH, Carl Roth, final concentration 1 mM). After time intervals of 0, 30 and 60 min for ‘9087, ‘7075, and PS75 or 0, 15, 30, 60 min for rotigotine and imipramine, respectively, 100 μL aliquots of the reaction mixtures were added to 100 μL ice-cold acetonitrile (containing 10 μM chlorpromazine as internal standard) to terminate enzymatic reactions. Precipitated protein was removed by centrifugation (1 min, 16,000 rcf) and the supernatants were analysed by HPLC/MS on a Thermo Scientific Dionex Ultimate 3000 HPLC system equipped with a Zorbax Eclipse XDB-C₈ column (4.6×150 mm, 5 μm), a DAD detector (210 nm, 230 nm, 254 nm, 310 nm), and a BRUKER amaZon SL mass spectrometer with ESI source. The following binary eluent system (methanol in water+0.1% (v/v) formic acid) was employed: 10% for 1 min, 10% to 100% in 20 min, 100% for 5 min, 100% to 10% in 2 min, 10% for 2 min, flow 0.4 mL/min. Per compound four (rotigotine, imipramine) or five (‘9087, ‘7075, PS75) independent experiments were performed. Control experiments were conducted in the absence of cofactor solution to determine non-specific binding to matrix. The integral (AUC) of the extracted ion chromatograms (EIC) was used to analyze the concentration of the remaining substrates. Concentrations were plotted in their logarithmic form as a function of the incubation time (in min) to calculate the elimination rate constant (k) and to determine the half-life (T_{1/2}) and intrinsic hepatic clearance (CLᵢₙₜ) for each compound with the following equations(95):

$${t}_{1 / 2} [ min ] = \frac{ln ( 2 )}{k} \\ {CL}_{int} [ \frac{μL}{min * mg ( protein )} ] = \frac{ln ( 2 )}{{t}_{1 / 2}} * \frac{V ( of incubation in μL )}{m ( of protein in incubation in mg )}$$

### In Vivo Methods

Animals and ethical compliance. Animal experiments were approved by the UCSF Institutional Animal Care and Use Committee and were conducted in accordance with the NIH Guide for the Care and Use of Laboratory animals (protocol #AN181214). Adult (8-10 weeks old) male C56BL/6 mice (strain #664) were purchased from the Jackson Laboratory. Mice were housed in cages on a standard 12:12 hour light/dark cycle with food and water ad libitum. The α_{2A}AR D79N mutant mice were purchased from Jackson (stock #2777), and 7-8 week-old females were used. Sample sizes were modelled on our previous studies and on studies using a similar approach, which were able to detect significant changes (96, 97). The animals were randomly assigned to treatment and control groups. Animals were initially placed into one cage and allowed to freely run for a few minutes. Then each animal was randomly picked up, injected with compound treatment or vehicle, and placed into a separate cylinder before the behavioral test.

In vivo compound preparation. All ligands were synthesized by Enamine (‘2998, '7075) or WuXi (‘9087, ‘4622, ‘0172) or in-house (PS75) and dissolved 30 minutes prior testing. Available salt forms were used to aid solubility: HCl for ‘9087 and ‘7075, TFA for ‘4622, ‘0172, and PS75. ‘9087, ‘4622 and ‘0172 were resuspended in 20% Kolliphor (Sigma-Aldrich; cat. #C5135) for s.c./i.p. injections. ‘2998, ‘7075, and PS75 were resuspended in 20% cyclodextran (Sigma-Aldrich; cat. #H107) for s.c./i.p injections. Atipamezole (Cayman Chemical Company; cat. #9001181) and Dexmedetomidine (Cayman Chemical Company; cat. #15581) were resuspended with NaCl 0.9% (Teknova; cat. #S5819) for s.c./i.p. injections. ‘9087 was formulated with 40% Captisol (Carbosynth; cat. #OC15979) for p.o. dosing.

Behavioral analyses. For all behavioral tests, the experimenter was always blind to treatment. Animals were first habituated for 1 hour in Plexiglas cylinders and then tested 30 minutes after subcutaneous injection of the α_{2A}AR compounds. The α_{2A}AR antagonist atipamezole (2 mg/kg, i.p.) was injected 15 minutes prior to s.c. injection of the α_{2A}AR agonists. The mechanical (Von Frey), thermal (Hargreaves, hotplate and tail flick) and ambulatory (rotarod) tests were conducted as described previously (98). Hindpaw mechanical thresholds were determined with von Frey filaments using the updown method (99). Hindpaw thermal sensitivity was measured with a radiant heat source (Hargreaves) or a 55° C. hotplate. For the tail flick assay, sensitivity was measured by immersing the tail into a 50° C. water bath for both WT and D79N mutant mice. For the ambulatory (rotarod) test, mice were first trained on an accelerating rotating rod, 3 times for 5 min, before testing with any compound.

Spared-nerve injury (SNI) model of neuropathic pain. Under isoflurane anesthesia, two of the three branches of the sciatic nerve were ligated and transected distally, leaving the sural nerve intact. Behavior was tested 7 to 14 days after injury and in situ hybridization was performed one week post-injury.

Complete Freund's Adjuvant (CFA). The CFA model of chronic inflammation was induced as described previously (100). Briefly, CFA (Sigma) was diluted 1:1 with saline and vortexed for 30 minutes. When fully suspended, we injected 20 μL of CFA into one hindpaw. Heat thresholds were measured before the injection (baseline) and 3 days after using the Hargreaves test.

Constipation assay. Mice had access to food and water ad libitum prior to the test. On the test day, mice received an i.p. injection of a solution (100 μL) containing saline, 10 mg/kg morphine, 30 μg/kg dexmedetomidine, or 5 mg/kg ‘9087 and then individually placed in a clean cage, with no access to food or water. Fecal pellets were collected and counted every hour, up to 6 hours.

Body weight measurement. The body weights were measured before, 24 hours, and 48 hours after mice received an i.p. injection of a solution (100 μL) containing dexmedetomidine (30 μg/kg) or ‘9087 (5, 10, or 20 mg/kg).

Pharmacokinetics. Pharmacokinetic experiments were performed by Bienta (Enamine Biology Services) in accordance with Enamine pharmacokinetic study protocols and Institutional Animal Care and Use Guidelines (protocol number 1-2/2020). Plasma pharmacokinetics and brain distribution for ‘9087, ‘2998, ‘4622, ‘7075, PS75, and CSF distribution for ‘7075, PS75, ‘9087, and ‘4622, were measured following a 10 mg/kg (i.p.) dose. Plasma and brain samples were also collected for ‘9087 following 10 mg/kg i.v. and 30 mg/kg p.o. (oral) dose to determine oral bioavailability. In each compound study, nine time points (5, 15, 30, 60, 120, 240, 360, 480 and 1440 min) were collected, each of the time point treatment group included 3 animals. There was also a control group of one animal. In the ‘9087, ‘7075, ‘4622 studies, male C57BL/6N mice were used, for PS75 CD-1 mice, and for ‘2998 male Balb/cAnN mice. For all compound studies the animals were randomly assigned to the treatment groups before the pharmacokinetic study; all animals were fasted for 4 h before dosing. For injections, ‘9087 was dissolved in Captisol—water (40%:60%, w/v), ‘4622 was dissolved in a 20% Kolliphor HS—physiological saline solution, and ‘7075, PS75, and ‘2998 were dissolved in a 20% 2-HPBCD—aqueous solution. The batches of working formulations were prepared 10 minutes prior to the in vivo study.

Mice were injected i.p. with 2,2,2-tribromoethanol at 150 mg/kg prior to drawing CSF and blood. CSF was collected under a stereomicroscope from cisterna magna using 1 mL syringes. Blood collection was performed from the orbital sinus in microtainers containing K3EDTA. Animals were sacrificed by cervical dislocation after the blood samples collection. Blood samples were centrifuged 10 min at 3000 rpm. Brain samples (right lobe) were weighed and transferred into 1.5 mL tubes. All samples were immediately processed, flash-frozen and stored at −70° C. until subsequent analysis.

Plasma samples (40 μL) were mixed with 200 μL of internal standard (IS) solution. After mixing by pipetting and centrifuging for 4 min at 6000 rpm, 4 μL of each supernatant was injected into the LC-MS/MS system. Solutions of internal standards were used to quantify compounds in the plasma samples. Brain samples (weight 200 mg±1 mg) were homogenized with 800 μL of an internal stock solution using zirconium oxide beads (115 mg±5 mg) in a Bullet Blender® homogenizer for 30 seconds at speed 8. After this, the samples were centrifuged for 4 min at 14,000 rpm, and supernatant was injected into LC-MS/MS system. CSF samples (2 μL) were mixed with 40 μL of an internal stock solution. After mixing by pipetting and centrifuging for 4 min at 6,000 rpm, 5 μL of each supernatant was injected into LC-MS/MS system.

Analyses of plasma, brain and CSF samples were conducted at Enamine/Bienta. The concentrations of compounds in plasma and brain samples were determined using high performance liquid chromatography/tandem mass spectrometry (HPLC-MS/MS). Data acquisition and system control was performed using Analyst 1.5.2 software (AB Sciex, Canada). The concentrations of the test compound below the lower limit of quantitation (LLOQ=10 ng/mL for plasma, 20 ng/g for brain and 5 ng/mL for CSF samples) were designated as zero. Pharmacokinetic data analysis was performed using noncompartmental, bolus injection or extravascular input analysis models in WinNonlin 5.2 (PharSight). Data below LLOQ were presented as missing to improve validity of T_{1/2} calculations.

Additional pharmacokinetic experiments were performed by Sai Life Sciences (Hyderabad, India) in accordance with the Sai Study Protocol SAIDMPK/PK-22-04-0340. Brain distribution of dexmedetomidine was measured following a 30 μg/kg i.p. dose, using normal saline 0.9% as its vehicle. Plasma distributions were also collected for PS75 following 10 mg/kg i.v. and 30 mg/kg p.o. (oral) dosing to determine oral bioavailability; both doses were formulated in 20% v/v HPCD in saline. Testing was done in C57BL/6 mice. For PS75, 24 mice were divided into 4 groups: 9 mice for i.v. dosing of the compound, 3 mice for i.v. dosing of the vehicle, 9 mice for p.o. dosing with the compound, and 3 mice for p.o. vehicle dosing; sparse sampling of three mice/time point for compound treated groups and 1 mouse/time point for vehicle groups was performed. For dexmedetomidine, 36 mice were included and split into two groups: 3 mice/time point for compound dosing, and 1 mouse/time point for vehicle only dosing. For PS75, blood samples (60 μL) were collected under light isoflurane anesthesia (Surgivet®) from retro orbital plexus from a set of 3 mice at 0.083, 0.25, 0.5, 1, 2, 4, 6, 8, and 24 hr. Immediately after blood collection, plasma was harvested by centrifugation at 4000 rpm, 10 min at 4° C. For dexmedetomidine, brain samples were collected at the same time points indicated above. Animals were sacrificed at respective time-points and brain samples were isolated and homogenized in ice-cold phosphate buffer saline (pH 7.4). Total homogenate volume was three times the tissue weight. Samples were stored at −70° C. until bioanalysis. All samples were processed for analysis by protein precipitation method and analyzed with fit-for-purpose LC-MS/MS method (LLOQ=3.61 ng/mL for plasma for PS75, LLOQ=0.86 ng/mL for brain for dexmedetomidine). The pharmacokinetic parameters were estimated using the noncompartmental analysis tool of Phoenix® WinNonlin software (v. 8.0).

Statistical analyses. Data from functional experiments of adrenergic and D_{2long} receptors were analyzed applying the algorithms for four parameter non-linear regression implemented in Prism 8.0 (GraphPad, San Diego, CA) to get dose-response curves representing EC₅₀ and Eₘₐₓ values. Mean values were derived by summarizing the results from each individual experiment to provide EC₅₀±s.e.m. and Eₘₐₓ±s.e.m. (or s.d. where indicated). Additional statistical analyses for FIGS. 4B-4G, FIGS. 4I-4J, FIG. 8, FIG. 15A, FIGS. 15C-15E, and FIGS. 17A-17B were performed with GraphPad Prism 9.0 (GraphPad Software Inc., San Diego). Data reported are means±s.e.m. or, in FIGS. 4B-4G, FIGS. 4I-4J, and FIGS. 17A-17B, single data points with means±s.e.m. Experiments of the compounds in the in vivo neuropathic, inflammatory, hot-plate, tail flick, and rotarod models were evaluated using unpaired two-tailed Student's t-test or one-way ANOVA with Dunnett's multiple comparison post-hoc test to determine differences between groups. Experiments for body weight and constipation were analyzed with a two-way ANOVA. Details of the analyses, including groups compared in statistical sets, number of animals per group, and p-values can be found in the figure legends.

TABLE 1
Binding affinity and functional properties of selected compounds in comparison to the
reference compounds norepinephrine, dexmedetomidine and clonidine to the human α_{2A}AR receptor.

| | Kᵢ for α_{2A}AR^{a} | | Gαᵢ^{b} | | G_{i/o}^{c} | | | | β-arrestin-2^{d} | | |
|---|---|---|---|---|---|---|---|---|---|---|---|
| | | | EC₅₀ | Eₘₐₓ | | EC₅₀ | Eₘₐₓ | | EC₅₀ | Eₘₐₓ | |
| com− | [nM ± | | [nM ± | [% ± | | [nM ± | [% ± | | [nM ± | [% ± | |
| pound | s.e.m.] | n | s.e.m.] | s.e.m.] | n | s.e.m.] | s.e.m.] | n | s.e.m.] | s.e.m.] | n |
| NorEpi | 4100 ± 1500 | 4 | 5.0 ± 0.94 | 100 | 17 | — | — | — | 450 ± 30 | 100 | 8 |
| DEX | 10 ± 1.5 | 8 | 0.077 ± 0.015 | 105 ± 4 | 7 | 0.057 ± 0.015 | 76 ± 3 | 2 | 1.3 ± 0.43 | 60 ± 3 | 5 |
| CLON | 84 ± 21^{e} | 2 | 2.4 ± 0.96 | 115 ± 4 | 5 | — | — | — | 17 ± 2.5 | 41 ± 3 | 5 |
| BRIM | — | — | — | — | — | 0.232 ± 0.043 | 107 ± 7 | 2 | — | — | — |
| ‘9087 | 51 ± 8.8 | 6 | 52 ± 24 | 60 ± 3 | 9 | 87 ± 13 | 42 ± 2 | 2 | — | <5 | 6 |
| ‘2998 | 180 ± 34 | 5 | 73 ± 14 | 61 ± 3 | 7 | — | — | — | 98 ± 32 | 8 ± 3 | 4 |
| ‘0172 | 260 ± 33 | 4 | 210 ± 38 | 95 ± 7 | 4 | — | — | — | 1700 ± 290 | 22 ± 2 | 3 |
| ‘4622 | 12 ± 3.6 | 4 | 9.7 ± 2.4 | 74 ± 4 | 6 | — | — | — | — | <5 | 3 |
| ‘7075 | 37 ± 5.1 | 5 | 4.1 ± 1.2 | 93 ± 5.2 | 9 | 18 ± 1.9 | 96 ± 2 | 2 | — | 10 ± 7 @ | 7 |
| | | | | | | | | | | 0.1 mM | |
| PS75 | 8.2 ± 0.48 | 4 | 4.8 ± 1.3 | 82 ± 4 | 7 | — | — | — | — | 15 ± 3 @ | 6 |
| | | | | | | | | | | 0.1 mM | |

^{a}Binding affinity to human α_{2A}AR as mean Kᵢ value ± s.e.m derived from 4−8 experiments each done in triplicate.
^{b}G-protein signaling monitored in a BRET-biosensor based assay in HEK293T cells expressing the human α_{2A}AR performed in duplicates.
^{c}G-protein signaling monitored in DiscoverX HitHunter cAMP assay expressing the human α_{2A}AR.
^{d}β-Arrestin-2 recruitment monitored in a BRET-biosensor based assay in HEK293T cells expressing the human α_{2A}AR performed in duplicates.
^{e}Kᵢ value ± SD derived from 2 experiments.
NorEpi, norepinephrine.
DEX, dexmedetomidine.
CLON, clonidine.
BRIM, brimonidine.
—, not determined.

TABLE 2
Functional properties in EMTA coupling panel of ‘9087 and its analogs in comparison
to norepinephrine, dexmedetomidine and brimonidine against the human α_{2A}AR.

| Endogenous | | | | | | | |
|---|---|---|---|---|---|---|---|
| G_{i/o}^{a} | | Norepinephrine | Dexmedetomidine | Brimonidine | ‘9087 | ‘7075 | PS75 |
| Emax | mean | 100 | 101 | 111 | 55 | 75 | 76 |
| | SEM | 1.99 | 2.28 | 2.71 | 1.63 | 1.66 | 1.22 |
| LogEC₅₀ | mean | −8.63 | −10.01 | −9.83 | −7.58 | −7.82 | −8.11 |
| | SEM | 0.05 | 0.06 | 0.07 | 0.07 | 0.05 | 0.03 |
| N | | 3 | 5 | 5 | 5 | 5 | 5 |
| Gi1 | | Norepinephrine | Dexmedetomidine | Brimonidine | ‘9087 | ‘7075 | PS75 |
| Emax | mean | 100 | 104 | 100 | 64 | 95 | 95 |
| | SEM | 2.42 | 0.92 | 1.09 | 1.87 | 0.97 | 0.91 |
| LogEC₅₀ | mean | −8.66 | −9.92 | −9.96 | −7.20 | −7.47 | −7.97 |
| | SEM | 0.06 | 0.02 | 0.02 | 0.05 | 0.02 | 0.02 |
| N | | 3 | 5 | 5 | 5 | 5 | 5 |
| Gi2 | | Norepinephrine | Dexmedetomidine | Brimonidine | ‘9087 | ‘7075 | PS75 |
| Emax | mean | 100 | 101 | 99 | 62 | 96 | 101 |
| | SEM | 2.25 | 1.23 | 1.12 | 2.63 | 0.92 | 1.17 |
| LogEC₅₀ | mean | −8.76 | −10.00 | −10.02 | −7.26 | −7.51 | −7.92 |
| | SEM | 0.06 | 0.02 | 0.02 | 0.08 | 0.02 | 0.02 |
| N | | 3 | 5 | 5 | 5 | 5 | 5 |
| Gi3 | | Norepinephrine | Dexmedetomidine | Brimonidine | ‘9087 | ‘7075 | PS75 |
| Emax | mean | 100 | 101 | 105 | 54 | 91 | 101 |
| | SEM | 1.91 | 1.51 | 2.40 | 2.71 | 1.58 | 2.75 |
| LogEC₅₀ | mean | −8.49 | −9.92 | −9.88 | −7.22 | −7.46 | −7.85 |
| | SEM | 0.05 | 0.03 | 0.05 | 0.10 | 0.04 | 0.05 |
| N | | 3 | 5 | 5 | 5 | 5 | 5 |
| GoA | | Norepinephrine | Dexmedetomidine | Brimonidine | ‘9087 | ‘7075 | PS75 |
| Emax | mean | 100 | 108 | 105 | 96 | 95 | 96 |
| | SEM | 3.86 | 1.46 | 1.58 | 1.94 | 3.55 | 1.20 |
| LogEC₅₀ | mean | −9.01 | −10.36 | −10.33 | −7.59 | −7.97 | −8.41 |
| | SEM | 0.07 | 0.03 | 0.04 | 0.04 | 33.55 | 0.03 |
| N | | 3 | 5 | 5 | 5 | 5 | 5 |
| GoB | | Norepinephrine | Dexmedetomidine | Brimonidine | ‘9087 | ‘7075 | PS75 |
| Emax | mean | 100 | 107 | 105 | 98 | 103 | 105 |
| | SEM | 3.43 | 1.60 | 1.98 | 1.77 | 2.58 | 1.66 |
| LogEC₅₀ | mean | −9.17 | −10.57 | −10.48 | −7.76 | −8.08 | −8.57 |
| | SEM | 0.09 | 0.04 | 0.05 | 0.04 | 0.04 | 0.04 |
| N | | 3 | 5 | 5 | 5 | 5 | 5 |
| Gz | | Norepinephrine | Dexmedetomidine | Brimonidine | ‘9087 | ‘7075 | PS75 |
| Emax | mean | 100 | 99 | 98 | 95 | 93 | 99 |
| | SEM | 4.38 | 1.46 | 1.52 | 2.45 | 1.44 | 0.99 |
| LogEC₅₀ | mean | −10.02 | −11.50 | −11.68 | −8.86 | −8.93 | −9.43 |
| | SEM | 0.09 | 0.04 | 0.04 | 0.05 | 0.03 | 0.03 |
| N | | 3 | 5 | 5 | 5 | 5 | 5 |
| Gs | | Norepinephrine | Dexmedetomidine | Brimonidine | ‘9087 | ‘7075 | PS75 |
| Emax | mean | 100 | 57 | 85 | <5 | <5 | <5 |
| | SEM | 3.82 | 4.21 | 3.92 | — | — | — |
| LogEC₅₀ | mean | −6.50 | −8.04 | −7.82 | — | — | — |
| | SEM | 0.08 | 0.17 | 0.10 | — | — | — |
| N | | 5 | 5 | 5 | 5 | 5 | 5 |
| G12 | | Norepinephrine | Dexmedetomidine | Brimonidine | 19087 | ‘7075 | PS75 |
| Emax | mean | 100 | 61 | 107 | <5 | <5 | <5 |
| | SEM | 2.48 | 2.26 | 4.00 | — | — | — |
| LogEC₅₀ | mean | −6.60 | −7.91 | −7.58 | — | — | — |
| | SEM | 0.07 | 0.09 | 0.09 | — | — | — |
| N | | 5 | 5 | 5 | 5 | 5 | 5 |
| G13 | | Norepinephrine | Dexmedetomidine | Brimonidine | ‘9087 | ‘7075 | PS75 |
| Emax | mean | 100 | 41 | 111 | <5 | <5 | <5 |
| | SEM | 1.39 | 1.04 | 3.19 | — | — | — |
| LogEC₅₀ | mean | −6.10 | −7.60 | −7.18 | — | — | — |
| | SEM | 0.02 | 0.06 | 0.07 | — | — | — |
| N | | 5 | 5 | 5 | 5 | 5 | 5 |
| Gq | | Norepinephrine | Dexmedetomidine | Brimonidine | ‘9087 | ‘7075 | PS75 |
| Emax | mean | 100 | 28 | 130 | <5 | <5 | <5 |
| | SEM | 1.70 | 1.59 | 3.77 | — | — | — |
| LogEC₅₀ | mean | −5.95 | −7.34 | −6.74 | — | — | — |
| | SEM | 0.03 | 0.14 | 0.07 | — | — | — |
| N | | 5 | 5 | 5 | 5 | 5 | 5 |
| G11 | | Norepinephrine | Dexmedetomidine | Brimonidine | ‘9087 | ‘7075 | PS75 |
| Emax | mean | 100 | 25 | 153 | <5 | <5 | <5 |
| | SEM | 3.22 | 1.44 | 10.12 | — | — | — |
| LogEC₅₀ | mean | −5.90 | −7.62 | −6.26 | — | — | — |
| | SEM | 0.06 | 0.14 | 0.16 | — | — | — |
| N | | 5 | 5 | 5 | 5 | 5 | 5 |
| G14 | | Norepinephrine | Dexmedetomidine | Brimonidine | ‘9087 | ‘7075 | PS75 |
| Emax | mean | 100 | 24 | 117 | <5 | <5 | <5 |
| | SEM | 3.48 | 0.94 | 4.06 | — | — | — |
| LogEC₅₀ | mean | −5.83 | −7.64 | −6.77 | — | — | — |
| | SEM | 0.06 | 0.10 | 0.08 | — | — | — |
| N | | 5 | 5 | 5 | 5 | 5 | 5 |
| G15 | | Norepinephrine | Dexmedetomidine | Brimonidine | ‘9087 | ‘7075 | PS75 |
| Emax | mean | 100 | 74 | 107 | <5 | <5 | <5 |
| | SEM | 0.02 | 0.02 | 0.04 | — | — | — |
| LogEC₅₀ | mean | −6.30 | −7.59 | −7.42 | — | — | — |
| | SEM | 0.94 | 0.75 | 1.65 | — | — | — |
| N | | 5 | 5 | 5 | 5 | 5 | 5 |
| barrestin1 | | Norepinephrine | Dexmedetomidine | Brimonidine | ‘9087 | ‘7075 | PS75 |
| Emax | mean | 100 | 30 | 103 | <5 | <5 | <5 |
| | SEM | 2.84 | 1.10 | 5.05 | — | — | — |
| LogEC₅₀ | mean | −5.80 | −7.89 | −6.17 | — | — | — |
| | SEM | 0.05 | 0.09 | 0.12 | — | — | — |
| N | | 5 | 5 | 5 | 5 | 5 | 5 |
| barrestin2 | | Norepinephrine | Dexmedetomidine | Brimonidine | ‘9087 | ‘7075 | PS75 |
| Emax | mean | 100 | 38 | 92 | <5 | <5 | <5 |
| | SEM | 1.53 | 0.94 | 2.58 | — | — | — |
| LogEC₅₀ | mean | −5.94 | −7.62 | −6.74 | — | — | — |
| | SEM | 0.02 | 0.06 | 0.07 | — | — | — |
| N | | 5 | 5 | 5 | 5 | 5 | 5 |

G-protein and β-Arrestin signaling for docking compounds in BRET-biosensor based assays in HEK293T cells expressing the human α_{2A}AR. All compound responses normalized to norepinephrine response.
^{a}Endogenous G_{i/o} protein family activation in the absence of heterologously expressed G proteins.
—, not determined.

TABLE 3
Binding affinity and functional properties of selected ′9087 analogs to
the human α_{2A}AR receptor.

| | | Gᵢ EC₅₀ ± SE^{b} | | |
|---|---|---|---|---|
| | | Eₘₐₓ ± SEM^{b} | β-Arr-2^{e} | Statistical |
| | | Mean logEC50 (SEM)^{c} | EC₅₀ | differences |
| | Kᵢ (nM)^{a} | Relative activity^{d} | Eₘₐₓ | for |
| Compound | n | n | n | Gᵢ activity^{f} |
| 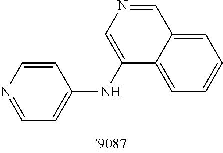 [SMILES: c1ccc2c(Nc3ccncc3)cncc2c1] | 51 ± 8.8 6 | 52 nM ± 24 60% ± 3 −7.585 (0.169) 9.354 (0.171) 9 | —nM <5% 6 | ′5879* PS84**** PS86**** PS75** ′7075**** |
| 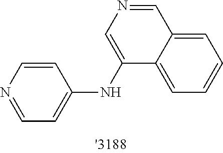 [SMILES: c1ccc2c(Nc3ccncc3)cncc2c1] | >10000 3 | —nM <5% ND ND 4 | — | — |
| 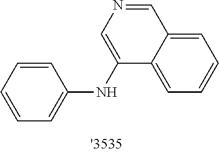 [SMILES: c1ccc(Nc2cncc3ccccc23)cc1] | >10000 3 | —nM 57% ± 9 @ 0.1 mM ND ND 4 | — | — |
| 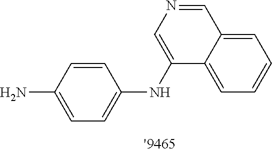 [SMILES: Nc1ccc(Nc2cncc3ccccc23)cc1] | 4600 ± 2100 3 | —nM <5% ND ND 3 | — | — |
| 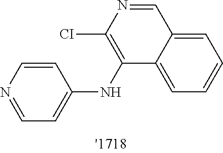 | 22 ± 6.7 3 | 14 nM ± 4.8 47% ± 2 −8.180 (0.296) 9.845 (0.294) 7 | —nM <5% 3 | PS84**** PS86**** |
| 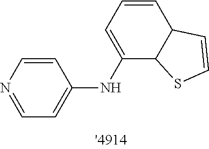 [SMILES: C1=CC2C=CSC2C(Nc2ccncc2)=C1] | 7.2 ± 1.0 4 | 9.1 nM ± 3.2 77% ± 2 −8.175 (0.183) 10.058 (0.184) 5 | —nM <5% 3 | PS71* PS84**** PS86**** |
| 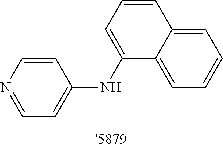 [SMILES: c1ccc2c(Nc3ccncc3)cccc2c1] | 8.8 ± 3.2 3 | 6.1 nM ± 1.2 84% ± 6 −8.251 (0.094) 10.170 (0.117) 5 | —nM 6% ± 1 @ 0.1 mM 4 | ′9087* PS71** PS84**** PS86**** |
| 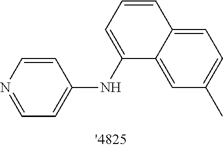 [SMILES: Cc1ccc2cccc(Nc3ccncc3)c2c1] | 19 ± 1.5 4 | 18 nM ± 5 94% ± 4 −7.850 (0.098) 9.823 (0.091) 9 | —nM 18% ± 9 @ 0.1 mM 4 | PS71* PS84**** PS86**** |
| 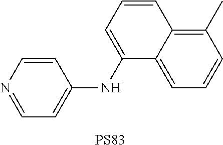 [SMILES: "PS83" ; Cc1cccc2c(Nc3ccncc3)cccc12] | 72 ± 15 3 | 13 nM ± 2.7 94% ± 9 −7.899 (0.093) 9.868 (0.073) 3 | —nM ± | PS84**** PS86**** |
| 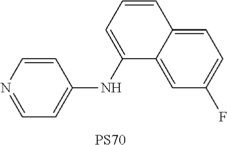 [SMILES: "PS70" ; Fc1ccc2cccc(Nc3ccncc3)c2c1] | 9.2 ± 0.99 3 | 36 nM ± 14 91% ± 3 −7.615 (0.175) 9.570 (0.171) 6 | —nM <5% 4 | PS84** PS86**** ′7075** |
| 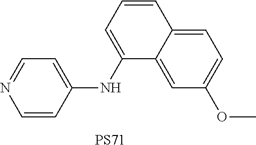 [SMILES: "PS71" ; COc1ccc2cccc(Nc3ccncc3)c2c1] | 240 ± 120 3 | 100 nM ± 19 85% ± 3 −7.020 (0.100) 8.948 (0.113) 4 | —nM <5% 4 | ′4914* ′5879** ′4825* PS84* PS86** PS75*** ′7075**** |
| 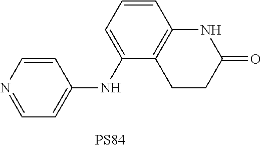 [SMILES: "PS84" ; O=C1CCc2c(cccc2Nc2ccncc2)N1] | 500 ± 87 4 | 840 nM ± 350 37% ± 4 −6.189 (0.250) 7.752 (0.255) 3 | — | ′9087**** ′1718**** ′4914**** ′5879**** ′4825**** PS83**** PS70**** PS71* PS75**** ′7075**** |
| 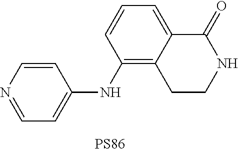 [SMILES: "PS86" ; O=C1NCCc2c(Nc3ccncc3)cccc21] | 1200 ± 560 3 | 3400 nM ± 940 91% ± 15 −5.494 (0.109) 7.442 (0.155) 3 | — | ′9087**** ′1718**** ′4914**** ′5879**** ′4825**** PS83**** PS70**** PS71** PS75 **** ′7075**** |
| 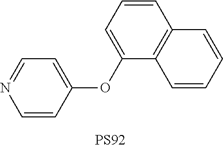 [SMILES: "PS92" ; c1ccc2c(Oc3ccncc3)cccc2c1] | 1800 ± 730 4 | —nM 43% ± 4 @ 0.1 mM ND ND 5 | — | — |
| 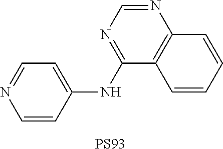 [SMILES: "PS93" ; c1ccc2c(Nc3ccncc3)ncnc2c1] | 6600 ± 1800 4 | —nM <5% ND ND 6 | — | — |
| 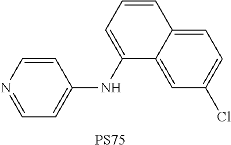 [SMILES: "PS75" ; Clc1ccc2cccc(Nc3ccncc3)c2c1] | 8.2 ± 0.48 4 | 4.8 nM ± 1.3 82% ± 4 −8.441 (0.148) 10.352 (0.149) 7 | —nM 15% ± 3 @ 0.1 mM 6 | ′9087** PS71*** PS84**** PS86**** |
| 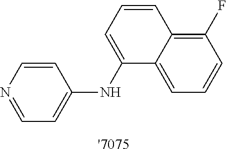 [SMILES: Fc1cccc2c(Nc3ccncc3)cccc12 ; "'7075"] | 37 ± 5.1 5 | 4.1 nM ± 1.2 93% ± 5.2 −8.509 (0.117) 10.471 (0.095) 9 | —nM 10% ± 7@ 0.1 mM 7 | ′9087**** PS70** PS71**** PS84**** PS86**** |

^{a}Binding affinity to human α_{2A}AR as mean Kᵢ value in [nM] ± SEM. ^{b}G-protein activation monitored in a BRET-biosensor based assay using the human α_{2A}AR. ^{c}logEC50 (means calculated from each replicate) of G-protein signaling monitored in a BRET-biosensor based assay using the human α_{2A}AR. ^{d}Relative activity, log(Eₘₐₓ/EC₅₀), of G-protein signaling monitored in a BRET-biosensor based assay using the human α_{2A}AR. ^{e}β-Arrestin-2 recruitment measured with a BRET-biosensor based assay and the human α_{2A}AR. ^{f}Differences of relative compound activities (log(Eₘₐₓ/EC₅₀) were analyzed by One-way ANOVA applying Tukey′s multiple comparisons test in PRISM 9.3.1. *p < 0.05, **p < 0.01, ***, p < 0.001; ****, p < 0.0001. Only compounds with statistically significant differences are listed. — , not determined.

TABLE 4
Binding affinity of ′9087 to other adrenergic receptors.^{a}

| α_{2C}AR | α_{1A}AR | α_{1B}AR | β₁AR | β₂AR |
|---|---|---|---|---|
| 0.027 ± 0.004 μM (3) | 1.3 ± 0.37 μM (4) | 7.4 ± 0.64 μM (3) | >10 μM (3) | >10 μM (3) |

^{a}Binding affinity to related adrenergic receptors as mean Kᵢ value in [μM ± s.e.m.] each done in triplicates; number of individual experiments are displayed in parentheses.

TABLE 5
Pharmacokinetic properties of docking agonists.

| | | | | | | Oral | Permeability |
|---|---|---|---|---|---|---|---|
| Com- | Routes | PK | | | | bio- | prediction |
| pound | (dose) | parameters^{a, b, c} | Plasma | Brain | CSF | availability^{d} | (kcal/mol)^{e} |
| ‘9087 | I.P. | Cmax | 1440 | 5960 | 447 | 152%^{f} | −2.207 |
| | (10 | (ng/ml) | 110000 | 439000 | 154000 | | |
| | mg/kg) | AUC | 0.50 | 9.85 | 26.83 | | |
| | | (ng*min/mL) | | | | | |
| | | T ½ (hr) | | | | | |
| | I.V. | Cmax | 2020 | 6590 | ND | | |
| | (10 | (ng/ml) | 105000 | 420000 | | | |
| | mg/kg) | AUC | 0.87 | 0.62 | | | |
| | | (ng*min/mL) | | | | | |
| | | T ½ (hr) | | | | | |
| | P.O. | Cmax | 2720 | 14000 | ND | | |
| | (30 | (ng/ml) | 506000 | 2540000 | | | |
| | mg/kg) | AUC | 1.25 | 1.06 | | | |
| | | (ng*min/mL) | | | | | |
| | | T ½ (hr) | | | | | |
| ‘7075 | I.P. | Cmax | 694 | 8570 | 412 | ND | −3.238 |
| (‘9087 | (10 | (ng/ml) | 57100 | 814000 | 12000 | | |
| analog) | mg/kg) | AUC | 0.64 | 0.73 | 0.22 | | |
| | | (ng*min/mL) | | | | | |
| | | T ½ (hr) | | | | | |
| PS75 | I.P. | Cmax | 679 | 9200 | 99.3 | 102%^{f} | ND |
| (‘9087 | (10 | (ng/ml) | 55600 | 888000 | 7790 | | |
| analog) | mg/kg) | AUC | 0.73 | 1.07 | 2.55 | | |
| | | (ng*min/mL) | | | | | |
| | | T ½ (hr) | | | | | |
| | I.V. | Cmax | 2586 | ND | ND | | |
| | (10 | (ng/ml) | 207,840 | | | | |
| | mg/kg) | AUC | 1.64 | | | | |
| | | (ng*min/mL) | | | | | |
| | | T ½ (hr) | | | | | |
| | P.O. | Cmax | 2390 | ND | ND | | |
| | (30 | (ng/ml) | 637,140 | | | | |
| | mg/kg) | AUC | 3.17 | | | | |
| | | (ng*min/mL) | | | | | |
| | | T ½ (hr) | | | | | |
| ‘4622 | I.P. | Cmax | 620 | 16200 | 44.2 | ND | −3.012 |
| | (10 | (ng/ml) | 34000 | 1160000 | 18400 | | |
| | mg/kg) | AUC | 1.01 | 0.88 | 14.5 | | |
| | | (ng*min/mL) | | | | | |
| | | T ½ (hr) | | | | | |
| ‘2998 | I.P. | Cmax | 2710 | 742 | n/a | ND | −1.121 |
| | (10 | (ng/ml) | 184000 | 51700 | | | |
| | mg/kg) | AUC | 0.4 | 0.46 | | | |
| | | (ng*min/mL) | | | | | |
| | | T ½ (hr) | | | | | |
| Dexmedet− | I.P. | Cmax | ND | 8 | ND | ND | ND |
| omidine | (30 | (ng/ml) | | 285 | | | |
| | μg/kg) | AUC | | ND | | | |
| | | (ng*min/mL) | | | | | |
| | | T ½ (hr) | | | | | |

^{a}Cmax, maximum concentration reached in mice plasma, brain, or CSF sample.
^{b}AUC, area under the concentration-time curve for exposure in mice plasma, brain, or CSF.
^{c}T ½, half-life of the compound in mice plasma, brain, or CSF.
^{d}Oral bioavailability, see methods for calculation using I.V. and P.O. dosing.
^{e}Delta-delta-G of insertion of compounds from water to membrane.
^{f}An oral bioavailability higher than 100% can occur with certain compound and pharmacokinetic conditions, including “non−linear PK” (metabolic saturation) at non-equal I.V. and P.O. doses and entero-hepatic recirculation.

### References for Example 3

1. A. S. Yekkirala, D. P. Roberson, B. P. Bean, C. J. Woolf, Nat. Rev. Drug Discov. 16, 545-564 (2017).
2. L. Manchikanti, S. Helm, B. Fellows, J. W. Janata, V. Pampati, J. S. Grider, M. V. Boswell, Pain Physician. 15, ES9-38 (2012).
3. J. Strang, N. D. Volkow, L. Degenhardt, M. Hickman, K. Johnson, G. F. Koob, B. D. L. Marshall, M. Tyndall, S. L. Walsh, Nat. Rev. Dis. Primer. 6, 3 (2020).
4. K. Rasmussen, D. A. White, J. B. Acri, Neuropsychopharmacology 44, 657-659 (2019).
5. A. B. Malmberg, L. R. Hedley, J. R. Jasper, J. C. Hunter, A. L. Basbaum, Br. J. Pharmacol. 132, 1827-1836 (2001).
6. P. P. Lakhlani, L. B. MacMillan, T. Z. Guo, B. A. McCool, D. M. Lovinger, M. Maze, L. E. Limbird, Proc. Natl. Acad. Sci. U.S.A 94, 9950-9955 (1997).
7. H. Buerkle, T. L. Yaksh, Br. J. Anaesth. 81, 208-215 (1998).
8. M. Anttila, J. Penttilä, A. Helminen, L. Vuorilehto, H. Scheinin, Br. J. Clin. Pharmacol. 56, 691-693 (2003).
9. D. Yuan, Z. Liu, J. Kaindl, S. Maeda, J. Zhao, X. Sun, J. Xu, P. Gmeiner, H.-W. Wang, B. K. Kobilka, Nat. Chem. Biol. 16, 507-512 (2020).
10. J. Lyu, S. Wang, T. E. Balius, I. Singh, A. Levit, Y. S. Moroz, M. J. O'Meara, T. Che, E. Algaa, K. Tolmachova, A. A. Tolmachev, B. K. Shoichet, B. L. Roth, J. J. Irwin, Nature 566, 224-229 (2019).
11. T. Sterling, J. J. Irwin, J. Chem. Inf Model. 55, 2324-2337 (2015).
12. J. J. Irwin, K. G. Tang, J. Young, C. Dandarchuluun, B. R. Wong, M. Khurelbaatar, Y. S. Moroz, J. Mayfield, R. A. Sayle, J. Chem. Inf Model. 60, 6065-6073 (2020).
13. R. M. Stein, H. J. Kang, J. D. McCorvy, G. C. Glatfelter, A. J. Jones, T. Che, S. Slocum, X.-P. Huang, O. Savych, Y. S. Moroz, B. Stauch, L. C. Johansson, V. Cherezov, T. Kenakin, J. J. Irwin, B. K. Shoichet, B. L. Roth, M. L. Dubocovich, Nature 579, 609-614 (2020).
14. A. Alon, J. Lyu, J. M. Braz, T. A. Tummino, V. Craik, M. J. O'Meara, C. M. Webb, D. S. Radchenko, Y. S. Moroz, X.-P. Huang, Y. Liu, B. L. Roth, J. J. Irwin, A. I. Basbaum, B. K. Shoichet, A. C. Kruse, Nature 600, 759-764 (2021).
15. C. Gorgulla, A. Boeszoermenyi, Z.-F. Wang, P. D. Fischer, P. W. Coote, K. M. Padmanabha Das, Y. S. Malets, D. S. Radchenko, Y. S. Moroz, D. A. Scott, K. Fackeldey, M. Hoffmann, I. Iavniuk, G. Wagner, H. Arthanari, Nature 580, 663-668 (2020).
16. A. A. Sadybekov, A. V. Sadybekov, Y. Liu, C. Iliopoulos-Tsoutsouvas, X.-P. Huang, J. Pickett, B. Houser, N. Patel, N. K. Tran, F. Tong, N. Zvonok, M. K. Jain, O. Savych, D. S. Radchenko, S. P. Nikas, N. A. Petasis, Y. S. Moroz, B. L. Roth, A. Makriyannis, V. Katritch, Nature 601, 452-459 (2022).
17. A. Manglik, H. Lin, D. K. Aryal, J. D. McCorvy, D. Dengler, G. Corder, A. Levit, R. C. Kling, V. Bernat, H. Hubner, X.-P. Huang, M. F. Sassano, P. M. Giguère, S. Löber, Da Duan, G. Scherrer, B. K. Kobilka, P. Gmeiner, B. L. Roth, B. K. Shoichet, Nature 537, 185-190 (2016).
18. M. Schuller, G. J. Correy, S. Gahbauer, D. Fearon, T. Wu, R. E. Díaz, I. D. Young, L. Carvalho Martins, D. H. Smith, U. Schulze-Gahmen, T. W. Owens, I. Deshpande, G. E. Merz, A. C. Thwin, J. T. Biel, J. K. Peters, M. Moritz, N. Herrera, H. T. Kratochvil, QCRG Structural Biology Consortium, A. Aimon, J. M. Bennett, J. Brandao Neto, A. E. Cohen, A. Dias, A. Douangamath, L. Dunnett, O. Fedorov, M. P. Ferla, M. R. Fuchs, T. J. Gorrie-Stone, J. M. Holton, M. G. Johnson, T. Krojer, G. Meigs, A. J. Powell, J. G. M. Rack, V. L. Rangel, S. Russi, R. E. Skyner, C. A. Smith, A. S. Soares, J. L. Wierman, K. Zhu, P. O'Brien, N. Jura, A. Ashworth, J. J. Irwin, M. C. Thompson, J. E. Gestwicki, F. von Delft, B. K. Shoichet, J. S. Fraser, I. Ahel, Sci. Adv. 7, eabf8711 (2021).
19. A. Rudling, R. Gustafsson, I. Almlöf, E. Homan, M. Scobie, U. Warpman Berglund, T. Helleday, P. Stenmark, J. Carlsson, J. Med. Chem. 60, 8160-8169 (2017).
20. A. Levit Kaplan, R. T. Strachan, J. M. Braz, V. Craik, S. Slocum, T. Mangano, V. Amabo, H. O'Donnell, P. Lak, A. I. Basbaum, B. L. Roth, B. K. Shoichet, J. Med. Chem. 65, 4201-4217 (2022).
21. C. de Graaf, A. J. Kooistra, H. F. Vischer, V. Katritch, M. Kuijer, M. Shiroishi, S. Iwata, T. Shimamura, R. C. Stevens, I. J. P. de Esch, R. Leurs, J. Med. Chem. 54, 8195-8206 (2011).
22. R. Kiss, B. Kiss, Á. Könczöl, F. Szalai, I. Jelinek, V. László, B. Noszál, A. Falus, G. M. Keserü, J. Med. Chem. 51, 3145-3153 (2008).
23. N. Patel, X. P. Huang, J. M. Grandner, L. C. Johansson, B. Stauch, J. D. McCorvy, Y. Liu, B. Roth, V. Katritch, eLife 9, e53779 (2020).
24. C. J. Langmead, S. P. Andrews, M. Congreve, J. C. Errey, E. Hurrell, F. H. Marshall, J. S. Mason, C. M. Richardson, N. Robertson, A. Zhukov, M. Weir, J. Med. Chem. 55, 1904-1909 (2012).
25. S. Wang, D. Wacker, A. Levit, T. Che, R. M. Betz, J. D. McCorvy, A. J. Venkatakrishnan, X.-P. Huang, R. O. Dror, B. K. Shoichet, B. L. Roth, Science 358, 381-386 (2017).
26. R. G. Coleman, M. Carchia, T. Sterling, J. J. Irwin, B. K. Shoichet, PLoS One 8, e75992 (2013).
27. K. Altosaar, P. Balaji, R. A. Bond, D. B. Bylund, S. Cotecchia, D. Devost, V. A. Doze, D. C. Eikenburg, S. Gora, E. Goupil, R. M. Graham, T. Hébert, J. P. Hieble, R. Hills, S. Kan, G. Machkalyan, M. C. Michel, K. P. Minneman, S. Parra, D. Perez, R. Sleno, R. Summers, P. Zylbergold, IUPHARBPS Guide Pharmacol. CITE. 2019 (2019), doi:10.2218/gtopdb/F4/2019.4.
28. A. Gaulton, L. J. Bellis, A. P. Bento, J. Chambers, M. Davies, A. Hersey, Y. Light, S. McGlinchey, D. Michalovich, B. Al-Lazikani, J. P. Overington, Nucleic Acids Res. 40, D1100-D1107 (2012).
29. H. S. Blaxall, T. J. Murphy, J. C. Baker, C. Ray, D. B. Bylund, J. Pharmacol. Exp. Ther. 259, 323-329 (1991).
30. P. B. M. W. M. Timmermans, P. A. Van Zwieten, J. Med. Chem. 25, 1389-1401 (1982).
31. B. V. Clineschmidt, L. M. Flataker, E. P. Faison, D. R. Haubrich, Arch. Int. Pharmacodyn. Ther. 244, 231-243 (1980).
32. S. Gu, M. S. Smith, Y. Yang, J. J. Irwin, B. K. Shoichet, J. Chem. Inf. Model. 61, 4331-4341 (2021).
33. V. Isberg, C. de Graaf, A. Bortolato, V. Cherezov, V. Katritch, F. H. Marshall, S. Mordalski, J.-P. Pin, R. C. Stevens, G. Vriend, D. E. Gloriam, Trends Pharmacol. Sci. 36, 22-31 (2015).
34. M. Congreve, S. P. Andrews, A. S. Doré, K. Hollenstein, E. Hurrell, C. J. Langmead, J. S. Mason, I. W. Ng, B. Tehan, A. Zhukov, M. Weir, F. H. Marshall, J. Med. Chem. 55, 1898-1903 (2012).
35. S. Bhattacharya, S. E. Hall, H. Li, N. Vaidehi, Biophys. J. 94, 2027-2042 (2008).
36. D. R. Weiss, S. Ahn, M. F. Sassano, A. Kleist, X. Zhu, R. Strachan, B. L. Roth, R. J. Lefkowitz, B. K. Shoichet, ACS Chem. Biol. 8, 1018-1026 (2013).
37. M. G. Eason, H. Kurose, B. D. Holt, J. R. Raymond, S. B. Liggett, J. Biol. Chem. 267, 15795-15801 (1992).
38. C. Avet, A. Mancini, B. Breton, C. Le Gouill, A. S. Hauser, C. Normand, H. Kobayashi, F. Gross, M. Hogue, V. Lukasheva, S. St-Onge, M. Carrier, M. Héroux, S. Morissette, E. B. Fauman, J.-P. Fortin, S. Schann, X. Leroy, D. E. Gloriam, M. Bouvier, eLife 11, e74101 (2022).
39. Y. Namkung, C. Le Gouill, V. Lukashova, H. Kobayashi, M. Hogue, E. Khoury, M. Song, M. Bouvier, S. A. Laporte, Nat. Commun. 7, 12178 (2016).
40. J. Xu, S. Cao, H. Hübner, D. Weikert, G. Chen, Q. Lu, D. Yuan, P. Gmeiner, Z. Liu, Y. Du, Sci. Adv. 8, eabj5347 (2022).
41. L. Qu, Q. Zhou, Y. Xu, Y. Guo, X. Chen, D. Yao, G. W. Han, Z.-J. Liu, R. C. Stevens, G. Zhong, D. Wu, S. Zhao, Cell Rep. 29, 2929-2935.e4 (2019).
42. F. M. Heydenreich, M. Marti-Solano, M. Sandhu, B. K. Kobilka, M. Bouvier, M. M. Babu, https://www.biorxiv.org/content/10.1101/2021.09.14.460253v1 (2021).
43. M. Masureel, Y. Zou, L.-P. Picard, E. van der Westhuizen, J. P. Mahoney, J. P. G. L. M. Rodrigues, T. J. Mildorf, R. O. Dror, D. E. Shaw, M. Bouvier, E. Pardon, J. Steyaert, R. K. Sunahara, W. I. Weis, C. Zhang, B. K. Kobilka, Nat. Chem. Biol. 14, 1059-1066 (2018).
44. M. Stanek, L.-P. Picard, M. F. Schmidt, J. M. Kaindl, H. Hubner, M. Bouvier, D. Weikert, P. Gmeiner, J. Med. Chem. 62, 5111-5131 (2019).
45. W. K. Kroeze, M. F. Sassano, X.-P. Huang, K. Lansu, J. D. McCorvy, P. M. Giguère, N. Sciaky, B. L. Roth, Nat. Struct. Mol. Biol. 22, 362-369 (2015).
46. H. Kodaira, H. Kusuhara, T. Fujita, J. Ushiki, E. Fuse, Y. Sugiyama, J. Pharmacol. Exp. Ther. 339, 935-944 (2011).
47. S. D. Shields, W. A. Eckert, A. I. Basbaum, J. Pain 4, 465-470 (2003).
48. R. Virtanen, J. M. Savola, V. Saano, Arch. Int. Pharmacodyn. Ther. 297, 190-204 (1989).
49. L. B. MacMillan, L. Hein, M. S. Smith, M. T. Piascik, L. E. Limbird, Science 273, 801-803 (1996).
50. P. P. Lakhlani, D. M. Lovinger, L. E. Limbird, Mol. Pharmacol. 50, 96-103 (1996).
51. B. P. Ceresa, L. E. Limbird, J. Biol. Chem. 269, 29557-29564 (1994).
52. J. C. Hunter, D. J. Fontana, L. R. Hedley, J. R. Jasper, R. Lewis, R. E. Link, R. Secchi, J. Sutton, R. M. Eglen, Br. J. Pharmacol. 122, 1339-1344 (1997).
53. S. A. Metz, J. B. Halter, R. Paul Robertson, Diabetes 27, 554-562 (1978).
54. S. S. Lichtenstein, C. Marinescu, S. F. Leibowitz, Brain Res. Bull. 13, 591-595 (1984).
55. L. S. Stone, J. P. German, K. F. Kitto, C. A. Fairbanks, G. L. Wilcox, PLoS One 9, e109903 (2014).
56. D. A. Erlanson, S. W. Fesik, R. E. Hubbard, W. Jahnke, H. Jhoti, Nat. Rev. Drug Discov. 15, 605-619 (2016).
57. E. Von Moo, K. Harpsøe, A. S. Hauser, I. Masuho, H. Bräuner-Osborne, D. E. Gloriam, K. A. Martemyanov, Cell Chem. Biol., S2451945621003147 (2021).
58. I. D. Kuntz, J. M. Blaney, S. J. Oatley, R. Langridge, T. E. Ferrin, J. Mol. Biol. 161, 269-288 (1982).
59. J. M. Word, S. C. Lovell, J. S. Richardson, D. C. Richardson, J. Mol. Biol. 285, 1735-1747 (1999).
60. S. J. Weiner, P. A. Kollman, D. A. Case, U. C. Singh, C. Ghio, G. Alagona, S. Profeta, P. Weiner, J. Am. Chem. Soc. 106, 765-784 (1984).
61. B. J. Bender, S. Gahbauer, A. Luttens, J. Lyu, C. M. Webb, R. M. Stein, E. A. Fink, T. E. Balius, J. Carlsson, J. Irwin, B. K. Shoichet, Nat. Protoc. 16, 4799-4832 (2021).
62. R. M. Stein, Y. Yang, T. E. Balius, M. J. O'Meara, J. Lyu, J. Young, K. Tang, B. K. Shoichet, J. J. Irwin, J. Chem. Inf Model. 61, 699-714 (2021).
63. M. M. Mysinger, D. R. Weiss, J. J. Ziarek, S. Gravel, A. K. Doak, J. Karpiak, N. Heveker, B. K. Shoichet, B. F. Volkman, Proc. Natl. Acad. Sci. U.S.A 109, 5517-5522 (2012).
64. B. K. Shoichet, A. R. Leach, I. D. Kuntz, Proteins 34, 4-16 (1999).
65. K. Gallagher, K. Sharp, Biophys. J. 75, 769-776 (1998).
66. M. M. Mysinger, B. K. Shoichet, J. Chem. Inf Model. 50, 1561-1573 (2010).
67. T. Rezai, J. E. Bock, M. V. Zhou, C. Kalyanaraman, R. S. Lokey, M. P. Jacobson, J. Am. Chem. Soc. 128, 14073-14080 (2006).
68. S. S. F. Leung, J. Mijalkovic, K. Borrelli, M. P. Jacobson, J. Chem. Inf. Model. 52, 1621-1636 (2012).
69. M. P. Jacobson, G. A. Kaminski, R. A. Friesner, C. S. Rapp, J. Phys. Chem. B 106, 11673-11680 (2002).
70. D. G. Gibson, L. Young, R.-Y. Chuang, J. C. Venter, C. A. Hutchison, H. O. Smith, Nat. Methods 6, 343-345 (2009).
71. O. H. Lowry, N. J. Rosebrough, A. L. Farr, R. J. Randall, J. Biol. Chem. 193, 265-275 (1951).
72. Y. Cheng, W. H. Prusoff, Biochem. Pharmacol. 22, 3099-3108 (1973).
73. X. M. Guan, T. S. Kobilka, B. K. Kobilka, J. Biol. Chem. 267, 21995-21998 (1992).
74. J. Quoyer, J. M. Janz, J. Luo, Y. Ren, S. Armando, V. Lukashova, J. L. Benovic, K. E. Carlson, S. W. Hunt, M. Bouvier, Proc. Natl. Acad. Sci. U.S.A 110, E5088-5097 (2013).
75. C. Galds, J. J. J. Van Durm, S. Schaak, S. Pontier, Y. Percherancier, M. Audet, H. Paris, M. Bouvier, Nat. Struct. Mol. Biol. 13, 778-786 (2006).
76. D. Möller, A. Banerjee, T. C. Uzuneser, M. Skultety, T. Huth, B. Plouffe, H. Hubner, C. Alzheimer, K. Friedland, C. P. Müller, M. Bouvier, P. Gmeiner, J. Med. Chem. 60, 2908-2929 (2017).
77. H. Liu, J. Hofmann, I. Fish, B. Schaake, K. Eitel, A. Bartuschat, J. Kaindl, H. Rampp, A. Banerjee, H. Hubner, M. J. Clark, S. G. Vincent, J. T. Fisher, M. R. Heinrich, K. Hirata, X. Liu, R. K. Sunahara, B. K. Shoichet, B. K. Kobilka, P. Gmeiner, Proc. Natl. Acad. Sci. U.S.A. 115, 12046-12050 (2018).
78. J. Xu, Y. Hu, J. Kaindl, P. Risel, H. Hubner, S. Maeda, X. Niu, H. Li, P. Gmeiner, C. Jin, B. K. Kobilka, Mol. Cell 75, 53-65.e7 (2019).
79. X. Liu, J. Kaindl, M. Korczynska, A. StMBel, D. Dengler, M. Stanek, H. Hubner, M. J. Clark, J. Mahoney, R. A. Matt, X. Xu, K. Hirata, B. K. Shoichet, R. K. Sunahara, B. K. Kobilka, P. Gmeiner, Nat. Chem. Biol. 16, 749-755 (2020).
80. S. Maeda, A. Koehl, H. Matile, H. Hu, D. Hilger, G. F. X. Schertler, A. Manglik, G. Skiniotis, R. J. P. Dawson, B. K. Kobilka, Nat. Commun. 9, 3712 (2018).
81. X. Huang, L. Zhang, Z. Wen, H. Chen, S. Li, G. Ji, C. Yin, F. Sun, Prog. Biophys. Mol. Biol. 156, 3-13 (2020).
82. D. N. Mastronarde, J. Struct. Biol. 152, 36-51 (2005).
83. S. Q. Zheng, E. Palovcak, J.-P. Armache, K. A. Verba, Y. Cheng, D. A. Agard, Nat. Methods 14, 331-332 (2017).
84. W. Jiang, F. Guo, Z. Liu, J. Struct. Biol. 180, 343-351 (2012).
85. A. Punjani, J. L. Rubinstein, D. J. Fleet, M. A. Brubaker, Nat. Methods 14, 290-296 (2017).
86. S. H. W. Scheres, J. Struct. Biol. 180, 519-530 (2012).
87. P. Emsley, B. Lohkamp, W. G. Scott, K. Cowtan, Acta Crystallogr. D Biol. Crystallogr. 66, 486-501 (2010).
88. W. J. Allen, R. C. Rizzo, J. Chem. Inf Model. 54, 518-529 (2014).
89. C. A. Flanagan, “GPCR-radioligand binding assays” in Methods in Cell Biology (Elsevier, 2016), vol. 132, pp. 191-215.
90. R. N. DeHaven, D. L. DeHaven-Hudkins, Curr. Protoc. Pharmacol. 8 (2000), doi:10.1002/0471141755.ph0104 s08.
91. E. C. Hulme, M. A. Trevethick, Br. J. Pharmacol. 161, 1219-1237 (2010).
92. X.-P. Huang, T. Mangano, S. Hufeisen, V. Setola, B. L. Roth, ASSAY Drug Dev. Technol. 8, 727-742 (2010).
93. C. Hiller, R. C. Kling, F. W. Heinemann, K. Meyer, H. Hubner, P. Gmeiner, J. Med. Chem. 56, 5130-5141 (2013).
94. J. Hellmann, M. Drabek, J. Yin, J. Gunera, T. Pröll, F. Kraus, C. J. Langmead, H. Hubner, D. Weikert, P. Kolb, D. M. Rosenbaum, P. Gmeiner, Proc. Natl. Acad. Sci. U.S.A 117, 18059-18067 (2020).
95. L. Di, C. Keefer, D. O. Scott, T. J. Strelevitz, G. Chang, Y.-A. Bi, Y. Lai, J. Duckworth, K. Fenner, M. D. Troutman, R. S. Obach, Eur. J. Med. Chem. 57, 441-448 (2012).
96. G. Scherrer, N. Imamachi, Y.-Q. Cao, C. Contet, F. Mennicken, D. O'Donnell, B. L. Kieffer, A. I. Basbaum, Cell 137, 1148-1159 (2009).
97. A. Muralidharan, A. Samoshkin, M. Convertino, M. H. Piltonen, P. Gris, J. Wang, C. Jiang, R. Klares, A. Linton, R. Ji, W. Maixner, N. V. Dokholyan, J. S. Mogil, L. Diatchenko, Br. J. Pharmacol. 178, 2709-2726 (2021).
98. C. G. Alvaro, J. M. Braz, M. Bernstein, K. A. Hamel, V. Craik, H. Yamanaka, A. I. Basbaum, PLoS One 15, e0226289 (2020).
99. S. R. Chaplan, F. W. Bach, J. W. Pogrel, J. M. Chung, T. L. Yaksh, J. Neurosci. Methods 53, 55-63 (1994).
100. Y. Q. Cao, P. W. Mantyh, E. J. Carlson, A.-M. Gillespie, C. J. Epstein, A. I. Basbaum, Nature 392, 390-394 (1998).

### Example 4: Additional Chemical Synthesis Data

Synthesis procedures were conducted using standard equipment and devices. Reactions were performed under inert atmosphere using nitrogen gas if not stated otherwise.

### Solvents

Solvents were purchased in the highest available purity grade from Acros and Fisher Scientific and used without further purification. Dry solvents for synthetic procedures were purchased and stored, whenever possible, in sealed septum bottles over molecular sieves.

### Chemicals

Chemicals for synthetic procedures were purchased from Angene Chemical, BLDpharm, Enamine, Sigma Aldrich and Synthonix in the highest available purity grade and used without further purification.

### NMR-Spectroscopy

NMR spectra were obtained on a Bruker Avance 400 (¹H at 400 MHz, ¹³C (DEPTQ) at 101 MHz) or a Bruker Avance 600 (¹H at 600 MHz, ¹³C (DEPTQ) at 151 MHz) spectrometer at 298K using the solvents indicated. Chemical shifts are reported relative to the solvent peak.

### High-Resolution Mass Spectrometry

ESI-TOF high mass accuracy and resolution experiments were performed on an AB Sciex Triple TOF660 Sciex, on a Bruker maXis MS or a Bruker timsTOF Pro.

### HPLC-MS

Analytical LCMS was performed on Thermo Scientific Dionex Ultimate 3000 HPLC system using DAD detection (230 nm; 254 nm) equipped with either a Kinetex 2.6 u mesh C8 100A (2.1×75 mm, 2.6 m) HPLC column or a Zorbax Eclipse XDB-C8 (4.6×150 mm, 5 m) HPLC column, using mass detection on a BRUKER amaZon SL mass spectrometer using ESI or APCI ionization source.

### Preparative RP-HPLC

Purification by preparative RP-HPLC was performed on AGILENT 1260 Preparative Series equipped with a VWD detector (210 nm, 230 nm; 254 nm, 280 nm) using a Zorbax Eclipse XDB-C8 21.2×150 mm column with 5 μm particles [C8], flow rate 10 mL/min, employing eluent systems as specified below.

### Analytical RP-HPLC

HPLC purity analyses were performed with an Agilent binary gradient system using UV detection (X=210, 220, 230, 254, and 280 nm) in combination with ChemStation software. A Zorbax Eclipse XDB-C₈ (4.6 mm×150 mm, 5 m) column was used with a flow rate of 0.5 mL/min in reversed-phase mode.
eluent system 1: methanol/H₂O+0.1% HCO₂H
   gradient: 10% for 3 min, 10% to 100% in 15 min, 100% for 6 min, 100% to 10% in 3 min, 10% for 3 min
eluent system 2: acetonitrile/H₂O+0.1% HCO₂H
   gradient: 5% for 3 min, 5% to 95% in 15 min, 95% for 6 min, 95% to 5% in 3 min, 5% for 3 min
      General procedure for the Coupling of bromo-1H-indazoles/bromo-2,3-dihydro-1H-indens/tert-butyl (2-bromopyridin-4-yl)carbamate (GP1)

In a microwave tube, the respective bromo-1H-indazole, bromo-2,3-dihydro-1H-inden or tert-butyl (2-bromopyridin-4-yl)carbamate and the respective aryl boronic acid pinacol ester were dissolved in dioxane. To the solution, [1,1′-Bis-(diphenylphosphino)-ferrocen]-dichloro-palladium(II) was added under nitrogen flow. An aqueous 1M solution of Na₂CO₃ was added. The reaction mixture was purged for further 3 minutes. The microwave tube was sealed and stirred at 80° C., 100° C. or in the microwave for 4 h at 80° C. After completion the reaction mixture was let cool to ambient temperature before it was used crude.

### General Procedure for the Deprotection of Boc-Protected Pyridinyl Carbamates or Dihydropyridine Carboxylates (GP2)

To the crude mixture of the previous Suzuki coupling trifluoroacetic acid (pH=1) was added dropwise at ambient temperature. The reaction mixture was stirred at 50° C. After completion, the solvent was evaporated. The product was dissolved in 1.0 ml MeOH, was mixed with a few drops of TFA, filtered and purified using preparative HPLC. All final products were obtained as TFA salts.

### 5-(Bicyclo[4.2.0]octa-1,3,5-trien-3-yl)-1,2,3,6-tetrahydropyridine (ZINC001240664622, WXVL_BT1618LQ2267)

2-(1-Phenylvinyl)pyridin-4-amine (ZINC001242890172, WXVL_BT2072LP0110)

### Enamine General Procedure 1

Halogenide (1 eq.), and boronic acid (1.5 eq.) were mixed in dioxane (appr. 0.5 m1 per 100 mg of product), and Pd(dppf)Cl₂ (0.05 eq.) (as stock solution in dioxane appr. 0.05 ml per 100 mg of product), and disodium carbonate (Na₂CO₃) (2.5 eq.) (as 1 M stock solution in water) were added in one portion in an inert atmosphere. The reaction mixture was sealed and heated with shaking for 16 hours at 100° C. The mixture was cooled to the ambient temperature and Trifluoroacetic acid was added dropwise until neutral pH. Then the solvent was evaporated under reduced pressure and the residue was dissolved in the DMSO (appr. 1 m1 per 100 mg of product). DMSO solution was treated with Scavenger SiliaMetS DMT, filtered, analyzed by LC/MS, and purified by HPLC. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. Yields: Z4767467059-24.7 mg (24.0%); Z4767470594-20.6 mg (19.9%); Z6071720022-65.8 mg (21.9%).

### Enamine General Procedure 2

Halogenide (1 eq.), boronic acid or pinacolate (1.2 eq.), were mixed in dry dioxane (appr. 0.7 ml per 100 mg of product) and then XPhos Pd G3 (0.05 eq.) (as 0.2 M solution in dioxane), and disodium carbonate (Na₂CO₃ (2.2 eq.) (as 1 M solution in water) were added in one portion in an inert atmosphere. The reaction mixture was sealed and heated for 16 hours at 90° C. The mixture was cooled to the ambient temperature and trifluoroacetic acid (TFA) was added dropwise until neutral pH. Then the solvent was evaporated under reduced pressure and the residue was dissolved in the DMSO (appr. 1 ml per 100 mg of product). DMSO solution was treated with Scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. Yields: Z2204874213-6.8 mg (6.8%); Z2724116078-14.7 mg (14.6%); Z4767467055-19.7 mg (22.6%).

### Enamine Procedure 3

5-Bromo-4-chloro-2,3-dihydro-1H-indene EN300-7461412 (104.0 mg, 0.449 mmol, 1 eq.) and tert-butyl 5-(tetramethyl-1,3,2-dioxaborolan-2-yl)-1,2,3,6-tetrahydropyridine-1-carboxylate EN300-170563 (166.7 mg, 0.539 mmol, 1.2 eq.) were mixed in dry dioxane (0.5 m1) and then XPhos Pd G3 (19.0 mg, 0.022 mmol, 0.05 eq.), and disodium carbonate (Na₂CO₃) (104.7 mg, 0.988 mmol, 2.2 eq.) were added in one portion in an inert atmosphere. The reaction mixture was sealed and heated with stirring for 16 hours at 90° C., cooled to ambient temperature and the cleavage cocktail (CC) (trifluoroacetic acid, triisopropylsilane, water (93:5:2; v/v), appr. 1 ml per 100 mg of product) was added in one portion. The mixture was stirred for 6 hours at ambient temperature and evaporated under reduced pressure and the residue was dissolved in the DMSO (appr. 1 ml up to 300 mg of product). DMSO solution was treated with scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. TFA was used as a mobile phase modifier. After HPLC 33.8 mg (21.6% yield) of pure compound Z4923386334 was obtained.

### Enamine Procedure 4

3-Chloro-5H,6H,7H-cyclopenta[c]pyridine-4-carbonitrile EN300-178115 (82.0 mg, 0.459 mmol, 1 eq.) and tert-butyl 5-(tetramethyl-1,3,2-dioxaborolan-2-yl)-1,2,3,6-tetrahydropyridine-1-carboxylate EN300-170563 (212.9 mg, 0.689 mmol, 1.5 eq.) were mixed in dry dioxane (0.5 ml) and then cataCXium® Pd G3 (16.7 mg, 0.023 mmol, 0.05 eq.), RuPhos Pd G4 (19.5 mg, 0.023 mmol, 0.05 eq.), disodium carbonate (Na₂CO₃) (121.6 mg, 1.148 mmol, 2.5 eq.) were added in one portion in an inert atmosphere. The reaction mixture was sealed and heated with stirring for 16 hours at 90° C., cooled to ambient temperature and the cleavage cocktail (CC) (trifluoroacetic acid, triisopropylsilane, water (93:5:2; v/v), appr. 1 m1 per 100 mg of product) was added in one portion. The mixture was stirred for 6 hours at ambient temperature and evaporated under reduced pressure and the residue was dissolved in the DMSO (appr. 1 ml up to 300 mg of product). DMSO solution was treated with scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. After HPLC 35.0 mg (34.2% yield) of pure compound Z4767467035 was obtained.

### Enamine General Procedure 5

Amine (1.2 eq.), halogenide 2 (1 eq.), and Cs₂CO₃ (2.5 eq.) were mixed in dry dioxane (appr. 0.7 ml per 100 mg of product). Then XantPhos Pd G4 (0.05 eq.) (as a stock solution in dioxane appr. 0.05 ml per 100 mg of product), and XantPhos (0.05 eq.) (as a stock solution in Dioxane appr. 0.05 ml per 100 mg of product) were added in one portion in an inert atmosphere. The reaction mixture was sealed and heated with shaking for 16 hours at 100° C. Then the mixture was cooled and trifluoroacetic acid (TFA) was added dropwise until neutral pH and the solvent was evaporated under reduced pressure, and the residue was dissolved in the DMSO (appr. 1 ml per 100 mg of product). DMSO solution was treated with Scavenger SiliaMetS DMT and filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. Yields: Z4767467012-11.7 mg (12.1%); Z1509600173-2.2 mg (1.8%).

### Enamine General Procedure 6

Amine (1 eq.), halogenide (1.1 eq.), and caesium carbonate (Cs₂CO₃) (2.5 eq.) were mixed in dry dioxane (appr. 0.5 ml per 100 mg of product). Then XantPhos Pd G4 (0.05 eq.) (as a stock solution in dioxane appr. 0.05 ml per 100 mg of product), and XantPhos (0.05 eq.) (as a stock solution in dioxane appr. 0.05 ml per 100 mg of product) were added in one portion in an inert atmosphere. The reaction mixture was sealed and heated with stirring for 16 hours at 100° C. Then the mixture was cooled to ambient temperature and the Cleavage cocktail (CC) (Trifluoroacetic acid, triisopropylsilane, water (93:5:2; v/v), appr. 1 ml per 100 mg of product) was added in one portion. The mixture was stirred for 6 hours at ambient temperature and the solvent was evaporated under reduced pressure and the residue was dissolved in the DMSO (appr. 1 ml up to 300 mg of product). DMSO solution was treated with scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. Yields: Z4767467010-19.5 mg (13.3%); Z4767467018-15.4 mg (10.0%); Z4507373641-51.8 mg (29.3%).

### Enamine Procedure 7

5-Bromopyridin-2-amine EN300-17910 (97.1 mg, 0.561 mmol, 1 eq.) was dissolved mixed in dry dioxane (0.7 ml). THF solution of LiHMDS (375.6 mg, 2.245 mmol, 4.0 eq.) was added dropwise to the solution. Then N,3-dimethylaniline EN300-59272 (204.0 mg, 1.683 mmol, 3.0 eq.), XPhos Pd G4 (24.1 mg, 0.028 mmol, 0.05 eq.), and XPhos (13.4 mg, 0.028 mmol, 0.05 eq.) were added in one portion in an inert atmosphere. The reaction mixture was sealed and heated with shaking for 24 hours at 85° C. Then the mixture was cooled and trifluoroacetic acid (TFA) was added dropwise until neutral pH and the solvent was evaporated under reduced pressure, and the residue was dissolved in the DMSO (appr. 1 m1 per 100 mg of product). DMSO solution was treated with Scavenger SiliaMetS DMT and filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. After HPLC 16.4 mg (13.7% yield) of pure compound Z4767467005 was obtained.

### Enamine Procedure 8

3-(Methylamino)phenol EN300-79043 (112.0 mg, 0.909 mmol, 1.5 eq.) was dissolved mixed in dry dioxane (0.7 ml). THF solution of LiHMDS (507.2 mg, 3.03 mmol, 5.0 eq.) was added dropwise to the solution. Then tert-butyl N-(2-chloropyridin-4-yl)carbamate EN300-201252 (138.6 mg, 0.03 mmol, 1.0 eq.), JohnPhos Pd G4 (20.7 mg, 0.03 mmol, 0.05 eq.), and JohnPhos (9.0 mg, 0.909 mmol, 0.05 eq.) were added in one portion in an inert atmosphere. The reaction mixture was sealed and heated with shaking for 24 hours at 85° C. Then the mixture was cooled to ambient temperature and the Cleavage cocktail (CC) (trifluoroacetic acid, triisopropylsilane, water (93:5:2; v/v), 1 ml) was added in one portion. The mixture was stirred for 6 hours at ambient temperature and the solvent was evaporated under reduced pressure and the residue was dissolved in the DMSO (1 ml). DMSO solution was treated with scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeOH (phase B) were used as an eluent. NH₄OH was used as a mobile phase modifier. After HPLC 5.4 mg (3.5% yield) of pure compound Z4767467044 was obtained.

### Enamine Procedure 9

4-Fluorophenol (85.0 mg, 0.7583 mmol, 1.5 eq.), tert-butyl N-(2-chloropyridin-4-yl)carbamate EN300-201252 (115.6 mg, 0.5055 mmol, 1.0 eq.), and Cs₂CO₃ (494.1 mg, 1.5175 mmol, 3.0 eq.) were mixed in dry NMP (0.5 ml) and then CuCl (10.0 mg, 0.1011 mmol, 0.2 eq.), and 2,2,6,6-tetramethylheptane-3,5-dione (37.3 mg, 0.2022 mmol, 0.4 eq.) were added in one portion in an inert atmosphere. The reaction mixture was sealed and heated with shaking for 48 hours at 120° C. Then the mixture was cooled to ambient temperature and the Cleavage cocktail (CC) (trifluoroacetic acid, triisopropylsilane, water (93:5:2; v/v), 1 m1) was added in one portion. The mixture was stirred for 6 hours at ambient temperature and the solvent was evaporated under reduced pressure and the residue was dissolved in the DMSO (1 ml). DMSO solution was treated with scavenger SiliaMetS DMT, filtered, analyzed by LC/MS, and transferred for HPLC purification. The purification was performed using Agilent 1260 Infinity systems equipped with DAD and mass-detector. Waters Sunfire C18 OBD Prep Column, 100 A, 5 μm, 19 mm×100 mm with SunFire C18 Prep Guard Cartridge, 100 A, 10 μm, 19 mm×10 mm was used. Deionized water (phase A) and HPLC-grade MeCN (phase B) were used as an eluent. Formic a was used as a mobile phase modifier. After HPLC 29.7 mg (18.5% yield) of pure compound Z4907706973 was obtained.

All chemicals and solvents for the synthesis of compounds were obtained from WuXi. 1H and 13C NMR spectra were acquired on Bruker Avance NEO 400 and quantum one 400 MHz spectrometers using DMSO-d6 as a solvent, LC/MS data were recorded on: The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was Acetonitrile. The column used for chromatography was a Xbridge Shield RP18 2.1*50 mm column (5 um particles). Detection methods are diode array (DAD) as well as positive electrospray ionization. MS range was 100-1000; The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was Acetonitrile. The column used for chromatography was a Xbridge Shield RP18 2.1*50 mm column (5 um particles). Detection methods are diode array (DAD) as well as positive electrospray ionization. MS range was 100-1000. Purity of compounds were assessed based on ¹H NMR and LC/MS data. Crude samples with product content below 90% were purified using masstriggered Agilent 1200 HPLC systems utilizing various gradients depending on a SlogP value of a particular compound.

### 5-(Bicyclo[4.2.0]octa-1,3,5-trien-3-yl)-1,2,3,6-tetrahydropyridine (ZINC001240664622, WXVL_BT1618LQ2267)

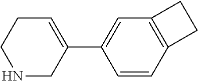 [SMILES: C1=C(c2ccc3c(c2)CC3)CNCC1]

Aromatic bromide (0.1 g, 393.58 umol, 1.0 eq) was dissolved in dioxane (2 mL) and H₂O (0.4 mL). A boronic acid/boronic acid pinacol ester (1.0 eq) was added to the solution together with K₃PO₄ (3.0 eq), (Boc)2O (1.0 eq) and Pd(dppf)Cl₂ (0.1 eq) under argon protection. The mixture was shaken at 80° C. for 8 hrs. LC-MS showed the desired product mass was detected. To the reaction mixture was added thiourea resin (0.5 g), and the mixture was stirred at 25° C. for another 16 hrs. Then the mixture was filtered through a celite pad, and the filtrate was concentrated to give the crude product. The residue was purified by prep-HPLC. Yield: 54.2%. ¹H NMR (400 MHz, DMSO-d₆): δ 2.40 (s(br), 2H), 3.14-3.20 (m, 6H), 3.90 (s(br), 2H), 6.20-6.24 (m, 1H), 7.08 (dd, J=7.7, 0.9 Hz, 1H), 7.14 (s(br), 1H), 7.22 (dd, J=7.7, 1.5 Hz, 1H), 8.79(s(br), 1H). ¹³C NMR (101 MHz, DMSO-d₆): δ, 22.40, 28.99, 29.03, 39.83, 43.17, 119.13, 121.27, 122.66, 123.70, 132.04, 136.91, 145.05, 145.57.

### tert-Butyl 5-(1H-indol-4-yl)-3,6-dihydropyridine-1(2H)-carboxylate (PS-NN04-Boc)

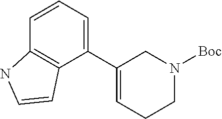 [SMILES: CC(C)(C)OC(=O)N1CCC=C(c2cccc3[nH]ccc23)C1]

tert-Butyl-5-(4,4,5,5-tetramethyl-1,3,2-dioxaborolan-2-yl)-3,6-dihydropyridine-1(2H)-carboxylate (32.0 mg, 103 μmol), 4-Bromoindole (9.36 μl, 74.6 μmol), Pd(dppf)Cl₂ (13.0 mg, 17.8 μmol) and Na₂CO₃ (21.9 mg, 207 μmol) were dissolved in dioxane (3 ml) and water (0.8 ml). The microwave tube was purged with nitrogen an heated for 2 h to 100° C. while stirring. The reaction progress was monitored using thin layer chromatography and LC/MS. After the reaction mixture had cooled down to room temperature the aqueous phase was washed with ethyl acetate (3×50 ml). The combined organic layer was dried over Na₂SO₄, filtered and concentrated under reduced pressure. Then the brown oil was purified by flash column chromatography on normal phase (eluent: ethyl acetate and isohexane (1:9)). Afterwards the collected fractions were concentrated under reduced pressure, the product was obtained as brown oil. ¹H NMR (400 MHz, DMSO-d₆): δ 11.19 (s, 1H), 7.43-7.29 (m, 2H), 7.06 (dd, J=8.1, 7.3 Hz, 1H), 6.88 (dd, J=7.3, 0.9 Hz, 1H), 6.54 (s, 1H), 6.13 (dt, J=4.3, 2.4 Hz, 1H), 4.25 (q, J=2.4 Hz, 2H), 3.53 (t, J=5.8 Hz, 2H), 2.30 (h, J=3.2 Hz, 2H), 1.43 (s, 9H). ¹³C NMR (101 MHz, DMSO-d₆) δ 136.71, 132.30, 125.78, 125.60, 123.93, 121.36, 116.97, 111.30, 101.03, 79.36, 60.23, 28.59, 21.25, 14.56; LC/MS (ESI+) m/z: 299.5 [M+H]+.

### 4-(1,2,5,6-tetrahydropyridin-3-yl)-1H-indole (PS-NN04)

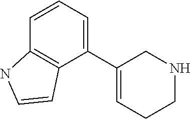 [SMILES: C1=C(c2cccc3[nH]ccc23)CNCC1]

tert-Butyl 5-(1H-indol-4-yl)-3,6-dihydropyridine-1(2H)-carboxylate (PS-NN04-Boc) was dissolved in HCl in dioxane (4 M) and stirred at room temperature for 2 h. The reaction was monitored using LC/MS. The compound was purified by preparative HPLC (column 2). The collected fractions were freeze-dried to isolate the title compound as a white solid (2.4 mg, 12%). ¹H NMR (600 MHz, Methanol-d4): δ 7.41-7.35 (m, 1H), 7.29 (d, J=3.2 Hz, 1H), 7.11 (t, J=7.7 Hz, 1H), 6.93 (d, J=7.3 Hz, 1H), 6.61-6.55 (m, 1H), 6.39-6.26 (m, 1H), 4.11 (q, J=2.2 Hz, 2H), 3.45 (t, J=6.3 Hz, 1H), 2.66 (q, J=2.9 Hz, 2H); ¹³C NMR (101 MHz, Methanol-d4) δ 132.98, 131.53, 126.23, 124.07, 122.20, 118.14, 112.51, 101.31, 45.55, 41.76, 23.12; LC/MS (ESI+) m/z: 199.04 [M+H]+.

### Tert-butyl 5-(3-oxo-2,3-dihydro-1H-inden-5-yl)-3,6-dihydropyridine-1(2H)-carboxylate (Compound 1): (TP NN02)

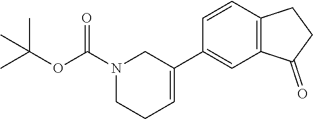 [SMILES: CC(C)(C)OC(=O)N1CCC=C(c2ccc3c(c2)C(=O)CC3)C1]

Compound 1 was prepared according to GP1 using 6-bromo-2,3-dihydro-1H-inden-1-one (15.8 mg, 74.6 μmol), tert-butyl 5-(4,4,5,5-tetramethyl-1,3,2-dioxaborolan-2-yl)-3,6-dihydropyridine-1(2H)-carboxylate (30.0 mg, 97.0 μmol), Pd(dppf)Cl₂ (2.73 mg, 3.73 μmol) and Na₂CO₃ (18.2 mg. 172 μmol) in 2.5 ml dioxane and 0.5 ml H₂O. The reaction was finished after 48 h. The reaction mixture was used crude. ESI-MS: 336.30 [M+Na]+; 214.70 [M_{deprotected}+H]⁺, 649.44 [2M+Na]⁺.

### Tert-butyl 5-(3-methyl-1H-inden-6-yl)-3,6-dihydropyridine-1(2H)-carboxylate (TP_NN07)

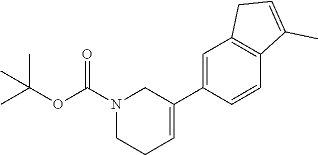 [SMILES: CC1=CCc2cc(C3=CCCN(C(=O)OC(C)(C)C)C3)ccc21]

TP_NN07 was prepared according to GP1 using 6-bromo-3-methyl-1H-indene (20.0 mg, 95.7 μmol), tert-butyl 5-(4,4,5,5-tetramethyl-1,3,2-dioxaborolan-2-yl)-3,6-dihydropyridine-1(2H)-carboxylate (38.5 mg, 124 μmol), Pd(dppf)Cl₂ (3.50 mg, 4.78 μmol) and Na₂CO₃ (23.3 mg, 220 μmol) in 2.5 ml dioxane and 0.5 ml H₂O. The reaction was finished after 21 h. The solvent was evaporated. The residue was dissolved in 1.0 ml MeOH and filtered. Purification by preparative HPLC (ACN/H₂O+0.1% TFA: 10% for 3 min, 10%→90% in 8 min, 90% for 8 min, 90%→10% in 1 min, 10% for 1 min) yielded 9.30 mg (29.9 μmol, 31%) of an off-white solid. ESI-MS: 211.05 [M_{deprotected}+H]⁺, 312.25 [M+H]⁺, 334.xx [M+Na]⁺, 623.56 [2M+H]⁺, 645.56 [2M+Na]⁺. ¹H NMR: (400 MHz, CDCl₃) δ 7.50 (s, 1H), 7.37-7.29 (m, 2H), 6.24-6.19 (m, 2H), 4.33 (s, 2H), 3.62-3.52 (m, 2H), 3.34 (s, 2H), 2.34 (s, 2H), 2.22-2.15 (m, 3H), 1.52 (s, 9H).

### 5-(3-Methyl-1H-inden-6-yl)-1,2,3,6-tetrahydropyridine (TP97)

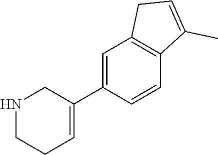 [SMILES: CC1=CCc2cc(C3=CCCNC3)ccc21]

TP97 was prepared according to GP2 using TP_NN07 (9.30 mg, 29.9 μmol) and trifluoroacetic acid. The reaction was finished after 22 h. Purification by preparative HPLC (ACN/H₂O+0.1% TFA: 10% for 3 min, 10%→65% in 10 min, 65%→90% in 1 min, 90% for 1 min, 90%→10% in 1 min, 10% for 1 min) yielded 2.60 mg (12.3 μmol, 41%) of an off-white solid. ESI-MS: 211.96 [M+H]⁺; 423.22 [2M+H]⁺. ¹H NMR: (400 MHz, CDCl₃) δ 7.38 (dd, J=1.7, 0.8 Hz, 1H), 7.29-7.26 (m, 1H), 7.22 (dd, J=7.9, 1.7 Hz, 1H), 6.25-6.21 (m, 2H), 3.37-3.26 (m, 4H), 2.59 (d, J=6.0 Hz, 2H), 2.16-2.14 (m, 3H), 1.41 (q, J=7.2 Hz, 2H). ¹³C NMR: 101 MHz, CDCl₃) δ 146.37, 144.85, 139.65, 133.83, 131.19, 129.82, 123.20, 120.93, 120.43, 118.91, 52.67, 43.41, 37.71, 22.15, 13.04. HR-MS (ESI): m/z [M+H]⁺ found 212.1434, calculated 212.1361 for C₁₄H₁₅NO.

### Purity:

| eluent system 1, t_{R} = 15.9 min: | eluent system 2, t_{R} = 14.3 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 5-(1,2,5,6-Tetrahydropyridin-3-yl)-2,3-dihydro-1H-inden-1-ol (Compound 2)

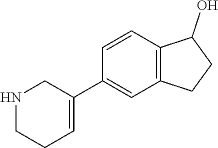 [SMILES: OC1CCc2cc(C3=CCCNC3)ccc21]

In a microwave tube compound 6 (28.9 mg, 135.6 μmol) and NaBH₄ (5.13 mg, 136 μmol) were dissolved in 0.5 ml EtOH and stirred at 100° C. for 17.5 h. Purification by preparative HPLC (ACN/H₂O+0.1% TFA: 10% for 3 min, 10%→42% in 8 min, 42%→90% in 1 min, 90% for 1 min, 90%→10% in 1 min, 10% for 1 min) yielded 14.1 mg (65.5 μmol, 48%) of a clear oil. ESI-MS: 215.92 [M+H]⁺. ¹H NMR: (400 MHz, DMSO-d₆) δ 7.33 (d, J=7.7 Hz, 1H), 7.27 (d, J=8.4 Hz, 2H), 6.32 (t, J=4.2, 2.0 Hz, 1H), 5.28 (s, 1H), 5.04 (t, J=6.7 Hz, 1H), 3.37 (s, 2H), 3.27-3.19 (m, 2H), 2.96-2.87 (m, 1H), 2.77-2.66 (m, 1H), 2.48-2.42 (m, 2H), 2.39-2.28 (m, 1H), 1.83-1.72 (m, 1H). ¹³C NMR: (101 MHz, DMSO-d₆) δ 146.76, 143.58, 137.27, 130.97, 124.77, 123.55, 121.92, 121.37, 74.49, 52.43, 43.00, 36.04, 29.75, 22.29. HR-MS (ESI): m/z [M+H]⁺ found 216.1383, calculated 216.1310 for C₁₄H₁₇NO.

### Purity:

| eluent system 1, t_{R} = 9.7 min: | eluent system 2, t_{R} = 8.1 min: |
|---|---|
| λ = 210 nm: 99.1% | λ = 210 nm: 99.3% |
| λ = 220 nm: 98.6% | λ = 220 nm: 99.2% |
| λ = 230 nm: 97.2% | λ = 230 nm: >99.9% |
| λ = 254 nm: 99.2% | λ = 254 nm: >99.9% |
| λ = 280 nm: 95.6% | λ = 280 nm: 98.0% |

### 6-(1,2,5,6-Tetrahydropyridin-3-yl)-2,3-dihydro-1H-inden-1-one (Compound 3)

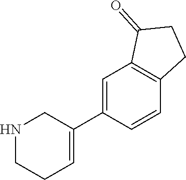 [SMILES: O=C1CCc2ccc(C3=CCCNC3)cc21]

Compound 3 was prepared according to GP2 using compound 1 and trifluoroacetic acid. The reaction was finished after 25 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 13.0 mg (61.0 μmol, 82%) of clear oil. ESI-MS: 214.08 [M+H]⁺. ¹H NMR: (400 MHz, DMSO-d 6) δ 7.80 (dd, J=8.2, 1.8 Hz, 1H), 7.64-7.60 (m, 2H), 6.47 (td, J=4.0, 2.0 Hz, 1H), 4.07 (s, 2H), 3.24 (d, J=6.2 Hz, 2H), 3.14-3.08 (m, 2H), 2.70-2.64 (m, 2H), 2.49 (s, 2H). ¹³C NMR: (101 MHz, DMSO-d₆) δ 206.67, 155.42, 137.48, 137.11, 131.85, 129.97, 127.81, 123.53, 119.09, 42.76, 39.88, 36.69, 25.69, 22.35. HR-MS (ESI): m/z [M+H]⁺ found 214.1226, calculated 214.1154 for C₁₄H₁₅NO.

### Purity:

| eluent system 1, t R = 11.7 min: | eluent system 2, t R = 10.7 min: |
|---|---|
| λ = 210 nm: 97.6% | λ = 210 nm: >99.9% |
| λ = 220 nm: >99.9% | λ = 220 nm: >99.9% |
| λ = 230 nm: 98.2% | λ = 230 nm: >99.9% |
| λ = 254 nm: >99.9% | λ = 254 nm: >99.9% |
| λ = 280 nm: 98.2% | λ = 280 nm: >99.9% |

### Tert-butyl 5-(1-oxo-2,3-dihydro-1H-inden-5-yl)-3,6-dihydropyridine-1(2H)-carboxylate (Compound 4): (TP88)

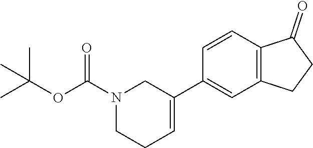 [SMILES: CC(C)(C)OC(=O)N1CCC=C(c2ccc3c(c2)CCC3=O)C1]

Compound 4 was prepared according to GP1 using 5-bromo-2,3-dihydro-1H-inden-1-one (20.0 mg, 94.8 μmol), tert-butyl 5-(4,4,5,5-tetramethyl-1,3,2-dioxaborolan-2-yl)-3,6-dihydropyridine-1(2H)-carboxylate (38.1 mg, 123 μmol), Pd(dppf)Cl₂ (3.47 mg, 4.74 μmol) and Na₂CO₃ (23.1 mg. 218 μmol) in 2.5 ml dioxane and 0.5 ml H₂O. The reaction was finished after 21 h. The reaction mixture was used crude. ESI-MS: 214.06 [M_{deprotected}+H]⁺; 258.10 [M_{deprotected}+2Na—H]⁺; 649.51 [2M+Na].

### Tert-butyl 5-(1-amino-2,3-dihydro-1H-inden-5-yl)-3,6-dihydropyridine-1(2H)-carboxylate (Compound 5): (TP89)

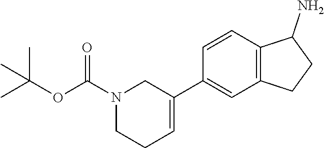 [SMILES: CC(C)(C)OC(=O)N1CCC=C(c2ccc3c(c2)CCC3N)C1]

Compound 5 was prepared according to GP1 using 5-bromo-2,3-dihydro-1H-inden-1-amine (20.0 mg, 94.3 μmol), tert-butyl 5-(4,4,5,5-tetramethyl-1,3,2-dioxaborolan-2-yl)-3,6-dihydropyridine-1(2H)-carboxylate (37.9 mg, 123 μmol), Pd(dppf)Cl₂ (3.45 mg, 4.71 μmol) and Na₂CO₃ (23.0 mg. 217 μmol) in 2.5 ml dioxane and 0.5 ml H₂O. The reaction was finished after 27 h. The reaction mixture was used crude. ESI-MS: 258.12 [M_{deprotected}+H]⁺; 629.48 [2M+H]⁺.

### 5-(1,2,5,6-Tetrahydropyridin-3-yl)-2,3-dihydro-1H-inden-1-one (Compound 6)

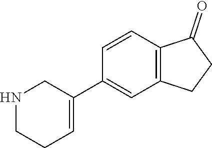 [SMILES: O=C1CCc2cc(C3=CCCNC3)ccc21]

Compound 6 was prepared according to GP2 using compound 4 and trifluoroacetic acid. The reaction was finished after 18 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 19.9 mg (93.3 μmol, 98%) of an off-white solid. ESI-MS: 213.95 [M+H]⁺. ¹H NMR: (400 MHz, DMSO-d₆) δ 7.66-7.62 (m, 2H), 7.52 (dd, J=8.2, 1.5 Hz, 1H), 6.59 (td, J=4.1, 2.0 Hz, 1H), 4.07 (q, J=2.2 Hz, 2H), 3.38 (s, 2H), 3.26 (t, J=6.1 Hz, 2H), 3.14-3.09 (m, 2H), 2.68-2.63 (m, 2H). ¹³C NMR: (101 MHz, DMSO-d₆) δ 206.16, 156.32, 143.83, 136.48, 125.56, 124.64, 123.60, 123.49, 42.71, 39.79, 36.55, 25.93, 22.52. HR-MS (ESI): m/z [M+H]⁺ found 214.1227, calculated 214.1154 for C₁₄H₁₅NO.

### Purity:

| eluent system 1, t_{R} = 11.0 min: | eluent system 2, t_{R} = 10.2 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 5-(1,2,5,6-Tetrahydropyridin-3-yl)-2,3-dihydro-1H-inden-1-amine (Compound 7)

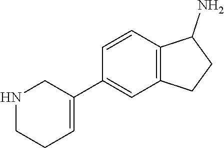 [SMILES: NC1CCc2cc(C3=CCCNC3)ccc21]

Compound 7 was prepared according to GP2 using compound 5 and trifluoroacetic acid. The reaction was finished after 72 h. Purification by preparative HPLC (ACN/H₂O+0.1% TFA: 10% for 3 min, 10%→55% in 9 min, 55%→90% in 1 min, 90% for 1 min, 90%→10% in 1 min, 10% for 1 min) yielded 18.8 mg (87.7 μmol, 93%) of a brown oil. ESI-MS: 214.95 [M+H]⁺ 429.23 [2M+H]⁺. ¹H NMR: (400 MHz, DMSO-d₆) δ 9.35 (s, 2H), 7.54 (d, J=7.9 Hz, 1H), 7.42-7.34 (m, 2H), 6.38 (t, J=3.9 Hz, 1H), 4.00 (s, 2H), 3.57 (s, 1H), 3.24 (t, J=6.2 Hz, 2H), 3.08 (ddd, J=21.5, 10.6, 6.3 Hz, 2H), 2.88 (dt, J=15.7, 7.4 Hz, 1H), 2.46-2.42 (m, 1H), 2.36-2.27 (m, 1H), 2.06-1.93 (m, 1H). ¹³C NMR: (101 MHz, DMSO-d₆) δ 145.02, 139.47, 138.83, 130.61, 125.37, 124.01, 122.95, 121.74, 54.83, 43.50, 42.83, 30.88, 30.27, 22.31. HR-MS (ESI): m/z [M+H]⁺ found 215.1544, calculated 215.1470 for C₁₄H₁₅NO.

### Purity:

| eluent system 1, t_{R} = 3.8 min: | eluent system 2, t_{R} = 4.2 min: |
|---|---|
| λ = 210 nm: 97.4% | λ = 210 nm: >99.9% |
| λ = 220 nm: >99.9% | λ = 220 nm: >99.9% |
| λ = 230 nm: 97.2% | λ = 230 nm: >99.9% |
| λ = 254 nm: >99.9% | λ = 254 nm: >99.9% |
| λ = 280 nm: >99.9% | λ = 280 nm: >99.9% |

### Tert-butyl 5-(6-chloro-2,3-dihydro-1H-inden-5-yl)-3,6-dihydropyridine-1(2H)-carboxylate (Compound 8): (TP_NN05)

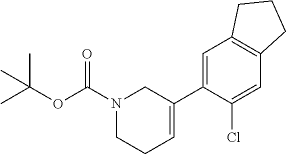 [SMILES: CC(C)(C)OC(=O)N1CCC=C(c2cc3c(cc2Cl)CCC3)C1]

Compound 8 was prepared according to GP1 using 5-bromo-6-chloro-2,3-dihydro-1H-indene (20.0 mg, 86.4 μmol), tert-butyl 5-(4,4,5,5-tetramethyl-1,3,2-dioxaborolan-2-yl)-3,6-dihydropyridine-1(2H)-carboxylate (34.7 mg, 112 μmol), Pd(dppf)Cl₂ (3.16 mg, 4.32 μmol) and Na₂CO₃ (21.1 mg. 199 μmol) in 2.5 ml dioxane and 0.5 ml H₂O. The reaction was finished after 21 h. The reaction mixture was used crude. ESI-MS: 335.45[M+H]⁺.

### 5-(6-chloro-2,3-dihydro-1H-inden-5-yl)-1,2,3,6-tetrahydropyridine (Compound 9): (TP98)

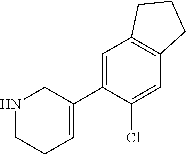 [SMILES: Clc1cc2c(cc1C1=CCCNC1)CCC2]

Compound 9 was prepared according to GP2 using comound 8 and trifluoroacetic acid. The reaction was finished after 21 h. Purification by preparative HPLC (ACN/H₂O+0.1% TFA: 10% for 3 min, 10%→70% in 11 min, 70%→90% in 0.5 min, 90% for 1 min, 90%→10% in 1 min, 10% for 1 min) yielded 20.1 mg (86.0 μmol, 100%) of a off-white solid. ESI-MS: 233.93 [M+H]⁺. ¹H NMR: (400 MHz, DMSO-d6) δ 7.34 (s, 1H), 7.15 (s, 1H), 5.91-5.86 (m, 1H), 3.80 (s, 2H), 3.25 (t, J=6.1 Hz, 2H), 2.85 (dt, J=9.8, 7.5 Hz, 4H), 2.48-2.40 (m, 2H), 2.03 (p, J=7.4 Hz, 2H). ¹³C NMR: (101 MHz, DMSO-d₆) δ 146.41, 143.80, 135.68, 131.25, 129.36, 126.67, 126.00, 125.73, 43.94, 32.52, 32.22, 25.69, 22.09. HR-MS (ESI): m/z [M+H]⁺ found 234.1045, calculated 234.0971 for C₁₄H₁₅NO.

### Purity:

| eluent system 1, t_{R} = 16.2 min: | eluent system 2, t_{R} = 14.5 min: |
|---|---|
| λ = 210 nm: 96.8% | λ = 210 nm: 93.1% |
| λ = 220 nm: 97.9% | λ = 220 nm: 94.6% |
| λ = 230 nm: 95.0% | λ = 230 nm: 95.4% |
| λ = 254 nm: 98.7% | λ = 254 nm: 95.4% |
| λ = 280 nm: 98.1% | λ = 280 nm: 95.6% |

### Tert-butyl 5-(1-methylene-2,3-dihydro-1H-inden-5-yl)-3,6-dihydropyridine-1(2H)-carboxylate (Compound 10): (TP103)

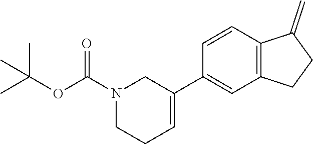 [SMILES: C=C1CCc2cc(C3=CCCN(C(=O)OC(C)(C)C)C3)ccc21]

Compound 10 was prepared according to GP1 using 5-bromo-1-methylene-2,3-dihydro-1H-indene (20.0 mg, 95.7 μmol), tert-butyl 5-(4,4,5,5-tetramethyl-1,3,2-dioxaborolan-2-yl)-3,6-dihydropyridine-1(2H)-carboxylate (38.5 mg, 124 μmol), Pd(dppf)Cl₂ (3.50 mg, 4.78 μmol) and Na₂CO₃ (23.3 mg. 220 μmol) in 2.5 ml dioxane and 0.5 ml H₂O. The reaction was finished after 26 h. The reaction mixture was used crude. ESI-MS: 212.09 [M_{deprotected}+H]⁺, 312.05 [M+H]⁺; 334.35 [M+Na]⁺; 645.54 [2M+Na]⁺.

### 5-(1-Methylene-2,3-dihydro-1H-inden-5-yl)-1,2,3,6-tetrahydropyridine (Compound 11): (TP105)

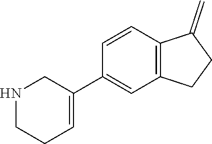 [SMILES: C=C1CCc2cc(C3=CCCNC3)ccc21]

Compound 11 was prepared according to GP2 using compound 10 and trifluoroacetic acid. The reaction was finished after 45 h. Purification by preparative HPLC (ACN/H₂O+0.1% TFA: 10% for 3 min, 10%→60% in 10 min, 60%→90% in 1 min, 90% for 1 min, 90%→10% in 1 min, 10% for 1 min) yielded 8.00 mg (37.9 μmol, 40%) of a yellow oil. ESI-MS: 211.95 [M+H]⁺ 423.23 [2M+H]⁺. ¹H NMR: (400 MHz, DMSO-d₆) δ 7.48 (d, J=1.7 Hz, 1H), 7.35 (d, J=1.6 Hz, 1H), 7.21 (d, J=8.0 Hz, 1H), 7.12 (d, J=8.0 Hz, 1H), 6.38-6.29 (m, 2H), 2.96 (h, J=9.8, 9.2 Hz, 2H), 2.41-2.10 (m, 2H), 1.68-1.48 (m, 6H). ¹³C NMR: (151 MHz, DMSO-d₆) δ 151.56, 143.41, 142.39, 136.29, 133.68, 124.31, 123.76, 121.72, 119.93, 118.41, 52.49, 43.05 (d, J=13.7 Hz), 42.24, 30.58, 22.33 (d, J=2.1 Hz). HR-MS (ESI): m/z [M+H]⁺ found 212.1434, calculated 212.1361 for C₁₄H₁₅NO.

### Purity:

| eluent system 1, t_{R} = 15.9 min: | eluent system 2, t_{R} = 13.9 min: |
|---|---|
| λ = 210 nm: >99.9% | λ = 210 nm: 97.7% |
| λ = 220 nm: >99.9% | λ = 220 nm: 95.5% |
| λ = 230 nm: >99.9% | λ = 230 nm: 98.2% |
| λ = 254 nm: 97.8% | λ = 254 nm: 94.6% |
| λ = 280 nm: >99.9% | λ = 280 nm: 97.5% |

### 5-(4-Chloro-2,3-dihydro-1H-inden-5-yl)-1,2,3,6-tetrahydropyridine (7074, Z4767467074, Z4923386334)—Enamine Procedure 3

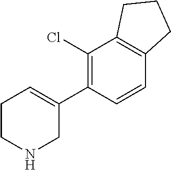 [SMILES: Clc1c(C2=CCCNC2)ccc2c1CCC2]

¹H NMR (400 MHz, DMSO-d₆) δ 2.05 (p, J=7.5 Hz, 2H), 2.44 (dp, J=8.6, 3.0, 2.3 Hz, 2H), 2.90 (t, J=7.5 Hz, 2H), 2.95 (t, J=7.5 Hz, 2H), 3.25 (t, J=6.1 Hz, 2H), 3.80 (d, J=2.3 Hz, 2H), 5.82-5.96 (m, 1H), 7.07 (d, J=7.6 Hz, 1H), 7.21 (d, J=7.6 Hz, 1H), 9.12 (s, 2H); ¹³C NMR (101 MHz, DMSO-d₆) δ 21.66, 24.21, 32.35, 33.12, 43.56, 123.07, 125.58, 127.92, 128.83, 130.50, 135.38, 142.78, 145.82.

### 4-Methyl-5-(1,2,5,6-tetrahydropyridin-3-yl)-1H-pyrrolo[2,3-b]pyridine (9661)

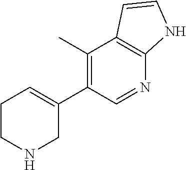 [SMILES: Cc1c(C2=CCCNC2)cnc2[nH]ccc12]

¹H NMR (400 MHz, DMSO-d₆) δ 2.39 (s, 2H), 2.46 (s, 3H), 3.18 (d, J=6.0 Hz, 2H), 3.66 (d, J=7.1 Hz, 2H), 5.76-5.80 (m, 1H), 6.50 (dd, J=3.5, 1.8 Hz, 1H), 7.43 (dd, J=3.4, 2.4 Hz, 1H), 7.93 (d, J=1.2 Hz, 1H), 8.29 (s, 1H), 11.58 (s, 1H); ¹³C NMR (101 MHz, DMSO-d₆) δ 15.75, 22.77, 45.82 (br), 98.83, 119.86, 125.33, 125.78, 127.08, 131.70, 136.09, 142.45, 147.33.

### 5-(3-Methylbenzofuran-5-yl)-1,2,3,6-tetrahydropyridine (9677)

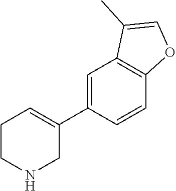 [SMILES: Cc1coc2ccc(C3=CCCNC3)cc12]

¹H NMR (400 MHz, DMSO-d₆) δ 2.21 (d, J=1.4 Hz, 3H), 2.34 (s, 2H), 3.00-3.07 (m, 2H), 3.83-3.89 (m, 2H), 6.27 (d, J=4.1 Hz, 1H), 7.37 (dd, J=8.6, 1.9 Hz, 1H), 7.50 (d, J=8.6 Hz, 1H), 7.59 (d, J=2.0 Hz, 1H), 7.76 (d, J=1.4 Hz, 1H), 8.37 (s, 1H); ¹³C NMR (151 MHz, DMSO-d₆) δ 7.54, 23.71, 40.54, 44.42, 111.07, 115.46, 115.74, 121.46, 121.74, 128.77, 133.48, 133.54, 142.61, 154.01.

### 5-(2,3-Dihydrobenzofuran-5-yl)-1,2,3,6-tetrahydropyridine (9700)

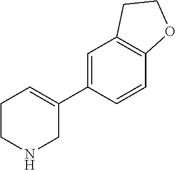 [SMILES: C1=C(c2ccc3c(c2)CCO3)CNCC1]

¹H NMR (400 MHz, DMSO-d₆) δ 2.29 (s, 2H), 2.97-3.02 (m, 2H), 3.16 (t, J=8.7 Hz, 2H), 3.73 (s, 2H), 4.52 (t, J=8.7 Hz, 2H), 6.04-6.20 (m, 1H), 6.72 (d, J=8.3 Hz, 1H), 7.11 (dd, J=8.3, 2.0 Hz, 1H), 7.28 (d, J=1.9 Hz, 1H), 8.36 (s, 1H); ¹³C NMR (101 MHz, DMSO-d₆) δ 23.44, 29.06, 40.32, 43.90, 71.09, 108.70, 120.01, 121.58, 124.40, 127.66, 131.08, 132.67, 159.27.

### 5-(Naphthalen-1-yl)-1,2,3,6-tetrahydropyridine (3461)

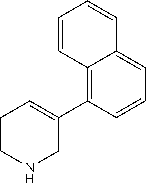 [SMILES: C1=C(c2cccc3ccccc23)CNCC1]

¹H NMR (400 MHz, DMSO-d₆) δ 2.33-2.43 (m, 2H), 3.09-3.22 (m, 1H), 3.66 (s, 2H), 5.77-5.91 (m, 1H), 7.29-7.44 (m, 1H), 7.44-7.52 (m, 1H), 7.52-7.58 (m, 2H), 7.84-7.92 (m, 1H), 7.92-7.98 (m, 1H), 7.99-8.11 (m, 1H), 8.32 (s, 1H).

### 5-(2,3-dihydro-1H-inden-4-yl)-1,2,3,6-tetrahydropyridine (WXVL_BT1618LQ0489, 3827)

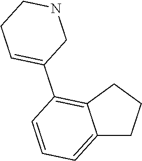 [SMILES: C1=C(c2cccc3c2CCC3)CNCC1]

Step 1: To a mixture of tert-butyl5-(4,4,5,5-tetramethyl-1,3,2-dioxaborolan-2-yl)-3,6-dihydro-2H-pyridine-1-carboxylate (0.15 g, 485.11 umol, 1 eq) and 4-iodoindane (142.08 mg, 582.13 umol, 80.90 uL, 1.2 eq) in dioxane (2 mL) and H₂O (0.4 mL) was added K₃PO₄ (205.95 mg, 970.22 umol, 2 eq) and Pd(dppf)Cl₂ (35.50 mg, 48.51 umol, 0.1 eq) in one portion at 25° C. under N₂. The suspension was degassed under vacuum and purged with N₂ three times. Then the mixture was warmed to 80° C. and stirred at 80° C. for 4 hrs under N₂. LCMS indicated tert-butyl 5-(4,4,5,5-tetramethyl-1,3,2-dioxaborolan-2-yl)-3,6-dihydro-2H-pyridine-1-carboxylate (0.15 g, 485.11 umol, 1 eq) was consumed completely and two new peaks formed. The reaction mixture were added thiourea resin (0.5 g), then the mixture were stirred at 25° C. for another 12 hrs. Then the mixture were filtered through a Celite pad, and the filtrate was concentrated to give the crude product. The residue was purified by pre-HPLC (column: 3_Phenomenex Luna C18 75*30 mm*3 um; mobile phase: [water (0.2% FA)-ACN]; B %: 50%-85%, 6 min) to give the product tert-butyl 5-indan-4-yl-3,6-dihydro-2H-pyridine-1-carboxylate (0.1 g, 255.51 umol, 52.67% yield, 76.5% purity) as Gray solid.

Step 2: To a mixture of tert-butyl 5-indan-4-yl-3,6-dihydro-2H-pyridine-1-carboxylate (0.1 g, 333.99 umol, 1 eq) in DCM (2.4 mL) was added TFA (477.42 mg, 4.19 mmol, 310.01 uL, 12.54 eq) in one portion at 0° C. The mixture was stirred at 0° C. for 1 hours. LC-MS showed tert-butyl 5-indan-4-yl-3,6-dihydro-2H-pyridine-1-carboxylate (0.1 g, 333.99 umol, 1 eq) was consumed completely and one main peak with desired mass was detected. The reaction mixture was concentrated under reduced pressure to give a residue. The residue was purified by prep-HPLC (column: Phenomenex Gemini-NX C18 75*30 mm*3 um; mobile phase: [water (10 mM NH₄HCO₃)-ACN]; B %: 1%-35%, 6 min) to give desired compound. QC LCMS (WXVL_BT1618LQ0489) showed the compound 5-indan-4-yl-1,2,3,6-tetrahydropyridine (0.06 g, 290.17 umol, 86.88% yield, 96.38% purity) was obtained as a white solid. Yield: 69.72%. ¹H NMR (MHz, CDCl₃) δ=9.46 (br s, 1H), 7.24-7.19 (m, 1H), 7.18-7.12 (m, 1H), 6.95 (br d, J=7.4 Hz, 1H), 5.96 (br s, 1H), 3.90 (br s, 2H), 3.38 (br s, 2H), 2.97-2.84 (m, 4H), 2.60 (br s, 2H), 2.06 (quin, J=7.3 Hz, 2H). LC/MS (ESI) m/z [M+H] calculated for C₁₄H₁₇N: 199.14; found: 200.1.

### 5-(6-Methyl-1H-indazol-5-yl)pyridin-2-amine—Method 9 (ZINC001242282998, Z4376630001)

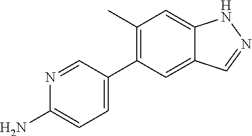 [SMILES: Cc1cc2[nH]ncc2cc1-c1ccc(N)nc1]

Halogenide (1 eq.), and boronic acid (1.3 eq.) were mixed in dioxane (0.5 mL per 100 mg of product), and Pd(dppf)Cl₂ (0.05 eq.) (as stock solution in dioxane 0.05 mL per 100 mg of product), and disodium carbonate (Na₂CO₃) (2.3 eq.) (as 1M stock solution in water) were added under inert atmosphere. The reaction mixture was sealed and heated for 16 hours at 100° C. while shaking. The mixture was cooled to ambient temperature and trifluoroacetic acid was added dropwise until neutral pH was reached. The solvent was evaporated under reduced pressure and the residue was dissolved in DMSO (1 mL per 100 mg of product). The resulting DMSO-solution was treated with Scavenger SiliaMetS DMT, filtered, analyzed by LC/MS, and purified by HPLC. ¹H NMR (400 MHz, DMSO-d6): δ 2.30 (s, 3H), 5.94 (s, 2H), 6.50 (dd, J=8.4, 0.8 Hz, 1H), 7.39 (dd, J=8.3, 2.5 Hz, 2H), 7.49 (s, 1H), 7.40 (s, 1H), 7.87 (dd, J=2.5, 0.7 Hz, 1H), 7.99 (d, J=1.0 Hz, 1H), 12.91 (s, 1H). ¹³C NMR (101 MHz, DMSO-d6): δ 21.40, 107.14, 110.39, 120.65, 121.67, 125.50, 132.42, 133.31, 134.28, 138.09, 139.54, 147.50, 158.51.

### Tert-Butyl(5-(isochinolin-5-yl) pyridin-2-yl) carbamate (PS-HT04-Boc)

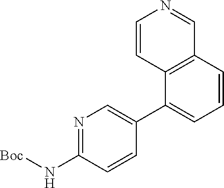 [SMILES: CC(C)(C)OC(=O)Nc1ccc(-c2cccc3cnccc23)cn1]

Na₂CO₃ (63.2 mg, 596 μmol), 5-bromoisoquinoline (31.8 mg, 153 μmol), tert-butyl(5-(4,4,5,5-tetramethyl-1,3,2-dioxaborolan-2-yl) pyridin-2-yl) carbamate (61. 0 mg, 191 mol) and Pd(dppf)Cl 2 (7.20 mg, 9.84 μmol) were weighed into a microwave tube. Dioxane (3 ml) and water (0.8 ml) were added. The reaction tube was tightly sealed and purged with nitrogen. The reaction mixture was then heated to 100° C. for 4 h. After cooling to room temperature, the reaction mixture was adjusted to a basic pH of 10 using NaOH solution (2M), then poured onto water (50 ml) and extracted with ethyl acetate (3×50 ml). The combined organic layer was dried over Na₂SO₄, filtered and concentrated on the rotary evaporator. The resulting residue was purified by normal phase column chromatography (isohexane/EtOAc=5:5) and the collected fractions were concentrated on the rotary evaporator. The remaining residue was dried under high vacuum. The product was isolated as a white crystalline solid (33.7 mg, 105 μmol, 72%). ¹H NMR (400 MHz, DMSO-d 6): δ 11.02 (s, 1H), 10.40 (d, J=0.9 Hz, 1H), 9.51 (d, J=6.0 Hz, 1H), 9.38 (dd, J=2.4, 0.9 Hz, 1H), 9.22-9.15 (m, 1H), 8.98 (dd, J=8.6, 0.9 Hz, 1H), 8.91 (dd, J=8.6, 2.4 Hz, 1H), 8.78 (d, J=2.2 Hz, 1H), 8.77 (s, 1H), 8.66 (ddd, J=6.0, 1.0 Hz, 1H), 2.50 (s, 9H). ¹³C NMR (101 MHz, DMSO-d 6) δ 152.95, 152.07, 148.00, 143.68, 139.18, 134.78, 133.25, 131.37, 128.61, 128.53, 127.67, 127.27, 117.60, 111.91, 79.79, 28.06. LC/MS (ESI+) m/z: 322.3 [M+H]⁺.

### 5-(Isochinolin-5-yl) pyridin-2-amine (PS-HT04)

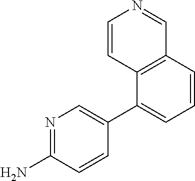 [SMILES: Nc1ccc(-c2cccc3cnccc23)cn1]

Tert-butyl(5-(isoquinolin-5-yl) pyridin-2-yl) carbamate (PS-HT04-Boc) (33.7 mg, 105 μmol) was dissolved in TFA (3 ml) in a round bottom flask and stirred for 7 h at room temperature. Subsequently, the solution was neutralized using a NaOH solution (2 M) and a basic pH of 10 was adjusted. The reaction mixture was poured onto water (50 ml) and extracted with ethyl acetate (3×50 ml). The combined organic layer was dried over Na₂SO₄, filtered and concentrated on the rotary evaporator. The resulting white residue was purified by normal phase column chromatography (DCM/MeOH=9:1) and the collected fractions were concentrated on the rotary evaporator. The remaining residue was dried under high vacuum. The product was isolated as a yellow solid (9.00 mg, 40.7 μmol, 39%). ¹H NMR (400 MHz, DMSO-d₆): δ 9.36 (d, J=1.0 Hz, 1H), 8.49 (d, J=6.0 Hz, 1H), 8.10 (dt, J=8.0, 1.2 Hz, 1H), 8.04 (dd, J=2.5, 0.8 Hz, 1H), 7.75-7.70 (m, 2H), 7.68 (dd, J=7.1, 1.5 Hz, 1H), 7.56 (dd, J=8.5, 2.5 Hz, 1H), 6.62 (dd, J=8.5, 0.8 Hz, 1H), 6.22 (s, 2H); ¹³C NMR (101 MHz, DMSO-d₆) δ 159.76, 153.32, 148.42, 143.78, 138.89, 136.45, 133.86, 131.08, 129.22, 127.75, 127.17, 122.50, 118.31, 108.11; LC/MS (ESI+) m/z: 222.0 [M+H]⁺; HRMS (ESI+) m/z calcd for C₁₄H₁₁N₃+H+: m/z 222.1026 [M+H]⁺, found: m/z 222.1024 [M+H]⁺.

### Tert-butyl (5-(quinolin-5-yl) pyridin-2-yl) carbamate (PS-HT06-Boc)

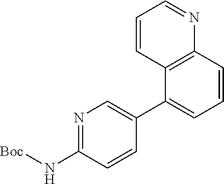 [SMILES: CC(C)(C)OC(=O)Nc1ccc(-c2cccc3ncccc23)cn1]

Na₂CO₃ (18.4 mg, 173.61 μmol), 5-bromoquinoline (22.0 mg, 106 μmol), tert-butyl (5-(4,4,5,5-tetramethyl-1,3,2-dioxaborolan-2-yl) pyridin-2-yl) carbamate (31.6 mg, 98.7 mol) and Pd(dppf)Cl₂ (5.30 mg, 7.24 μmol) were weighed into a microwave tube. Dioxane (3 ml) and water (0.8 ml) were added. The reaction tube was tightly sealed and purged with nitrogen. The reaction mixture was then heated to 100° C. for 6 h. After cooling to room temperature, the reaction mixture was adjusted to a basic pH of 10 using NaOH solution (2 M), then poured onto water (50 ml) and extracted with ethyl acetate (3×50 ml). The combined organic layer was dried over Na₂SO₄, filtered, and concentrated on the rotary evaporator. The resulting residue was purified by normal phase column chromatography (isohexane/EtOAc=5:5) and the collected fractions were concentrated on the rotary evaporator. The remaining residue was dried under high vacuum. The product was used for the synthesis of HT06 without further purification. ¹H NMR (400 MHz, DMSO-d₆): δ 10.01 (s, 1H), 8.95 (dd, J=4.1, 1.7 Hz, 1H), 8.37 (dd, J=2.4, 0.9 Hz, 1H), 8.20 (ddd, J=8.6, 1.7, 0.9 Hz, 1H), 8.08 (d, J=8.5 Hz, 1H), 7.97 (dd, J=8.6, 0.9 Hz, 1H), 7.90 (dd, J=8.6, 2.4 Hz, 1H), 7.85 (dd, J=8.5, 7.1 Hz, 1H), 7.59 (dd, J=7.1, 1.2 Hz, 1H), 7.55 (dd, J=8.6, 4.1 Hz, 1H), 1.51 (s, 9H). ¹³C NMR (101 MHz, DMSO-d₆) δ 153.29, 152.47, 151.05, 148.51, 139.75, 136.83, 133.86, 129.64, 129.39, 129.25, 128.01, 126.48, 122.38, 112.32, 80.22, 28.51. LC/MS (ESI+) m/z: 322.3 [M+H]⁺.

### 5-(Quinolin-5-yl)pyridin-2-amine (PS-HT06)

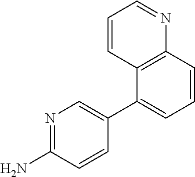 [SMILES: Nc1ccc(-c2cccc3ncccc23)cn1]

Tert-butyl (5-(quinolin-5-yl) pyridin-2-yl) carbamate (PS-HT06-Boc) was dissolved in TFA (2 ml) in a round bottom flask and stirred for 18 h at room temperature. Subsequently, the solution was neutralized using a NaOH solution (2 M) and a basic pH of 10 was adjusted. The reaction mixture was poured onto water (50 ml) and extracted with ethyl acetate (3×50 m1). The combined organic layer was dried over Na₂SO₄, filtered and concentrated on a rotary evaporator. The resulting yellow residue was purified by normal phase column chromatography (DCM/MeOH=9:1) and the collected fractions were concentrated on a rotary evaporator. The product was isolated as a white solid (7.28 mg, 31%). ¹H NMR (400 MHz, DMSO-d₆): δ 8.92 (dd, J=4.1, 1.7 Hz, 1H), 8.25 (ddd, J=8.6, 1.7, 0.9 Hz, 1H), 8.02 (dd, J=2.5, 0.8 Hz, 1H), 8.00 (dt, J=8.5, 1.1 Hz, 1H), 7.79 (dd, J=8.5, 7.1 Hz, 1H), 7.56-7.51 (m, 2H), 7.50 (dd, J=6.0, 1.2 Hz, 1H), 6.61 (dd, J=8.5, 0.8 Hz, 1H), 6.20 (s, 2H); ¹³C NMR (101 MHz, DMSO-d₆) δ 150.84, 148.54, 139.02, 138.10, 134.08, 129.66, 128.50, 127.32, 126.62, 122.01, 108.05, not all carbon-atoms were detected in DEPTQ-experiment due to low signal intensity; LC/MS (ESI+) m/z: 222.0 [M+H]⁺; HRMS (ESI+) m/z calcd for C₁₄H₁₁N₃+H+m/z 222.1026 [M+H]⁺, found: m/z 222.1023 [M+H]⁺.

### Tert-butyl (5-(7-nitro-1H-indazol-5-yl)pyridin-2-yl)carbamate (Compound 1): (TP67)

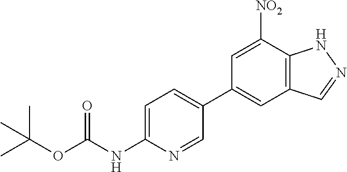 [SMILES: CC(C)(C)OC(=O)Nc1ccc(-c2cc([N+](=O)[O-])c3[nH]ncc3c2)cn1]

Compound 1 was prepared according to GP1 using 5-bromo-7-nitro-1H-indazole (20.0 mg, 82.6 μmol), tert-butyl (5-(4,4,5,5-tetramethyl-1,3,2-dioxaborolan-2-yl)pyridin-2-yl)carbamate (34.4 mg, 107 μmol), Pd(dppf)Cl₂ (3.02 mg, 4.13 μmol) and Na₂CO₃ (20.1 mg. 190 μmol) in 2.5 ml dioxane and 0.5 ml H₂O. The reaction was finished after 39 h. The reaction mixture was used crude. ESI-MS: 356.15 [M+H]⁺, 255.97 [M_{deprotected}+H]⁺.

### Tert-butyl (5-(7-chloro-1H-indazol-5-yl)pyridin-2-yl)carbamate (Compound 2): (TP68)

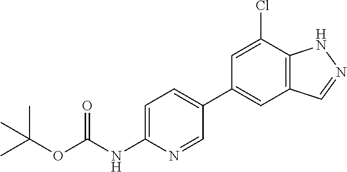 [SMILES: CC(C)(C)OC(=O)Nc1ccc(-c2cc(Cl)c3[nH]ncc3c2)cn1]

Compound 2 was prepared according to GP1 using 5-bromo-7-chloro-1H-indazole (20.0 mg, 86.4 μmol), tert-butyl (5-(4,4,5,5-tetramethyl-1,3,2-dioxaborolan-2-yl)pyridin-2-yl)carbamate (36.0 mg, 112.3 μmol), Pd(dppf)Cl₂ (3.16 mg, 4.32 μmol) and Na₂CO₃ (21.6 mg. 199 μmol) in 2.5 ml dioxane and 0.5 ml H₂O. The reaction was finished after 25 h. The reaction mixture was used crude. ESI-MS: 345.22 [M+H]⁺, 244.96 [M_{deprotected}+H]⁺.

### Tert-butyl (5-(6-chloro-1H-indazol-5-yl)pyridin-2-yl)carbamate (Compound 3): (TP69)

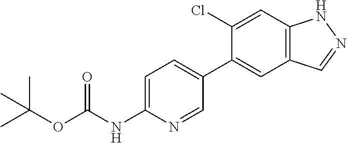 [SMILES: CC(C)(C)OC(=O)Nc1ccc(-c2cc3cn[nH]c3cc2Cl)cn1]

Compound 3 was prepared according to GP1 using 5-bromo-6-chloro-1H-indazole (20.0 mg, 86.4 μmol), tert-butyl (5-(4,4,5,5-tetramethyl-1,3,2-dioxaborolan-2-yl)pyridin-2-yl)carbamate (36.0 mg, 112.3 μmol), Pd(dppf)Cl₂ (3.16 mg, 4.32 μmol) and Na₂CO₃ (21.6 mg, 199 μmol) in 2.5 ml dioxane and 0.5 ml H₂O. The reaction was finished after 24 h. The reaction mixture was used crude. ESI-MS: 345.17 [M+H]⁺, 244.96 [M_{deprotected}+H]⁺.

### 5-(7-Nitro-1H-indazol-5-yl)pyridin-2-amine (Compound 4): (TP70)

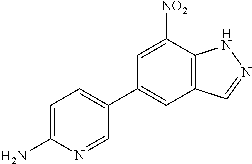 [SMILES: Nc1ccc(-c2cc([N+](=O)[O-])c3[nH]ncc3c2)cn1]

Compound 4 was prepared according to GP2 using compound 1 and trifluoroacetic acid. The reaction was finished after 1 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 8.00 mg (31.3 μmol, 38%) of a yellow solid. ESI-MS: 255.98 [M+H]⁺. ¹H NMR: (400 MHz, DMSO-d₆) δ 14.08 (s, 1H), 8.62 (d, J=1.6 Hz, 1H), 8.60 (d, J=1.6 Hz, 1H), 8.48 (q, J=2.2 Hz, 2H), 8.38 (dt, J=9.2, 2.2 Hz, 1H), 7.98 (d, J=42.3 Hz, 2H), 7.06 (dd, J=9.2, 3.5 Hz, 1H). ¹³C NMR: (101 MHz, DMSO-d₆) δ 154.40, 142.58, 136.56, 135.71, 132.89, 131.82, 128.23 (d, J=16.1 Hz), 127.35, 123.39, 121.97, 113.63. HR-MS (ESI): m/z [M+H]⁺ found 256.0828, calculated 256.0756 for C₁₂H₉N₅O₂.

### Purity:

| eluent system 1, t_{R} = 12.4 min: | eluent system 2, t_{R} = 11.1 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: 99.8% | λ = 254 nm: >99.9% |
| λ = 280 nm: 99.7% | λ = 280 nm: >99.9% |

### Tert-butyl (5-(6-fluoro-1H-indazol-5-yl)pyridin-2-yl)carbamate (Compound 5): (TP71)

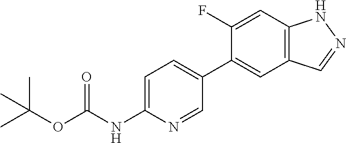 [SMILES: CC(C)(C)OC(=O)Nc1ccc(-c2cc3cn[nH]c3cc2F)cn1]

Compound 5 was prepared according to GP1 using 5-bromo-6-fluoro-1H-indazole (20.0 mg, 93.0 μmol), tert-butyl (5-(4,4,5,5-tetramethyl-1,3,2-dioxaborolan-2-yl)pyridin-2-yl)carbamate (38.7 mg, 121 μmol), Pd(dppf)Cl₂ (3.40 mg, 4.65 μmol) and Na₂CO₃ (22.7 mg. 214 μmol) in 2.5 ml dioxane and 0.5 ml H₂O. The reaction was finished after 41 h. The reaction mixture was used crude. ESI-MS: 329.16 [M+H]⁺, 228.95 [M_{deprotected}+H]⁺.

### 5-(7-Chloro-1H-indazol-5-yl)pyridin-2-amine (Compound 6): (TP73)

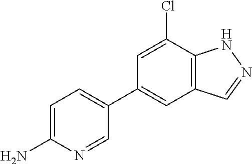 [SMILES: Nc1ccc(-c2cc(Cl)c3[nH]ncc3c2)cn1]

Compound 6 was prepared according to GP2 using compound 2 and trifluoroacetic acid. The reaction was finished after 15 h. Purification by preparative HPLC (ACN/H₂O+0.1% TFA: 10% for 3 min, 10%→55% in 9 min, 55%→90% in 1 min, 90% for 1 min, 90%→10% in 1 min, 10% for 1 min) yielded 6.30 mg (25.8 μmol, 30%) of an off-white solid. ESI-MS: 244.96 [M+H]⁺. ¹H NMR: (400 MHz, DMSO-d₆) δ 9.66 (s, 1H) 8.37 (dd, J=9.1, 2.3 Hz, 1H), 8.34 (s, 1H), 8.27 (s, 1H), 8.11 (s, 2H), 8.05 (s, 1H), 7.80 (d, J=1.5 Hz, 1H), 7.08 (dd, J=9.2, 0.8 Hz, 1H). ¹³C NMR: (151 MHz, DMSO-d₆) δ 154.23, 142.50, 135.65, 135.25, 129.29, 4.50, 124.35, 117.50, 113.43. HR-MS (ESI): m/z [M+H]⁺ found 245.0589, calculated 245.0516 for C₁₂H₉C₁N₄.

### Purity:

| eluent system 1, t_{R} = 13.1 min: | eluent system 2, t_{R} = 11.6 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 5-(6-Chloro-1H-indazol-5-yl)pyridin-2-amine (Compound 7): (TP74)

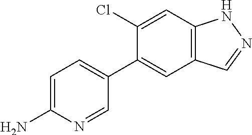 [SMILES: Nc1ccc(-c2cc3cn[nH]c3cc2Cl)cn1]

Compound 7 was prepared according to GP2 using compound 3 and trifluoroacetic acid. The reaction was finished after 24 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 7.40 mg (30.2 μmol, 35%) of an off-white solid. ESI-MS: 244.96 [M+H]⁺. ¹H NMR: (400 MHz, DMSO-d₆) δ 13.38 (s, 1H), 8.22 (s, 2H), 8.19 (d, J=1.0 Hz, 1H), 8.08 (dd, J=2.5, 1.1 Hz, 1H), 8.05 (d, J=2.2 Hz, 1H), 7.90 (d, J=0.6 Hz, 1H), 7.82 (dd, J=1.0, 0.6 Hz, 1H), 7.06 (dd, J=8.9, 1.0 Hz, 1H). ¹³C NMR: (101 MHz, DMSO-d₆) δ 154.25, 145.55, 137.05, 134.55, 130.52, 127.24, 124.19, 123.61, 122.46, 112.57, 111.48. HR-MS (ESI): m/z [M+H]⁺ found 245.0589, calculated 245.0516 for C₁₂H₉ClN₄.

### Purity:

| eluent system 1, t_{R} = 13.0 min: | eluent system 2, t_{R} = 11.6 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 5-(6-Fluoro-1H-indazol-5-yl)pyridin-2-amine (Compound 8): (TP75)

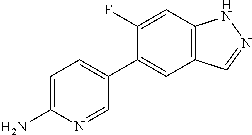 [SMILES: Nc1ccc(-c2cc3cn[nH]c3cc2F)cn1]

Compound 8 was prepared according to GP2 using compound 5 and trifluoroacetic acid. The reaction was finished after 120 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 13.1 mg (57.4 μmol, 62%) of an off-white solid. ESI-MS: 228.92 [M+H]⁺. ¹H NMR: (400 MHz, DMSO-d₆) δ 13.33 (s, 1H), 8.21 (s, 2H), 8.17 (s, 2H), 8.13 (dt, J=9.2, 2.1 Hz, 1H), 7.95 (d, J=7.5 Hz, 1H), 7.54-7.49 (m, 1H), 7.06 (d, J=9.2 Hz, 1H). ¹³C NMR: (101 MHz, DMSO-d₆) δ 160.00, 157.57, 154.10, 144.65, 140.02, 136.17, 122.47 (d, J=5.0 Hz), 120.85, 120.45, 118.13 (d, J=18.0 Hz), 13.44 (d, J=2.7 Hz), 96.99 (d, J=27.3 Hz). HR-MS (ESI): m/z [M+H]⁺ found 229.0880, calculated 229.0811 for C₁₂H₉FN₄.

### Purity:

| eluent system 1, t_{R} = 12.3 min: | eluent system 2, t_{R} = 11.0 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### Tert-butyl (5-(6-nitro-1H-indazol-5-yl)pyridin-2-yl)carbamate (Compound 9): (TP76)

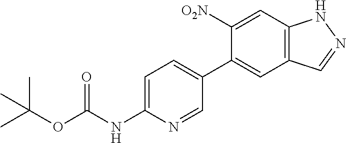 [SMILES: CC(C)(C)OC(=O)Nc1ccc(-c2cc3cn[nH]c3cc2[N+](=O)[O-])cn1]

Compound 9 was prepared according to GP1 using 5-bromo-6-nitro-1H-indazole (30.0 mg, 124 μmol), tert-butyl (5-(4,4,5,5-tetramethyl-1,3,2-dioxaborolan-2-yl)pyridin-2-yl)carbamate (51.6 mg, 161 μmol), Pd(dppf)Cl₂ (4.53 mg, 6.20 μmol) and Na₂CO₃ (30.2 mg. 285 μmol) in 2.5 ml dioxane and 0.5 ml H₂O. The reaction was finished after 115 h. The reaction mixture was used crude. ESI-MS: 255.95 [M_{deprotected}+H]⁺.

### Tert-butyl (5-(6-methoxy-1H-indazol-5-yl)pyridin-2-yl)carbamate (Compound 10): (TP77)

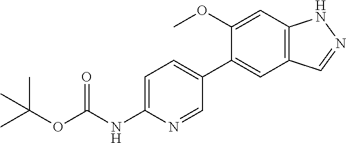 [SMILES: COc1cc2[nH]ncc2cc1-c1ccc(NC(=O)OC(C)(C)C)nc1]

Compound 10 was prepared according to GP1 using 5-bromo-6-methoxy-1H-indazole (30.0 mg, 132 μmol), tert-butyl (5-(4,4,5,5-tetramethyl-1,3,2-dioxaborolan-2-yl)pyridin-2-yl)carbamate (55.0 mg, 172 μmol), Pd(dppf)Cl₂ (4.83 mg, 6.61 μmol) and Na₂CO₃ (32.2 mg. 304 μmol) in 2.5 ml dioxane and 0.5 ml H₂O. The reaction was finished after 115 h. The reaction mixture was used crude. ESI-MS: 40.95 [M_{deprotected}+H+].

### 5-(6-Nitro-1H-indazol-5-yl)pyridin-2-amine (Compound 11): (TP78)

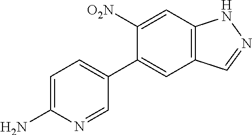 [SMILES: Nc1ccc(-c2cc3cn[nH]c3cc2[N+](=O)[O-])cn1]

Compound 11 was prepared according to GP2 using compound 9 and trifluoroacetic acid. The reaction was finished after 97 h. Purification by preparative HPLC (ACN/H₂O+0.1% TFA: 10% for 3 min, 10%→55% in 10 min, 55%→90% in 1 min, 90% for 1 min, 90%→10% in 1 min, 10% for 1 min) yielded 17.1 mg (67.0 μmol, 54%) of a yellow solid. ESI-MS: 255.96 [M+H]⁺. ¹H NMR: (400 MHz, DMSO-d₆) δ 13.92 (s, 1H), 8.44-8.41 (m, 1H), 8.37 (d, J=1.1 Hz, 1H), 8.26 (s, 2H), 8.09 (d, J=2.2 Hz, 1H), 7.99 (d, J=0.5 Hz, 1H), 7.95 (dd, J=9.2, 2.2 Hz, 1H), 7.04 (d, J=9.1 Hz, 1H). ¹³C NMR: (101 MHz, DMSO-d₆) δ 153.89, 147.12, 145.02, 134.75 (d, J=12.7 Hz), 124.94, 124.90, 123.61, 122.35, 113.42, 109.01. HR-MS (ESI): m/z [M+H]⁺ found 256.0829, calculated 256.0756 for C₁₂H₉N₅O₂.

### Purity:

| eluent system 1, t_{R} = 13.5 min: | eluent system 2, t_{R} = 7.7 min: |
|---|---|
| λ = 210 nm: >99.9% | λ = 210 nm: 96.5% |
| λ = 220 nm: >99.9% | λ = 220 nm: 97.0% |
| λ = 230 nm: >99.9% | λ = 230 nm: 97.0% |
| λ = 254 nm: >99.9% | λ = 254 nm: 97.2% |
| λ = 280 nm: >99.9% | λ = 280 nm: 98.0% |

### 5-(6-Methoxy-1H-indazol-5-yl)pyridin-2-amine (Compound 12): (TP79)

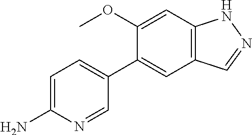 [SMILES: COc1cc2[nH]ncc2cc1-c1ccc(N)nc1]

Compound 12 was prepared according to GP2 using compound 10 and trifluoroacetic acid. The reaction was finished after 18.5 h. Purification by preparative HPLC (ACN/H₂O+0.1% TFA: 10% for 3 min, 10%→50% in 8 min, 50%→90% in 1 min, 90% for 1 min, 90%→10% in 1 min, 10% for 1 min) yielded 26.9 mg (112 μmol, 85%) of an off-white solid. ESI-MS: 240.95 [M+H]⁺. ¹H NMR: (400 MHz, DMSO-d₆) δ 13.03 (s, 1H), 8.20 (s, 2H), 8.11 (dd, J=9.2, 2.2 Hz, 1H), 8.05 (d, J=2.1 Hz, 1H), 8.02 (d, J=1.0 Hz, 1H), 7.72 (s, 1H), 7.09 (d, J=0.9 Hz, 1H), 7.04 (dd, J=9.2, 1.3 Hz, 1H), 3.86 (s, 3H). ¹³C NMR: (101 MHz, DMSO-d₆) δ 156.19, 153.43, 146.23, 141.25, 134.97, 134.17, 123.62, 122.14, 120.15, 117.77, 113.03, 91.51, 56.27. HR-MS (ESI): m/z [M+H]⁺ found 241.1079, calculated 241.1011 for C₁₃H₁₂N₄O.

### Purity:

| eluent system 1, t_{R} = 13.0 min: | eluent system 2, t_{R} = 11.2 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### Tert-butyl (5-(4-fluoro-1H-indazol-5-yl)pyridin-2-yl)carbamate (Compound 13): (TP82)

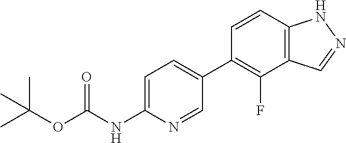 [SMILES: CC(C)(C)OC(=O)Nc1ccc(-c2ccc3[nH]ncc3c2F)cn1]

Compound 13 was prepared according to GP1 using 5-bromo-4-fluoro-1H-indazole (20.0 mg, 93.0 μmol), tert-butyl (5-(4,4,5,5-tetramethyl-1,3,2-dioxaborolan-2-yl)pyridin-2-yl)carbamate (38.7 mg, 121 μmol), Pd(dppf)Cl₂ (3.40 mg, 4.65 μmol) and Na₂CO₃ (22.7 mg. 214 μmol) in 2.5 ml dioxane and 0.5 ml H₂O. The reaction was finished after 18 h. The reaction mixture was used crude. ESI-MS: 329.13 [M+H]⁺, 228.94 [M_{deprotected}+H]⁺.

### Tert-butyl (5-(4-chloro-1H-indazol-5-yl)pyridin-2-yl)carbamate (Compound 14): (TP83)

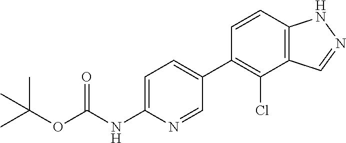 [SMILES: CC(C)(C)OC(=O)Nc1ccc(-c2ccc3[nH]ncc3c2Cl)cn1]

Compound 14 was prepared according to GP1 using 5-bromo-4-chloro-1H-indazole (20.0 mg, 86.4 μmol), tert-butyl (5-(4,4,5,5-tetramethyl-1,3,2-dioxaborolan-2-yl)pyridin-2-yl)carbamate (36.0 mg, 112 μmol), Pd(dppf)Cl₂ (3.16 mg, 4.32 μmol) and Na₂CO₃ (21.1 mg. 199 μmol) in 2.5 ml dioxane and 0.5 ml H₂O. The reaction was finished after 18 h. The reaction mixture was used crude. ESI-MS: 345.14 [M+H]⁺, 244.94 [M_{deprotected}+H]⁺.

### 5-(4-Fluoro-1H-indazol-5-yl)pyridin-2-amine (Compound 15): (TP84)

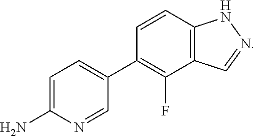 [SMILES: Nc1ccc(-c2ccc3[nH]ncc3c2F)cn1]

Compound 15 was prepared according to GP2 using compound 13 and trifluoroacetic acid. The reaction was finished after 8 h. Purification by preparative HPLC (ACN/H₂O+0.1% TFA: 10% for 3 min, 10%→55% in 10 min, 55%→90% in 1 min, 90% for 1 min, 90%→10% in 1 min, 10% for 1 min) yielded 13.6 mg (59.6 μmol, 64%) of an off-white solid. ESI-MS: 228.94 [M+H]⁺. ¹H NMR: (400 MHz, DMSO-d₆) δ 13.57 (s, 1H), 8.28 (s, 1H), 8.20 (d, J=2.1 Hz, 1H), 8.19-8.14 (m, 1H), 8.12 (s, 2H), 7.52-7.48 (m, 2H), 7.08 (d, J=9.1 Hz, 1H). ¹³C NMR: (101 MHz, DMSO-d₆) δ 153.92, 153.13, 150.63, 144.62 (d, J=3.0 Hz), 136.29, 128.38 (d, J=2.2 Hz), 120.16, 113.64, 112.91 (d, J=10.6 Hz), 107.95. HR-MS (ESI): m/z [M+H]⁺ found 229.0882, calculated 229.0811 for C₁₄H₁₅N₀.

### Purity:

| eluent system 1, t_{R} = 12.7 min: | eluent system 2, t_{R} = 11.91 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 5-(4-Chloro-1H-indazol-5-yl)pyridin-2-amine (Compound 16): (TP85)

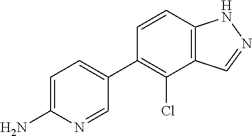 [SMILES: Nc1ccc(-c2ccc3[nH]ncc3c2Cl)cn1]

Compound 16 was prepared according to GP2 using compound 14 and trifluoroacetic acid. The reaction was finished after 23 h. Purification by preparative HPLC (ACN/H₂O+0.1% TFA: 10% for 3 min, 10%→55% in 10 min, 55%→90% in 1 min, 90% for 1 min, 90%→10% in 1 min, 10% for 1 min) yielded 9.40 mg (38.4 μmol, 44%) of an off-white solid. ESI-MS: 244.93 [M+H]⁺. ¹H NMR: (400 MHz, DMSO-d₆) δ 13.59 (s, 1H), 8.22-8.21 (m, 1H), 8.11 (d, J=2.2 Hz, 1H), 8.06 (dd, J=9.1, 2.3 Hz, 1H), 8.05 (s, 2H), 7.65 (dd, J=8.5, 1.0 Hz, 1H), 7.43 (d, J=8.5 Hz, 1H), 7.04 (d, J=9.1 Hz, 1H). ¹³C NMR: (101 MHz, DMSO-d₆) δ 154.26, 145.30, 137.52, 132.92, 129.43, 126.49, 123.41, 123.23 (d, J=1.8 Hz), 123.02, 112.86, 110.41. HR-MS (ESI): m/z [M+H]⁺ found 245.0587, calculated 245.0516 for C₁₄H₁₅NO.

### Purity:

| eluent system 1, t_{R} = 13.6 min: | eluent system 2, t_{R} = 11.5 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### Tert-butyl (5-(7-(trifluoromethyl)-1H-indazol-5-yl)pyridin-2-yl)carbamate (Compound 17): (TP86)

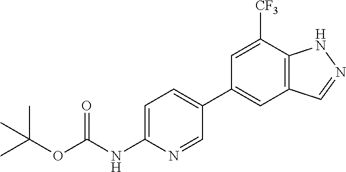 [SMILES: CC(C)(C)OC(=O)Nc1ccc(-c2cc(C(F)(F)F)c3[nH]ncc3c2)cn1]

Compound 17 was prepared according to GP1 using 5-bromo-7-(trifluoromethyl)-1H-indazole (20.0 mg, 75.7 μmol), tert-butyl (5-(4,4,5,5-tetramethyl-1,3,2-dioxaborolan-2-yl)pyridin-2-yl)carbamate (31.4 mg, 98.1 μmol), Pd(dppf)Cl₂ (2.76 mg, 3.77 μmol) and Na₂CO₃ (18.4 mg. 174 μmol) in 2.5 ml dioxane and 0.5 ml H₂O. The reaction was finished after 95 h. The reaction mixture was used crude. ESI-MS: 379.12 [M+H]⁺; 278.96 [M_{deprotected}+H]⁺; 323.07 [M_{deprotected}2Na—H]⁺.

### 5-(7-(Trifluoromethyl)-1H-indazol-5-yl)pyridin-2-amine (Compound 18): (TP87)

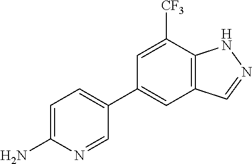 [SMILES: Nc1ccc(-c2cc(C(F)(F)F)c3[nH]ncc3c2)cn1]

Compound 18 was prepared according to GP2 using compound 17 and trifluoroacetic acid. The reaction was finished after 17 h. Purification by preparative HPLC (ACN/H₂O+0.1% TFA: 10% for 3 min, 10%→60% in 10 min, 60%→90% in 1 min, 90% for 1 min, 90%→10% in 1 mmin, 10% for 1 min) yielded 26.5 mg (70.0 μmol, 93%) of light-orange solid. ESI-MS: 244.93 [M+H]⁺. ¹H NMR: (400 MHz, DMSO-d₆) δ 13.82 (s, 1H), 8.43 (d, J=2.2 Hz, 1H), 8.40 (d, J=2.1 Hz, 2H), 8.38 (d, J=2.7 Hz, 1H), 8.12 (s, 2H), 8.04 (t, J=1.3 Hz, 1H), 7.08 (d, J=9.2 Hz, 1H). ¹³C NMR: (101 MHz, DMSO-d₆) δ 154.00, 143.07, 134.69, 129.62, 128.35, 127.53, 126.40, 125.65, 124.95, 124.25, 123.50, 123.01 (q, J=4.8 Hz), 113.85. HR-MS (ESI): m/z [M+H]⁺ found 245.0587, calculated 245.0516 for C₁₄H₁₅NO.

### Purity:

| eluent system 1, t_{R} = 13.6 min: | eluent system 2, t_{R} = 12.2 min: |
|---|---|
| λ = 210 nm: 95.1% | λ = 210 nm: 94.2% |
| λ = 220 nm: 95.3% | λ = 220 nm: 96.2% |
| λ = 230 nm: 95.0% | λ = 230 nm: 95.7% |
| λ = 254 nm: 95.1% | λ = 254 nm: 94.3% |
| λ = 280 nm: 94.3% | λ = 280 nm: 94.0% |

### 3-(4-Methyl-1H-indazol-5-yl)pyridin-2-amine (5193, Z4767470594)—Enamine Procedure 1

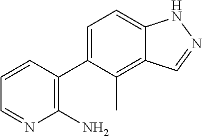 [SMILES: Cc1c(-c2cccnc2N)ccc2[nH]ncc12]

¹H NMR (400 MHz, DMSO-d₆) δ 2.34 (s, 3H), 5.41 (s, 2H), 6.66 (dd, J=7.2, 5.1 Hz, 1H), 7.09 (d, J=8.5 Hz, 1H), 7.26 (dd, J=7.2, 1.9 Hz, 1H), 7.39-7.48 (m, 1H), 7.97 (dd, J=5.0, 1.9 Hz, 1H), 8.15-8.21 (m, 1H), 13.08 (s, 1H); ¹³C NMR (151 MHz, DMSO-d6) δ 15.87, 107.99, 112.42, 120.53, 124.14, 127.84, 128.34, 128.66, 133.01, 138.44, 139.28, 146.05, 156.83.

### 3-Methyl-5-(4-methyl-1H-indazol-5-yl)pyridin-2-amine (5649, Z4767470563, Z4767467055)—Enamine Procedure 2

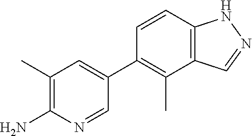 [SMILES: Cc1cc(-c2ccc3[nH]ncc3c2C)cnc1N]

¹H NMR (400 MHz, DMSO-d₆) δ 2.10 (s, 3H), 2.47 (s, 3H), 3.17 (s, 1H), 5.73 (s, 2H), 7.16 (d, J=8.5 Hz, 1H), 7.27 (dd, J=2.3, 1.0 Hz, 1H), 7.37 (d, J=8.5 Hz, 1H), 7.78 (dd, J=2.4, 0.7 Hz, 1H), 8.11 (dd, J=15.7, 1.0 Hz, 1H), 13.01 (s, 1H); ¹³C NMR (151 MHz, DMSO-d₆) δ 16.45, 17.04, 107.53, 115.12, 124.22, 125.48, 126.90, 128.70, 129.87, 132.81, 138.20, 138.83, 145.29, 156.85.

### 3-(Benzo[d]thiazol-5-yl)pyridin-2-amine (8723, Z2204874213)—Enamine Procedure 2

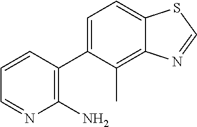 [SMILES: Cc1c(-c2cccnc2N)ccc2scnc12]

¹H NMR (400 MHz, DMSO-d₆) δ 5.67 (s, 2H), 6.69 (dd, J=7.3, 4.9 Hz, 1H), 7.41 (dd, J=7.3, 1.9 Hz, 1H), 7.55 (dd, J=8.3, 1.7 Hz, 1H), 7.98 (dd, J=4.9, 1.8 Hz, 1H), 8.11 (dd, J=1.7, 0.6 Hz, 1H), 8.24 (dd, J=8.3, 0.6 Hz, 1H), 9.44 (s, 1H); ¹³C NMR (101 MHz, DMSO-d₆) δ 113.11, 119.96, 122.79, 122.93, 126.20, 132.57, 136.51, 137.91, 147.27, 153.62, 156.69, 156.78.

### 3-(1H-Indazol-5-yl)pyridin-2-amine (9241, Z3484489528, Z4767467059)—Enamine Procedure 1

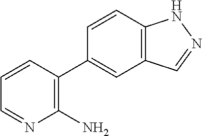 [SMILES: Nc1ncccc1-c1ccc2[nH]ncc2c1]

¹H NMR (400 MHz, DMSO-d₆) δ 5.53 (s, 2H), 6.66 (dd, J=7.3, 4.9 Hz, 1H), 7.35 (dd, J=7.3, 1.9 Hz, 1H), 7.39 (dd, J=8.6, 1.6 Hz, 1H), 7.61 (d, J=8.6 Hz, 1H), 7.79 (dd, J=1.6, 1.0 Hz, 1H), 7.95 (dd, J=4.9, 1.9 Hz, 1H), 8.10 (d, J=1.0 Hz, 1H), 13.13 (s, 1H); ¹³C NMR (101 MHz, DMSO-d₆) δ 110.57, 113.09, 120.26, 121.07, 123.35, 127.02, 130.26, 133.78, 137.67, 139.18, 146.66, 156.85.

### 2-(1-Phenylvinyl)pyridin-4-amine (ZINC001242890172, WXVL_BT2072LP0110)

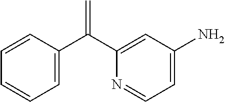

Aromatic bromide (0.1 g, 393.58 umol, 1.0 eq) was dissolved in dioxane (2 mL) and H₂O (0.4 mL). A boronic acid/boronic acid pinacol ester (1.0 eq) was added to the solution together with K₃PO₄ (3.0 eq), (Boc)₂₀ (1.0 eq) and Pd(dppf)Cl₂ (0.1 eq) under argon protection. The mixture was shaken at 80° C. for 8 hrs. LC-MS showed the desired product mass was detected. To the reaction mixture was added thiourea resin (0.5 g), and the mixture was stirred at 25° C. for another 16 hrs. Then the mixture was filtered through a celite pad, and the filtrate was concentrated to give the crude product. The residue was purified by prep-HPLC. Yield: 32.5%. ¹H NMR (400 MHz, DMSO-d₆): δ 5.40 (d, J=2.0 Hz, 1H), 5.84 (d, J=2.0 Hz, 1H), 6.00 (s, 2H), 6.39 (s, 1H), 6.37-6.43 (m, 1H), 7.27-7.41 (m, 5H), 7.79-8.02 (m, 1H). ¹³C NMR (151 MHz, DMSO-d₆): δ 107.67, 107.74, 116.05, 127.44, 128.10, 128.12, 140.53, 149.10, 149.41, 154.66, 157.53.

### tert-Butyl 5-(1H-indol-4-yl)-3,6-dihydropyridine-1(2H)-carboxylate (PS-NN04-Boc)

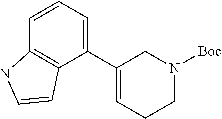 [SMILES: CC(C)(C)OC(=O)N1CCC=C(c2cccc3[nH]ccc23)C1]

tert-Butyl-5-(4,4,5,5-tetramethyl-1,3,2-dioxaborolan-2-yl)-3,6-dihydropyridine-1(2H)-carboxylate (32.0 mg, 103 μmol), 4-Bromoindole (9.36 μl, 74.6 μmol), Pd(dppf)Cl₂ (13.0 mg, 17.8 μmol) and Na₂CO₃ (21.9 mg, 207 μmol) were dissolved in dioxane (3 ml) and water (0.8 ml). The microwave tube was purged with nitrogen an heated for 2 h to 100° C. while stirring. The reaction progress was monitored using thin layer chromatography and LC/MS. After the reaction mixture had cooled down to room temperature the aqueous phase was washed with ehtyl acetate (3×50 ml). The combined organic layer was dried over Na₂SO₄, filtered and concentrated under reduced pressure. Then the brown oil was purified by flash column chromatography on normal phase (eluent: ethyl acetate and isohexane (1:9)). Afterwards the collected fractions were concentrated under reduced pressure, the product was obtained as brown oil. ¹H NMR (400 MHz, DMSO-d₆): δ 11.19 (s, 1H), 7.43-7.29 (m, 2H), 7.06 (dd, J=8.1, 7.3 Hz, 1H), 6.88 (dd, J=7.3, 0.9 Hz, 1H), 6.54 (s, 1H), 6.13 (dt, J=4.3, 2.4 Hz, 1H), 4.25 (q, J=2.4 Hz, 2H), 3.53 (t, J=5.8 Hz, 2H), 2.30 (h, J=3.2 Hz, 2H), 1.43 (s, 9H); ¹³C NMR (101 MHz, DMSO-d₆) δ 136.71, 132.30, 125.78, 125.60, 123.93, 121.36, 116.97, 111.30, 101.03, 79.36, 60.23, 28.59, 21.25, 14.56; LC/MS (ESI+) m/z: 299.5 [M+H]⁺.

### 4-(1,2,5,6-tetrahydropyridin-3-yl)-1H-indole (PS-NN04)

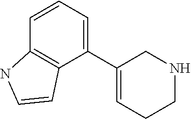 [SMILES: C1=C(c2cccc3[nH]ccc23)CNCC1]

tert-Butyl 5-(1H-indol-4-yl)-3,6-dihydropyridine-1(2H)-carboxylate (PS-NN04-Boc) was dissolved in HCl in dioxane (4 M) and stirred at room temperature for 2 h. The reaction was monitored using LC/MS. The compound was purified by preparative HPLC (column 2). The collected fractions were freeze-dried to isolate the title compound as a white solid (2.4 mg, 12%). ¹H NMR (600 MHz, Methanol-d4): δ 7.40-7.35 (m, 1H), 7.28 (d, J=3.2 Hz, 1H), 7.10 (t, J=7.7 Hz, 1H), 6.92 (d, J=7.3 Hz, 1H), 6.60-6.57 (m, 1H), 6.30 (dd, J=4.1, 2.2 Hz, 1H), 4.10 (q, J=2.2 Hz, 2H), 3.44 (t, J=6.3 Hz, 1H), 2.65 (q, J=2.9 Hz, 2H); ¹³C NMR (101 MHz, Methanol-d₄) δ 131.58, 130.13, 124.83, 122.67, 120.81, 116.75, 111.11, 99.91, 44.15, 40.36, 21.72; LC/MS (ESI+) m/z: 199.04 [M+H]⁺.

### Tert-butyl (2-(4-fluorobenzyl)pyridin-4-yl)carbamate (Compound 1): (TP_NN03)

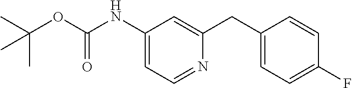 [SMILES: CC(C)(C)OC(=O)Nc1ccnc(Cc2ccc(F)cc2)c1]

Compound 1 was prepared according to GP1 using 2-(4-fluorobenzyl)-4,4,5,5-tetramethyl-1,3,2-dioxaborolane (20.0 mg, 84.7 μmol), tert-butyl (2-bromopyridin-4-yl)carbamate (30.1 mg, 110 μmol), Pd(dppf)Cl₂ (3.10 mg, 4.24 μmol) and Na₂CO₃ (20.7 mg. 195 μmol) in 2.5 ml dioxane and 0.5 ml H₂O. The reaction was finished after 22 h. The reaction mixture was used crude. ESI-MS: 202.94 [M_{deprotected}+H]⁺; 246.94 [M_{deprotected}+2Na—H]⁺, 303.04 [M+H]⁺.

### 2-(4-Fluorobenzyl)pyridin-4-amine (Compound 2): (TP94)

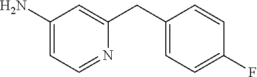 [SMILES: Nc1ccnc(Cc2ccc(F)cc2)c1]

Compound 2 was prepared according to GP2 using TP_NN03 and trifluoroacetic acid. The reaction was finished after 20 h. Purification by preparative HPLC (ACN/H₂O+0.1% TFA: 10% for 3 min, 10%→60% in 10 min, 60%→90% in 1 min, 90% for 1 min, 90%→10% in 1 min, 10% for 1 min) yielded 1.30 mg (6.43 μmol, 7%) of a brown oil. ESI-MS: 202.87 [M+H]⁺; 246.92 [M+2Na—H]⁺. ¹H NMR: (600 MHz, DMSO-d₆) δ 8.05 (d, J=7.0 Hz, 1H), 7.94 (s, 2H), 7.38-7.34 (m, 2H), 7.25-7.19 (m, 2H), 6.68 (dd, J=7.0, 2.4 Hz, 1H), 6.49 (d, J=2.4 Hz, 1H), 4.08 (s, 2H). ¹³C NMR: (151 MHz, DMSO-d₆) δ 162.67, 160.41, 153.65, 140.46, 132.84 (d, J=3.1 Hz), 131.56 (d, J=8.3 Hz), 116.10 (d, J=21.3 Hz), 108.02 (d, J=35.4 Hz), 37.49. m/z [M+H]⁺ found 203.0997, calculated 203.0906 for C₁₄H₁₅NO.

### Purity:

| eluent system 1, t_{R} = 11.8 min: | eluent system 2, t_{R} = 9.9 min: |
|---|---|
| λ = 210 nm: 96.4% | λ = 210 nm: 95.0% |
| λ = 220 nm: >99.9% | λ = 220 nm: >99.9% |
| λ = 230 nm: >99.9% | λ = 230 nm: 96.4% |
| λ = 254 nm: 90.2% | λ = 254 nm: 91.2% |
| λ = 280 nm: 92.6% | λ = 280 nm: 94.0% |

### Tert-butyl (2-(1-(3-fluorophenyl)vinyl)pyridin-4-yl)carbamate (TP_NN04) (Compound 3)

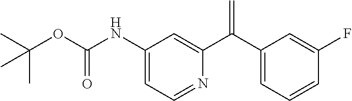 [SMILES: C=C(c1cccc(F)c1)c1cc(NC(=O)OC(C)(C)C)ccn1]

Compound 3 was prepared according to GP1 using 2-(1-(3-fluorophenyl)vinyl)-4,4,5,5-tetramethyl-1,3,2-dioxaborolane (20.0 mg, 73.2 μmol), tert-butyl (2-bromopyridin-4-yl)carbamate (23.6 mg, 95.2 μmol), Pd(dppf)Cl₂ (2.68 mg, 3.66 μmol) and Na₂CO₃ (17.9 mg. 186 μmol) in 2.5 ml dioxane and 0.5 ml H₂O. The reaction was finished after 21 h. The reaction mixture was used crude. ESI-MS: 214.87 [M_{deprotected}+H]⁺; 258.94 [M_{deprotected}+2Na—H]⁺; 315.06 [M+H]⁺.

### Tert-butyl (2-(1-(4-fluorophenyl)vinyl)pyridin-4-yl)carbamate (Compound 4): (TP_NN06)

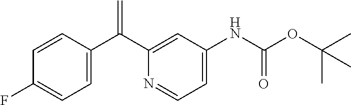 [SMILES: C=C(c1ccc(F)cc1)c1cc(NC(=O)OC(C)(C)C)ccn1]

Compound 4 was prepared according to GP1 using 2-(1-(4-fluorophenyl)vinyl)-4,4,5,5-tetramethyl-1,3,2-dioxaborolane (20.0 mg, 80.6 μmol), tert-butyl (2-bromopyridin-4-yl)carbamate (28.6 mg, 105 μmol), Pd(dppf)Cl₂ (2.95 mg, 4.03 μmol) and Na₂CO₃ (19.7 mg. 185 μmol) in 2.5 ml dioxane and 0.5 ml H₂O. The reaction was finished after 21 h. The reaction mixture was used crude. ESI-MS: 214.86 [M_{deprotected}+H]⁺; 315.05 [M+H]⁺; 651.24 [2M+Na]⁺.

### 2-(1-(3-Fluorophenyl)vinyl)pyridin-4-amine (Compound 5): (TP95)

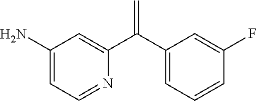 [SMILES: C=C(c1cccc(F)c1)c1cc(N)ccn1]

Compound 5 was prepared according to GP2 using compound 3 and trifluoroacetic acid. The reaction was finished after 22 h. Purification by preparative HPLC (ACN/H₂O+0.1% TFA: 10% for 3 min, 10%→60% in 10 min, 60%→90% in 1 min, 90% for 1 min, 90%→10% in 1 min, 10% for 1 min) yielded 10.4 mg (48.5 μmol, 66%) of a light-yellow oil. ESI-MS: 214.87 [M+H]⁺. ¹H NMR: (400 MHz, DMSO-d₆) δ 8.11 (d, J=7.0 Hz, 1H), 8.09 (s, 2H), 7.54-7.48 (m, 1H), 7.36-7.26 (m, 2H), 7.24-7.19 (m, 1H), 6.81 (dd, J=7.0, 2.4 Hz, 1H), 6.65 (d, J=2.4 Hz, 1H), 6.07 (s, 1H), 5.96 (s, 1H). ¹³C NMR: (101 MHz, DMSO-d₆) δ 163.96, 161.54, 160.51, 149.97, 140.97, 139.79 (d, J=7.8 Hz), 131.39 (d, J=8.5 Hz), 124.59 (d, J=2.8 Hz), 123.25, 116.34 (d, J=21.1 Hz), 115.18 (d, J=22.5 Hz), 109.02, 108.59. m/z [M+H]⁺ found 215.0979, calculated 215.0906 for C₁₄H₁₅NO.

### Purity:

| eluent system 1, t_{R} = 13.1 min: | eluent system 2, t_{R} = 11.4 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### 2-(1-(4-Fluorophenyl)vinyl)pyridin-4-amine (Compound 6): (TP96)

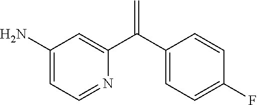 [SMILES: C=C(c1ccc(F)cc1)c1cc(N)ccn1]

Compound was prepared according to GP2 using compound 4 and trifluoroacetic acid. The reaction was finished after 22 h. Purification by preparative HPLC (ACN/H₂O+0.1% TFA: 10% for 3 min, 10%→60% in 10 min, 60%→90% in 1 min, 90% for 1 min, 90%→10% in 1 min, 10% for 1 min) yielded 16.4 mg (76.6 μmol, 95%) of a yellow oil. ESI-MS: 214.87 [M+H]⁺. ¹H NMR: (400 MHz, DMSO-d₆) δ 8.15 (s, 2H), 8.11 (d, J=7.0 Hz, 1H), 7.48-7.43 (m, 2H), 7.34-7.27 (m, 2H), 6.81 (ddd, J=7.0, 2.5, 0.8 Hz, 1H), 6.63 (d, J=2.4 Hz, 1H), 5.97 (s, 1H), 5.90 (s, 1H). ¹³C NMR: (101 MHz, DMSO-d₆) δ 164.17, 161.72, 160.51, 150.34, 141.05, 140.93, 130.65 (d, J=8.5 Hz), 122.12, 116.35, 116.14, 108.91, 108.56. HR-MS (ESI): m/z [M+H]⁺ found 215.0979, calculated 215.0906 for C₁₄H₁₅NO.

### Purity:

| eluent system 1, t_{R} = 12.5 min: | eluent system 2, t_{R} = 13.1 min: |
|---|---|
| λ = 210 nm: >99.9% | λ = 210 nm: >99.9% |
| λ = 220 nm: >99.9% | λ = 220 nm: >99.9% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >99.9% |

### tert-butyl (2-(1-(4-(trifluoromethyl)phenyl)vinyl)pyridin-4-yl)carbamate (TP100) (Compound 7)

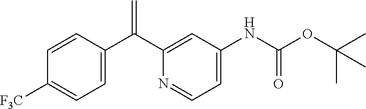 [SMILES: C=C(c1ccc(C(F)(F)F)cc1)c1cc(NC(=O)OC(C)(C)C)ccn1]

Compound 7 was prepared according to GP1 using 4,4,5,5-tetramethyl-2-(1-(4-(trifluoromethyl)phenyl)vinyl)-1,3,2-dioxaborolane (20.0 mg, 67.1 μmol), tert-butyl (2-bromopyridin-4-yl)carbamate (23.8 mg, 87.2 μmol), Pd(dppf)Cl₂ (2.45 mg, 3.35 μmol) and Na₂CO₃ (16.4 mg, 154 μmol) in 2.5 ml dioxane and 0.5 ml H₂O. The reaction was finished after 66 h. The reaction mixture was used crude. ESI-MS: 365.11 [M+H]⁺, 387.17 [M+Na]⁺.

### 2-(1-(4-(Trifluoromethyl)phenyl)vinyl)pyridin-4-amine (Compound 8) (TP101)

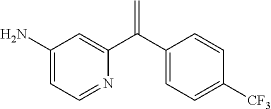 [SMILES: C=C(c1ccc(C(F)(F)F)cc1)c1cc(N)ccn1]

Compound 8 was prepared according to GP2 using compound 7 and trifluoroacetic acid. The reaction was finished after 20 h. Purification by preparative HPLC (ACN/H₂O+0.1% TFA: 10% for 3 min, 10%→60% in 10 min, 60%→90% in 1 min, 90% for 1 min, 90%→10% in 1 min, 10% for 1 min) yielded 18.5 mg (70.0 μmol, 96%) of a yellow-brown oil. ESI-MS: 264.94 [M+H]⁺. ¹H NMR: (400 MHz, DMSO-d₆) δ 8.13 (d, J=7.0 Hz, 1H), 7.83 (d, J=8.0 Hz, 2H), 7.64 (d, J=8.0 Hz, 2H), 6.83 (dd, J=7.0, 2.4 Hz, 1H), 6.64 (d, J=2.4 Hz, 1H), 6.12 (s, 1H), 6.07 (s, 1H), 3.74-3.42 (m, 2H). ¹³C NMR: (101 MHz, DMSO-d₆) δ 160.54, 149.73, 141.06, 140.90, 129.34, 126.20 (t, J=3.8 Hz), 124.17, 109.05, 108.60. HR-MS (ESI): m/z [M+H]⁺ found 265.0949, calculated 265.0874 for C₁₄H₁₅NO.

### Purity:

| eluent system 1, t_{R} = 15.4 min: | eluent system 2, t_{R} = 14.3 min: |
|---|---|
| λ = 210 nm: >99.9% | λ = 210 nm: 95.7% |
| λ = 220 nm: >99.9% | λ = 220 nm: 95.6% |
| λ = 230 nm: >99.9% | λ = 230 nm: >99.9% |
| λ = 254 nm: >99.9% | λ = 254 nm: >99.9% |
| λ = 280 nm: >99.9% | λ = 280 nm: >98.4% |

### Tert-butyl (2-(1-(4-chlorophenyl)vinyl)pyridin-4-yl)carbamate (TP102) (Compound 9)

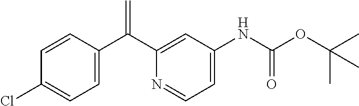 [SMILES: C=C(c1ccc(Cl)cc1)c1cc(NC(=O)OC(C)(C)C)ccn1]

Compound 9 was prepared according to GP1 using 2-(1-(4-chlorophenyl)vinyl)-4,4,5,5-tetramethyl-1,3,2-dioxaborolane (20.0 mg, 75.6 μmol), tert-butyl (2-bromopyridin-4-yl)carbamate (26.8 mg, 98.3 μmol), Pd(dppf)Cl₂ (2.77 mg, 3.78 μmol) and Na₂CO₃ (18.4 mg. 174 μmol) in 2.5 ml dioxane and 0.5 ml H₂O. The reaction was finished after 44 h. The reaction mixture was used crude. ESI-MS: 331.12 [M+H]⁺.

### 2-(1-(4-Chlorophenyl)vinyl)pyridin-4-amine (Compound 10): (TP104)

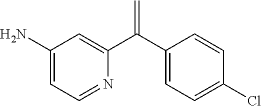 [SMILES: C=C(c1ccc(Cl)cc1)c1cc(N)ccn1]

Compound 10 was prepared according to GP2 using compound 9 and trifluoroacetic acid. The reaction was finished after 45 h. Purification by preparative HPLC (ACN/H₂O+0.1% TFA: 10% for 3 min, 10%→90% in 15 min, 90% for 1 min, 90%→10% in 1 min, 10% for 1 min) yielded 8.10 mg (35.1 μmol, 46%) of a yellow oil. ESI-MS: 230.91 [M+H]⁺. ¹H NMR: (400 MHz, DMSO-d₆) δ 8.11 (d, J=7.0 Hz, 1H), 8.05 (s, 2H), 7.54 (d, J=8.4 Hz, 2H), 7.46-7.37 (m, 2H), 6.81 (dd, J=7.0, 2.4 Hz, 1H), 6.63 (d, J=2.4 Hz, 1H), 6.02 (s, 1H), 5.94 (s, 1H). ¹³C NMR: (101 MHz, DMSO-d₆) δ 160.50, 150.08, 140.95, 136.33, 134.23, 130.25, 129.34, 122.67, 108.97, 108.57. HR-MS (ESI): m/z [M+H]⁺ found 231.0686, calculated 231.0611 for C₁₄H₁₅NO.

### Purity:

| eluent system 1, t_{R} = 13.7 min: | eluent system 2, t_{R} = 12.8 min: |
|---|---|
| λ = 210 nm: 95.1% | λ = 210 nm: 96.7% |
| λ = 220 nm: 96.8% | λ = 220 nm: 97.4% |
| λ = 230 nm: 97.2% | λ = 230 nm: 97.0% |
| λ = 254 nm: 98.7% | λ = 254 nm: 98.7% |
| λ = 280 nm: 99.2% | λ = 280 nm: 97.2% |

### N²-(3-Chlorophenyl)-N²-methylpyridine-2,4-diamine (2029, Z4910230830, Z4507373641)—Enamine Procedure 6

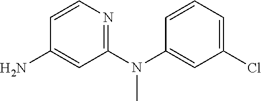 [SMILES: CN(c1cccc(Cl)c1)c1cc(N)ccn1]

¹H NMR (400 MHz, DMSO-d₆) δ 3.34 (s, 3H), 5.74 (d, J=2.1 Hz, 1H), 6.31 (dd, J=7.1, 2.1 Hz, 1H), 7.33-7.38 (m, 1H), 7.41 (s, 2H), 7.46-7.50 (m, 1H), 7.55 (d, J=4.1 Hz, 1H), 7.55 (d, J=5.8 Hz, 1H), 7.59 (d, J=7.1 Hz, 1H), 11.92 (s, 1H); ¹³C NMR (101 MHz, DMSO-d₆) δ 39.62, 89.86, 103.44, 125.68, 126.96, 127.73, 131.98, 134.29, 137.14, 144.87, 152.26, 159.13.

### N-(3,4-Dimethylphenyl)pyridin-2-amine (4472, Z54146123, Z4767467012)—Enamine Procedure 5

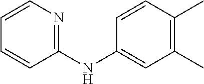

¹H NMR (400 MHz, DMSO-d₆) δ 2.15 (s, 3H), 2.18 (s, 3H), 6.67 (ddd, J=7.1, 5.0, 1.0 Hz, 1H), 6.77 (ddd, J=8.4, 0.9 Hz, 0.9 Hz, 1H), 6.98-7.02 (m, 1H), 7.36-7.38 (m, 1H), 7.40 (dd, J=8.1, 2.4 Hz, 1H), 7.50 (ddd, J=8.4, 7.1, 2.0 Hz, 1H), 8.11 (ddd, J=5.0, 2.0, 0.8 Hz, 1H), 8.79 (s, 1H); ¹³C NMR (151 MHz, DMSO-d₆) δ 18.65, 19.73, 110.16, 113.70, 115.82, 119.59, 128.00, 129.48, 135.95, 137.01, 139.38, 147.26, 156.13.

### 3-Methyl-N¹-(pyridin-2-yl)benzene-1,4-diamine (5822, Z1509730185, Z4767467010)—Enamine Procedure 6

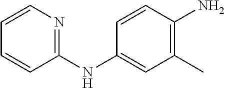

¹H NMR (400 MHz, DMSO-d₆) δ 2.04 (s, 3H), 4.60 (s, 2H), 6.55 (d, J=8.5 Hz, 1H), 6.56 (dd, J=7.1, 1.0 Hz, 1H), 6.61 (d, J=8.5 Hz, 1H), 7.04-7.10 (m, 2H), 7.41 (ddd, J=8.5, 7.1, 2.0 Hz, 1H), 8.01 (ddd, J=5.0, 2.0, 0.8 Hz, 1H), 8.33 (s, 1H); ¹³C NMR (101 MHz, DMSO-d₆) δ 17.71, 108.65, 112.64, 114.34, 119.24, 121.57, 122.78, 130.79, 136.86, 141.43, 147.43, 157.14.

N⁵-methyl-N⁵-(m-tolyl)pyridine-2,5-diamine (9878, Z905109318, Z4767467005) —Enamine procedure 7

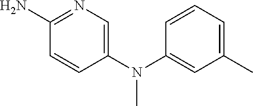 [SMILES: Cc1cccc(N(C)c2ccc(N)nc2)c1]

¹H NMR (400 MHz, DMSO-d₆) δ 2.18 (s, 3H), 3.11 (s, 3H), 5.90 (s, 2H), 6.38-6.44 (m, 2H), 6.48 (dd, J=8.7, 0.7 Hz, 1H), 6.49 (ddd, J=7.4, 1.6, 0.8 Hz, 1H), 6.96-7.05 (m, 1H), 7.21 (dd, J=8.7, 2.7 Hz, 1H), 7.75 (dd, J=2.7, 0.7 Hz, 1H); ¹³C NMR (151 MHz, DMSO-d₆) δ 21.41, 40.35, 108.58, 111.09, 114.35, 118.02, 128.65, 134.41, 135.94, 137.81, 145.87, 149.84, 157.33.

### 2-(4-Fluorophenoxy)pyridin-4-amine (4783, Z4907706973)—Enamine Procedure 9

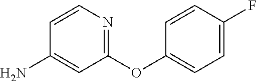 [SMILES: Nc1ccnc(Oc2ccc(F)cc2)c1]

¹H NMR (400 MHz, DMSO-d₆) δ 5.84 (d, J=2.0 Hz, 1H), 6.45 (ddd, J=6.7, 3.1, 2.0 Hz, 1H), 7.32-7.34 (m, 2H), 7.35-7.36 (m, 2H), 7.81 (dd, J=6.4, 2.0 Hz, 1H); ¹³C NMR (151 MHz, DMSO-d₆) δ 91.38, 105.67, 116.85 (d, ³J_{C-F}=23.5 Hz), 123.13 (d, ²J_{C-F}=8.6 Hz), 141.36, 148.61, 159.59 (d, J_{C-F}=238.9 Hz), 160.39, 161.31.

### 3-((4-Aminopyridin-2-yl)(methyl)amino)phenol (9448, Z4879658604, Z4767467044)—Enamine Procedure 8

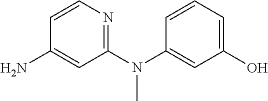 [SMILES: CN(c1cccc(O)c1)c1cc(N)ccn1]

¹H NMR (400 MHz, DMSO-d₆) δ 3.27 (s, 3H), 5.72 (d, J=1.9 Hz, 1H), 6.02 (dd, J=6.0, 1.9 Hz, 1H), 6.06 (s, 1H), 6.59-6.62 (m, 1H), 6.63-6.68 (m, 1H), 7.14-7.24 (m, 1H), 7.63 (d, J=6.0 Hz, 1H), 9.55 (s, 1H); ¹³C NMR (151 MHz, DMSO-d₆) δ 40.43, 91.58, 102.53, 112.31, 112.50, 116.10, 130.24, 147.28, 155.90, 158.35.

### 3-Fluoro-N¹-methyl-N¹-(pyridin-2-yl)benzene-1,4-diamine (6254, Z4907692923, Z4767467018)—Enamine Procedure 6

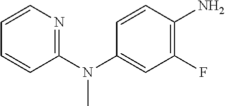 [SMILES: CN(c1ccc(N)c(F)c1)c1ccccn1]

¹H NMR (400 MHz, DMSO-d₆) δ 3.34 (s, 3H), 6.62 (d, J=9.0 Hz, 1H), 6.74-6.80 (m, 1H), 6.81-6.91 (m, 2H), 7.06 (dd, J=12.5, 2.2 Hz, 1H), 7.60-7.68 (m, 1H), 8.04 (ddd, J=5.6, 1.9, 0.8 Hz, 1H); ¹³C NMR (151 MHz, DMSO-d₆) δ 8.60, 112.50, 113.96 (d, ²J_{C-F}=18.9 Hz), 116.65 (d, ³J_{C-F}=5.8 Hz), 123.27 (d, ⁴J_{C-F}=2.7 Hz), 150.37 (d, J_{C-F}=239.0 Hz).

### 3-Chloro-N¹-(pyridin-2-yl)benzene-1,4-diamine (7502, Z1509600173)—Enamine Procedure 5

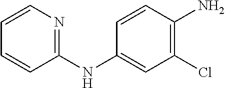 [SMILES: Nc1ccc(Nc2ccccn2)cc1Cl]

¹H NMR (400 MHz, DMSO-d₆) δ 4.93 (s, 2H), 6.63 (ddd, J=7.1, 5.0, 1.0 Hz, 1H), 6.66 (ddd, J=8.5, 1.0 Hz, 1H), 6.73 (d, J=8.6 Hz, 1H), 7.15 (dd, J=8.6, 2.5 Hz, 1H), 7.47 (ddd, J=8.4, 7.1, 2.0 Hz, 1H), 7.68 (d, J=2.4 Hz, 1H), 8.07 (ddd, J=5.0, 2.0, 0.8 Hz, 1H), 8.65 (s, 1H); ¹³C NMR (151 MHz, DMSO-d₆) δ 109.66, 113.32, 115.69, 116.91, 119.45, 119.70, 131.94, 136.96, 138.83, 147.23, 156.29.

### Example 5: Biological Data

TABLE 6
Gi recruitment.

| Compound | | EC₅₀ | Eₘₐₓ |
|---|---|---|---|
| ID | Structure | (nM) | (%) |
| 9087 | 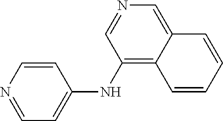 [SMILES: c1ccc2c(Nc3ccncc3)cncc2c1] | 52 agonist analgesic in vivo | n/a |
| 5879 | 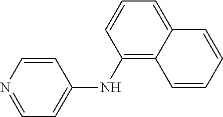 [SMILES: c1ccc2c(Nc3ccncc3)cccc2c1] | 5.8 agonist | 85 |
| 4487 | 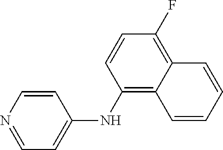 [SMILES: Fc1ccc(Nc2ccncc2)c2ccccc12] | 62 agonist | n/a |
| 9204 | 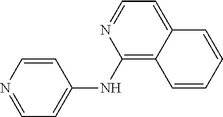 [SMILES: c1ccc2c(Nc3ccncc3)nccc2c1] | 1500 agonist | n/a |
| 9506 | 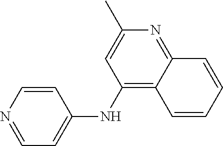 [SMILES: Cc1cc(Nc2ccncc2)c2ccccc2n1] | 950 weak agonist | n/a |
| 4825 | 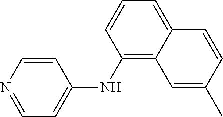 [SMILES: Cc1ccc2cccc(Nc3ccncc3)c2c1] | 15 agonist | 94 |
| 2813 | 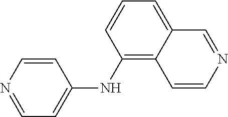 [SMILES: c1cc(Nc2ccncc2)c2ccncc2c1] | 260 weak agonist | n/a |
| 1718 | 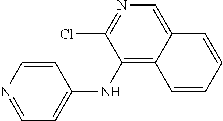 [SMILES: Clc1ncc2ccccc2c1Nc1ccncc1] | 8 agonist | 45 |
| 4914 | 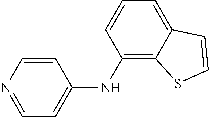 [SMILES: c1cc(Nc2ccncc2)c2sccc2c1] | 6.8 agonist | 76 |
| 3084 | 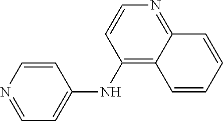 [SMILES: c1ccc2c(Nc3ccncc3)ccnc2c1] | 460 | n/a |
| PS86 | 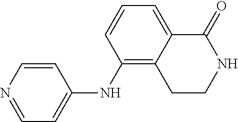 [SMILES: O=C1NCCc2c(Nc3ccncc3)cccc21] | 3200 agonist | n/a |
| PS77 | 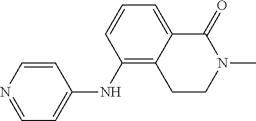 [SMILES: CN1CCc2c(Nc3ccncc3)cccc2C1=O] | 1200 agonist | n/a |
| PS85 | 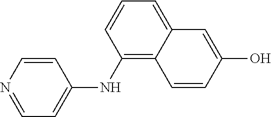 [SMILES: Oc1ccc2c(Nc3ccncc3)cccc2c1] | 87 agonist | n/a |
| PS84 | 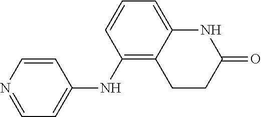 [SMILES: O=C1CCc2c(cccc2Nc2ccncc2)N1] | 450 agonist | n/a |
| PS62 | 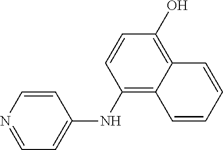 [SMILES: Oc1ccc(Nc2ccncc2)c2ccccc12] | 1200 agonist | n/a |
| 7075 | 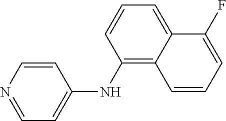 [SMILES: Fc1cccc2c(Nc3ccncc3)cccc12] | 4.1 agonist analgesic in vivo | 89 |
| PS70 | 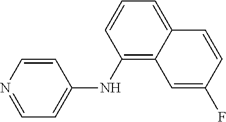 [SMILES: Fc1ccc2cccc(Nc3ccncc3)c2c1] | 23 agonist | 89 |
| PS78 | 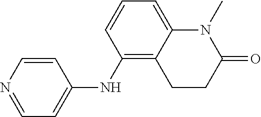 [SMILES: CN1C(=O)CCc2c(Nc3ccncc3)cccc21] | 43 agonist | n/a |
| PS71 | 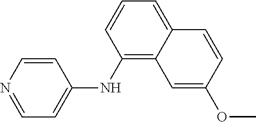 [SMILES: COc1ccc2cccc(Nc3ccncc3)c2c1] | 89 agonist | n/a |
| PS72 | 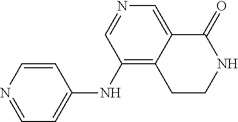 [SMILES: O=C1NCCc2c(Nc3ccncc3)cncc21] | n/a | n/a |
| PS74 | 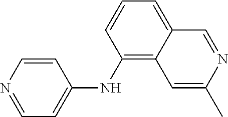 [SMILES: Cc1cc2c(Nc3ccncc3)cccc2cn1] | 450 agonist | n/a |
| PS79 | 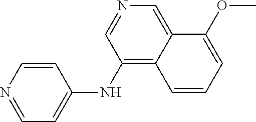 [SMILES: COc1cccc2c(Nc3ccncc3)cncc12] | 100 agonist | n/a |
| PS76 | 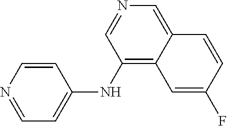 [SMILES: Fc1ccc2cncc(Nc3ccncc3)c2c1] | 89 agonist | n/a |
| PS73 | 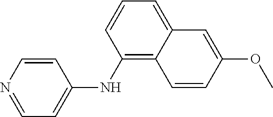 [SMILES: COc1ccc2c(Nc3ccncc3)cccc2c1] | 50 agonist | n/a |
| PS75 | 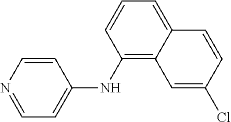 [SMILES: Clc1ccc2cccc(Nc3ccncc3)c2c1] | 4.8 agonist analgesic in vivo | 79 |
| PS83 | 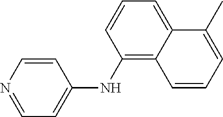 [SMILES: Cc1cccc2c(Nc3ccncc3)cccc12] | 12 agonist | 94 |
| PS82 | 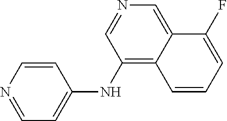 [SMILES: Fc1cccc2c(Nc3ccncc3)cncc12] | 230 agonist | n/a |
| GM17 | 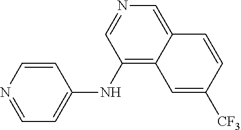 [SMILES: FC(F)(F)c1ccc2cncc(Nc3ccncc3)c2c1] | n/a | n/a |
| GM18 | 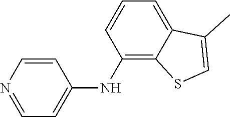 [SMILES: Cc1csc2c(Nc3ccncc3)cccc12] | n/a | n/a |
| GM14 | 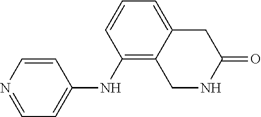 [SMILES: O=C1Cc2cccc(Nc3ccncc3)c2CN1] | n/a | n/a |
| 9835 | 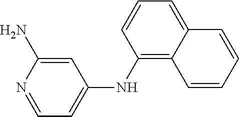 [SMILES: Nc1cc(Nc2cccc3ccccc23)ccn1] | n/a | n/a |
| 3916 | 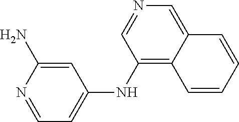 [SMILES: Nc1cc(Nc2cncc3ccccc23)ccn1] | n/a | n/a |
| GM19 | 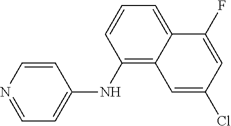 [SMILES: Fc1cc(Cl)cc2c(Nc3ccncc3)cccc12] | n/a | n/a |
| GM20 | 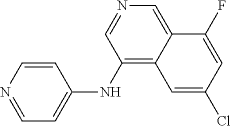 [SMILES: Fc1cc(Cl)cc2c(Nc3ccncc3)cncc12] | n/a | n/a |

TABLE 7
Gi recruitment.

| | | G protein | Arrestin |
|---|---|---|---|
| Compound | | EC₅₀ (nM), | EC₅₀ (nM), |
| ID | Structure | Eₘₐₓ (%) | Eₘₐₓ (%) |
| 4622 | 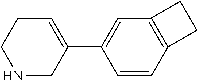 [SMILES: C1=C(c2ccc3c(c2)CC3)CNCC1] | 45 nM, 76% | <5% |
| 3461 | 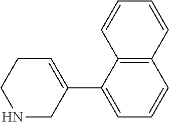 [SMILES: C1=C(c2cccc3ccccc23)CNCC1] | 450 nM | n/a |
| 3827 | 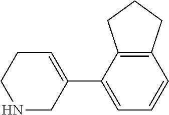 [SMILES: C1=C(c2cccc3c2CCC3)CNCC1] | 390 nM | n/a |
| 3486 | 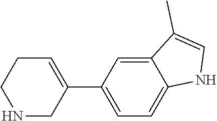 [SMILES: Cc1c[nH]c2ccc(C3=CCCNC3)cc12] | n/a | n/a |
| 9700 | 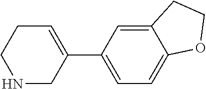 [SMILES: C1=C(c2ccc3c(c2)CCO3)CNCC1] | 800 nM | n/a |
| 9678 | 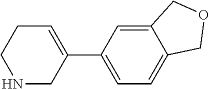 [SMILES: C1=C(c2ccc3c(c2)COC3)CNCC1] | n/a | n/a |
| 9661 | 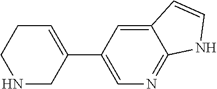 [SMILES: C1=C(c2cnc3[nH]ccc3c2)CNCC1] | 680 nM | n/a |
| 9677 | 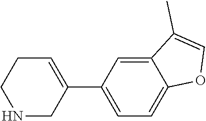 [SMILES: Cc1coc2ccc(C3=CCCNC3)cc12] | 170 nM | n/a |
| 7074 | 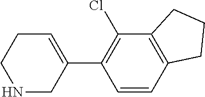 [SMILES: Clc1c(C2=CCCNC2)ccc2c1CCC2] | 480 nM | n/a |
| 9853 | 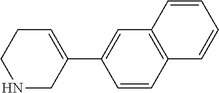 [SMILES: C1=C(c2ccc3ccccc3c2)CNCC1] | n/a | n/a |
| PS-NN04 | 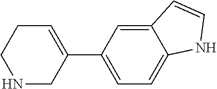 [SMILES: C1=C(c2ccc3[nH]ccc3c2)CNCC1] | 130 nM, 63% | <10% |
| TP90 | 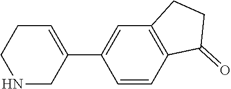 [SMILES: O=C1CCc2cc(C3=CCCNC3)ccc21] | <5% | <5% |
| TP80 | 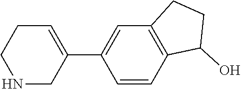 [SMILES: OC1CCc2cc(C3=CCCNC3)ccc21] | 370 nM, −28% | <5% |
| TP98 | 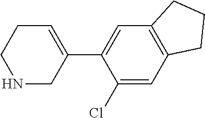 [SMILES: Clc1cc2c(cc1C1=CCCNC1)CCC2] | <10% | <5% |
| TP105 | 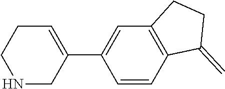 [SMILES: C=C1CCc2cc(C3=CCCNC3)ccc21] | <5% | <5% |
| TP97 | 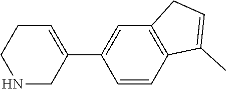 [SMILES: CC1=CCc2cc(C3=CCCNC3)ccc21] | <10% | <5% |
| TP81 | 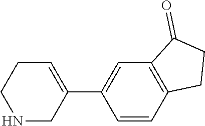 [SMILES: O=C1CCc2ccc(C3=CCCNC3)cc21] | 3000 nM, 79% | <10% |
| TP92 | 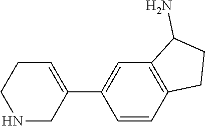 [SMILES: NC1CCc2ccc(C3=CCCNC3)cc21] | 18% at 100 μM | <5% |
| PS-NN06 | 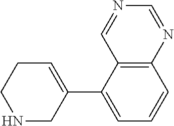 [SMILES: C1=C(c2cccc3ncncc23)CNCC1] | <10% | <10% |
| PS-NN05 | 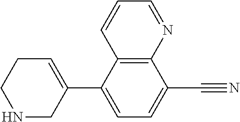 [SMILES: N#Cc1ccc(C2=CCCNC2)c2cccnc12] | <5% | <10% |

TABLE 8
Gi recruitment.

| | | G protein | Arrestin |
|---|---|---|---|
| Compound | | EC₅₀ (nM), | EC₅₀ (nM), |
| ID | Structure | Eₘₐₓ (%) | Eₘₐₓ (%) |
| 2998 | 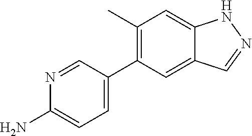 [SMILES: Cc1cc2[nH]ncc2cc1-c1ccc(N)nc1] | 89 nM, 60% | <10% |
| 5193 | 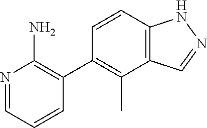 [SMILES: Cc1c(-c2cccnc2N)ccc2[nH]ncc12] | 3100 nM | n/a |
| 5649 | 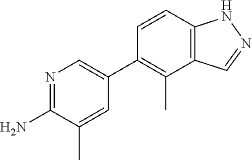 [SMILES: Cc1cc(-c2ccc3[nH]ncc3c2C)cnc1N] | 1000 nM | n/a |
| 9241 | 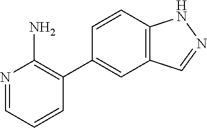 [SMILES: Nc1ncccc1-c1ccc2[nH]ncc2c1] | 2100 nM | n/a |
| 6279 | 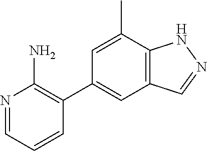 [SMILES: Cc1cc(-c2cccnc2N)cc2cn[nH]c12] | n/a | n/a |
| 8596 | 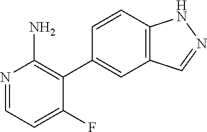 [SMILES: Nc1nccc(F)c1-c1ccc2[nH]ncc2c1] | n/a | n/a |
| 8056 | 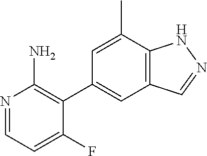 [SMILES: Cc1cc(-c2c(F)ccnc2N)cc2cn[nH]c12] | n/a | n/a |
| 5646 | 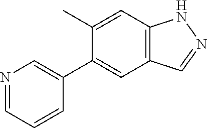 [SMILES: Cc1cc2[nH]ncc2cc1-c1cccnc1] | n/a | n/a |
| 2300 | 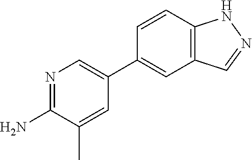 [SMILES: Cc1cc(-c2ccc3[nH]ncc3c2)cnc1N] | n/a | n/a |
| 3752 | 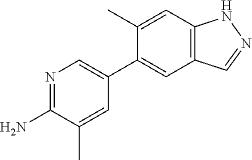 [SMILES: Cc1cc2[nH]ncc2cc1-c1cnc(N)c(C)c1] | n/a | n/a |
| 8723 | 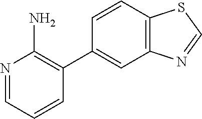 [SMILES: Nc1ncccc1-c1ccc2scnc2c1] | 1300 nM | n/a |
| TP73 | 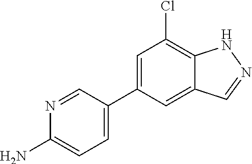 [SMILES: Nc1ccc(-c2cc(Cl)c3[nH]ncc3c2)cn1] | 8700 nM, 27% | <10% |
| TP87 | 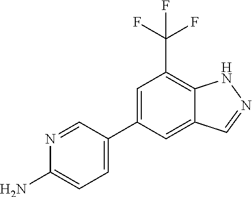 [SMILES: Nc1ccc(-c2cc(C(F)(F)F)c3[nH]ncc3c2)cn1] | 27% at 100 μM | <10% |
| TP70 | 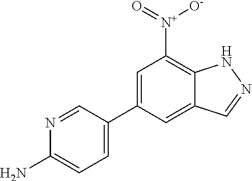 [SMILES: Nc1ccc(-c2cc([N+](=O)[O-])c3[nH]ncc3c2)cn1] | 3200 nM, −27% | <10% |
| TP74 | 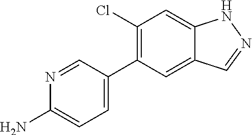 [SMILES: Nc1ccc(-c2cc3cn[nH]c3cc2Cl)cn1] | 200 nM, 64% | <10% |
| TP75 | 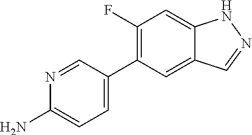 [SMILES: Nc1ccc(-c2cc3cn[nH]c3cc2F)cn1] | 89 nM, 18% | <10% |
| TP78 | 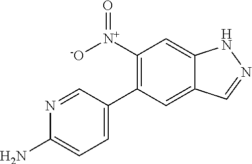 | 910 nM, 51% | <10% |
| TP79 | 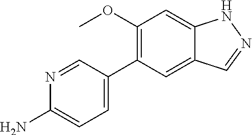 [SMILES: COc1cc2[nH]ncc2cc1-c1ccc(N)nc1] | <6% | <10% |
| BSC2998-E02 (Z6071720022) | 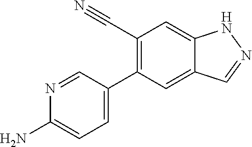 [SMILES: N#Cc1cc2[nH]ncc2cc1-c1ccc(N)nc1] | 2900 nM, 14% | 25% at 100 μM |
| TP84 | 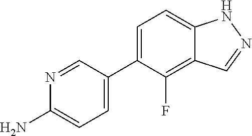 [SMILES: Nc1ccc(-c2ccc3[nH]ncc3c2F)cn1] | 1100 nM, 49% | <10% |
| TP85 | 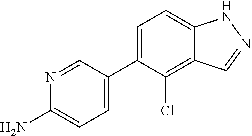 [SMILES: Nc1ccc(-c2ccc3[nH]ncc3c2Cl)cn1] | 220 nM, 81% | 34% at 100 μM |

TABLE 9
Gi recruitment.

| | | G protein | Arrestin |
|---|---|---|---|
| Compound | | EC₅₀ (nM), | EC₅₀ (nM), |
| ID | Structure | Eₘₐₓ (%) | Eₘₐₓ (%) |
| 0172 | 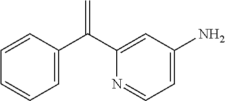 | 130 nM, 110% | 8500 nM, 23% |
| 9689 | 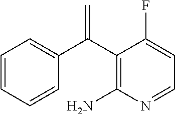 [SMILES: C=C(c1ccccc1)c1c(F)ccnc1N] | 7300 nM | n/a |
| 9280 | 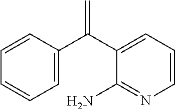 | 2600 nM | n/a |
| 6254 | 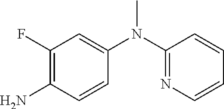 [SMILES: CN(c1ccc(N)c(F)c1)c1ccccn1] | 5400 nM | n/a |
| 4472 | 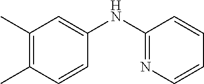 | 3300 nM | n/a |
| 9878 | 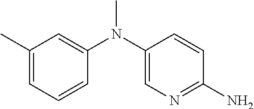 [SMILES: Cc1cccc(N(C)c2ccc(N)nc2)c1] | 1100 nM | n/a |
| 4783 | 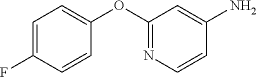 [SMILES: Nc1ccnc(Oc2ccc(F)cc2)c1] | 580 nM | n/a |
| 5364 | 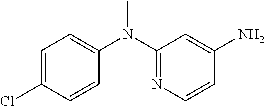 [SMILES: CN(c1ccc(Cl)cc1)c1cc(N)ccn1] | n/a | n/a |
| 2029 | 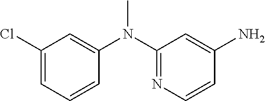 [SMILES: CN(c1cccc(Cl)c1)c1cc(N)ccn1] | 1400 nM | n/a |
| 3732 | 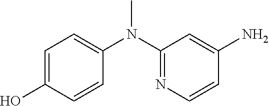 [SMILES: CN(c1ccc(O)cc1)c1cc(N)ccn1] | n/a | n/a |
| 7502 | 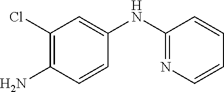 [SMILES: Nc1ccc(Nc2ccccn2)cc1Cl] | 460 nM | n/a |
| 7686 | 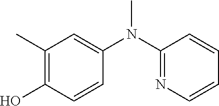 [SMILES: Cc1cc(N(C)c2ccccn2)ccc1O] | n/a | n/a |
| 5822 | 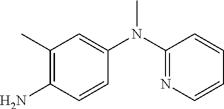 [SMILES: Cc1cc(N(C)c2ccccn2)ccc1N] | 1100 nM | n/a |
| 9448 | 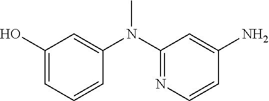 [SMILES: CN(c1cccc(O)c1)c1cc(N)ccn1] | 2900 nM | n/a |
| 5825 | 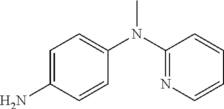 | n/a | n/a |
| 7821 | 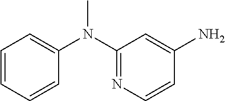 | n/a | n/a |
| 7344 | 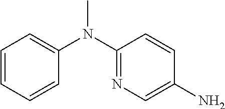 | n/a | n/a |
| TP95 | 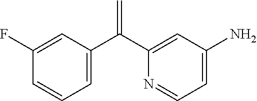 [SMILES: C=C(c1cccc(F)c1)c1cc(N)ccn1] | 93 nM, 122% | 4200 nM, 28% |
| TP96 | 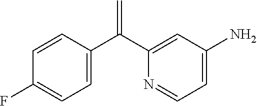 [SMILES: C=C(c1ccc(F)cc1)c1cc(N)ccn1] | 590 nM, 73% | <10% |
| TP104 | 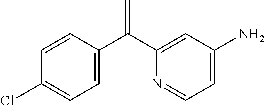 [SMILES: C=C(c1ccc(Cl)cc1)c1cc(N)ccn1] | 5200 nM, 29% | <5% |
| TP101 | 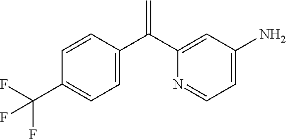 [SMILES: C=C(c1ccc(C(F)(F)F)cc1)c1cc(N)ccn1] | <5% | <5% |
| TP94 | 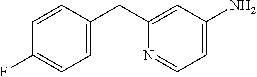 [SMILES: Nc1ccnc(Cc2ccc(F)cc2)c1] | 5000 nM, 19% | <10% |

What is claimed is:

1. A method of treating pain in a subject in need thereof, said method comprising administering to the subject in need thereof a therapeutically effective of a compound, or a pharmaceutically acceptable salt thereof, having the formula: 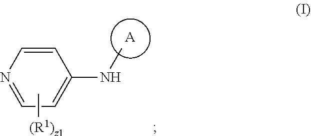
Ring A is substituted or unsubstituted aryl, substituted or unsubstituted heteroaryl, substituted or unsubstituted cycloalkyl, or substituted or unsubstituted heterocycloalkyl;
R¹ is independently halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

2. The method of claim 1, wherein the compound has the formula: 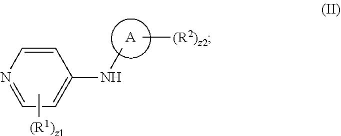
wherein
Ring A is aryl, heteroaryl, cycloalkyl, or heterocycloalkyl;
R² is independently halogen, oxo, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R² substituents may optionally be joined to form a substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 15.

3. The method of claim 2, wherein Ring A is aryl or heteroaryl.

4. The method of claim 2, wherein the compound has the formula: 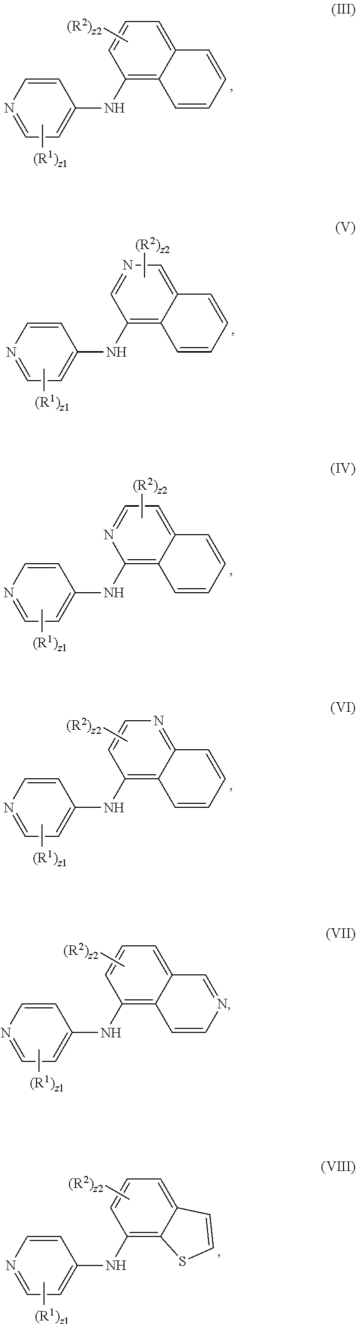 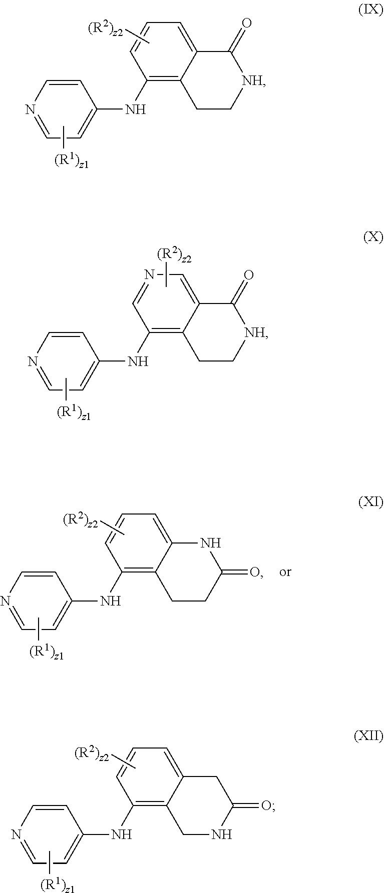
wherein
z2 is an integer from 0 to 8.

5. The method of claim 2, wherein z2 is 0 or 1.

6. The method of claim 2, wherein R² is independently halogen, oxo, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

7. The method of claim 2, wherein R² is independently halogen, oxo, —CF₃, —OR^{2D}, or unsubstituted C₁-C₄ alkyl.

8. The method of claim 2, wherein R² is independently —F, —Cl, oxo, —CF₃, —OH, —OCH₃, or unsubstituted methyl.

9. The method of claim 1, wherein z1 is 0.

10. The method of claim 1, wherein the compound is:

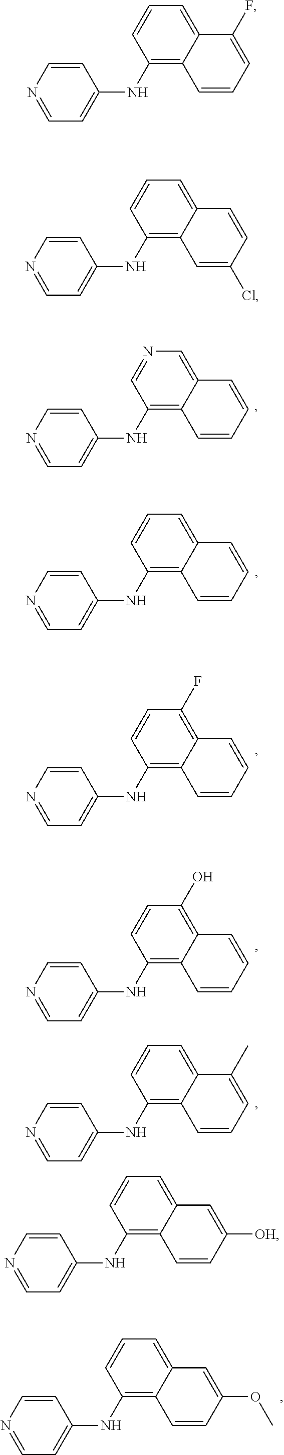

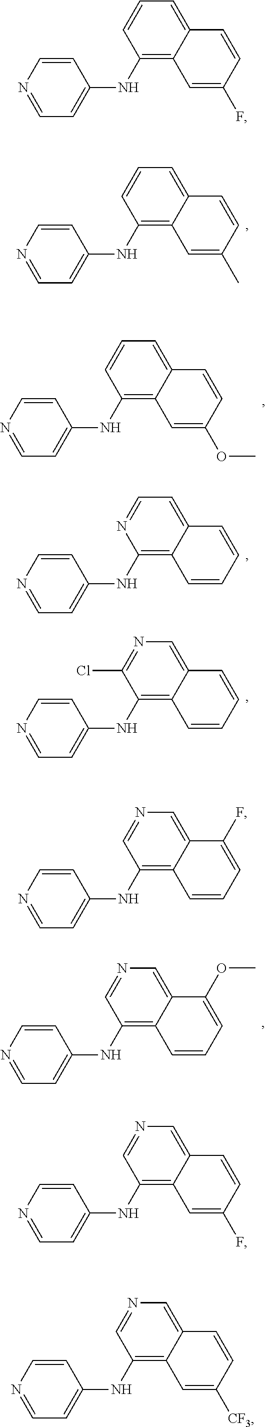

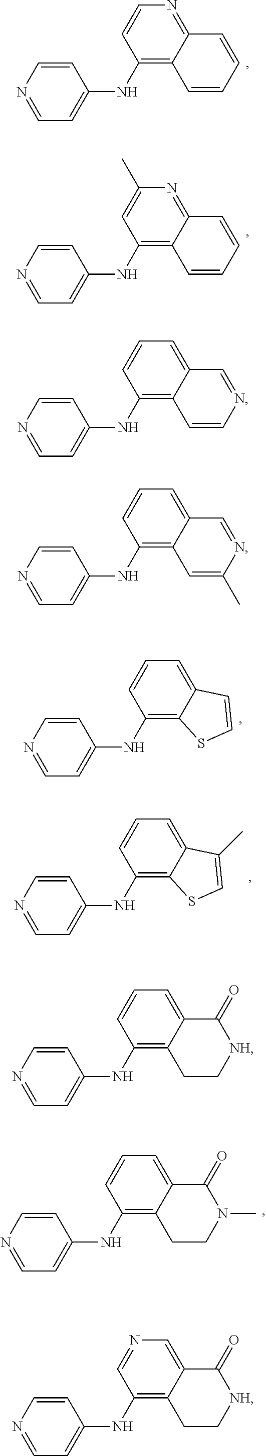

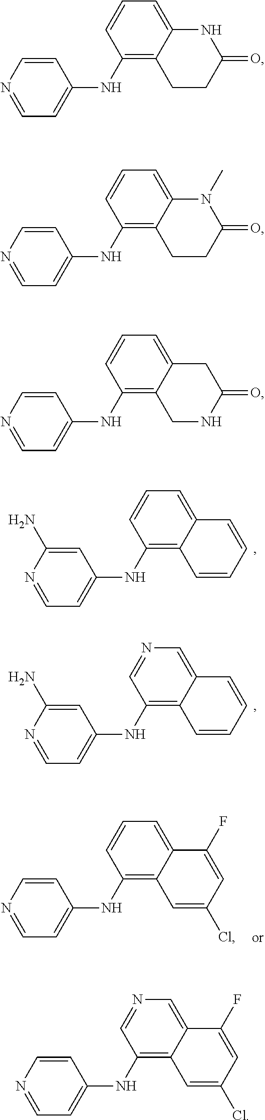

11. The method of claim 1, wherein the pain is post-operative pain.

12. The method of claim 11, wherein the post-operative pain is pain after a hysterectomy.

13. The method of claim 11, wherein the post-operative pain is pediatric post-operative pain.

14. The method of claim 11, further comprising administering a second agent.

15. The method of claim 14, wherein the second agent is an opioid.

16. The method of claim 14, wherein the second agent is bupivacaine.

17. The method of claim 1, wherein the pain is neuropathic pain.

18. The method of claim 17, wherein the neuropathic pain is post-traumatic neuropathic pain.

19. The method of claim 17, wherein the neuropathic pain is diabetic neuropathic pain.

20. The method of claim 17, wherein the neuropathic pain is post-herpetic neuralgia.

21. The method of claim 17, wherein the neuropathic pain is chemotherapy-induced pain.

22. The method of claim 17, wherein the neuropathic pain is phantom limb pain.

23. The method of claim 1, wherein the pain is inflammatory pain.

24. The method of claim 1, wherein the pain is opioid refractory pain.

25. The method of claim 1, wherein the pain is a rebound headache.

26. The method of claim 1, wherein the pain is migraine pain.

27. The method of one of claims 1 to 26, wherein the compound is administered systemically.

28. The method of one of claims 1 to 26, wherein the compound is administered topically.

29. The method of one of claims 1 to 26, wherein the compound is administered intrathecally.

30. A method of increasing the level of activity of α_{2A} adrenergic receptor in a cell, said method comprising contacting the cell with an effective of a compound, or a pharmaceutically acceptable salt thereof, having the formula: 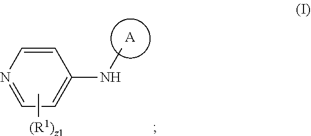
Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

31. The method of claim 30, wherein the compound binds to D128, V129, T133, 1205, S215, S219, W402, F405, F406, Y409, F427, or Y431 of α_{2A} adrenergic receptor.

32. The method of claim 30, wherein the compound binds noncovalently to D128, V129, T133, 1205, S215, S219, W402, F405, F406, Y409, F427, or Y431 of α_{2A} adrenergic receptor.

33. A pharmaceutical composition comprising a compound, or a pharmaceutically acceptable salt thereof, and a pharmaceutically acceptable excipient, wherein the compound has the formula: 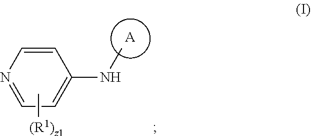
Ring A is substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl;
X¹ is independently —F, —Cl, —Br, or —I;
n1 is an integer from 0 to 4;
m1 and v1 are independently 1 or 2; and
z1 is an integer from 0 to 4.

34. A compound, or a pharmaceutically acceptable salt thereof, having the formula: 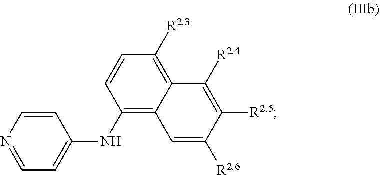
wherein
R^{2.3} is hydrogen, halogen, —OR^{2D}, or unsubstituted alkyl;
R^{2.4} is hydrogen, —OR^{2D}, or unsubstituted alkyl,
R^{2.5} is hydrogen, halogen, —OR^{2D}, or unsubstituted alkyl;
R^{2.6} is hydrogen, halogen, —OR^{2D}, or unsubstituted alkyl; and
R^{2D} is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein at least one of R^{2.3}, R^{2.4}, R^{2.5}, and R^{2.6} is not hydrogen.

35. The compound of claim 34, wherein R^{2.3} is —F or —OH.

36. The compound of claim 34, wherein R^{2.4} is unsubstituted C₁-C₄ alkyl.

37. The compound of claim 34, wherein R^{2.5} is —OH or —OCH₃.

38. The compound of claim 34, wherein R^{2.6} is —F, —Cl, —OCH₃, or unsubstituted C₁-C₄ alkyl.

39. The compound of claim 34, having the formula:

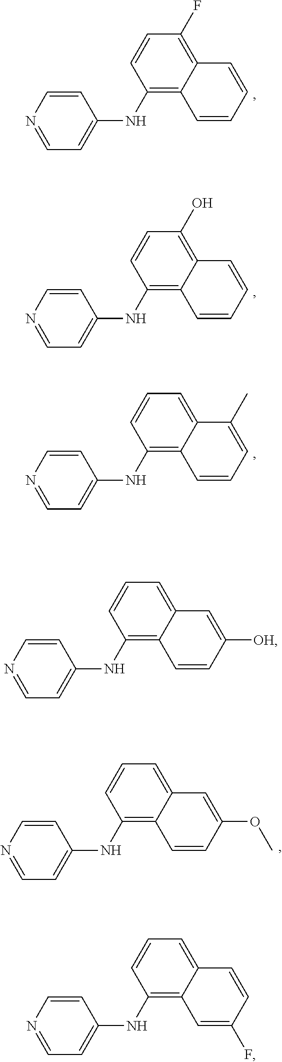 [SMILES: Fc1ccc(Nc2ccncc2)c2ccccc12 ; Oc1ccc(Nc2ccncc2)c2ccccc12 ; Cc1cccc2c(Nc3ccncc3)cccc12 ; Oc1ccc2c(Nc3ccncc3)cccc2c1 ; COc1ccc2c(Nc3ccncc3)cccc2c1 ; Fc1ccc2cccc(Nc3ccncc3)c2c1]

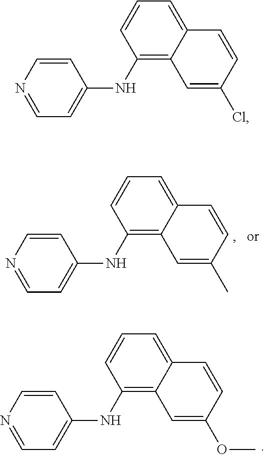 [SMILES: Clc1ccc2cccc(Nc3ccncc3)c2c1 ; Cc1ccc2cccc(Nc3ccncc3)c2c1 ; COc1ccc2cccc(Nc3ccncc3)c2c1]

40. A compound, or a pharmaceutically acceptable salt thereof, having the formula: 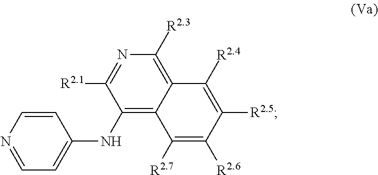
wherein
R^{2.1}, R^{2.3}, R^{2.4}, R^{2.5}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OC₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.3}, R^{2.4}, R^{2.5}, R^{2.6}, and R^{2.7} is not hydrogen;
wherein R^{2.1} is not —C₁; and
wherein R^{2.4} is not —C(O)OR^{2C}.

41. The compound of claim 40, wherein R^{2.4} is —F or —OCH₃.

42. The compound of claim 40, wherein R^{2.6} is —F, —Cl, or —CF₃.

43. The compound of claim 40, having the formula:

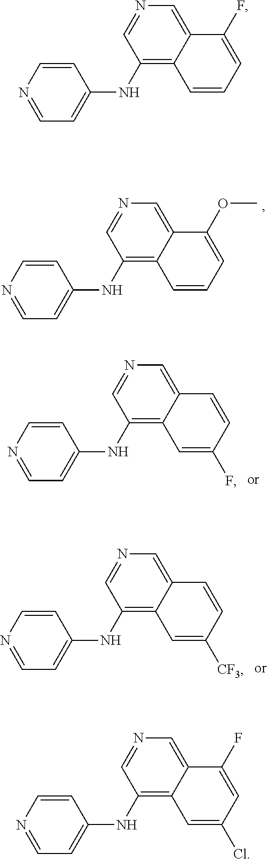

44. A compound, or a pharmaceutically acceptable salt thereof, having the formula: 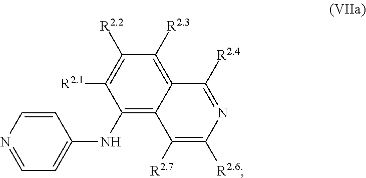
wherein
R^{2.1}, R^{2.2}, R^{2.3}, R^{2.4}, R^{2.6}, and R^{2.7} are independently hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
wherein at least one of R^{2.1}, R^{2.2}, R^{2.3}, R^{2.4}, R^{2.6}, and R^{2.7} is not hydrogen.

45. The compound of claim 44, wherein R^{2.6} is unsubstituted C₁-C₄ alkyl.

46. The compound of claim 44, having the formula:

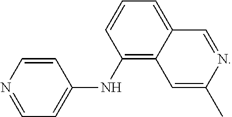 [SMILES: Cc1cc2c(Nc3ccncc3)cccc2cn1]

47. A compound, or a pharmaceutically acceptable salt thereof, having the formula: 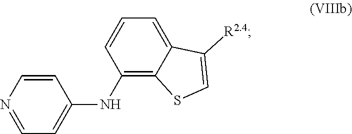
wherein
R^{2.4} is halogen, —CX²₃, —CHX²₂, —CH₂X², —OCX²₃, —OCH₂X², —OCHX²₂, —CN, —SOₙ₂R^{2D}—SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

48. The compound of claim 47, wherein R^{2.4} is unsubstituted C₁-C₄ alkyl.

49. The compound of claim 47, having the formula:

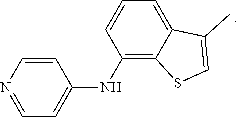 [SMILES: Cc1csc2c(Nc3ccncc3)cccc12]

50. A compound, or a pharmaceutically acceptable salt thereof, having the formula: 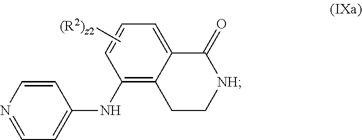
wherein
R² is independently halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

51. The compound of claim 50, having the formula: 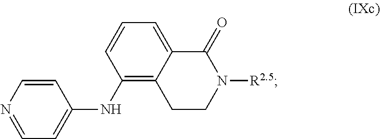
wherein
R^{2.5} is hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

52. The compound of claim 51, wherein R^{2.5} is hydrogen or unsubstituted C₁-C₄ alkyl.

53. The compound of claim 50, having the formula:

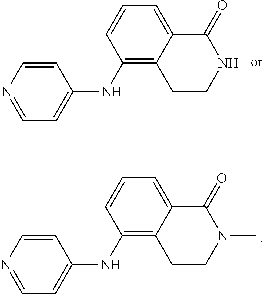 [SMILES: O=C1NCCc2c(Nc3ccncc3)cccc21 ; "or" ; CN1CCc2c(Nc3ccncc3)cccc2C1=O]

54. A compound, or a pharmaceutically acceptable salt thereof, having the formula: 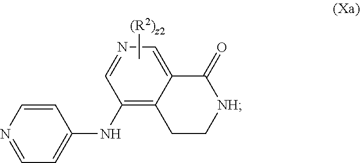
wherein
R² is independently halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 7.

55. The compound of claim 54, having the formula: 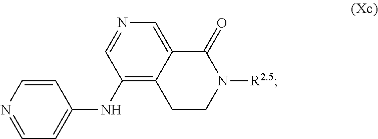
R^{2.5} is hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

56. The compound of claim 55, wherein R^{2.5} is hydrogen or unsubstituted C₁-C₄ alkyl.

57. The compound of claim 54, having the formula:

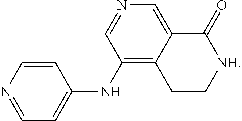 [SMILES: O=C1NCCc2c(Nc3ccncc3)cncc21]

58. A compound, or a pharmaceutically acceptable salt thereof, having the formula: 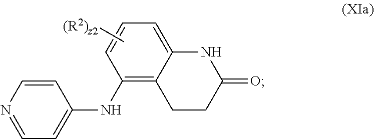
wherein
R² is independently halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

59. The compound of claim 58, having the formula: 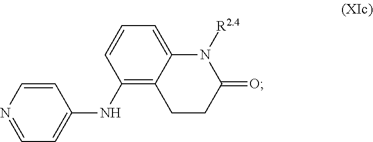
wherein
R^{2.4} is hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

60. The compound of claim 59, wherein R^{2.4} is hydrogen or unsubstituted C₁-C₄ alkyl.

61. The compound of claim 58, having the formula:

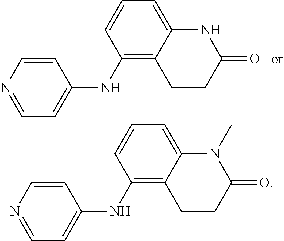

62. A compound, or a pharmaceutically acceptable salt thereof, having the formula: 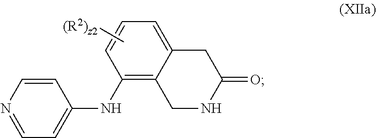
wherein
R² is independently halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}—NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R^{2A}, R^{2B}, R^{2C}, and R^{2D} are each independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X² is independently —F, —Cl, —Br, or —I;
n2 is an integer from 0 to 4;
m2 and v2 are independently 1 or 2; and
z2 is an integer from 0 to 8.

63. The compound of claim 62, having the formula: 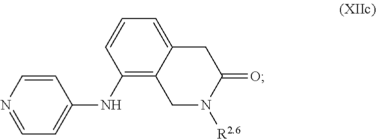
wherein
R^{2.6} is hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

64. The compound of claim 63, wherein R^{2.6} is hydrogen or unsubstituted C₁-C₄ alkyl.

65. The compound of claim 62, having the formula:

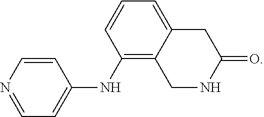 [SMILES: O=C1Cc2cccc(Nc3ccncc3)c2CN1]

66. A compound, or a pharmaceutically acceptable salt thereof, having the formula: 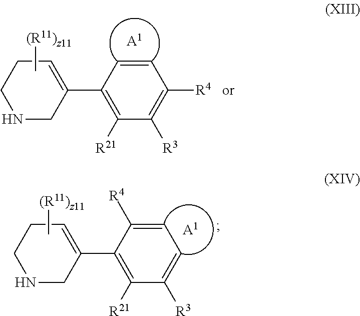
wherein
Ring A¹ is a substituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCI₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z11 is an integer from 0 to 8.

67. The compound of claim 66, wherein ring A¹ is oxo-substituted cycloalkyl.

68. The compound of claim 66, wherein ring A¹ is a substituted cycloalkyl, wherein the substituent is oxo, halogen, —NH₂, —OH, substituted or unsubstituted C₁-C₄ alkyl, or substituted or unsubstituted 2 to 4 membered heteroalkyl.

69. The compound of claim 66, wherein ring A¹ is a substituted C₃-C₈ cycloalkyl.

70. The compound of claim 66, wherein ring A¹ is a substituted C₃-C₆ cycloalkyl.

71. The compound of claim 66, wherein ring A¹ is a substituted cyclopentyl.

72. The compound of claim 66, wherein z11 is 0.

73. The compound of claim 66, wherein R²¹ is halogen, —NH₂, —OH, substituted or unsubstituted C₁-C₄ alkyl, or substituted or unsubstituted 2 to 4 membered heteroalkyl.

74. The compound of claim 66, wherein R²¹ is halogen, —NH₂, —OH, or unsubstituted methyl.

75. The compound of claim 66, wherein R³ is halogen, —NH₂, —OH, substituted or unsubstituted C₁-C₄ alkyl, or substituted or unsubstituted 2 to 4 membered heteroalkyl.

76. The compound of claim 66, wherein R³ is halogen, —NH₂, —OH, or unsubstituted methyl.

77. The compound of claim 66, wherein R⁴ is halogen, —NH₂, —OH, substituted or unsubstituted C₁-C₄ alkyl, or substituted or unsubstituted 2 to 4 membered heteroalkyl.

78. The compound of claim 66, wherein R⁴ is halogen, —NH₂, —OH, or unsubstituted methyl.

79. The compound of claim 66, wherein the compound is:

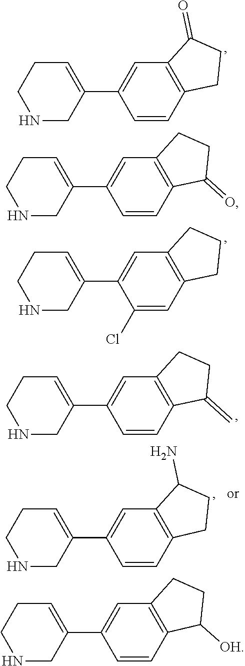

80. A pharmaceutical composition comprising the compound of one of claims 66 to 79 and a pharmaceutically acceptable excipient.

81. A method of treating pain in a subject in need thereof, said method comprising administering to the subject a therapeutically effective amount of a compound having the formula: 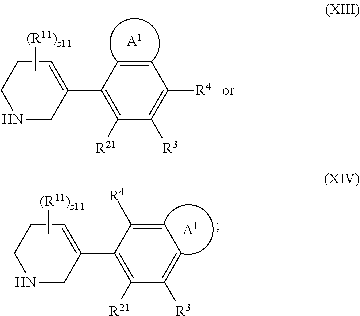
or a pharmaceutically acceptable salt thereof, wherein
Ring A¹ is a substituted or unsubstituted cycloalkyl;
R¹¹ is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²¹, R³, and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z11 is an integer from 0 to 8.

82. The method of claim 81, wherein the compound is not:

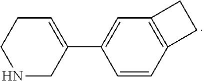 [SMILES: C1=C(c2ccc3c(c2)CC3)CNCC1]

83. The method of claim 81, wherein the pain is post-operative pain.

84. The method of claim 83, wherein the post-operative pain is pain after a hysterectomy.

85. The method of claim 83, wherein the post-operative pain is pediatric post-operative pain.

86. The method of claim 83, further comprising administering a second agent.

87. The method of claim 86, wherein the second agent is an opioid.

88. The method of claim 86, wherein the second agent is bupivacaine or gabapentin.

89. The method of claim 81, wherein the pain is neuropathic pain.

90. The method of claim 89, wherein the neuropathic pain is post-traumatic neuropathic pain.

91. The method of claim 89, wherein the neuropathic pain is diabetic neuropathic pain.

92. The method of claim 89, wherein the neuropathic pain is post-herpetic neuralgia.

93. The method of claim 89, wherein the neuropathic pain is chemotherapy-induced pain.

94. The method of claim 89, wherein the neuropathic pain is phantom limb pain.

95. The method of claim 81, wherein the pain is inflammatory pain.

96. The method of claim 95, wherein the inflammatory pain is associated with rheumatoid arthritis, ankylosing spondylitis, osteoarthritis, bursitis, tendinitis, or acute gouty arthritis.

97. The method of claim 81, wherein the pain is opioid refractory pain.

98. The method of claim 81, wherein the pain is a rebound headache.

99. The method of claim 81, wherein the pain is migraine pain.

100. The method of claim 81, wherein the pain is adiposis dolorosa.

101. The method of claim 81, wherein the pain is a burn pain.

102. The method of claim 81, wherein the pain is cluter headaches.

103. The method of claim 81, wherein the pain is associated with central pain conditions following stroke.

104. The method of claim 81, wherein the pain is a musculoskeletal pain.

105. The method of one of claims 81 to 104, wherein the compound is administered systemically.

106. The method of one of claims 81 to 104, wherein the compound is administered topically.

107. The method of one of claims 81 to 104, wherein the compound is administered intrathecally.

108. The method of one of claims 81 to 104, wherein the compound is administered orally.

109. The method of one of claims 81 to 104, wherein the compound is administered intravenously.

110. A compound, or a pharmaceutically acceptable salt thereof, having the formula: 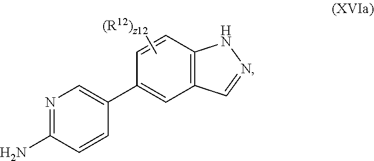
wherein
R¹² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z12 is an integer from 0 to 8;
with the proviso that R¹² is not methyl.

111. The compound of claim 110, wherein R¹² is not C₁-C₄-alkyl.

112. The compound of claim 110, wherein R¹² is not unsubstituted C₁-C₄-alkyl.

113. The compound of claim 110, wherein R¹² is halogen, —CN, —NO₂, substituted C₁-C₄ alkyl, or substituted or unsubstituted 2 to 4 membered heteroalkyl.

114. The compound of claim 110, wherein R¹² is halogen, —CN, —NO₂, —CF₃, or —OCH₃.

115. The compound of claim 110, wherein z12 is 1.

116. The compound of claim 110, wherein the compound is:

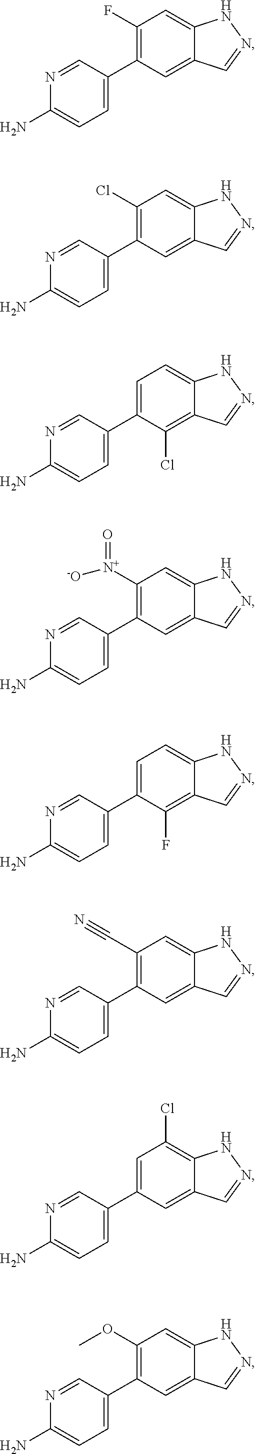

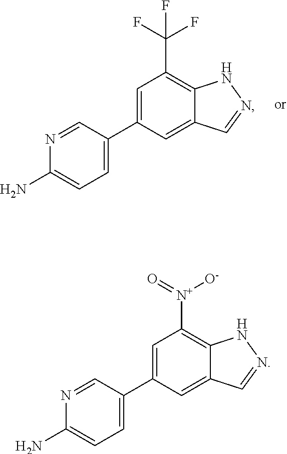 [SMILES: Nc1ccc(-c2cc(C(F)(F)F)c3[nH]ncc3c2)cn1 ; "or" ; Nc1ccc(-c2cc([N+](=O)[O-])c3[nH]ncc3c2)cn1]

117. A pharmaceutical composition comprising the compound of one of claims 110 to 116 and a pharmaceutically acceptable excipient.

118. A method of treating pain in a subject in need thereof, said method comprising administering to the subject a therapeutically effective amount of a compound having the formula: 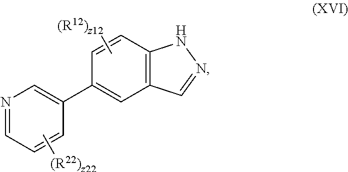
or a pharmaceutically acceptable salt thereof, wherein
each R¹² and R²² are independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

119. The method of claim 118, wherein the compound is not:

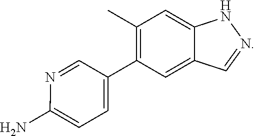 [SMILES: Cc1cc2[nH]ncc2cc1-c1ccc(N)nc1]

120. The method of claim 118, wherein the pain is post-operative pain.

121. The method of claim 120, wherein the post-operative pain is pain after a hysterectomy.

122. The method of claim 120, wherein the post-operative pain is pediatric post-operative pain.

123. The method of claim 120, further comprising administering a second agent.

124. The method of claim 123, wherein the second agent is an opioid.

125. The method of claim 123, wherein the second agent is bupivacaine.

126. The method of claim 118, wherein the pain is neuropathic pain.

127. The method of claim 126, wherein the neuropathic pain is post-traumatic neuropathic pain.

128. The method of claim 126, wherein the neuropathic pain is diabetic neuropathic pain.

129. The method of claim 126, wherein the neuropathic pain is post-herpetic neuralgia.

130. The method of claim 126, wherein the neuropathic pain is chemotherapy-induced pain.

131. The method of claim 126, wherein the neuropathic pain is phantom limb pain.

132. The method of claim 118, wherein the pain is inflammatory pain.

133. The method of claim 132, wherein the inflammatory pain is associated with rheumatoid arthritis, ankylosing spondylitis, osteoarthritis, bursitis, tendinitis, or acute gouty arthritis.

134. The method of claim 118, wherein the pain is opioid refractory pain.

135. The method of claim 118, wherein the pain is a rebound headache.

136. The method of claim 118, wherein the pain is migraine pain.

137. The method of claim 118, wherein the pain is adiposis dolorosa.

138. The method of claim 118, wherein the pain is a burn pain.

139. The method of claim 118, wherein the pain is cluster headaches.

140. The method of claim 118, wherein the pain is associated with central pain conditions following stroke.

141. The method of claim 118, wherein the pain is a musculoskeletal pain.

142. The method of one of claims 118 to 141, wherein the compound is administered systemically.

143. The method of one of claims 118 to 141, wherein the compound is administered topically.

144. The method of one of claims 118 to 141, wherein the compound is administered intrathecally.

145. The method of one of claims 118 to 141, wherein the compound is administered orally.

146. The method of one of claims 118 to 141, wherein the compound is administered intravenously.

147. A compound, or a pharmaceutically acceptable salt thereof, having the formula: 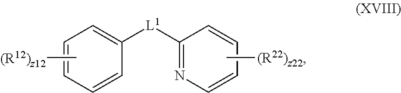
wherein
L¹ is substituted or unsubstituted C₁-C₃ alkylene;
each R¹² and R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5; and
z22 is an integer from 0 to 4.

148. The compound of claim 147, wherein L¹ is unsubstituted C₁-C₂ alkylene.

149. The compound of claim 147, wherein L¹ is unsubstituted ethenylene.

150. The compound of claim 147, wherein R¹² is halogen, substituted or unsubstituted C₁-C₄ alkyl, or substituted or unsubstituted 2 to 4 membered heteroalkyl.

151. The compound of claim 147, wherein R¹² is —F, —Cl, —Br, —I, or —OCF₃.

152. The compound of claim 147, wherein z12 is 1.

153. The compound of claim 147, wherein R²² is —NH₂.

154. The compound of claim 147, wherein z22 is 1.

155. The compound of claim 147, wherein the compound is:

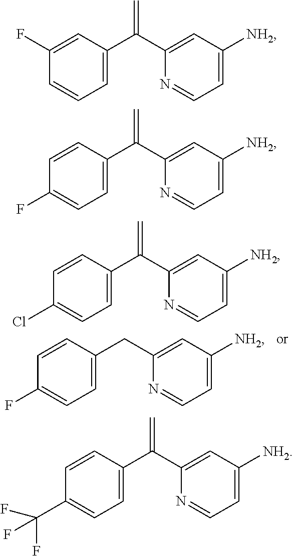

156. A pharmaceutical composition comprising the compound of one of claims 147 to 155 and a pharmaceutically acceptable excipient.

157. A method of treating pain in a subject in need thereof, said method comprising administering to the subject a therapeutically effective amount of a compound having the formula: 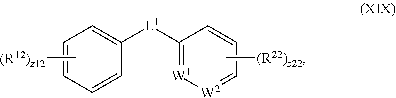
or a pharmaceutically acceptable salt thereof, wherein
L¹ is —O—, —NR¹⁰—, or substituted or unsubstituted alkylene;
W¹ is N or CR³;
W² is N or CR⁴;
each R¹² and R²² is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z12 is an integer from 0 to 5;
z22 is an integer from 0 to 4;
R³ and R⁴ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹⁰ is hydrogen or unsubstituted C₁-C₄ alkyl;
wherein at least one of W¹ or W² is N.

158. The method of claim 157, wherein the compound is not:

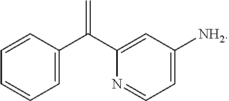

159. The method of claim 157, wherein W¹ is N.

160. The method of claim 157, wherein W² is CH.

161. The method of claim 157, wherein L¹ is substituted or unsubstituted C₁-C₃ alkylene.

162. The method of claim 157, wherein L¹ is unsubstituted C₁-C₂ alkylene.

163. The method of claim 157, wherein L¹ is unsubstituted ethenylene.

164. The method of claim 157, wherein R¹² is independently halogen, substituted or unsubstituted C₁-C₄ alkyl, or substituted or unsubstituted 2 to 4 membered heteroalkyl.

165. The method of claim 157, wherein R¹² is independently —F, —Cl, —Br, —I, or —OCF₃.

166. The method of claim 157, wherein z12 is 1.

167. The method of claim 157, wherein R²² is —NH₂.

168. The method of claim 157, wherein z22 is 1.

169. The method of claim 157, wherein R³ and R⁴ are hydrogen.

170. The method of claim 157, wherein the pain is post-operative pain.

171. The method of claim 170, wherein the post-operative pain is pain after a hysterectomy.

172. The method of claim 170, wherein the post-operative pain is pediatric post-operative pain.

173. The method of claim 170, further comprising administering a second agent.

174. The method of claim 173, wherein the second agent is an opioid.

175. The method of claim 173, wherein the second agent is bupivacaine or gabapentin.

176. The method of claim 157, wherein the pain is neuropathic pain.

177. The method of claim 176, wherein the neuropathic pain is post-traumatic neuropathic pain.

178. The method of claim 176, wherein the neuropathic pain is diabetic neuropathic pain.

179. The method of claim 176, wherein the neuropathic pain is post-herpetic neuralgia.

180. The method of claim 176, wherein the neuropathic pain is chemotherapy-induced pain.

181. The method of claim 176, wherein the neuropathic pain is phantom limb pain.

182. The method of claim 157, wherein the pain is inflammatory pain.

183. The method of claim 182, wherein the inflammatory pain is associated with rheumatoid arthritis, ankylosing spondylitis, osteoarthritis, bursitis, tendinitis, or acute gouty arthritis.

184. The method of claim 157, wherein the pain is opioid refractory pain.

185. The method of claim 157, wherein the pain is a rebound headache.

186. The method of claim 157, wherein the pain is migraine pain.

187. The method of claim 157, wherein the pain is adiposis dolorosa.

188. The method of claim 157, wherein the pain is a burn pain.

189. The method of claim 157, wherein the pain is cluster headaches.

190. The method of claim 157, wherein the pain is associated with central pain conditions following stroke.

191. The method of one of claims 157 to 190, wherein the compound is administered systemically.

192. The method of one of claims 157 to 190, wherein the compound is administered topically.

193. The method of one of claims 157 to 190, wherein the compound is administered intrathecally.

194. The method of one of claims 157 to 190, wherein the compound is administered orally.

195. The method of one of claims 157 to 190, wherein the compound is administered intravenously.


SEQUENCE LISTING (1 sequence)

SEQ ID NO: 1 - protein, 465 aa, synthetic construct
  REGION 1..465: note = Synthetic polypeptide
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV